(19) United States
(12) Patent Application Publication
(10) Pub. No.: US 2026/0234610 A1
(43) Pub. Date: Aug. 13, 2026

(54) NOVEL CAS12A PROTEIN, VARIANT OF NOVEL CAS12A PROTEIN, AND USE THEREOF FOR EUKARYOTIC GENOME EDITING

(71) Applicant: NSAGE CORP., Incheon (KR)

(72) Inventors: Bong Hee LEE, Incheon (KR); Delger BAYARSAIKHAN, Incheon (KR); Govigerel BAYARSAIKHAN, Incheon (KR); Okjae KOO, Gyeonggi-do (KR); Hyun A KANG, Incheon (KR); Jae Suk LEE, Incheon (KR)

(21) Appl. No.: 19/157,969

(22) PCT Filed: Sep. 19, 2024
(86) PCT No.: PCT/KR2024/096211

§ 371 Date: Aug. 19, 2025

(30) Foreign Application Priority Data
Sep. 20, 2023 (KR) 10-2023-0125571
Mar. 22, 2024 (KR) 10-2024-0039702

(51) Int. Cl. C12N 15/11; C12N 9/22

(52) U.S. Cl. CPC C12N 15/111; C12N 9/226; C12N 15/11; C12N 2310/20

(57) ABSTRACT

Proposed is a novel CRISPR/Cas12a system capable of editing eukaryotic genomes. The novel CRISPR/Cas12a system includes a novel Cas12a protein or a variant thereof (or a nucleic acid for encoding the same); and a programmable guide RNA (or a nucleic acid encoding the same). Herein, the Cas12a protein or the variant thereof can be linked to one or more nuclear localization signals. Using the novel CRISPR/Cas12a system disclosed and the method for editing eukaryotic genomes, a target nucleic acid in a eukaryotic genome can be edited. Therefore, the novel CRISPR/Cas12a system can be used in techniques in various contexts that require eukaryotic genome editing.

### TECHNICAL FIELD

The present disclosure covers techniques related to a CRISPR/Cas system, especially the CRISPR/Cas12a system, and a method for editing eukaryotic genomes using the same.

### BACKGROUND ART

### CRISPR/Cas12a System

### CRISPR/Cas12a System Overview

A CRISPR/Cas12a system is also called a CRISPR/Cpf1 system. The CRISPR/Cas12a system is a subtype A CRISPR/Cas system of Type V within Class 2. The CRISPR/Cas12a system was first reported by Feng Zhang's research team at the Broad Institute. The CRISPR/Cas12a system is an extensively examined CRISPR/Cas system along with a CRISPR/Cas9 system.

The CRISPR/Cas12a system, like the CRISPR/Cas9 system, is known to exhibit target-specific cleavage activity for double-stranded nucleic acids. Therefore, the CRISPR/Cas12a system has high utility for gene editing in eukaryotic cells. Compared to the CRISPR/Cas9 system, the CRISPR/Cas12a system has the following characteristics: 1) its Cas12a protein has only one nucleic acid cleavage domain (RuvC domain); 2) its guide RNA is made only from crRNA; and 3) the CRISPR/Cas12a system cleaves nucleic acids with sticky ends, resulting in occurrence of 4-5 nucleotide overhangs upon gene cleavage.

Compared to the CRISPR/Cas9 system, the CRISPR/Cas12a system has a simple guide RNA structure and has a low off-target cleavage rate, enabling more precise gene editing. Accordingly, many researchers are actively researching ways to utilize the CRISPR/Cas12a system.

Herein below, this CRISPR/Cas12a system and its components are described in more detail.

### CRISPR/Cas12a Classification

The CRISPR/Cas12a system belongs to a subtype A of CRISPR/Cas system of type V within Class 2 and is also called a CRISPR/Cpf1 system.

### CRISPR/Cas12a System Composition and Function

The CRISPR/Cas12a system includes a Cas12a protein and a guide RNA thereof. The Cas12a protein and the guide RNA act as a complex and exhibit target-specific nucleic acid cleavage activity and collateral cleavage activity. The Cas12a protein recognizes a PAM sequence and functions to cleave a nucleic acid of a target sequence targeted by the guide RNA. The guide RNA functions to target the nucleic acid of the target sequence.

### Cas12a Protein Structure

A Cas12a protein can be broadly divided into a REC (RECognition) lobe and a NUC (NUClease) lobe. The NUC lob is further divided into a RuvC domain, a NUC domain, a WED domain, a PI (PAM Interacting) domain, and a BH (Bridge Helix) domain. The specific structure of the Cas12a protein has already been reported by several researchers and is described in detail in the conventional literature of Paul et. al (CRISPR-Cas12a: Functional overview and applications. Biomedical Journal, Vol. 43, Issue 1, p. 8-17, 2020). Among these, WED II, WED III, REC1, and PI domains play an important role in allowing the Cas12a protein to recognize the PAM sequence. Meanwhile, the domains that play an important role in the Cas12a protein cleaving the nucleic acid of the target sequence are the RuvC domain and the NUC domain.

### Guide RNA Structure

Unlike a guide RNA of the CRISPR/Cas9 system, the guide RNA of the CRISPR/Cas12a system is made from a single RNA molecule and is called crRNA. The guide RNA includes a direct repeat (DR) and a guide domain. Specifically, in the guide RNA, the DR and the guide domain are sequentially connected from the 3′ end to the 5′ end. The DR is a portion involved in forming a complex by inducing an interaction between the guide RNA and the Cas12a protein. The guide domain is a portion that binds to the nucleic acid of the target sequence and enables the CRISPR/Cas12a system to exhibit target-specific cleavage activity and collateral cleavage activity.

### Target-specific Nucleic Acid Cleavage Activity of CRISPR/Cas12a System and PAM (Protospacer Adjacent Motif)

The CRISPR/Cas12a system has target-specific nucleic acid cleavage activity. Two conditions are required to exhibit this target-specific nucleic acid cleavage activity. First, there should be a base sequence of a certain length in a nucleic acid that can be recognized by the Cas12a protein. Second, there should be a sequence around the base sequence of a certain length, herein the sequence can bind complementarily to the guide domain included in the guide RNA. When the two conditions are satisfied, 1) the Cas12a protein recognizes the base sequence of a certain length, and 2) the guide domain binds complementarily to the portion of the sequence around the base sequence of a certain length, then the nucleic acid cleavage activity appears. At this time, the nucleotide sequence of a certain length recognized by the Cas12a protein is called a protospacer adjacent motif (PAM) sequence.

The PAM sequence is a unique sequence determined by the Cas12a protein. When the PAM sequence of a Cas12a protein is known, using this, it is possible to design a CRISPR/Cas12a system that targets a nucleic acid of a predetermined target sequence adjacent to the PAM sequence.

### DISCLOSURE

### Technical Problem

In this present disclosure, the technical task is to discover a novel Cas12a protein that can be used for editing eukaryotic genomes among several candidate Cas12a proteins, and furthermore, to disclose a novel Cas12a variant, which is generated by introducing an additional mutation into the novel Cas12a protein. Furthermore, the technical task is to present a CRISPR/Cas12a system that can be used for editing actual eukaryotic genomes, including a novel Cas12a protein or a variant thereof.

### Technical Solution

To solve the technical challenges, the inventors of this present disclosure:
1) discovered, with several candidate Cas12a proteins, a CRISPR/Cas12a system discovered that recognized and effectively bound to double-stranded nucleic acids in the genomes, and in this process, the inventors newly specified guide RNA scaffolds that had previously been incorrectly disclosed,
2) prepared a variant of the Cas12a protein, based on the novel Cas12a protein, by introducing a new mutation that improves a PAM recognition ability,
3) prepared a CRISPR/Cas12a system including the Cas12a protein or the variant of the Cas12a protein, and
4) demonstrated that the CRISPR/Cas12a system could edit the eukaryotic genomes.

Accordingly, the present disclosure discloses a novel CRISPR/Cas12a system that has been proven to be effective and a method for editing eukaryotic genomes using the same.

Furthermore, by applying the method for editing the eukaryotic genomes, a method for editing a nucleic acid in the cytoplasm and a method for editing multiple target nucleic acids are also disclosed.

### Advantageous Effects

Using a novel CRISPR/Cas12a system disclosed in the present disclosure and a method for editing eukaryotic genomes, a target nucleic acid in the eukaryotic genomes can be edited. Therefore, the novel CRISPR/Cas12a system can be used in various contexts that require eukaryotic genome editing.

### DESCRIPTION OF DRAWINGS

FIG. 1 shows evaluation results of CRISPR enzyme expressed according to, as described in Experimental Example 1.4, wherein (a) shows ethidium bromide staining results visualized after actual restriction enzyme digestion, and (b) shows the numerical labels for each position in (a), and the meaning of each number label is shown in Table 3 of Experimental Example 2.2;

FIG. 2 shows results of a T7E1 assay according to Experimental Example 2.1, wherein each number represents a CRISPR/Cas12a complex of the labels shown in Table 1, Lba is a positive control and represents an experimental result with the CRISPR/LbaCas12a complex, and WT is a negative control without the CRISPR/Cas12a complex;

FIG. 3 shows results of analyzing the pattern of indels occurring in endogenous EMX1 gene of HEK cells after introducing a CRISPR/Cas12a complex including the Cas12a protein with ID 218 into the cells, according to Experimental Example 2.3, wherein as a result of NGS analysis, several mutations with a high number of reads are shown, in each mutant sequence, the underlined parts represent protospacer sequences, and the parts marked with “-” indicate nucleotides deleted as a result of gene editing;

FIG. 4 shows results of analyzing the pattern of indels occurring in the endogenous EMX1 gene of the HEK cells after introducing a CRISPR/Cas12a complex including the LbaCas12a protein as a positive control into the cells, according to Experimental Example 2.3, wherein, as a result of NGS analysis, several mutations with a high number of reads are shown, in each mutant sequence, the underlined parts represent protospacer sequences, and the parts marked with “-” indicate nucleotides deleted as a result of gene editing;

FIG. 5 schematically shows a CRISPR enzyme composition according to Experimental Example 3, wherein the numbers (1. to 7.) in front of each schematic diagram indicate the composition of their corresponding NLS1 to NLS7, and the specific sequences are listed in Table 5 of Experimental Example 3.1;

FIG. 6 shows evaluation results of the CRISPR enzyme, expressed according to Experimental Example 3.1, by IPTG induction, wherein labels (NLS1 to NLS7) listed in the experimental results represent CRISPR enzymes of each composition listed in Table 5;

FIG. 7 shows evaluation results of the CRISPR enzyme, expressed according to Experimental Example 3.1, by SDS-PAGE, wherein labels (NLS1 to NLS7) listed in the experimental results represent CRISPR enzymes of each composition listed in Table 5;

FIG. 8 shows results of testing an endogenous EMX1 gene editing activity of a CRISPR/Cas12a complex by T7E1 assay according to Experimental Example 3.3, wherein #1 to #7 correspond to NLS1 to NLS7 in Table 5, respectively;

FIG. 9 shows evaluation results of evaluating the CRISPR enzyme expressed according to Experimental Example 4, as described in Experimental Example 1.4, wherein each numeric label represents the CRISPR/Cas12a complex of its corresponding label in Table 8 of Experimental Example 4.1;

FIG. 10 shows evaluation results of the CRISPR enzyme, expressed according to Experimental Example 4, by SDS-PAGE analysis as described in Experimental Example 1.3, wherein each numeric label represents the CRISPR/Cas12a complex of its corresponding label in Table 8 of Experimental Example 4.1;

FIG. 11 shows results of analyzing the pattern of indels occurring in the endogenous EMX1 gene of HEK293T cells after introducing a CRISPR/Cas12a complex including an NLS5 CRISPR enzyme into the cells, according to Experimental Example 3.4, wherein, as a result of NGS analysis, several mutations with a high number of reads are shown, and in each mutant sequence, the underlined parts represent protospacer sequences, and the parts marked with “-” indicate nucleotides deleted as a result of gene editing;

FIG. 12 shows results of analyzing the pattern of indels occurring in the endogenous MTAP gene of HEK293T cells after introducing a CRISPR/Cas12a complex including the Cas12a protein with ID 218 into the cells, according to Experimental Example 4, wherein, as a result of NGS analysis, several mutations with a high number of reads are shown, and in each mutant sequence, the underlined parts represent protospacer sequences, and the parts marked with “-” indicate nucleotides deleted as a result of gene editing;

FIG. 13 shows results of analyzing the pattern of indels occurring in the endogenous MTAP gene of HEK293T cells after introducing a CRISPR/Cas12a complex including the Cas12a protein with ID 233 into the cells, according to Experimental Example 4, wherein, as a result of NGS analysis, several mutations with a high number of reads are shown, and in each mutant sequence, the underlined parts represent protospacer sequences, and the parts marked with “-” indicate nucleotides deleted as a result of gene editing;

FIG. 14 shows results of analyzing the pattern of indels occurring in the endogenous MTAP gene of HEK293T cells after introducing a CRISPR/Cas12a complex including the Cas12a protein with ID 236 into the cells, according to Experimental Example 4, wherein, as a result of NGS analysis, several mutations with a high number of reads are shown, and in each mutant sequence, the underlined parts represent protospacer sequence, and the parts marked with “-” indicate nucleotides deleted as a result of gene editing;

FIG. 15 shows results of analyzing the pattern of indels occurring in the endogenous DYRK1A gene of HEK293T cells after introducing a CRISPR/Cas12a complex including the Cas12a protein with ID 218 into the cells, according to Experimental Example 4, wherein, as a result of NGS analysis, several mutations with a high number of reads are shown, and in each mutant sequence, the underlined parts represent protospacer sequences, and the parts marked with “-” indicate nucleotides deleted as a result of gene editing;

FIG. 16 shows results of analyzing the pattern of indels occurring in the endogenous DYRK1A gene of HEK293T cells after introducing a CRISPR/Cas12a complex including the Cas12a protein with ID 233 into the cells, according to Experimental Example 4, wherein, as a result of NGS analysis, several mutations with a high number of reads are shown, and in each mutant sequence, the underlined parts represent protospacer sequences, and the parts marked with “-” indicate nucleotides deleted as a result of gene editing;

FIG. 17 shows results of analyzing the pattern of indels occurring in the endogenous DYRK1A gene of HEK293T cells after introducing a CRISPR/Cas12a complex including the Cas12a protein with ID 236 into the cells, according to Experimental Example 4, wherein, as a result of NGS analysis, several mutations with a high number of reads are shown, and in each mutant sequence, the underlined parts represent protospacer sequences, and the parts marked with “-” indicate nucleotides deleted as a result of gene editing;

FIG. 18 shows an in-vitro nucleic acid cleavage activity of each CRISPR/Cas12a complex according to Experimental Example 4.3, wherein (+++) indicates the highest cleavage activity, (++) indicates medium cleavage activity, (+) indicates low cleavage activity, and (−) indicates no cleavage activity;

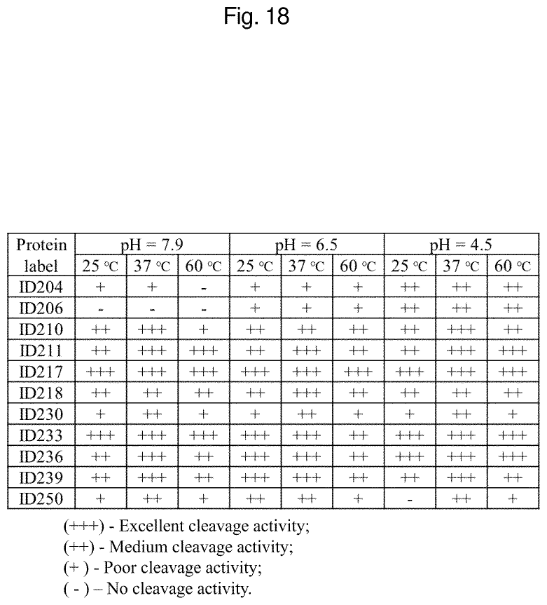

FIG. 19 shows an AlphaFold-simulated diagram illustrating a structure of the CRISPR/Cas12a system, including a Cas12a protein having an amino acid sequence of SEQ ID NO: 1, when the CRISPR/Cas12a system forms a complex by binding to its target nucleic acid;

FIG. 20 shows an enlarged view of interaction portions between a CRISPR/Cas12a system and a PAM where the CRISPR/Cas12a system including a Cas12a protein having an amino acid sequence of SEQ ID NO: 1 binds to its target sequence, and then forms a complex, wherein positions E155, K537, and G531, which are likely to interact with the positions of PAM sequence (DT, DT, DT, and DA), are indicated;

FIG. 21 shows results of eukaryotic genome editing activity experiments according to Experimental Example 5, wherein the DYRK1A gene of HEK cells was targeted, and the indel introduction frequency for each CRISPR/Cas12a system is shown, and the composition of each label in the chart is disclosed in the table of Experimental Example 5.2;

FIG. 22 shows results of the eukaryotic genome editing activity experiments according to Experimental Example 5, wherein the MTAP gene of HEK cells was targeted, and the indel introduction frequency for each CRISPR/Cas12a system is shown, and the composition of each label in the chart is disclosed in the table of Experimental Example 5.2;

FIG. 23 shows results of the eukaryotic genome editing activity experiments according to Experimental Example 5, wherein the VEGFA gene of HEK cells was targeted, and the indel introduction frequency for each CRISPR/Cas12a system is shown, and the composition of each label in the chart is disclosed in the table of Experimental Example 5.2;

FIG. 24 shows results of the eukaryotic genome editing activity experiments according to Experimental Example 5, wherein the FANCF gene of HEK cells was targeted, and the indel introduction frequency for each CRISPR/Cas12a system is shown, and the composition of each label in the chart is disclosed in the table of Experimental Example 5.2;

FIG. 25 shows results of the eukaryotic genome editing activity experiments according to Experimental Example 5, wherein the CFRT gene of HEK cells was targeted, and the indel introduction frequency for each CRISPR/Cas12a system is shown, and the composition of each label in the chart is disclosed in the table of Experimental Example 5.2;

FIG. 26 shows a violin plot plotted with an entire eukaryotic gene editing activity data according to Experimental Example 5, wherein the results are shown for the Cas12a protein having an amino acid sequence of SEQ ID NO: 1, and also for a Cas12a protein with an E155R mutation introduced into the amino acid sequence;

FIG. 27 shows an AlphaFold-simulated diagram illustrating a structure of a CRISPR/Cas12a system, including a Cas12a protein of SEQ ID NO: 3, when the CRISPR/Cas12a system forms a complex by binding to its target nucleic acid;

FIG. 28 shows an enlarged view of interaction portions between a CRISPR/Cas12a system and a PAM where the CRISPR/Cas12a system including a Cas12a protein having an amino acid sequence of SEQ ID NO: 3 binds to its target sequence, and then forms a complex, wherein positions Q190, S579, and Y585, which are likely to interact with the positions of PAM sequence (DT, DT, DT, and DA), are indicated;

FIG. 29 shows results of eukaryotic genome editing activity experiments according to Experimental Example 6, wherein the DYRK1A gene of HEK cells was targeted, and the indel introduction frequency for each CRISPR/Cas12a system is shown, and the composition of each label in the chart is disclosed in the table of Experimental Example 6.2;

FIG. 30 shows results of the eukaryotic genome editing activity experiments according to Experimental Example 6, wherein the CFTR gene of HEK cells was targeted, and the indel introduction frequency for each CRISPR/Cas12a system is shown, and the composition of each label in the chart is disclosed in the table of Experimental Example 6.2;

FIG. 31 shows results of the eukaryotic genome editing activity experiments according to Experimental Example 6, wherein the VEGFA gene of HEK cells was targeted, and the indel introduction frequency for each CRISPR/Cas12a system is shown, and the composition of each label in the chart is disclosed in the table of Experimental Example 6.2;

FIG. 32 shows a violin plot plotted with an entire eukaryotic gene editing activity data according to Experimental Example 6, wherein the results are shown for the Cas12a protein having an amino acid sequence of SEQ ID NO: 3, and also for a Cas12a protein with the Q190R and S579R mutations introduced into the amino acid sequence;

FIG. 33 shows a part of an AlphaFold-simulated diagram illustrating a structure of a CRISPR/Cas12a system having an amino acid sequence of SEQ ID NO: 1, when the CRISPR/Cas12a system forms a complex by binding to its target nucleic acid, wherein position E791, predicted to contribute to increasing the stability of a CRISPR/Cas12a complex, is indicated;

FIG. 34 shows a part of an AlphaFold-simulated diagram illustrating a structure of a CRISPR/Cas12a system having an amino acid sequence of SEQ ID NO: 1, when the CRISPR/Cas12a system forms a complex by binding to its target nucleic acid, wherein position N526, predicted to interact significantly with the target nucleic acid is indicated;

FIG. 35 shows result of aligning the amino acid sequence of SEQ ID NO: 1 with that of AsCas12a (AsCpf1) in the conventional literature, wherein it was obtained that positions N526 and E791 respectively corresponded to M537 and F870, which have been found in the conventional literature to contribute to improving nucleic acid cleavage activity, and this supports reliability of the result obtained through structural analysis;

FIG. 36 shows a part of an AlphaFold-simulated diagram illustrating a structure of a CRISPR/Cas12a system having an amino acid sequence of SEQ ID NO: 1, when the CRISPR/Cas12a system forms a complex by binding to its target nucleic acid, wherein position W354, predicted to contribute to increased R-loop dwell time during nucleic acid cleavage of a CRISPR/Cas12a complex, is indicated;

FIG. 37 shows a result of aligning the amino acid sequence of SEQ ID NO: 1 with that of AsCas12a (AsCpf1) in the conventional literature, wherein it was obtained that the position W354 corresponded to position W355, which have been found in the conventional literature to contribute to the increased R-loop dwell time, and this supports reliability of the result obtained through structural analysis;

FIG. 38 shows results of eukaryotic genome editing activity experiments according to Experimental Example 7, wherein the FANCF gene of HEK cells was targeted, and the indel introduction frequency for each CRISPR/Cas12a system is shown, and the composition of each label in the chart is disclosed in the table of Experimental Example 7.2;

FIG. 39 shows results of the eukaryotic genome editing activity experiments according to Experimental Example 7, wherein the VEGFA gene of HEK cells was targeted, and the indel introduction frequency for each CRISPR/Cas12a system is shown, and the composition of each label in the chart is disclosed in the table of Experimental Example 7.2;

FIG. 40 shows results of the eukaryotic genome editing activity experiments according to Experimental Example 7, wherein the Site3 portion of HEK cells was targeted, and the indel introduction frequency for each CRISPR/Cas12a system is shown, and the composition of each label in the chart is disclosed in the table of Experimental Example 7.2;

FIG. 41 shows results of the eukaryotic genome editing activity experiments according to Experimental Example 7, wherein the MTAP gene of HEK cells was targeted, and the indel introduction frequency for each CRISPR/Cas12a system is shown, and the composition of each label in the chart is disclosed in the table of Experimental Example 7.2;

FIG. 42 shows results of the eukaryotic genome editing activity experiments according to Experimental Example 7, wherein the CFTR gene of HEK cells was targeted, and the indel introduction frequency for each CRISPR/Cas12a system is shown, and the composition of each label in the chart is disclosed in the table of Experimental Example 7.2;

FIG. 43 shows results of the eukaryotic genome editing activity experiments according to Experimental Example 7, wherein the DYRK1A gene of HEK cells was targeted, and the indel introduction frequency for each CRISPR/Cas12a system is shown, and the composition of each label in the chart is disclosed in the table of Experimental Example 7.2;

FIG. 44 shows results of eukaryotic genome editing activity experiments according to Experimental Example 8, wherein the FANCF gene of HEK cells was targeted, and the indel introduction frequency for each CRISPR/Cas12a system is shown, and the composition of each label in the chart is disclosed in the table of Experimental Example 8.1;

FIG. 45 shows results of the eukaryotic genome editing activity experiments according to Experimental Example 8, wherein the VEGFA gene of HEK cells was targeted, and the indel introduction frequency for each CRISPR/Cas12a system is shown, and the composition of each label in the chart is disclosed in the table of Experimental Example 8.1;

FIG. 46 shows results of the eukaryotic genome editing activity experiments according to Experimental Example 8, wherein the CFTR gene of HEK cells was targeted, and the indel introduction frequency for each CRISPR/Cas12a system is shown, and the composition of each label in the chart is disclosed in the table of Experimental Example 8.1;

FIG. 47 shows results of the eukaryotic genome editing activity experiments according to Experimental Example 8, wherein the MTAP gene of HEK cells was targeted, and the indel introduction frequency for each CRISPR/Cas12a system is shown, and the composition of each label in the chart is disclosed in the table of Experimental Example 8.1;

FIG. 48 shows results of the eukaryotic genome editing activity experiments according to Experimental Example 8, wherein the DYRK1A gene of HEK cells was targeted, and the indel introduction frequency for each CRISPR/Cas12a system is shown, and the composition of each label in the chart is disclosed in the table of Experimental Example 8.1;

FIG. 49 shows results of the eukaryotic genome editing activity experiments according to Experimental Example 8, wherein the Site3 gene of HEK cells was targeted, and the indel introduction frequency for each CRISPR/Cas12a system is shown, and the composition of each label in the chart is disclosed in the table of Experimental Example 8.1;

FIG. 50 shows a diagram schematically showing a process of self-maturation after transcription of multiple guide RNA sequences linked to one promoter in the CRISPR/Cas12a system; and

FIG. 51 shows results of eukaryotic genome editing activity experiments according to Experimental Example 9, wherein the experiments were conducted by simultaneously targeting the VEGFA, MTAP, and DYRK1A, and the graphs on the left for each target show editing results obtained by introducing the actual CRISPR/Cas12a system, and the dots on the right show a negative control in which nothing was added.

### BEST MODE

Hereinafter, the best mode for carrying out the present disclosure is exemplarily disclosed. It includes some, but not all, implementations of the present disclosure disclosed herein. The embodiments described in this paragraph are merely examples, and only the embodiments described in this paragraph should not be construed as the “best form of the present disclosure”. Those skilled in the art will be able to think of many variations and more preferred implementations of the examples described in this paragraph. Such content should also be considered to be included in the best form for carrying out the present disclosure.

The present disclosure discloses a method for editing a target nucleic acid in the eukaryotic cell genome, the method including the following:
delivering a composition for editing the eukaryotic cell genome into the eukaryotic cell,
wherein the composition for editing the eukaryotic cell genome includes the following:
a Cas12a protein linked to one or more nuclear localization signals (NLS) or a nucleic acid encoding the Cas12a protein linked to one or more nuclear localization signals; and
a guide RNA or a nucleic acid encoding the guide RNA,
wherein, the guide RNA includes a guide domain and a scaffold,
wherein, the guide domain targets a target nucleic acid, and
wherein, the Cas12a protein and the scaffold are in a combination selected from the following:
a Cas12a protein having an amino acid sequence selected from SEQ ID NOs: 1, and 264 to 274, and a scaffold having a nucleic acid sequence of SEQ ID NO: 33; or
a Cas12a protein having an amino acid sequence selected from SEQ ID NOs: 3, and 275 to 279, and a scaffold having a nucleic acid sequence of SEQ ID NO: 35.

In one embodiment,
the Cas12a protein linked to one or more nuclear localization signals has a structure as follows:

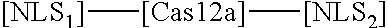

wherein, the NLS₁ has an amino acid sequence selected from SEQ ID NOs: 67 to 94 or is absent, and
the Cas12a is the Cas12a protein described above, and
wherein the NLS₂ has an amino acid sequence selected from SEQ ID NOs: 67 to 94 or is absent, and
at least one of NLS₁ or NLS₂ is present.

In another embodiment,
the structure of the Cas12a protein linked to one or more nuclear localization signals is as follows:
the NLS₁ may be represented by an amino acid sequence of SEQ ID NO: 67, and the NLS₂ may be represented by an amino acid sequence of SEQ ID NO: 67; or
the NLS~1 may be absent, and the NLS₂ may be represented by an amino acid sequence of SEQ ID NO: 70.

In a further embodiment,
the composition for editing the eukaryotic cell genome may include a CRISPR/Cas12a complex in which a Cas12a protein linked to one or more nuclear localization signals is bound with a guide RNA.

In a yet further embodiment,
the composition for editing the eukaryotic cell genome may include a nucleic acid encoding a Cas12a protein linked to one or more nuclear localization signals, and a nucleic acid encoding a guide RNA.

This present disclosure discloses a composition for editing eukaryotic genomes including the following:
a Cas12a protein linked to one or more nuclear localization signals (NLS) or a nucleic acid encoding the Cas12a protein linked to one or more nuclear localization signals; and
a guide RNA or a nucleic acid encoding the guide RNA,
wherein, the guide RNA includes a scaffold and a guide domain, and
the 3′ end of the scaffold is linked to the 5′ end of the guide domain,
wherein, the guide RNA binds with the Cas12a protein to form a complex,
the guide domain targets a predetermined target nucleic acid, and
the Cas12a protein and the scaffold are selected from the following combinations:
a Cas12a protein having an amino acid sequence of SEQ ID NO: 1 and a scaffold having a nucleic acid sequence of SEQ ID NO: 33;
a Cas12a protein having an amino acid sequence of SEQ ID NO: 3 and a scaffold having a nucleic acid sequence of SEQ ID NO: 35; or
a Cas12a protein having an amino acid sequence of SEQ ID NO: 2 and a scaffold having a nucleic acid sequence of SEQ ID NO: 34.

In a still yet further embodiment,
the composition may include a complex in which the Cas12a protein and the guide RNA are bound.

In a still yet further embodiment,
the composition may include a nucleic acid encoding the Cas12a protein, and a nucleic acid encoding the guide RNA.

In a still yet further embodiment,
the Cas12a protein linked to one or more nuclear localization signals has a structure as follows:

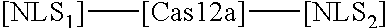

wherein, the NLS₁ has an amino acid sequence selected from SEQ ID NOs: 67 to 94, or is absent, and
the Cas12a is the Cas12a protein described above, and
the NLS₂ has an amino acid sequence selected from SEQ ID NOs: 67 to 94 or is absent, and
at least one of NLS₁ or NLS₂ is present.

This present disclosure discloses a CRISPR/Cas12a composition, including:
a Cas12a protein or a nucleic acid encoding the Cas12a protein; and
a guide RNA or a nucleic acid encoding the guide RNA,
wherein, the guide RNA includes a scaffold and a guide domain, and
the 3′ end of the scaffold is linked to the 5′ end of the guide domain, and
wherein, the guide RNA binds with the Cas12a protein to form a complex,
the guide domain targets a predetermined target nucleic acid, and
the Cas12a protein and the scaffold are selected from the following combinations:
a Cas12a protein having an amino acid sequence selected from SEQ ID NOs: 264 to 274, and a scaffold having a nucleic acid sequence of SEQ ID NO: 33; or
a Cas12a protein having an amino acid sequence selected from SEQ ID NOs: 275 to 279, and a scaffold having a nucleic acid sequence of SEQ ID NO: 35.

In a still yet further embodiment,
the composition may include a complex in which the Cas12a protein and the guide RNA are bound.

In a still yet further embodiment,
the composition may include a nucleic acid encoding the Cas12a protein and a nucleic acid encoding the guide RNA.

In a still yet further embodiment,
the Cas12a protein may further include one or more nuclear localization signals, and
the Cas12a protein may have a structure as follows:

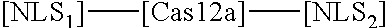

wherein the NLS₁ has an amino acid sequence selected from SEQ ID NOs: 67 to 94, or is absent, and
the Cas12a is the Cas12a protein described above, and
the NLS₂ has an amino acid sequence selected from SEQ ID NOs: 67 to 94 or is absent, and
at least one of NLS₁ or NLS₂ is present.

The present disclosure discloses a method for editing two or more target nucleic acids in the eukaryotic cell genome, the method including the following:
(a) delivering a Cas12a protein linked to one or more nuclear localization signals or a nucleic acid encoding the Cas12a protein linked to one or more nuclear localization signals into the eukaryotic cell; and
(b) delivering a vector construct into the eukaryotic cell,
wherein, the vector construct has a nucleic acid encoding a first guide RNA and a nucleic acid encoding a second guide RNA, and
the first guide RNA includes a first guide domain and a first scaffold,
the first guide domain targets a first target nucleic acid within the eukaryotic cell genome, the second guide RNA includes a second guide domain and a second scaffold,
the second guide domain targets a second target nucleic acid within the eukaryotic cell genome, and
the DNA encoding the first guide RNA is operably linked to a promoter, and
the nucleic acid encoding the second guide RNA is directly linked to the 3′ end of the first guide RNA,
wherein, the (a) and the (b) are performed simultaneously or in any order, and
the Cas12a protein, the first scaffold, and the second scaffold are selected from the following combinations:
a Cas12a protein having an amino acid sequence selected from SEQ ID NOs: 1, and 264 to 274, a first scaffold having a nucleic acid sequence of SEQ ID NO: 33, and a second scaffold having a nucleic acid sequence of SEQ ID NO: 33; or
a Cas12a protein having an amino acid sequence selected from SEQ ID NOs: 3, and 275 to 279, a first scaffold having a nucleic acid sequence of SEQ ID NO: 35, and a second scaffold having a nucleic acid sequence of SEQ ID NO: 35.

### MODE FOR DISCLOSURE

Hereinafter, with reference to the attached drawings, the content of the disclosure will be described in more detail through specific implementation embodiments and examples. It should be noted that the attached drawings include some, but not all, embodiments of the disclosure. The subject matter of the present disclosure disclosed by this specification may be implemented in various ways and is not limited to the specific implementation examples described herein. These implementation examples should be viewed as being provided to satisfy the legal requirements applicable to this specification. Those skilled in the art to which the present disclosure disclosed in this specification pertains will be able to think of many variations and other implementation examples of the present disclosure disclosed in this specification. Accordingly, it should be understood that the content of the present disclosure disclosed in this specification is not limited to the specific embodiments described herein, and that modifications and other embodiments thereof are also included within the scope of the claims.

### Term Definition

### About

As used herein, the term “about” means quantity, level, value, number, frequency, percentage, dimension, size, amount, weight, or length that vary by 30%, 25%, 20%, 15%, 10%, 9%, 8%, 7%, 6%, 5%, 4%, 3%, 2%, 1%, or 0% based on reference quantity, level, value, number, frequency, percentage, dimension, size, amount, weight, or length.

### Amino Acid Sequence Notation

Unless otherwise stated, when describing an amino acid sequence in this specification, the amino acid one-letter notation or three-letter notation is used, and is written in the direction from the N-terminal to the C-terminal. For example, when written as PAAK, it refers to a peptide in which proline, alanine, alanine, and lysine are linked in order from the N-terminal to the C-terminal. For another example, when written as Thr-Leu-Lys, it refers to a peptide in which Threonine, Leucine, and Lysine are sequentially connected from the N-terminal to the C-terminal. In the case of amino acids that cannot be expressed with the one-letter notation, they are written using other letters and are further supplemented and explained.

The notation method for each amino acid is as follows: Alanine (Ala, A); Arginine (Arg, R); Asparagine (Asn, N); Aspartic acid (Asp, D); Cysteine (Cys, C); Glutamic acid (Glu, E); Glutamine (Gln, Q); Glycine (Gly, G); Histidine (His, H); Isoleucine (Ile, I); Leucine (Leu, L); Lysine (Lys K); Methionine (Met, M); Phenylalanine (Phe, F); Proline (Pro, P); Serine (Ser, S); Threonine (Thr, T); Tryptophan (Trp, W); Tyrosine (Tyr, Y); and Valine (Val, V).

### Nucleic Acid Sequence Notation

The symbols A, T, C, G, and U used herein are interpreted as meanings understood by those skilled in the art. Depending on the context and technology, the symbols may be appropriately interpreted as a base, nucleoside, or nucleotide on DNA or RNA. For example, when the symbols refer to bases, the symbols may be interpreted as adenine (A), thymine (T), cytosine (C), guanine (G), or uracil (U), respectively. When the symbols refer to nucleosides, the symbols may be interpreted as adenosine (A), thymidine (T), cytidine (C), guanosine (G), or uridine (U), respectively. When the symbols refer to nucleotides in a sequence, the symbols are required to be interpreted to mean nucleotides including each of the nucleosides.

### Target Gene or Target Nucleic Acid

“Target gene” or “target nucleic acid” as used in this specification basically refers to a gene or nucleic acid in a cell that is the target of gene editing. The target gene or target nucleic acid may be used interchangeably and may refer to the same target. Unless otherwise stated, the target gene or target nucleic acid may refer to either a gene or nucleic acid native to the target cell or a gene or nucleic acid derived from an external source and is not particularly limited as long as it can be the subject of gene editing. The target gene or target nucleic acid may be single-stranded DNA, double-stranded DNA, and/or RNA. In addition, the term includes all meanings that can be recognized by those skilled in the art, and the term can be appropriately interpreted depending on the context.

### Target Sequence

“Target sequence” as used in this specification refers to a specific sequence recognized by a CRISPR/Cas complex to cleave a target gene or target nucleic acid. The target sequence may be appropriately selected depending on the purpose. Specifically, a “target sequence” is a sequence contained within a target gene or target nucleic acid sequence. The target sequence refers to a sequence that is complementary to a spacer sequence included in the guide RNA or engineered guide RNA provided in this specification. Generally, the spacer sequence is determined considering a sequence of the target gene or target nucleic acid and a PAM sequence recognized by an effector protein of the CRISPR/Cas system. The target sequence may refer only to a specific strand that binds complementarily to the guide RNA of the CRISPR/Cas complex. Alternatively, the target sequence may refer to an entire target double-strand including a specific strand portion, which is appropriately interpreted depending on the context. In addition, the term includes all meanings that can be recognized by those skilled in the art, and the term can be appropriately interpreted depending on the context.

### Vector

As used in this specification, “vector” refers collectively to all substances capable of transporting genetic material into a cell, unless otherwise specified. For example, the vector may be a DNA molecule containing the genetic material of interest, for example, a nucleic acid encoding an effector protein of the CRISPR/Cas system, and/or a nucleic acid encoding a guide RNA, but is not limited thereto. The term includes all meanings that can be recognized by those skilled in the art, and the term can be appropriately interpreted depending on the context.

### Limitations of Conventional Techniques

### Difficulties in Discovering CRISPR/Cas12a System Capable of Editing Eukaryotic Genes

Currently, various bacterial-derived CRISPR/Cas systems have been studied and continuously discovered. However, a CRISPR/Cas system is basically derived from a prokaryotic immune system. Therefore, the CRISPR/Cas system works well in prokaryotic cells, but it does not work well in eukaryotic cells due to various factors. Many studies have also been disclosed to utilize the CRISPR/Cas system for eukaryotic gene editing purposes (for example, research on linking NLS to Cas protein). However, even using this research, it is not possible to ensure that “all” CRISPR/Cas systems work in the eukaryotic environment.

Even when a new CRISPR/Cas system is discovered and its composition is specified, it is impossible to predict whether the CRISPR/Cas system may edit genes in eukaryotic cells based solely on information about its composition. Therefore, for the CRISPR/Cas system to be used for editing eukaryotic genes, verification through actual experiments is essential.

### CRISPR/Cas12a Systems Including Cas12a Proteins Disclosed in KR 2023-0111399 A

Korean Application Publication No. KR 2023-0111399 A (hereinafter referred to as Conventional Document 1) discloses dozens of new CRISPR/Cas12a systems. The Conventional Document 1 discloses each Cas12a protein, guide RNA, and PAM sequence recognized by the Cas12a protein, all of which constitute the novel CRISPR/Cas12a system, and also discloses in-vitro nucleic acid cleavage activity. However, little evidence was provided as to whether the CRISPR/Cas12a systems with the composition would functionally operate in eukaryotic cells and exhibit gene editing activity. In other words, looking solely at the disclosure of Conventional Document 1, it is unknown whether each CRISPR/Cas12a system may target-specifically recognize double-stranded nucleic acids and stably bind to them. Furthermore, as a result of closely examining the contents of Conventional Document 1, the inventors of this specification found that the guide RNA of a CRISPR/Cas12a system, especially a direct repeat thereof, was incorrectly specified.

### Novel CRISPR/Cas12a Systems Capable of Editing Eukaryotic Genomes

### Overview of Novel CRISPR/Cas12a Systems Capable of Editing Eukaryotic Genomes

This specification discloses novel CRISPR/Cas12a systems capable of editing eukaryotic genomes. The novel CRISPR/Cas12a systems include novel Cas12a proteins or variants thereof; and programmable guide RNAs. Wherein, the Cas12a proteins or the variants thereof may be linked to one or more nuclear localization signals. Based on the CRISPR/Cas12a systems disclosed in Conventional Document 1 above, the inventors of this specification 1) found direct repeats of the “correct” guide RNAs that functionally operate, 2) selected novel CRISPR/Cas12a systems that can be used for editing eukaryotic genomes among the disclosed CRISPR/Cas12a systems, 3) identified PAM-interacting regions of the Cas12a proteins included in the novel CRISPR/Cas12a systems, and designed additional mutations at the corresponding positions, and 4) developed CRISPR/Cas12a systems that could edit intended regions in the eukaryotic genomes through actual experiments. Some of the CRISPR/Cas12a systems disclosed in this specification include the Cas12a proteins, which were disclosed in Conventional Document 1, but whose eukaryotic genome editing activity has not been revealed. Some of the CRISPR/Cas12a systems disclosed herein include variants of the Cas12a proteins. The components of the CRISPR/Cas12a systems function by forming CRISPR/Cas12a complexes, which means RNA-protein complexes. The CRISPR/Cas12a systems are used in slightly different forms as needed, including CRISPR/Cas12a complexes, CRISPR/Cas12a component expression vectors, and CRISPR/Cas12a compositions. Herein below, the novel Cas12a proteins, the variants thereof, and programmable guide RNAs will be described in more detail based on the CRISPR/Cas12a complexes.

### Components of the CRISPR/Cas12a Complex #1—Novel Cas12a Proteins

In this specification, novel Cas12a proteins are disclosed. Specifically, the Cas12a proteins having an amino acid sequence selected from SEQ ID NO: 1, SEQ ID NO: 3, and SEQ ID NO: 2 are disclosed.

### Components of the CRISPR/Cas12a Complex #2—Variants of Novel Cas12a Proteins

This specification discloses previously undisclosed variants of Cas12a proteins that may edit eukaryotic genomes. These variants are variants of the Cas12a proteins, as discovered by introducing mutations to the novel Cas12a proteins specified above to improve the function of interacting with a PAM. It is already well known that Cas proteins interact with and recognize PAM sequence portions, and that Cas proteins are important for inducing “target-specific activity” while working together with guide domains of the guide RNAs. Accordingly, the inventors of this specification took ideas from conventional research and attempted to obtain Cas12a protein variants with an improved function of interacting with a PAM by 1) specifying positions where the variants of the Cas12a proteins interacted with the PAM, and 2) introducing mutations at the corresponding positions. However, not all mutations described in conventional literature led to increased gene editing efficiency. Accordingly, the inventors of this specification conducted careful experiments, and through them, these inventors specified mutations that improved nucleobase editing activity, mutations that improved activity only for specific targets, and mutations that were “usable” while exhibiting nucleobase editing activity on a variety of targets. The variants are described in detail in the “Novel Variants of Cas12a Proteins” section.

### Components of the CRISPR/Cas12a Complex #3—Nuclear Localization Signals

To use the CRISPR/Cas12a complexes for editing eukaryotic genomes, the complexes are required to be able to enter the eukaryotic cell nucleus. For the CRISPR/Cas12a complexes to enter the eukaryotic cell nucleus, nuclear localization signals (NLS) may be linked to the Cas12a proteins. The nuclear localization signals are not otherwise limited in type or composition as long as they deliver the complexes to the nucleus of a eukaryotic cell, and any known composition may be used. For example, a nuclear localization signal may be SV40. One or more nuclear localization signals may be linked to the Cas12a proteins above. The nuclear localization signals may be linked to the Cas12a proteins directly or via an amino acid linker. The specific compositions of the Cas12a proteins to which the nuclear localization signals are linked are described in detail in the “Possible Examples of Present Disclosure” section.

### Components of the CRISPR/Cas12a Complex #4—Programmable Guide RNAs

The CRISPR/Cas12a complex includes a programmable guide RNA. The programmable guide RNA includes 1) a scaffold, which is a portion that importantly interacts with a Cas12a protein, and 2) a guide domain designed to match a target region. It is known that CRISPR/Cas12a systems function solely with crRNAs, without requiring tracrRNAs. The scaffold of the programmable guide RNA essentially includes a direct repeat (DR) of the crRNA. The inventors of this specification found that the direct repeats in the guide RNAs of the novel CRISPR/Cas12a systems disclosed in the conventional literature were incorrectly specified. The inventors redefined these and invented CRISPR/Cas12a systems that were fully functional. A scaffold may further include additional components that contribute to improving gene editing efficiency in addition to a direct repeat. A guide domain may target a target sequence and is appropriately designed depending on a target nucleic acid to be edited. Specific definitions of a guide domain sequence, target nucleic acid, target site, target sequence, and their relationships are explained in more detail in the relevant section.

Features of the CRISPR/Cas12a System #1—Differences from Content Disclosed in Conventional Literature

As mentioned above, Conventional Document 1 (Korean Publication KR 2023-0111399 A) discloses a novel ortholog of Cas12a proteins. The Conventional Document 1 discloses amino acid sequences of novel Cas12a proteins, PAM sequences recognized by the novel Cas12a proteins, and direct repeat sequences for the novel Cas12a proteins. However, in Conventional Literature 1, only small portions of the novel CRISPR/Cas12a systems were tested for intracellular gene editing activity. For most of the novel CRISPR/Cas12a systems, no evidence was provided as to whether the systems were capable of gene editing in eukaryotic cells. On the other hand, the inventors of this specification reviewed data on the novel Cas12a proteins and their guide RNAs disclosed in Conventional Document 1. Through additional research, it was found that the direct repeat sequences of the guide RNAs were incorrectly specified, and the inventors re-specified the direct repeat sequences to the correct sequences. In addition, the inventors of this specification demonstrated through experiments that eukaryotic genes could be edited using Cas12a proteins having an amino acid sequence selected from SEQ ID NOs: 1 to 3 among the novel Cas12a proteins disclosed in Conventional Document 1. Furthermore, the inventors clearly defined a composition for editing eukaryotic genes using a Cas12a protein, thereby completing the invention of the novel CRISPR/Cas12a systems.

### Features of the CRISPR/Cas12a System #2—Specific New Guide RNAs

The inventors of this specification reviewed data on the novel Cas12a proteins and their corresponding guide RNAs disclosed in Conventional Document 1. Through additional research, these inventors found that the direct repeat sequences of the guide RNAs had been incorrectly specified. The inventors corrected and redefined the direct repeat sequences. Through experiments, the inventors found that Cas12a proteins having an amino acid sequence selected from SEQ ID NOs: 1 to 3 and guide RNAs that operated together with the Cas12a proteins exhibited gene editing activity in eukaryotic cells. Specifically, sequences for the Cas12a proteins and sequences for direct repeats, which could interact with the sequences of the Cas12a proteins, to form complexes discovered by the inventors are as follows:

a Cas12a protein having an amino acid sequence of SEQ ID NO: 1 and a direct repeat having an amino acid sequence of SEQ ID NO: 33 (or similar sequence);

a Cas12a protein having an amino acid sequence of SEQ ID NO: 2 and a direct repeat having an amino acid sequence of SEQ ID NO: 34 (or similar sequence); and

a Cas12a protein having an amino acid sequence of SEQ ID NO: 3 and a direct repeat having an amino acid sequence of SEQ ID NO: 35 (or similar sequence).

The Cas12a proteins and the direct repeat-including guide RNAs in such combinations may interact to form complexes and exhibit target-specific nucleic acid cleavage activity.

### Features of the CRISPR/Cas12a System #3—Development of Variants of Novel Cas12a Proteins

This specification discloses variants of the Cas12a proteins with a new composition that has not been previously known. The Cas12a protein variants are prepared based on the sequence of the novel Cas12a proteins discovered above by 1) introducing a mutation at the positions that interact with a PAM (PAM recognition site mutation), 2) introducing a mutation that increase additional nucleic acid cleavage efficiency (Ultra Nuclease mutation), or 3) appropriately combining the mutations. The inventors of this specification revealed that the variants of the Cas12a proteins could also be used for eukaryotic genome editing, and that some of them exhibited more improved gene editing efficiency than the novel Cas12a proteins.

### Variants of Novel Cas12a Proteins

### Overview of Variants of Novel Cas12a Proteins

The inventors herein disclose variants of the novel Cas12a proteins. The variants of the Cas12a protein are prepared based on the sequences of the novel Cas12a proteins discovered above by 1) introducing a mutation at the positions that interact with a PAM (PAM recognition site mutation), 2) introducing a mutation that increase additional nucleic acid cleavage efficiency (Ultra Nuclease mutation), or 3) appropriately combining the mutations. For example, the variants of the Cas12a proteins have an amino acid sequence selected from SEQ ID NOs: 264 to 279. Each mutation was inspired by previous literature. Specifically, structures of the novel Cas12a proteins were analyzed using a simulation model, and each of the mutation positions was discovered by matching and comparing the mutations with their corresponding amino acid sequences of the Cas12a proteins that initiated the mutations. As a result of confirming actual gene editing in the cases of each of the mutations or a combination of the mutations, not all mutations disclosed in conventional literature improved gene editing efficiency. Through careful analysis, the inventors of this specification specified variants that showed overall better efficiency than the wild-type Cas12a protein, exhibited better editing efficiency for specific targets, or exhibited editing efficiency above a certain level and could be used for eukaryotic genome editing.

### PAM Recognition Site Mutation #1—Overview

This specification discloses variants of the novel Cas12a proteins, prepared by introducing the PAM recognition site mutation. Specifically, the mutation is associated with the PAM-interacting positions. For example, a PAM recognition site mutation for an amino acid sequence of SEQ ID NO: 1 is a mutation in which the residues at positions E155, G531, K537, or any combination thereof are replaced with a different amino acid. As another example, a PAM recognition site mutation for an amino acid sequence of SEQ ID NO: 3 is a mutation in which the residues at positions Q190, S579, Y585, or any combination thereof are replaced with a different amino acid. Wherein, the mutant amino acid is preferably arginine, which has a strong (+) charge, but is not limited thereto. The inventors of this specification took an idea from conventional literature and improved target nucleic acid editing activity by further introducing the PAM recognition site mutation into the novel Cas12a proteins. The inventors conducted careful experiments, and through them, these inventors specified mutations that improved gene editing activity, mutations that improved activity only for specific targets, and mutations that were “usable” by showing gene editing activity above a certain level on a variety of targets.

### PAM Recognition Site Mutation #2—Principle of Improving Gene Editing Efficiency Through Mutations

It is already well known that Cas proteins interact with and recognize PAM sequence portions, and that Cas proteins are important for inducing “target-specific activity” while working together with the guide domains of guide RNAs. According to conventional literature (Kleinstiver B P, Sousa A A, Walton R T, Tak Y E, Hsu J Y, Clement K, Welch M M, Horng J E, Malagon-Lopez J, Scarfò I, Maus M V, Pinello L, Aryee M J, Joung J K. Engineered CRISPR-Cas12a variants with increased activities and improved targeting ranges for gene, epigenetic and base editing. Nat Biotechnol. 2019 March; 37 (3): 276-282. doi: 10.1038/s41587-018-0011-0. Epub 2019 Feb. 11. Erratum in: Nat Biotechnol. 2020 July; 38 (7): 901. doi: 10.1038/s41587-020-0587-z. PMID: 30742127; PMCID: PMC6401248.), when specific residues, which interact with the PAM sequence portions, of the Cas12a proteins are replaced with residues with stronger activity, the ability to recognize target nucleic acids is improved. Specifically, the conventional literature specified three PAM-interacting regions in AsCas12a protein, and found that when the residues in the regions were replaced with arginine having a strong (+) charge, both gene editing activity and base editing activity increased. The inventors of this specification took an idea from the literature and attempted to improve target nucleic acid editing activity by further introducing the PAM binding site mutation into the novel Cas12a proteins. Specifically, key residues interacting with the PAM sequences were identified through structural analysis, “PAM binding site mutations” that replaced the key residues with amino acids having a strong (+) charge were introduced into the Cas12a proteins, and then, whether the mutations affected gene editing efficiency was confirmed. Actual experimental results showed that not all PAM binding site mutations resulted in improved gene editing activity. Herein below, a method for predicting a structure and a process of specifying a position for introducing a PAM binding site are explained in more detail.

### PAM Recognition Site Mutation #3—Structure Prediction Using AlphaFold

The inventors of this specification used an AlphaFold model and analyzed respective structures when the Cas12a protein having an amino acid sequence of SEQ ID NO: 1 and the Cas12a protein having an amino acid sequence of SEQ ID NO: 3 bound to their corresponding target nucleic acids. As a result of the analysis, it was possible to select the positions of key residues interacting with the PAM sequences. Specifically, positions E155, G531, and K537 in the amino acid sequence of SEQ ID NO: 1 were positions that significantly interacted with the PAM sequences. In addition, positions Q190, S579, and Y585 in the amino acid sequence of SEQ ID NO: 3 were positions that significantly interacted with the PAM sequences. The inventors of this specification predicted that when the key residues were replaced with appropriate amino acids, they would bind more strongly to the PAM sequences and, as a result, contribute to improving gene editing efficiency. Therefore, the inventors of this specification attempted to improve gene editing efficiency by replacing each of the key residues with arginine having a strong (+) charge. Specifically, in the novel Cas12a proteins, for each position derived above, mutations in all possible combinations (mutating only one position, two positions, or all three positions) were introduced, thereby Cas12a proteins were prepared. Furthermore, CRISPR/Cas12a systems, each including one of the Cas12a proteins, were prepared, and the gene editing activity of each case was measured.

### Ultra Nuclease Mutation #1—Overview

This specification discloses novel Cas12a proteins, prepared by introducing a Ultra Nuclease mutation. Specifically, the mutation is related to DNA binding activity of Cas12a proteins or to a function of stabilizing the CRISPR/Cas12a complexes. For example, an Ultra Nuclease mutation for an amino acid sequence of SEQ ID NO: 1 is a mutation in which the residues at positions N526, E791, or any combination thereof is replaced with a different amino acid. Wherein, a mutant amino acid is preferably arginine for N526 and leucine for E791, but is not limited thereto. The inventors of this specification took an idea from conventional literature and further introduced the Ultra Nuclease mutation into the novel Cas12a protein to improve the target nucleic acid editing activity. The inventors conducted careful experiments, and through them, these inventors specified mutations that improved gene editing activity, mutations that improved activity only for specific targets, and mutations that were “usable” by showing gene editing activity above a certain level on a variety of targets.

### Ultra Nuclease Mutation #2—Principle of Improving Gene Editing Efficiency Through Mutations

The inventors of this specification referred to the conventional literature (Zhang, L., Zuris, J. A., Viswanathan, R. et al. AsCas12a ultra nuclease facilitates the rapid generation of therapeutic cell medicines. Nat Commun 12, 3908 (2021). https://doi.org/10.1038/s41467-021-24017-8; Hsiung, C. C S., Wilson, C. M., Sambold, N. A. et al. Engineered CRISPR-Cas12a for higher-order combinatorial chromatin perturbations. Nat Biotechnol (2024). https://doi.org/10.1038/s41587-024-02224-0), and discovered additional mutations. The conventional literature is a study on mutations that increase nucleic acid cleavage efficiency of the CRISPR/Cas12a systems. In the conventional literature, the inventors of the present disclosure interpreted that a M537R mutation of the AsCas12a protein is a mutation that increases DNA binding activity of the Cas12a proteins, thereby further enhancing PAM interaction with the Cas12a proteins, and believed that a F870L mutation is a mutation that further stabilizes the CRISPR/Cas12a complexes. Accordingly, the inventors speculated that gene editing efficiency would increase when a mutation corresponding to the mutation (hereinafter, referred to as Ultra Nuclease mutation) was applied to the novel Cas12a proteins. Through sequence alignment and structural analysis, the inventors found amino acid positions in the novel Cas12a that corresponded to the mutation positions of AsCas12a introduced in the previous study, and designed a Ultra Nuclease mutation to replace the residues in the positions with arginine or leucine. Then, by introducing the Ultra Nuclease mutation into the novel Cas12a proteins, Cas12a proteins were prepared, and by preparing CRISPR/Cas12a systems including the novel Cas12a proteins, eukaryotic genome editing efficiency was confirmed.

### Random Combination of Each Mutation

The inventors of this specification predicted that 1) the PAM recognition site mutation and the Ultra Nuclease mutation did not overlap in the positions where the mutations were introduced, and 2) each position was involved in performing a different function, so synergy could occur when the mutations were combined with each other. Therefore, by randomly combining the PAM mutation and Ultra Nuclease mutation, Cas12a protein variants were prepared, and by preparing CRISPR/cas12a systems including the Cas12a protein variants, eukaryotic genome editing efficiency was confirmed.

### Not all Modifications Result in Increased Editing Efficiency

As a result of the experiments, not all mutations improved gene editing efficiency (see experimental examples). Through careful experiments, the inventors identified and specified variants able to be used, the variants including 1) variants that showed a higher gene editing effect than the novel Cas12a proteins, 2) variants that were effective solely for specific sequences, and 3) variants that showed a certain level of gene editing effect. For example, the Cas12a proteins having an amino acid sequence selected from SEQ ID NO: 264, SEQ ID NOS: 269 to 270, and SEQ ID NOs: 275 to 277 showed high gene editing efficiency compared to the Cas12a proteins having an amino acid sequence selected from SEQ ID NOs: 1 to 3. As another example, the Cas12a proteins having an amino acid sequence selected from SEQ ID NOs: 265 to 268, SEQ ID NOs: 271 to 274, and SEQ ID NOs: 278 to 279 showed a gene editing effect at a sufficiently usable level when included in the CRISPR/Cas12a system.

### Programmable Guide RNAs

### Overview of Programmable Guide RNAs

The CRISPR/Cas12a systems disclosed herein include programmable guide RNAs (or nucleic acids encoding the same). The programmable guide RNAs may interact with Cas12a proteins to form RNA-protein complexes. The programmable guide RNAs 1) interact with the Cas12a proteins to form complexes, and 2) guide the RNA-protein complexes to the target sequences around the PAM sequences, enabling gene editing to occur at intended positions. Among these, portions designed to interact with the Cas12a proteins to form complexes may be categorized into scaffolds, and portions designed to guide the RNA-protein complexes to the target sequences may be categorized into guide domains. Herein below, each portion will be described in detail.

### Scaffold

The programmable guide RNAs include their corresponding scaffolds. The scaffolds may interact with and bind to their Cas12a proteins and facilitate the formation of complexes between the programmable guide RNAs and the Cas12a proteins. The scaffolds include direct repeats of crRNAs. The inventors of this specification specify and disclose the direct repeats that functionally bind to the Cas12a proteins disclosed in this specification to form complexes. For example, when a Cas12a protein has an amino acid sequence of SEQ ID NO: 1 or a variant has an amino acid sequence of SEQ ID NO: 1, a direct repeat has a nucleic acid sequence of SEQ ID NO: 33. As another example, when a Cas12a protein has an amino acid sequence of SEQ ID NO: 3 or a variant has an amino acid sequence of SEQ ID NO: 3, a direct repeat has a nucleic acid sequence of SEQ ID NO: 35. In addition to the direct repeats, the scaffolds may further include additional RNA domains that provide structural stability to the programmable guide RNAs. For example, a scaffold may further include a stem-loop. The specific composition of each of the scaffolds is described in the “Possible Examples of Present Disclosure” section.

### Guide Domains

The programmable guide RNAs include guide domains. The guide domains target their target nucleic acids and guide RNA-protein complexes to bind to the target nucleic acids. The guide domains are designed with sequences related to the target nucleic acids and enable CRISPR/Cas12a complexes to edit nucleic acids at intended positions. For example, a guide domain is designed as a sequence complementary to all or part of a target strand of a target nucleic acid. In other words, the guide domain is designed as a sequence homologous to all or part of a non-target strand of a target nucleic acid. When a Cas12a protein recognizes the PAM sequence of a non-target strand of a target nucleic acid, and a guide domain binds complementarily to a target strand of the target nucleic acid around the PAM sequence, the CRISPR/Cas12a complex formed may function by stably binding to a corresponding position. The specific composition of each of the guide domains is described in the “Possible Examples of Present Disclosure” section.

### Programmable Guide RNA Structure and Additional RNA Sequences

The CRISPR/Cas systems described in this specification belong to CRISPR/Cas12a systems. Accordingly, programmable guide RNA structures are also identical to those for known CRISPR/Cas12a systems. Specifically, the programmable guide RNAs have a structure in which a scaffold and a guide domain are connected sequentially from the 5′ end to the 3′ end. The 3′ end of the scaffolds and the 5′ end of the guide domains may be connected directly or via an arbitrary RNA linker. Furthermore, additional RNA sequences may be connected to the 5′ end, 3′ end, or both ends of the RNAs in which the scaffolds and guide domains are linked together. The additional RNA sequences may function to provide stability to the programmable guide RNAs or increase the efficiency of the guide domains binding to the target sequences.

### CRISPR/Cas12a Systems

### CRISPR/Cas12a System Overview

This specification discloses CRISPR/Cas12a systems. CRISPR/Cas12a complexes are those, which functionally edit nucleobases, formed by binding the Cas12a proteins with programmable guide RNAs described above. As biotechnology technology advances, it is possible to deliver a nucleic acid encoding a protein and a nucleic acid encoding RNA into a cell and express the respective protein and RNA within the cell to form a CRISPR/Cas12a complex. Therefore, the term “CRISPR/Cas12a systems” used herein encompasses 1) a CRISPR/Cas12a complex, 2) a vector capable of expressing each component of a CRISPR/Cas12a complex (CRISPR/Cas12a vector), and 3) a CRISPR/Cas12a composition.

### CRISPR/Cas12a Complexes

The CRISPR/Cas12a systems may refer to CRISPR/Cas12a complexes. The CRISPR/Cas12a complexes refer to RNA-protein complexes formed by binding Cas12a proteins described above or Cas12a proteins linked to one or more nuclear localization signals with programmable guide RNAs. The CRISPR/Cas12a complexes functionally recognize target nucleic acids and edit nucleobases at intended target positions.

### CRISPR/Cas12a Vectors

The CRISPR/Cas12a systems may refer to CRISPR/Cas12a vectors. The CRISPR/Cas12a vectors refer to vectors that may express each component of CRISPR/Cas12a complexes. CRISPR/Cas12a vectors have nucleic acids encoding Cas12a proteins described above or Cas12a proteins linked to one or more nuclear localization signals with nucleic acids encoding programmable guide RNAs. The CRISPR/Cas12a vectors are designed to express Cas12a proteins and programmable guide RNAs when delivered to cells. When the CRISPR/Cas12a vectors are delivered into cells, the Cas12a proteins and programmable guide RNAs are expressed within the cells, and they are bound to form CRISPR/Cas12a complexes and function as intended. In addition to the nucleic acids encoding each of the components, the CRISPR/Cas12a vectors include components to ensure appropriate expression within cells. The vectors can be constructed by appropriately utilizing known techniques. Specific vector composition examples are described in detail in the “Possible Examples of Present Disclosure” section.

### CRISPR/Cas12a Compositions

The base editing systems may refer to CRISPR/Cas12a compositions. The CRISPR/Cas12a compositions include CRISPR/Cas12a proteins or nucleic acids encoding the CRISPR/Cas12a proteins; and programmable guide RNAs or nucleic acids encoding the programmable guide RNAs. The CRISPR/Cas12a compositions encompass CRISPR/Cas12a complexes, CRISPR/Cas12a vectors, and various types, implemented using the techniques described above. For example, a CRISPR/Cas12a composition may include an mRNA encoding a CRISPR/Cas12a protein, and a programmable guide RNA.

### Method for Editing Eukaryotic Genomes

### Overview of Method for Editing Eukaryotic Genomes

This specification discloses a method for editing eukaryotic genomes. The method of editing eukaryotic genomes includes a process of delivering a CRISPR/Cas12a system among those described above into the eukaryotic cell. The CRISPR/Cas12a system targets its predetermined target nucleic acid in the eukaryotic genome and may edit the target nucleic acid or a portion adjacent to it.

### CRISPR/Cas12a Systems for Editing Eukaryotic Genomes

The method of editing eukaryotic genomes uses a CRISPR/Cas12a system for editing eukaryotic genomes. The CRISPR/Cas12a system is designed for editing eukaryotic genomes, and each component is as follows: a Cas12a protein with one or more nuclear localization signals linked; and a programmable guide RNA that targets a predetermined target nucleic acid in the eukaryotic genome. As mentioned above, the CRISPR/Cas12a system may be implemented in various forms, and the specific details are described in the paragraph above.

### Cells Targeted for Genome Editing

In the method, cells subject to genome editing are eukaryotic cells. The eukaryotic cells encompass several types of cells, and are not otherwise limited as long as the CRISPR/Cas12a systems may operate. For example, the cells may be plant cells, non-human animal cells, or human cells.

### Method for Delivering CRISPR/Cas12a Systems

The method of editing eukaryotic genomes is performed by delivering a CRISPR/Cas12a system for editing eukaryotic genomes to the eukaryotic cell. The delivery process is performed to make contact or induce contact between a CRISPR/Cas12a complex and the cell's genome within the target cell. As long as the purpose is achieved, the delivery method is not otherwise limited. Those skilled in the art may deliver a CRISPR/Cas12a system for editing eukaryotic genomes to the eukaryotic cell using known methods. Depending on the specific realization of a CRISPR/Cas12a system, the delivery method may vary appropriately. For example, when a CRISPR/Cas12a system is a CRISPR/Cas12a complex, a delivery process may be a process of packaging the CRISPR/Cas12a complex into a lipid nanoparticle and then delivering it.

### Genome Editing Method Performance Environment

The genome editing method may be performed in any environment as long as a CRISPR/Cas12a system for editing eukaryotic genomes may be delivered to the cell. For example, the method may be performed in an in vitro, in vivo, or ex vivo environment.

### Method for Editing Nucleic Acids in Cytoplasm

### Overview of Method for Editing Nucleic Acids in Cytoplasm

This specification discloses a method for editing nucleic acids contained in the cytoplasm. The method of editing nucleic acids in the cytoplasm includes a process of delivering a CRISPR/Cas12a system described above into the cell. The CRISPR/Cas12a system targets a nucleic acid within the cell and may edit a nucleic acid or a portion adjacent to it.

### Nuclear Localization Signals not Included

The method of editing nucleic acids in the cytoplasm is similar to the method of editing eukaryotic genomes described above. However, the composition of a CRISPR/Cas12a system used is different. Specifically, a CRISPR/Cas12a system used in a method for editing intracytoplasmic nucleic acids does not include nuclear localization signals. In other words, the nuclear localization signals are not linked to a Cas12a protein included in the CRISPR/Cas12a system. This is because the purpose of the method is to make a CRISPR/Cas12a system operate in the cytoplasm rather than the cell nucleus.

### Method for Editing Multiple Target Nucleic Acids

### Overview of Method for Editing Multiple Target Nucleic Acids

This specification discloses a method for editing two or more target nucleic acids. The method includes delivering Cas12a proteins or nucleic acids encoding the same; and a vector construct encoding multiple guide RNAs into a cell. When the method is performed, the following is induced: 1) Multiple guide RNAs encoded in a vector construct are expressed within a cell, 2) Each of the expressed guide RNAs binds with the Cas12a proteins (or Cas12a proteins expressed from the encoding nucleic acids) to form CRISPR/Cas12a complexes, and 3) A nucleic acid targeted by each guide RNA is edited.

### Vector Construct Encoding Multiple Guide RNAs

The method uses a vector construct encoding multiple guide RNAs. The structure satisfies the following features:
1) A vector construct contains nucleic acids encoding two or more guide RNAs.
2) The nucleic acids each encoding the guide RNAs are directly linked without requiring components such as T6 terminators.
3) One promoter is operably linked to the nucleic acids encoding two or more guide RNAs.

It is known that the CRISPR/Cas12a complexes operate solely with crRNAs without requiring tracrRNAs. In addition, it is known that the guide RNAs are transcribed from the nucleic acids or genes encoding them, and without the involvement of tracrRNAs during the maturation process, the guide RNAs are independently cleaved and undergo a maturation process. When applying this fact, it is possible to design a construct that encodes multiple guide RNAs as shown above. The vector construct contains nucleic acids encoding two or more guide RNAs, and there is no need to distinguish each encoding nucleic acid as additional components such as spacers and terminators. In addition, even when transcription is performed with only one promoter operatively linked to a nucleic acid encoding two or more guide RNAs, the guide RNAs are separated into individual guide RNA and independently expressed during the maturation process.

### Method for Editing Multiple Target Nucleic Acids Using Vector Constructs Encoding Multiple Guide RNAs

The method includes delivering a vector construct encoding multiple guide RNAs; and Cas12a proteins or nucleic acids encoding the same to the cell. The Cas12a proteins may or may not be linked to nuclear localization signals depending on whether target nucleic acids are located in the nucleus or the cytoplasm. When the method is performed, the following is induced: 1) Multiple guide RNAs encoded in a vector construct are expressed within the cell, 2) Each of the expressed guide RNAs binds with the corresponding Cas12a proteins (or Cas12a proteins expressed from the encoding nucleic acids) to form CRISPR/Cas12a complexes, and 3) A nucleic acid targeted by each guide RNA is edited. The delivery method is not otherwise limited as long as the process may be performed by delivering the vector construct and the Cas12a proteins or nucleic acids encoding the same, into the cell. For example, the method may be performed by appropriately selecting a known method.

### POSSIBLE EXAMPLES OF PRESENT DISCLOSURE

### Novel Cas12a Proteins

### Example 1, Novel Cas12a Proteins

Cas12a proteins have a sequence selected from the following:

(SEQ ID NO: 1)
KKIDNFTGCYSLSRTLRFKLIPQGKTQEHIELKRLLDEDEKRAED
YKKAKKIIDKYHITFIDRILHNIKLTELQSYIELDEVSNKDEKQK
KELLKIEGLLRKEISNSFKKDDEYKKIFGKEIIETILPEYLDDQE
EIEIIKSFKGFSTAFVGYWENRKNLYTDEEIGSSIAYRAINDNLP
KFMRNASIFKSVEKIFSEEELREIKKEVLNDEYDVQDFFRKDFYD
FLLPQDGIDIYNAILGGLVKEDGTKIRGLNEYINLYNQKNKEKNA
KLKPLFKQVLTESESKSFYIDEFAKDEEVIEAFQNTFAEEGEVCL
AIDQIQKLFNQYEIYDSNGIFIKNGLAVTALANDIYGSWSYIKEK
WNTLYDAENTGRTSKESESYITKRENIYKKIESFSLNEIETITEG
EEKLLSEISNIISEKIQLYFEKYNSAKYLLNSDYKLDKKLQKNNK
VVEVIKELMDSVKDIERYLKPFMGTGKEEGRDEYFYSEFENTLEV
LSGIDRLYNKIRNYVTKEPFSKKKFKLYFQNPQFMGGWDRNKEAD
YRVALLRKKQKYYLAIMDKSDSKCLQRVEQGNGDLQKMNYKLISG
ASKSLPHVFFSKKYLSNNNVEDEILRIYKSKTFIKGESFDIDDCH
KLINYYKESIKSHPWGSAFDFSFSDTNEYADIHTFYQEVDDMGYK
IVFDNVSEEAVNKLVDEGKLYLFQIYNKDFSEKSHGTENLHTMYF
KSLFDENNRGNIRLCGGAEMFLRKASLKKQELVVHPANVPMKNKN
KNNPKKTTTLPYDVYKDKRYSEDQYEIHIPIIINKATTNFYKLNL
EIRKLLREDTNPYVIGIDRGERNLLYMVVIDGKGRIVEQYSINEI
VNECKGITIKTDYHSLLDEKEKERMAARKSWKTVENIKELKEGYI
SQVVHKICELVRKYDAVIALEDLNSGFKNSRVKVEKQVYQKFEKM
LIDKLNYMVDKKIPWYENGGVLKGYQLTNKFESFKKMGTQNGMMF
YIPAWLTSKIDPSTGFVNLLRTRYKSVQDSKNFINKISSIKYDKD
EEMFCFAIDYSNFEHTNADYKKKWILYSNGERIKHFRNPEKNSEF
DYKIINLTTEFKNLFEEYNIAYELGEDIKEQINVINEKKFFEKFM
STVTLMLQMRNSITGRTDIDYLISPVKNDTGNFYDSRNFEALENA
VLPKDADANGAYNIARKVLWAIEQFRESDESKLEKVSIAISNKKW
LEYAQK;
(SEQ ID NO: 2)
TFYNIILKKYKNIEGKMNQNTSISQFTGLYPVSKTLRFELKPMGK
TLEKIKETGIIENDKRRHNDYFDAKKIIDTYHKYFIDAALSKFSR
IDWNPLKEAIEGSLDKSDASKKKLEKIQTEFRKKIAKALTTHDHY
KELTASTPKDLFLKVFPDHFGKQPAIDTFDGFSSYFTGFQENRQN
IYSDEAISTAIPYRLVHDNFPKFLSNIEVYKTLKDNAPSVLSDAE
NELKDFLNGKSLANIFELNAYNDVLTQSGIDFFNQVIGGISGEGG
EKKTRGINEFSNLYRQQHPEFAQKRLATKMIPLYKQILSDRETKS
FILESYSTDSQVQESVKEFFESQILNCDIAGRKVNVLKELSSLIK
RITEFDLGSIYVNQEELSNISLELFKSWNTINAVLFKDAENRIGS
AEKAANKKKIDAWMKSNEFSIATLNLAIAESDSEEISRVKIESYW
NDFEAKVQSILCGDNRRNLDEFLSATFNENNALREDSEIIGKLKA
FLDALIEIMHSIKPLISDAENRDLSFYNELMPLYDQLSLVVPLYN
KIRNYATQKLTESEKFKLNFDCPTLADGWDQNKEKDNKSILLRKD
GLYYLGVMNTNDMPKIEEINALGNEDCYEKMIYKQFDCLKQIPKC
TTQTKAAKAHFEAGKTEDFVIKDKSFNGPFVISEYIWKLNNYVWN
GEKFVLKFGDADKRPKQFQMGYYKETNDLLGYKKALADWIDFCKK
FVKTYISASGYNYDFLDSDKYNSLDEFFSYLKTICYKITFSKIPT
SQIDEWVNEGKLFLFQIYNKDFAPGAKGSPNLHTLYWKSVFSPEN
LKDVVVKLNGEAELFYRPSSVKKPYSHKVGEKLVNRIGKDGLPLP
ESVFGELFRYFNGKLDGELSDEAKKYLDVAVVKDVKHEIVKDRRY
TQDKFEFHVPLTLNFKADSKNEYMNERVRHFLKDNPDVNIIGIDR
GERHLLYMTLINQKGEILKQKSFNIVESVNYQAKLIQREKERDAA
RRSWSSVGKIKDLKEGFLSQVIHEITTTMIENNAIVVLEDLNFGF
KRGRFCVERQVYQKFEKMLIDKLNYLVFKNKPEGDVGGVLKGYQL
AEKFDSFQKLGKQSGFLFYIPAAYTSKIDPTTGFANLFNMTELTS
AEKKKEFLSHFEDITYDGKNDRFLFSFDYKNFKCFQTDYIKKWTV
YSQGKRIVYDKESKSAKEISPVEIIKAALAKQNIALTDQLDVLSA
INSAEASPKSASFFGDICYAFEKTLQMRNSIPNTDEDYLVSPVLN
KKGEFYDSRSCGDTLPKNADANGAYHIALKGLYLIKNVFDAGGKD
LKISHEDWFKFAQSRNS;
and
(SEQ ID NO: 3)
AFVICYNTYSRTYLEKEIISMKTIESFCGQKKGYSRSITLRNRLI
PIGKTEENIRKLKLLDKDIDRSKAYVEVKALIDDFHRVFIEDVLS
KTELPWEPLYDQFELYQNEKDKQKKNKIKKDLEELQKGLRKSIVK
QFKADDRFDKLFKKELLTEFVPSVIKNDDSGTISDKQAALDIFKG
FATYFTGFHQNRQNMYSEEAQSTAISNRIVNENFPKFYANIQTFK
YLEENFPQIISDTEQSLSDFLGDKKLKDIFSIDGFNKVLSQSGID
FYNTVIGGISEEAGTQKVQGLNEKINLASQQLSSEEKHKLKKKMT
VLYKQILSDRSTASFIPVGFEKSEEVYDSVKEFKELSLDKNIAAI
NTIFSRDDYDLTRIFVPAKEITEFSLKLFGHWSILQDGLFLLGKD
NAKKDLSEKQITELIKEIAKKDYSLAELQNAYERWTKENDIPVEK
TVKNYFRLAELRTDEKTKEKNFTDIQKELEKAFVQIDFDKKENLI
KEKEAATPIKNFLDEVQNLFHYLKLVDYRGEEEKDSDFYAKYDQV
LQALAEIIPLYNKVRNFVTKKPNEVKKVKLNFECSSFLSGWGTDY
GTKEAHIFIDDGKYYLGIVNEKLSKEDIKFLEEKSPRMIKKVVYD
FQKPDNKNTPRLFIRSKGTSYAPSVSQYDLPIESVIEIYDKGLFK
TEYRKVNPSVYKESLVKMIDYFKLGFTRHESYKHYNFSWKDSKDY
NDISEFYADVMNSCYQLKFEYINYDNLLSLVDAGKLFLFQIYNKD
FSTGKDGANGSTGKKNLHTLYWENLFSEENLKDICLKLNGEAELF
WRDVNPNIKNICHKKGSILVNRTTSDGKVIPEDIYQEIYKFKNPD
KQEKDFKISDEAKALLENGKVVCKEAKFDITKDRHFTQQTYLFHC
PITMSFKAPEITGRKFNEKVQSILKANPDVKIIGLDRGERHLIYL
SLINQKGEIELQKTLNLVDQVRNDKTVSVNYQEKLVHKEGERDKA
RKNWQSISNIKELKEGYLSNVVHEIAKLMVENNAIVVMEDLNFGF
KRGRFAVERQVYQKFENMLIEKLNYLVFKDKAVTEPGGVLNAYQL
TDKSANVSDVGKQCGWIFYVPAAYTSKIDPKTGFANLFYTAGLTN
VEKKKEFFDKFEAIRYDSKTDSFVLSFDYKDFSDNADYKKKWSLY
SRGERLVFSKAEKNVISVNPTENLKALFDKQGINWKSEENFIDQI
NAVQAERENVSFFDGLYRSFTAILQMRNSVPNSSKMEDDYLISPV
MAEDGTFYDSRKEAAKGKDEQGKWISKLPVDADANGAYHIALKGL
YLLQNDFNVNDKGYIDNISNADWFEFAQEKNYAK.

### Variants of Novel Cas12a Proteins

### Example 2, Variants of Novel Cas12a Proteins

Variants of Cas12a proteins have an amino acid sequence in which one or more amino acids are substituted, inserted, or deleted based on the amino acid sequence selected from the following (hereinafter referred to as the reference amino acid sequence):
SEQ ID NOs: 1 to 3.

### Example 3, Modification Basis

In Example 2, the variants of the Cas12a proteins, compared to the Cas12a proteins having the reference amino acid sequences, have:
an improved ability to bind to a protospacer adjacent motif (PAM);
an improved ability to bind to DNA;
an improved ability to stably maintain a CRISPR/Cas12a complex; or
improvements obtained by arbitrarily combining the abilities in the contents.

### Example 4, Novel Cas12a Variants, Mutation Positions

In any of Examples 2 to 3, the variants of the Cas12a proteins include a mutation selected from the following:
E155R, G531R, K537R, S1137A, N526R, E791L, or any combination thereof, based on the amino acid sequence of SEQ ID NO: 1; and
Q190R, S579R, Y585R, or any combination thereof, based on the amino acid sequence of SEQ ID NO: 2.

### Example 5, Novel Cas12a Variants, Sequences

In any of Examples 2 to 4, the variants of the Cas12a proteins have an amino acid sequence selected from the following:

(SEQ ID NO: 264)
KKIDNFTGCYSLSRTLRFKLIPQGKTQEHIELKRLLDEDEKRAED
YKKAKKIIDKYHITFIDRILHNIKLTELQSYIELDEVSNKDEKQK
KELLKIEGLLRKEISNSFKKDDEYKKIFGKEIIETILPEYLDDQE
EIEIIKSFKGFSTAFVGYWRNRKNLYTDEEIGSSIAYRAINDNLP
KFMRNASIFKSVEKIFSEEELREIKKEVLNDEYDVQDFFRKDFYD
FLLPQDGIDIYNAILGGLVKEDGTKIRGLNEYINLYNQKNKEKNA
KLKPLFKQVLTESESKSFYIDEFAKDEEVIEAFQNTFAEEGEVCL
AIDQIQKLFNQYEIYDSNGIFIKNGLAVTALANDIYGSWSYIKEK
WNTLYDAENTGRTSKESESYITKRENIYKKIESFSLNEIETITEG
EEKLLSEISNIISEKIQLYFEKYNSAKYLLNSDYKLDKKLQKNNK
VVEVIKELMDSVKDIERYLKPFMGTGKEEGRDEYFYSEFENTLEV
LSGIDRLYNKIRNYVTKEPFSKKKFKLYFQNPQFMGGWDRNKEAD
YRVALLRKKQKYYLAIMDKSDSKCLQRVEQGNGDLQKMNYKLISG
ASKSLPHVFFSKKYLSNNNVEDEILRIYKSKTFIKGESFDIDDCH
KLINYYKESIKSHPWGSAFDFSFSDTNEYADIHTFYQEVDDMGYK
IVFDNVSEEAVNKLVDEGKLYLFQIYNKDFSEKSHGTENLHTMYF
KSLFDENNRGNIRLQGGAEMFLRKASLKKQELVVHPANVPMKNKN
KNNPKKTTTLPYDVYKDKRYSEDQYEIHIPIIINKATTNFYKLNL
EIRKLLREDTNPYVIGIDRGERNLLYMVVIDGKGRIVEQYSINEI
VNECKGITIKTDYHSLLDEKEKERMAARKSWKTVENIKELKEGYI
SQVVHKICELVRKYDAVIALEDLNSGFKNSRVKVEKQVYQKFEKM
LIDKLNYMVDKKIPWYENGGVLKGYQLTNKFESFKKMGTQNGMMF
YIPAWLTSKIDPSTGFVNLLRTRYKSVQDSKNFINKISSIKYDKD
EEMFCFAIDYSNFEHTNADYKKKWILYSNGERIKHFRNPEKNSEF
DYKIINLTTEFKNLFEEYNIAYELGEDIKEQINVINEKKFFEKFM
STVTLMLQMRNSITGRTDIDYLISPVKNDTGNFYDSRNFEALENA
VLPKDADANGAYNIARKVLWAIEQFRESDESKLEKVSIAISNKKW
LEYAQK;
(SEQ ID NO: 265)
KKIDNFTGCYSLSRTLRFKLIPQGKTQEHIELKRLLDEDEKRAEDY
KKAKKIIDKYHITFIDRILHNIKLTELQSYIELDEVSNKDEKQKK
ELLKIEGLLRKEISNSFKKDDEYKKIFGKEIIETILPEYLDDQEE
IEIIKSFKGFSTAFVGYWENRKNLYTDEEIGSSIAYRAINDNLPK
FMRNASIFKSVEKIFSEEELREIKKEVLNDEYDVQDFFRKDFYDF
LLPQDGIDIYNAILGGLVKEDGTKIRGLNEYINLYNQKNKEKNAK
LKPLFKQVLTESESKSFYIDEFAKDEEVIEAFQNTFAEEGEVCLA
IDQIQKLFNQYEIYDSNGIFIKNGLAVTALANDIYGSWSYIKEKW
NTLYDAENTGRTSKESESYITKRENIYKKIESFSLNEIETITEGE
EKLLSEISNIISEKIQLYFEKYNSAKYLLNSDYKLDKKLQKNNKV
VEVIKELMDSVKDIERYLKPFMGTGKEEGRDEYFYSEFENTLEVL
SGIDRLYNKIRNYVTKEPFSKKKFKLYFQNPQFMRGWDRNKEADY
RVALLRKKQKYYLAIMDKSDSKCLQRVEQGNGDLQKMNYKLISGA
SKSLPHVFFSKKYLSNNNVEDEILRIYKSKTFIKGESFDIDDCHK
LINYYKESIKSHPWGSAFDFSFSDTNEYADIHTFYQEVDDMGYKI
VFDNVSEEAVNKLVDEGKLYLFQIYNKDFSEKSHGTENLHTMYFK
SLFDENNRGNIRLCGGAEMFLRKASLKKQELVVHPANVPMKNKNK
NNPKKTTTLPYDVYKDKRYSEDQYEIHIPIIINKATTNFYKLNLE
IRKLLREDTNPYVIGIDRGERNLLYMVVIDGKGRIVEQYSINEIV
NECKGITIKTDYHSLLDEKEKERMAARKSWKTVENIKELKEGYIS
QVVHKICELVRKYDAVIALEDLNSGFKNSRVKVEKQVYQKFEKML
IDKLNYMVDKKIPWYENGGVLKGYQLTNKFESFKKMGTQNGMMFY
IPAWLTSKIDPSTGFVNLLRTRYKSVQDSKNFINKISSIKYDKDE
EMFCFAIDYSNFEHTNADYKKKWILYSNGERIKHFRNPEKNSEFD
YKIINLTTEFKNLFEEYNIAYELGEDIKEQINVINEKKFFEKFMS
TVTLMLQMRNSITGRTDIDYLISPVKNDTGNFYDSRNFEALENAV
LPKDADANGAYNIARKVLWAIEQFRESDESKLEKVSIAISNKKWL
EYAQK;
(SEQ ID NO 266)
KKIDNFTGCYSLSRTLRFKLIPQGKTQEHIELKRLLDEDEKRAED
YKKAKKIIDKYHITFIDRILHNIKLTELQSYIELDEVSNKDEKQK
KELLKIEGLLRKEISNSFKKDDEYKKIFGKEIIETILPEYLDDQE
EIEIIKSFKGFSTAFVGYWRNRKNLYTDEEIGSSIAYRAINDNLP
KFMRNASIFKSVEKIFSEEELREIKKEVLNDEYDVQDFFRKDFYD
FLLPQDGIDIYNAILGGLVKEDGTKIRGLNEYINLYNQKNKEKNA
KLKPLFKQVLTESESKSFYIDEFAKDEEVIEAFQNTFAEEGEVCL
AIDQIQKLFNQYEIYDSNGIFIKNGLAVTALANDIYGSWSYIKEK
WNTLYDAENTGRTSKESESYITKRENIYKKIESFSLNEIETITEG
EEKLLSEISNIISEKIQLYFEKYNSAKYLLNSDYKLDKKLQKNNK
VVEVIKELMDSVKDIERYLKPFMGTGKEEGRDEYFYSEFENTLEV
LSGIDRLYNKIRNYVTKEPFSKKKFKLYFQNPQFMRGWDRNKEAD
YRVALLRKKQKYYLAIMDKSDSKCLQRVEQGNGDLQKMNYKLISG
ASKSLPHVFFSKKYLSNNNVEDEILRIYKSKTFIKGESFDIDDCH
KLINYYKESIKSHPWGSAFDFSFSDTNEYADIHTFYQEVDDMGYK
IVFDNVSEEAVNKLVDEGKLYLFQIYNKDFSEKSHGTENLHTMYF
KSLFDENNRGNIRLQGGAEMFLRKASLKKQELVVHPANVPMKNKN
KNNPKKTTTLPYDVYKDKRYSEDQYEIHIPIIINKATTNFYKLNL
EIRKLLREDTNPYVIGIDRGERNLLYMVVIDGKGRIVEQYSINEI
VNECKGITIKTDYHSLLDEKEKERMAARKSWKTVENIKELKEGYI
SQVVHKICELVRKYDAVIALEDLNSGFKNSRVKVEKQVYQKFEKM
LIDKLNYMVDKKIPWYENGGVLKGYQLTNKFESFKKMGTQNGMMF
YIPAWLTSKIDPSTGFVNLLRTRYKSVQDSKNFINKISSIKYDKD
EEMFCFAIDYSNFEHTNADYKKKWILYSNGERIKHFRNPEKNSEF
DYKIINLTTEFKNLFEEYNIAYELGEDIKEQINVINEKKFFEKFM
STVTLMLQMRNSITGRTDIDYLISPVKNDTGNFYDSRNFEALENA
VLPKDADANGAYNIARKVLWAIEQFRESDESKLEKVSIAISNKKW
LEYAQK;
(SEQ ID NO: 267)
KKIDNFTGCYSLSRTLRFKLIPQGKTQEHIELKRLLDEDEKRAED
YKKAKKIIDKYHITFIDRILHNIKLTELQSYIELDEVSNKDEKQK
KELLKIEGLLRKEISNSFKKDDEYKKIFGKEIIETILPEYLDDQE
EIEIIKSFKGFSTAFVGYWRNRKNLYTDEEIGSSIAYRAINDNLP
KFMRNASIFKSVEKIFSEEELREIKKEVLNDEYDVQDFFRKDFYD
FLLPQDGIDIYNAILGGLVKEDGTKIRGLNEYINLYNQKNKEKNA
KLKPLFKQVLTESESKSFYIDEFAKDEEVIEAFQNTFAEEGEVCL
AIDQIQKLFNQYEIYDSNGIFIKNGLAVTALANDIYGSWSYIKEK
WNTLYDAENTGRTSKESESYITKRENIYKKIESFSLNEIETITEG
EEKLLSEISNIISEKIQLYFEKYNSAKYLLNSDYKLDKKLQKNNK
VVEVIKELMDSVKDIERYLKPFMGTGKEEGRDEYFYSEFENTLEV
LSGIDRLYNKIRNYVTKEPFSKKKFKLYFQNPQFMGGWDRNREAD
YRVALLRKKQKYYLAIMDKSDSKCLQRVEQGNGDLQKMNYKLISG
ASKSLPHVFFSKKYLSNNNVEDEILRIYKSKTFIKGESFDIDDCH
KLINYYKESIKSHPWGSAFDFSFSDTNEYADIHTFYQEVDDMGYK
IVFDNVSEEAVNKLVDEGKLYLFQIYNKDFSEKSHGTENLHTMYF
KSLFDENNRGNIRLCGGAEMFLRKASLKKQELVVHPANVPMKNKN
KNNPKKTTTLPYDVYKDKRYSEDQYEIHIPIIINKATTNFYKLNL
EIRKLLREDTNPYVIGIDRGERNLLYMVVIDGKGRIVEQYSINEI
VNECKGITIKTDYHSLLDEKEKERMAARKSWKTVENIKELKEGYI
SQVVHKICELVRKYDAVIALEDLNSGFKNSRVKVEKQVYQKFEKM
LIDKLNYMVDKKIPWYENGGVLKGYQLTNKFESFKKMGTQNGMMF
YIPAWLTSKIDPSTGFVNLLRTRYKSVQDSKNFINKISSIKYDKD
EEMFCFAIDYSNFEHTNADYKKKWILYSNGERIKHFRNPEKNSEF
DYKIINLTTEFKNLFEEYNIAYELGEDIKEQINVINEKKFFEKFM
STVTLMLQMRNSITGRTDIDYLISPVKNDTGNFYDSRNFEALENA
VLPKDADANGAYNIARKVLWAIEQFRESDESKLEKVSIAISNKKW
LEYAQK;
(SEQ ID NO: 268)
KKIDNFTGCYSLSRTLRFKLIPQGKTQEHIELKRLLDEDEKRAED
YKKAKKIIDKYHITFIDRILHNIKLTELQSYIELDEVSNKDEKQK
KELLKIEGLLRKEISNSFKKDDEYKKIFGKEIIETILPEYLDDQE
EIEIIKSFKGFSTAFVGYWENRKNLYTDEEIGSSIAYRAINDNLP
KFMRNASIFKSVEKIFSEEELREIKKEVLNDEYDVQDFFRKDFYD
FLLPQDGIDIYNAILGGLVKEDGTKIRGLNEYINLYNQKNKEKNA
KLKPLFKQVLTESESKSFYIDEFAKDEEVIEAFQNTFAEEGEVCL
AIDQIQKLFNQYEIYDSNGIFIKNGLAVTALANDIYGSWSYIKEK
WNTLYDAENTGRTSKESESYITKRENIYKKIESFSLNEIETITEG
EEKLLSEISNIISEKIQLYFEKYNSAKYLLNSDYKLDKKLQKNNK
VVEVIKELMDSVKDIERYLKPFMGTGKEEGRDEYFYSEFENTLEV
LSGIDRLYNKIRNYVTKEPFSKKKFKLYFQNPQFMGGWDRNKEAD
YRVALLRKKQKYYLAIMDKSDSKCLQRVEQGNGDLQKMNYKLISG
ASKSLPHVFFSKKYLSNNNVEDEILRIYKSKTFIKGESFDIDDCH
KLINYYKESIKSHPWGSAFDFSFSDTNEYADIHTFYQEVDDMGYK
IVFDNVSEEAVNKLVDEGKLYLFQIYNKDFSEKSHGTENLHTMYF
KSLFDENNRGNIRLCGGAEMFLRKASLKKQELVVHPANVPMKNKN
KNNPKKTTTLPYDVYKDKRYSEDQYEIHIPIIINKATTNFYKLNL
EIRKLLREDTNPYVIGIDRGERNLLYMVVIDGKGRIVEQYSINEI
VNECKGITIKTDYHSLLDEKEKERMAARKSWKTVENIKELKEGYI
SQVVHKICELVRKYDAVIALEDLNSGFKNSRVKVEKQVYQKFEKM
LIDKLNYMVDKKIPWYENGGVLKGYQLTNKFESFKKMGTQNGMMF
YIPAWLTSKIDPSTGFVNLLRTRYKSVQDSKNFINKISSIKYDKD
EEMFCFAIDYSNFEHTNADYKKKWILYSNGERIKHFRNPEKNSEF
DYKIINLTTEFKNLFEEYNIAYELGEDIKEQINVINEKKFFEKFM
STVTLMLQMRNAITGRTDIDYLISPVKNDTGNFYDSRNFEALENA
VLPKDADANGAYNIARKVLWAIEQFRESDESKLEKVSIAISNKKW
LEYAQK;
(SEQ ID NO: 269)
KKIDNFTGCYSLSRTLRFKLIPQGKTQEHIELKRLLDEDEKRAED
YKKAKKIIDKYHITFIDRILHNIKLTELQSYIELDEVSNKDEKQK
KELLKIEGLLRKEISNSFKKDDEYKKIFGKEIIETILPEYLDDQE
EIEIIKSFKGFSTAFVGYWENRKNLYTDEEIGSSIAYRAINDNLP
KFMRNASIFKSVEKIFSEEELREIKKEVLNDEYDVQDFFRKDFYD
FLLPQDGIDIYNAILGGLVKEDGTKIRGLNEYINLYNQKNKEKNA
KLKPLFKQVLTESESKSFYIDEFAKDEEVIEAFQNTFAEEGEVCL
AIDQIQKLFNQYEIYDSNGIFIKNGLAVTALANDIYGSWSYIKEK
WNTLYDAENTGRTSKESESYITKRENIYKKIESFSLNEIETITEG
EEKLLSEISNIISEKIQLYFEKYNSAKYLLNSDYKLDKKLQKNNK
VVEVIKELMDSVKDIERYLKPFMGTGKEEGRDEYFYSEFENTLEV
LSGIDRLYNKIRNYVTKEPFSKKKFKLYFQNPQFMGGWDRNKEAD
YRVALLRKKQKYYLAIMDKSDSKCLQRVEQGNGDLQKMNYKLISG
ASKSLPHVFFSKKYLSNNNVEDEILRIYKSKTFIKGESFDIDDCH
KLINYYKESIKSHPWGSAFDFSFSDTNEYADIHTFYQEVDDMGYK
IVFDNVSEEAVNKLVDEGKLYLFQIYNKDFSEKSHGTENLHTMYF
KSLFDENNRGNIRLCGGAEMFLRKASLKKQELVVHPANVPMKNKN
KNNPKKTTTLPYDVYKDKRYSEDQYLIHIPIIINKATTNFYKLNL
EIRKLLREDTNPYVIGIDRGERNLLYMVVIDGKGRIVEQYSINEI
VNECKGITIKTDYHSLLDEKEKERMAARKSWKTVENIKELKEGYI
SQVVHKICELVRKYDAVIALEDLNSGFKNSRVKVEKQVYQKFEKM
LIDKLNYMVDKKIPWYENGGVLKGYQLTNKFESFKKMGTQNGMMF
YIPAWLTSKIDPSTGFVNLLRTRYKSVQDSKNFINKISSIKYDKD
EEMFCFAIDYSNFEHTNADYKKKWILYSNGERIKHFRNPEKNSEF
DYKIINLTTEFKNLFEEYNIAYELGEDIKEQINVINEKKFFEKFM
STVTLMLQMRNSITGRTDIDYLISPVKNDTGNFYDSRNFEALENA
VLPKDADANGAYNIARKVLWAIEQFRESDESKLEKVSIAISNKKW
LEYAQK;
(SEQ ID NO: 270)
KKIDNFTGCYSLSRTLRFKLIPQGKTQEHIELKRLLDEDEKRAED
YKKAKKIIDKYHITFIDRILHNIKLTELQSYIELDEVSNKDEKQK
KELLKIEGLLRKEISNSFKKDDEYKKIFGKEIIETILPEYLDDQE
EIEIIKSFKGFSTAFVGYWRNRKNLYTDEEIGSSIAYRAINDNLP
KFMRNASIFKSVEKIFSEEELREIKKEVLNDEYDVQDFFRKDFYD
FLLPQDGIDIYNAILGGLVKEDGTKIRGLNEYINLYNQKNKEKNA
KLKPLFKQVLTESESKSFYIDEFAKDEEVIEAFQNTFAEEGEVCL
AIDQIQKLFNQYEIYDSNGIFIKNGLAVTALANDIYGSWSYIKEK
WNTLYDAENTGRTSKESESYITKRENIYKKIESFSLNEIETITEG
EEKLLSEISNIISEKIQLYFEKYNSAKYLLNSDYKLDKKLQKNNK
VVEVIKELMDSVKDIERYLKPFMGTGKEEGRDEYFYSEFENTLEV
LSGIDRLYNKIRNYVTKEPFSKKKFKLYFQNPQFMGGWDRNKEAD
YRVALLRKKQKYYLAIMDKSDSKCLQRVEQGNGDLQKMNYKLISG
ASKSLPHVFFSKKYLSNNNVEDEILRIYKSKTFIKGESFDIDDCH
KLINYYKESIKSHPWGSAFDFSFSDTNEYADIHTFYQEVDDMGYK
IVFDNVSEEAVNKLVDEGKLYLFQIYNKDFSEKSHGTENLHTMYF
KSLFDENNRGNIRLQGGAEMFLRKASLKKQELVVHPANVPMKNKN
KNNPKKTTTLPYDVYKDKRYSEDQYLIHIPIIINKATTNFYKLNL
EIRKLLREDTNPYVIGIDRGERNLLYMVVIDGKGRIVEQYSINEI
VNECKGITIKTDYHSLLDEKEKERMAARKSWKTVENIKELKEGYI
SQVVHKICELVRKYDAVIALEDLNSGFKNSRVKVEKQVYQKFEKM
LIDKLNYMVDKKIPWYENGGVLKGYQLTNKFESFKKMGTQNGMMF
YIPAWLTSKIDPSTGFVNLLRTRYKSVQDSKNFINKISSIKYDKD
EEMFCFAIDYSNFEHTNADYKKKWILYSNGERIKHFRNPEKNSEF
DYKIINLTTEFKNLFEEYNIAYELGEDIKEQINVINEKKFFEKFM
STVTLMLQMRNSITGRTDIDYLISPVKNDTGNFYDSRNFEALENA
VLPKDADANGAYNIARKVLWAIEQFRESDESKLEKVSIAISNKKW
LEYAQK;
(SEQ ID NO: 271)
KKIDNFTGCYSLSRTLRFKLIPQGKTQEHIELKRLLDEDEKRAED
YKKAKKIIDKYHITFIDRILHNIKLTELQSYIELDEVSNKDEKQK
KELLKIEGLLRKEISNSFKKDDEYKKIFGKEIIETILPEYLDDQE
EIEIIKSFKGFSTAFVGYWRNRKNLYTDEEIGSSIAYRAINDNLP
KFMRNASIFKSVEKIFSEEELREIKKEVLNDEYDVQDFFRKDFYD
FLLPQDGIDIYNAILGGLVKEDGTKIRGLNEYINLYNQKNKEKNA
KLKPLFKQVLTESESKSFYIDEFAKDEEVIEAFQNTFAEEGEVCL
AIDQIQKLFNQYEIYDSNGIFIKNGLAVTALANDIYGSWSYIKEK
WNTLYDAENTGRTSKESESYITKRENIYKKIESFSLNEIETITEG
EEKLLSEISNIISEKIQLYFEKYNSAKYLLNSDYKLDKKLQKNNK
VVEVIKELMDSVKDIERYLKPFMGTGKEEGRDEYFYSEFENTLEV
LSGIDRLYNKIRNYVTKEPFSKKKFKLYFQRPQFMGGWDRNKEAD
YRVALLRKKQKYYLAIMDKSDSKCLQRVEQGNGDLQKMNYKLISG
ASKSLPHVFFSKKYLSNNNVEDEILRIYKSKTFIKGESFDIDDCH
KLINYYKESIKSHPWGSAFDFSFSDTNEYADIHTFYQEVDDMGYK
IVFDNVSEEAVNKLVDEGKLYLFQIYNKDFSEKSHGTENLHTMYF
KSLFDENNRGNIRLQGGAEMFLRKASLKKQELVVHPANVPMKNKN
KNNPKKTTTLPYDVYKDKRYSEDQYLIHIPIIINKATTNFYKLNL
EIRKLLREDTNPYVIGIDRGERNLLYMVVIDGKGRIVEQYSINEI
VNECKGITIKTDYHSLLDEKEKERMAARKSWKTVENIKELKEGYI
SQVVHKICELVRKYDAVIALEDLNSGFKNSRVKVEKQVYQKFEKM
LIDKLNYMVDKKIPWYENGGVLKGYQLTNKFESFKKMGTQNGMMF
YIPAWLTSKIDPSTGFVNLLRTRYKSVQDSKNFINKISSIKYDKD
EEMFCFAIDYSNFEHTNADYKKKWILYSNGERIKHFRNPEKNSEF
DYKIINLTTEFKNLFEEYNIAYELGEDIKEQINVINEKKFFEKFM
STVTLMLQMRNSITGRTDIDYLISPVKNDTGNFYDSRNFEALENA
VLPKDADANGAYNIARKVLWAIEQFRESDESKLEKVSIAISNKKW
LEYAQK;
(SEQ ID NO: 272)
KKIDNFTGCYSLSRTLRFKLIPQGKTQEHIELKRLLDEDEKRAED
YKKAKKIIDKYHITFIDRILHNIKLTELQSYIELDEVSNKDEKQK
KELLKIEGLLRKEISNSFKKDDEYKKIFGKEIIETILPEYLDDQE
EIEIIKSFKGFSTAFVGYWENRKNLYTDEEIGSSIAYRAINDNLP
KFMRNASIFKSVEKIFSEEELREIKKEVLNDEYDVQDFFRKDFYD
FLLPQDGIDIYNAILGGLVKEDGTKIRGLNEYINLYNQKNKEKNA
KLKPLFKQVLTESESKSFYIDEFAKDEEVIEAFQNTFAEEGEVCL
AIDQIQKLFNQYEIYDSNGIFIKNGLAVTALANDIYGSWSYIKEK
WNTLYDAENTGRTSKESESYITKRENIYKKIESFSLNEIETITEG
EEKLLSEISNIISEKIQLYFEKYNSAKYLLNSDYKLDKKLQKNNK
VVEVIKELMDSVKDIERYLKPFMGTGKEEGRDEYFYSEFENTLEV
LSGIDRLYNKIRNYVTKEPFSKKKFKLYFQRPQFMGGWDRNKEAD
YRVALLRKKQKYYLAIMDKSDSKCLQRVEQGNGDLQKMNYKLISG
ASKSLPHVFFSKKYLSNNNVEDEILRIYKSKTFIKGESFDIDDCH
KLINYYKESIKSHPWGSAFDFSFSDTNEYADIHTFYQEVDDMGYK
IVFDNVSEEAVNKLVDEGKLYLFQIYNKDFSEKSHGTENLHTMYF
KSLFDENNRGNIRLCGGAEMFLRKASLKKQELVVHPANVPMKNKN
KNNPKKTTTLPYDVYKDKRYSEDQYEIHIPIIINKATTNFYKLNL
EIRKLLREDTNPYVIGIDRGERNLLYMVVIDGKGRIVEQYSINEI
VNECKGITIKTDYHSLLDEKEKERMAARKSWKTVENIKELKEGYI
SQVVHKICELVRKYDAVIALEDLNSGFKNSRVKVEKQVYQKFEKM
LIDKLNYMVDKKIPWYENGGVLKGYQLTNKFESFKKMGTQNGMMF
YIPAWLTSKIDPSTGFVNLLRTRYKSVQDSKNFINKISSIKYDKD
EEMFCFAIDYSNFEHTNADYKKKWILYSNGERIKHFRNPEKNSEF
DYKIINLTTEFKNLFEEYNIAYELGEDIKEQINVINEKKFFEKFM
STVTLMLQMRNSITGRTDIDYLISPVKNDTGNFYDSRNFEALENA
VLPKDADANGAYNIARKVLWAIEQFRESDESKLEKVSIAISNKKW
LEYAQK;
(SEQ ID NO: 273)
KKIDNFTGCYSLSRTLRFKLIPQGKTQEHIELKRLLDEDEKRAED
YKKAKKIIDKYHITFIDRILHNIKLTELQSYIELDEVSNKDEKQK
KELLKIEGLLRKEISNSFKKDDEYKKIFGKEIIETILPEYLDDQE
EIEIIKSFKGFSTAFVGYWENRKNLYTDEEIGSSIAYRAINDNLP
KFMRNASIFKSVEKIFSEEELREIKKEVLNDEYDVQDFFRKDFYD
FLLPQDGIDIYNAILGGLVKEDGTKIRGLNEYINLYNQKNKEKNA
KLKPLFKQVLTESESKSFYIDEFAKDEEVIEAFQNTFAEEGEVCL
AIDQIQKLFNQYEIYDSNGIFIKNGLAVTALANDIYGSWSYIKEK
WNTLYDAENTGRTSKESESYITKRENIYKKIESFSLNEIETITEG
EEKLLSEISNIISEKIQLYFEKYNSAKYLLNSDYKLDKKLQKNNK
VVEVIKELMDSVKDIERYLKPFMGTGKEEGRDEYFYSEFENTLEV
LSGIDRLYNKIRNYVTKEPFSKKKFKLYFQRPQFMGGWDRNKEAD
YRVALLRKKQKYYLAIMDKSDSKCLQRVEQGNGDLQKMNYKLISG
ASKSLPHVFFSKKYLSNNNVEDEILRIYKSKTFIKGESFDIDDCH
KLINYYKESIKSHPWGSAFDFSFSDTNEYADIHTFYQEVDDMGYK
IVFDNVSEEAVNKLVDEGKLYLFQIYNKDFSEKSHGTENLHTMYF
KSLFDENNRGNIRLCGGAEMFLRKASLKKQELVVHPANVPMKNKN
KNNPKKTTTLPYDVYKDKRYSEDQYLIHIPIIINKATTNFYKLNL
EIRKLLREDTNPYVIGIDRGERNLLYMVVIDGKGRIVEQYSINEI
VNECKGITIKTDYHSLLDEKEKERMAARKSWKTVENIKELKEGYI
SQVVHKICELVRKYDAVIALEDLNSGFKNSRVKVEKQVYQKFEKM
LIDKLNYMVDKKIPWYENGGVLKGYQLTNKFESFKKMGTQNGMMF
YIPAWLTSKIDPSTGFVNLLRTRYKSVQDSKNFINKISSIKYDKD
EEMFCFAIDYSNFEHTNADYKKKWILYSNGERIKHFRNPEKNSEF
DYKIINLTTEFKNLFEEYNIAYELGEDIKEQINVINEKKFFEKFM
STVTLMLQMRNSITGRTDIDYLISPVKNDTGNFYDSRNFEALENA
VLPKDADANGAYNIARKVLWAIEQFRESDESKLEKVSIAISNKKW
LEYAQK;
(SEQ ID NO: 274)
KKIDNFTGCYSLSRTLRFKLIPQGKTQEHIELKRLLDEDEKRAED
YKKAKKIIDKYHITFIDRILHNIKLTELQSYIELDEVSNKDEKQK
KELLKIEGLLRKEISNSFKKDDEYKKIFGKEIIETILPEYLDDQE
EIEIIKSFKGFSTAFVGYWRNRKNLYTDEEIGSSIAYRAINDNLP
KFMRNASIFKSVEKIFSEEELREIKKEVLNDEYDVQDFFRKDFYD
FLLPQDGIDIYNAILGGLVKEDGTKIRGLNEYINLYNQKNKEKNA
KLKPLFKQVLTESESKSFYIDEFAKDEEVIEAFQNTFAEEGEVCL
AIDQIQKLFNQYEIYDSNGIFIKNGLAVTALANDIYGSWSYIKEK
WNTLYDAENTGRTSKESESYITKRENIYKKIESFSLNEIETITEG
EEKLLSEISNIISEKIQLYFEKYNSAKYLLNSDYKLDKKLQKNNK
VVEVIKELMDSVKDIERYLKPFMGTGKEEGRDEYFYSEFENTLEV
LSGIDRLYNKIRNYVTKEPFSKKKFKLYFQRPQFMGGWDRNKEAD
YRVALLRKKQKYYLAIMDKSDSKCLQRVEQGNGDLQKMNYKLISG
ASKSLPHVFFSKKYLSNNNVEDEILRIYKSKTFIKGESFDIDDCH
KLINYYKESIKSHPWGSAFDFSFSDTNEYADIHTFYQEVDDMGYK
IVFDNVSEEAVNKLVDEGKLYLFQIYNKDFSEKSHGTENLHTMYF
KSLFDENNRGNIRLQGGAEMFLRKASLKKQELVVHPANVPMKNKN
KNNPKKTTTLPYDVYKDKRYSEDQYEIHIPIIINKATTNFYKLNL
EIRKLLREDTNPYVIGIDRGERNLLYMVVIDGKGRIVEQYSINEI
VNECKGITIKTDYHSLLDEKEKERMAARKSWKTVENIKELKEGYI
SQVVHKICELVRKYDAVIALEDLNSGFKNSRVKVEKQVYQKFEKM
LIDKLNYMVDKKIPWYENGGVLKGYQLTNKFESFKKMGTQNGMMF
YIPAWLTSKIDPSTGFVNLLRTRYKSVQDSKNFINKISSIKYDKD
EEMFCFAIDYSNFEHTNADYKKKWILYSNGERIKHFRNPEKNSEF
DYKIINLTTEFKNLFEEYNIAYELGEDIKEQINVINEKKFFEKFM
STVTLMLQMRNSITGRTDIDYLISPVKNDTGNFYDSRNFEALENA
VLPKDADANGAYNIARKVLWAIEQFRESDESKLEKVSIAISNKKW
LEYAQK;
(SEQ ID NO: 275)
AFVICYNTYSRTYLEKEIISMKTIESFCGQKKGYSRSITLRNRLI
PIGKTEENIRKLKLLDKDIDRSKAYVEVKALIDDFHRVFIEDVLS
KTELPWEPLYDQFELYQNEKDKQKKNKIKKDLEELQKGLRKSIVK
QFKADDRFDKLFKKELLTEFVPSVIKNDDSGTISDKQAALDIFKG
FATYFTGFHRNRQNMYSEEAQSTAISNRIVNENFPKFYANIQTFK
YLEENFPQIISDTEQSLSDFLGDKKLKDIFSIDGFNKVLSQSGID
FYNTVIGGISEEAGTQKVQGLNEKINLASQQLSSEEKHKLKKKMT
VLYKQILSDRSTASFIPVGFEKSEEVYDSVKEFKELSLDKNIAAI
NTIFSRDDYDLTRIFVPAKEITEFSLKLFGHWSILQDGLFLLGKD
NAKKDLSEKQITELIKEIAKKDYSLAELQNAYERWTKENDIPVEK
TVKNYFRLAELRTDEKTKEKNFTDIQKELEKAFVQIDFDKKENLI
KEKEAATPIKNFLDEVQNLFHYLKLVDYRGEEEKDSDFYAKYDQV
LQALAEIIPLYNKVRNFVTKKPNEVKKVKLNFECSSFLRGWGTDY
GTKEAHIFIDDGKYYLGIVNEKLSKEDIKFLEEKSPRMIKKVVYD
FQKPDNKNTPRLFIRSKGTSYAPSVSQYDLPIESVIEIYDKGLFK
TEYRKVNPSVYKESLVKMIDYFKLGFTRHESYKHYNFSWKDSKDY
NDISEFYADVMNSCYQLKFEYINYDNLLSLVDAGKLFLFQIYNKD
FSTGKDGANGSTGKKNLHTLYWENLFSEENLKDICLKLNGEAELF
WRDVNPNIKNICHKKGSILVNRTTSDGKVIPEDIYQEIYKFKNPD
KQEKDFKISDEAKALLENGKVVCKEAKFDITKDRHFTQQTYLFHC
PITMSFKAPEITGRKFNEKVQSILKANPDVKIIGLDRGERHLIYL
SLINQKGEIELQKTLNLVDQVRNDKTVSVNYQEKLVHKEGERDKA
RKNWQSISNIKELKEGYLSNVVHEIAKLMVENNAIVVMEDLNFGF
KRGRFAVERQVYQKFENMLIEKLNYLVFKDKAVTEPGGVLNAYQL
TDKSANVSDVGKQCGWIFYVPAAYTSKIDPKTGFANLFYTAGLTN
VEKKKEFFDKFEAIRYDSKTDSFVLSFDYKDFSDNADYKKKWSLY
SRGERLVFSKAEKNVISVNPTENLKALFDKQGINWKSEENFIDQI
NAVQAERENVSFFDGLYRSFTAILQMRNSVPNSSKMEDDYLISPV
MAEDGTFYDSRKEAAKGKDEQGKWISKLPVDADANGAYHIALKGL
YLLQNDFNVNDKGYIDNISNADWFEFAQEKNYAK;
(SEQ ID NO: 276)
AFVICYNTYSRTYLEKEIISMKTIESFCGQKKGYSRSITLRNRLI
PIGKTEENIRKLKLLDKDIDRSKAYVEVKALIDDFHRVFIEDVLS
KTELPWEPLYDQFELYQNEKDKQKKNKIKKDLEELQKGLRKSIVK
QFKADDRFDKLFKKELLTEFVPSVIKNDDSGTISDKQAALDIFKG
FATYFTGFHRNRQNMYSEEAQSTAISNRIVNENFPKFYANIQTFK
YLEENFPQIISDTEQSLSDFLGDKKLKDIFSIDGFNKVLSQSGID
FYNTVIGGISEEAGTQKVQGLNEKINLASQQLSSEEKHKLKKKMT
VLYKQILSDRSTASFIPVGFEKSEEVYDSVKEFKELSLDKNIAAI
NTIFSRDDYDLTRIFVPAKEITEFSLKLFGHWSILQDGLFLLGKD
NAKKDLSEKQITELIKEIAKKDYSLAELQNAYERWTKENDIPVEK
TVKNYFRLAELRTDEKTKEKNFTDIQKELEKAFVQIDFDKKENLI
KEKEAATPIKNFLDEVQNLFHYLKLVDYRGEEEKDSDFYAKYDQV
LQALAEIIPLYNKVRNFVTKKPNEVKKVKLNFECSSFLSGWGTDY
GTKEAHIFIDDGKYYLGIVNEKLSKEDIKFLEEKSPRMIKKVVYD
FQKPDNKNTPRLFIRSKGTSYAPSVSQYDLPIESVIEIYDKGLFK
TEYRKVNPSVYKESLVKMIDYFKLGFTRHESYKHYNFSWKDSKDY
NDISEFYADVMNSCYQLKFEYINYDNLLSLVDAGKLFLFQIYNKD
FSTGKDGANGSTGKKNLHTLYWENLFSEENLKDICLKLNGEAELF
WRDVNPNIKNICHKKGSILVNRTTSDGKVIPEDIYQEIYKFKNPD
KQEKDFKISDEAKALLENGKVVCKEAKFDITKDRHFTQQTYLFHC
PITMSFKAPEITGRKFNEKVQSILKANPDVKIIGLDRGERHLIYL
SLINQKGEIELQKTLNLVDQVRNDKTVSVNYQEKLVHKEGERDKA
RKNWQSISNIKELKEGYLSNVVHEIAKLMVENNAIVVMEDLNFGF
KRGRFAVERQVYQKFENMLIEKLNYLVFKDKAVTEPGGVLNAYQL
TDKSANVSDVGKQCGWIFYVPAAYTSKIDPKTGFANLFYTAGLTN
VEKKKEFFDKFEAIRYDSKTDSFVLSFDYKDFSDNADYKKKWSLY
SRGERLVFSKAEKNVISVNPTENLKALFDKQGINWKSEENFIDQI
NAVQAERENVSFFDGLYRSFTAILQMRNSVPNSSKMEDDYLISPV
MAEDGTFYDSRKEAAKGKDEQGKWISKLPVDADANGAYHIALKGL
YLLQNDFNVNDKGYIDNISNADWFEFAQEKNYAK;
(SEQ ID NO: 277)
AFVICYNTYSRTYLEKEIISMKTIESFCGQKKGYSRSITLRNRLI
PIGKTEENIRKLKLLDKDIDRSKAYVEVKALIDDFHRVFIEDVLS
KTELPWEPLYDQFELYQNEKDKQKKNKIKKDLEELQKGLRKSIVK
QFKADDRFDKLFKKELLTEFVPSVIKNDDSGTISDKQAALDIFKG
FATYFTGFHQNRQNMYSEEAQSTAISNRIVNENFPKFYANIQTFK
YLEENFPQIISDTEQSLSDFLGDKKLKDIFSIDGFNKVLSQSGID
FYNTVIGGISEEAGTQKVQGLNEKINLASQQLSSEEKHKLKKKMT
VLYKQILSDRSTASFIPVGFEKSEEVYDSVKEFKELSLDKNIAAI
NTIFSRDDYDLTRIFVPAKEITEFSLKLFGHWSILQDGLFLLGKD
NAKKDLSEKQITELIKEIAKKDYSLAELQNAYERWTKENDIPVEK
TVKNYFRLAELRTDEKTKEKNFTDIQKELEKAFVQIDFDKKENLI
KEKEAATPIKNFLDEVQNLFHYLKLVDYRGEEEKDSDFYAKYDQV
LQALAEIIPLYNKVRNFVTKKPNEVKKVKLNFECSSFLRGWGTDY
GTKEAHIFIDDGKYYLGIVNEKLSKEDIKFLEEKSPRMIKKVVYD
FQKPDNKNTPRLFIRSKGTSYAPSVSQYDLPIESVIEIYDKGLFK
TEYRKVNPSVYKESLVKMIDYFKLGFTRHESYKHYNFSWKDSKDY
NDISEFYADVMNSCYQLKFEYINYDNLLSLVDAGKLFLFQIYNKD
FSTGKDGANGSTGKKNLHTLYWENLFSEENLKDICLKLNGEAELF
WRDVNPNIKNICHKKGSILVNRTTSDGKVIPEDIYQEIYKFKNPD
KQEKDFKISDEAKALLENGKVVCKEAKFDITKDRHFTQQTYLFHC
PITMSFKAPEITGRKFNEKVQSILKANPDVKIIGLDRGERHLIYL
SLINQKGEIELQKTLNLVDQVRNDKTVSVNYQEKLVHKEGERDKA
RKNWQSISNIKELKEGYLSNVVHEIAKLMVENNAIVVMEDLNFGF
KRGRFAVERQVYQKFENMLIEKLNYLVFKDKAVTEPGGVLNAYQL
TDKSANVSDVGKQCGWIFYVPAAYTSKIDPKTGFANLFYTAGLTN
VEKKKEFFDKFEAIRYDSKTDSFVLSFDYKDFSDNADYKKKWSLY
SRGERLVFSKAEKNVISVNPTENLKALFDKQGINWKSEENFIDQI
NAVQAERENVSFFDGLYRSFTAILQMRNSVPNSSKMEDDYLISPV
MAEDGTFYDSRKEAAKGKDEQGKWISKLPVDADANGAYHIALKGL
YLLQNDFNVNDKGYIDNISNADWFEFAQEKNYAK;
(SEQ ID NO: 278)
AFVICYNTYSRTYLEKEIISMKTIESFCGQKKGYSRSITLRNRLI
PIGKTEENIRKLKLLDKDIDRSKAYVEVKALIDDFHRVFIEDVLS
KTELPWEPLYDQFELYQNEKDKQKKNKIKKDLEELQKGLRKSIVK
QFKADDRFDKLFKKELLTEFVPSVIKNDDSGTISDKQAALDIFKG
FATYFTGFHRNRQNMYSEEAQSTAISNRIVNENFPKFYANIQTFK
YLEENFPQIISDTEQSLSDFLGDKKLKDIFSIDGFNKVLSQSGID
FYNTVIGGISEEAGTQKVQGLNEKINLASQQLSSEEKHKLKKKMT
VLYKQILSDRSTASFIPVGFEKSEEVYDSVKEFKELSLDKNIAAI
NTIFSRDDYDLTRIFVPAKEITEFSLKLFGHWSILQDGLFLLGKD
NAKKDLSEKQITELIKEIAKKDYSLAELQNAYERWTKENDIPVEK
TVKNYFRLAELRTDEKTKEKNFTDIQKELEKAFVQIDFDKKENLI
KEKEAATPIKNFLDEVQNLFHYLKLVDYRGEEEKDSDFYAKYDQV
LQALAEIIPLYNKVRNFVTKKPNEVKKVKLNFECSSFLSGWGTDR
GTKEAHIFIDDGKYYLGIVNEKLSKEDIKFLEEKSPRMIKKVVYD
FQKPDNKNTPRLFIRSKGTSYAPSVSQYDLPIESVIEIYDKGLFK
TEYRKVNPSVYKESLVKMIDYFKLGFTRHESYKHYNFSWKDSKDY
NDISEFYADVMNSCYQLKFEYINYDNLLSLVDAGKLFLFQIYNKD
FSTGKDGANGSTGKKNLHTLYWENLFSEENLKDICLKLNGEAELF
WRDVNPNIKNICHKKGSILVNRTTSDGKVIPEDIYQEIYKFKNPD
KQEKDFKISDEAKALLENGKVVCKEAKFDITKDRHFTQQTYLFHC
PITMSFKAPEITGRKFNEKVQSILKANPDVKIIGLDRGERHLIYL
SLINQKGEIELQKTLNLVDQVRNDKTVSVNYQEKLVHKEGERDKA
RKNWQSISNIKELKEGYLSNVVHEIAKLMVENNAIVVMEDLNFGF
KRGRFAVERQVYQKFENMLIEKLNYLVFKDKAVTEPGGVLNAYQL
TDKSANVSDVGKQCGWIFYVPAAYTSKIDPKTGFANLFYTAGLTN
VEKKKEFFDKFEAIRYDSKTDSFVLSFDYKDFSDNADYKKKWSLY
SRGERLVFSKAEKNVISVNPTENLKALFDKQGINWKSEENFIDQI
NAVQAERENVSFFDGLYRSFTAILQMRNSVPNSSKMEDDYLISPV
MAEDGTFYDSRKEAAKGKDEQGKWISKLPVDADANGAYHIALKGL
YLLQNDFNVNDKGYIDNISNADWFEFAQEKNYAK;
and
(SEQ ID NO: 279)
AFVICYNTYSRTYLEKEIISMKTIESFCGQKKGYSRSITLRNRLI
PIGKTEENIRKLKLLDKDIDRSKAYVEVKALIDDFHRVFIEDVLS
KTELPWEPLYDQFELYQNEKDKQKKNKIKKDLEELQKGLRKSIVK
QFKADDRFDKLFKKELLTEFVPSVIKNDDSGTISDKQAALDIFKG
FATYFTGFHRNRQNMYSEEAQSTAISNRIVNENFPKFYANIQTFK
YLEENFPQIISDTEQSLSDFLGDKKLKDIFSIDGFNKVLSQSGID
FYNTVIGGISEEAGTQKVQGLNEKINLASQQLSSEEKHKLKKKMT
VLYKQILSDRSTASFIPVGFEKSEEVYDSVKEFKELSLDKNIAAI
NTIFSRDDYDLTRIFVPAKEITEFSLKLFGHWSILQDGLFLLGKD
NAKKDLSEKQITELIKEIAKKDYSLAELQNAYERWTKENDIPVEK
TVKNYFRLAELRTDEKTKEKNFTDIQKELEKAFVQIDFDKKENLI
KEKEAATPIKNFLDEVQNLFHYLKLVDYRGEEEKDSDFYAKYDQV
LQALAEIIPLYNKVRNFVTKKPNEVKKVKLNFECSSFLRGWGTDR
GTKEAHIFIDDGKYYLGIVNEKLSKEDIKFLEEKSPRMIKKVVYD
FQKPDNKNTPRLFIRSKGTSYAPSVSQYDLPIESVIEIYDKGLFK
TEYRKVNPSVYKESLVKMIDYFKLGFTRHESYKHYNFSWKDSKDY
NDISEFYADVMNSCYQLKFEYINYDNLLSLVDAGKLFLFQIYNKD
FSTGKDGANGSTGKKNLHTLYWENLFSEENLKDICLKLNGEAELF
WRDVNPNIKNICHKKGSILVNRTTSDGKVIPEDIYQEIYKFKNPD
KQEKDFKISDEAKALLENGKVVCKEAKFDITKDRHFTQQTYLFHC
PITMSFKAPEITGRKFNEKVQSILKANPDVKIIGLDRGERHLIYL
SLINQKGEIELQKTLNLVDQVRNDKTVSVNYQEKLVHKEGERDKA
RKNWQSISNIKELKEGYLSNVVHEIAKLMVENNAIVVMEDLNFGF
KRGRFAVERQVYQKFENMLIEKLNYLVFKDKAVTEPGGVLNAYQL
TDKSANVSDVGKQCGWIFYVPAAYTSKIDPKTGFANLFYTAGLTN
VEKKKEFFDKFEAIRYDSKTDSFVLSFDYKDFSDNADYKKKWSLY
SRGERLVFSKAEKNVISVNPTENLKALFDKQGINWKSEENFIDQI
NAVQAERENVSFFDGLYRSFTAILQMRNSVPNSSKMEDDYLISPV
MAEDGTFYDSRKEAAKGKDEQGKWISKLPVDADANGAYHIALKGL
YLLQNDFNVNDKGYIDNISNADWFEFAQEKNYAK.

### Nuclear Localization Signals; NLSs

### Example 6, Nuclear Localization Signals

Nuclear Localization Signals; NLSs.

### Example 7, Nuclear Localization Signals, Sequences

In Example 6, nuclear localization signals have an amino acid sequence selected from the following or are any combination of one or more amino acid sequences selected from the following:

(SEQ ID NO: 69)
PKKKRKVKRPAATKKAGQAKKKK;
(SEQ ID NO: 70)
KRPAATKKAGQAKKKKPAAKRVKLDGGGGSGGGGSGGGGSPAAKRVKLD;
(SEQ ID NO: 71)
PKKKRKVPKKKRKVPKKKRKV;
(SEQ ID NO: 72)
PAAKRVKLD;
(SEQ ID NO: 73)
RQRRNELKRSP;
(SEQ ID NO: 74)
NQSSNFGPMKGGNFGGRSSGPYGGGGQYFAKPRNQGGY;
(SEQ ID NO: 75)
RMRIZFKNKGKDTAELRRRRVEVSVELRKAKKDEQILKRRNV;
(SEQ ID NO: 76)
VSRKRPRP;
(SEQ ID NO: 77)
PPKKARED;
(SEQ ID NO: 78)
PQPKKKPL;
(SEQ ID NO: 79)
SALIKKKKKMAP;
(SEQ ID NO: 80)
DRLRR;
(SEQ ID NO: 81)
PKQKKRK;
(SEQ ID NO: 82)
RKLKKKIKKL;
(SEQ ID NO: 83)
REKKKFLKRR;
(SEQ ID NO: 84)
KRKGDEVDGVDEVAKKKSKK;
(SEQ ID NO: 85)
RKCLQAGMNLEARKTKK;
(SEQ ID NO: 86)
PKTKRKV;
(SEQ ID NO: 87)
PAAKTKRKVLD;
(SEQ ID NO: 88)
PAAKTKRLD;
(SEQ ID NO: 89)
KRXXXXXXXXXPKTKRKV;
(SEQ ID NO: 90)
KRXXXXXXXXXXKKKKLD;
(SEQ ID NO: 91)
AAAKKKLD;
(SEQ ID NO: 92)
PAAKKKKLD;
(SEQ ID NO: 93)
PAAKKKK;
and
(SEQ ID NO: 94)
PAAKRVKLD.

### Example 8, Nuclear Localization Signals, Meaning of Combination

In Example 7, “any combination of one or more amino acid sequences” means that one or more amino acid sequences are linked directly or via a linker selected from the following:

(SEQ ID NO: 95)
GGGGSGGGGSGGGGS;
(SEQ ID NO: 96)
GGGGSGGGGS;
(SEQ ID NO: 97)
GGGGS;
(SEQ ID NO: 98)
GGGGGG;
(SEQ ID NO: 99)
GGGGGGGG;
(SEQ ID NO: 100)
EAAAK;
(SEQ ID NO: 101)
EAAAKEAAAK;
(SEQ ID NO: 102)
EAAAKEAAAKEAAAK;
(SEQ ID NO: 103)
AEAAAKEAAAKEAAAKEAAAKALEAEAAAKEAAAKEAAAKEAAAKA;
(SEQ ID NO: 104)
PAPAP;
(SEQ ID NO: 105)
AEAAAKEAAAKA;
(SEQ ID NO: 106)
GGGGSGGGGGGGGSGGGGS;
(SEQ ID NO: 107)
VSQTSKLTRAETVFPDV;
(SEQ ID NO: 108)
PLGLWA;
(SEQ ID NO: 109)
RVLAEA;
(SEQ ID NO: 110)
EDVVCCSMSY;
(SEQ ID NO: 111)
GGIEGRGS;
(SEQ ID NO: 112)
TRHRQPRQWE;
(SEQ ID NO: 113)
AGNRVRRSVG;
(SEQ ID NO: 114)
RRRRRRRRR;
and
(SEQ ID NO: 115)
GFLG.

Cas12a Proteins with Nuclear Localization Signals Linked

### Example 9, Cas12a Proteins with Nuclear Localization Signals Linked

Cas12a proteins with nuclear localization signals linked, the Cas12a proteins including the following:
any Cas12a protein of Example 1 or any Cas12a protein variant of Examples 2 to 5; and
one or more nuclear localization signals selected from Examples 6 to 8.

### Example 10, Linkage Relationship

In Example 9, the Cas12a proteins or the Cas12a protein variants are directly linked to one or more nuclear localization signals or are linked via amino acid sequences of SEQ ID NOs: 95 to 115.

### Example 11, Structural Formula

Cas12a proteins with nuclear localization signals linked, have the following structure:

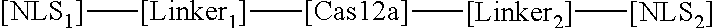

wherein, the NLS₁ is a nuclear localization signal from any of Examples 6 to 8 or is absent,
the NLS₂ is a nuclear localization signal from any of Examples 6 to 8 or is absent,
the Linker₁ is a linker having an amino acid sequence selected from SEQ ID NOs: 67 to 115 or is absent, and
the Linker₂ is a linker having an amino acid sequence selected from SEQ ID NOs: 67 to 115 or is absent, and
the Cas12a is any Cas12a protein of Example 1 or any Cas12a protein variant of Examples 2 to 5, and
the Cas12a protein with the nuclear localization signals linked includes one or more nuclear localization signals.

### Example 12, Sequences

In any of Examples 9 to 11, the Cas12a proteins with the nuclear localization signals linked include an amino acid sequence selected from SEQ ID NOs: 280 to 298.

### Programmable Guide RNAs

### Example 13, Programmable Guide RNAs

Each of the programmable guide RNAs includes the following:
a scaffold,
wherein, the scaffold may interact with any Cas12a protein from Example 1 or a variant of the Cas12a protein from any of Examples 2 to 5 to form a complex; and
a guide domain,
wherein, the guide domain is designed to target a target nucleic acid.

### Example 14, Scaffold

In Example 13, the scaffold includes a direct repeat (DR), and
the direct repeat has a nucleic
acid sequence selected from UAAUUUCUACUAAGUGUAGAU (SEQ ID NO: 33), AAAAUUUCUACUCUUGUAGAU (SEQ ID NO: 34), and UAAAUUUCUACUGUUGUAGAU (SEQ ID NO: 35).

### Example 15, Meaning of Targeting Target Nucleic Acids

In any of Examples 13 to 14, the meaning of the statement “guide domain targets a target nucleic acid” means the following:
a target nucleic acid is a double-stranded DNA including a target strand and a non-target strand, and
the guide domain and the target nucleic acid have a relationship selected from the following:
the nucleic acid sequence of a guide domain is a sequence complementary to all or part of the nucleic acid sequence of a target strand;
a guide domain hybridizes to or binds complementarily with all or part of a target strand;
the nucleic acid sequence of a guide domain is equivalent to the nucleic acid sequence of all or part of a non-target strand; or
any combination of the relationships.

### Example 16, Guide Domain Length

In any of Examples 13 to 15, the guide domain has a length selected from the following:
12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, or 30 nt; or

a length within a range of the two numbers described above, for example, 16 nt to 23 nt.

### Example 17, Mismatches Included

In Example 15, a guide domain hybridizes to or binds complementarily with all or part of its target strand, and
the nucleic acid sequence of a guide domain and the nucleic acid sequence in the portion of a target strand that hybridizes to or binds complementarily with the guide domain have a relationship selected from the following:
a relationship in which the nucleic acid sequences are completely complementary to each other;
a relationship in which the nucleic acid sequences are complementary except for 1, 2, 3, 4, or 5 mismatches;
a relationship in which one or more RNA budge portions that do not hybridize to the target strand are present in the nucleic acid sequence of the guide domain;
a relationship in which one or more DNA bulge portions that do not hybridize to the guide domain are present in the nucleic acid sequence of the target strand; or
any combination of the above relationships.

### Example 18, Guide Domain Sequence Homology

In Example 15, the nucleic acid sequence of a guide domain includes a nucleic acid sequence selected from the following:
a nucleic acid sequence that is 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more, or 100% complementary to all or part of the nucleic acid sequence of a target strand; or
a nucleic acid sequence that is 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more, or 100% homologous, identical, or equivalent to all or part of the nucleic acid sequence of a non-target strand.

### Example 19, Possibility of Including Stem-Loop

In any of Examples 13 to 18, a scaffold includes a stem-loop having a sequence of SEQ ID NO: 49.

### Example 20, Structure of Guide RNAs

In any of Examples 13 to 19,
the programmable guide RNAs may have a structure in which scaffolds and guide domains
are connected sequentially from the 5′ end to the 3′ end.
wherein, the 3′ end of the scaffolds and the 5′ end of the guide domains are connected directly or via arbitrary RNA sequences.

### Example 21, Structural Formula Limited

In any of Examples 13 to 20, the programmable guide RNAs have the following structure:

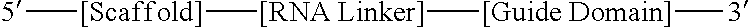

wherein, the RNA linker is any RNA sequence or is absent, and
in the absence of the RNA linker, the structure is equivalent to the following structure:

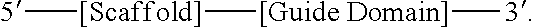

### Example 22, Additional Domains Included

In Example 21, the programmable guide RNAs further include one or more additional domains, and
the additional domains are RNAs and are connected to the 5′ end, the 3′ end, or both the 5′ and 3′ ends of the programmable guide RNAs.

### Example 23, Stem-Loop Included

In Example 22, the programmable guide RNAs further include a stem-loop, and the stem-loop is connected to the 5′ end of the programmable guide RNAs.

### Example 24, Stem-Loop Sequences

In Example 23, the stem-loop includes the nucleic acid sequence of SEQ ID NO: 49.

### CRISPR/Cas12a Complexes

### Example 25, CRISPR/Cas12a Complexes

A CRISPR CRISPR/Cas12a Complex includes the following:
a Cas12a protein from Example 1, a variant of the Cas12a protein from any of Examples 2 to 5, or a Cas12a protein with a nuclear localization signal linked from any of Examples 9 to 12 (hereinafter, collectively referred to as Cas12a protein); and
a programmable guide RNA from any of Examples 13 to 24,
wherein the programmable guide RNA binds with a Cas12a protein to form a CRISPR/Cas12a complex, and
the CRISPR/Cas12a complex has the function of cleaving a target nucleic acid targeted by a guide domain of the programmable guide RNA, or a nucleic acid adjacent thereto.

Hereinafter, the CRISPR/Cas12a complex is also referred to as an RNA-protein complex or ribonucleoprotein (RNP).

### Example 26, Cas12a Protein and Scaffold Combinations

In Example 25,
the Cas12a protein and the scaffold of the programmable guide RNA are selected from the following combinations:
a protein having an amino acid sequence selected from SEQ ID NO: 1 and SEQ ID NOS: 264 to 274 or a protein having the amino acid sequence and with nuclear localization signals linked among Cas12a proteins, and a scaffold having a direct repeat sequence of SEQ ID NO: 33;
a protein having an amino acid sequence selected from SEQ ID NO: 3 and SEQ ID NOS: 275 to 279 or a protein having the amino acid sequence and with nuclear localization signals linked among Cas12a proteins, and a scaffold having a direct repeat sequence of SEQ ID NO: 35; or
a protein having an amino acid of SEQ ID NO: 2 or a protein having the amino acid sequence and with a nuclear localization signal linked among Cas12a proteins, and a scaffold having a direct repeat sequence of SEQ ID NO: 34.

### CRISPR/Cas12a Vectors

### Example 27, CRISPR/Cas12a Vectors

A CRISPR/Cas12a system component expression vector includes the following:
a nucleic acid encoding any of the following: a Cas12a protein from Example 1, a variant of the Cas12a protein from any of Examples 2 to 5, or a Cas12a protein with a nuclear localization signal linked from any of Examples 9 to 12 (hereinafter, collectively referred to as Cas12a protein); and
a nucleic acid encoding a programmable guide RNA from any of Examples 13 to 24,
wherein, the programmable guide RNA binds with a Cas12a protein to form a CRISPR/Cas12a complex, and
the CRISPR/Cas12a complex has the function of cleaving a target nucleic acid, targeted by a guide domain of the programmable guide RNA, or a nucleic acid adjacent thereto.

### Example 28, Cas12a Encoding DNAs

In Example 27, the nucleic acid encoding a Cas12a protein includes a nucleic acid sequence selected from SEQ ID NOs: 17 to 19.

### Example 29, Additional Components Included

In any of Examples 27 to 28, the component expression vector further includes one or more selected from the following:
a promoter; enhancer; intron; polyadenylation signal; Kozak consensus sequence; Internal Ribosome Entry Site (IRES); splice acceptor; 2A sequence; and replication origin.

### Example 30, Promoter Addition

In Example 29, the promoter includes one or more selected from the following:
SV40 early promoter; mouse mammary tumor virus long terminal repeat (LTR) promoter; adenovirus major late promoter (Ad MLP); herpes simplex virus (HSV) promoter; cytomegalovirus (CMV) promoter, such as the CMV immediate early promoter region (CMVIE); Rous sarcoma virus (RSV) promoter; human U6 small nuclear promoter (U6) (Miyagishi et al.; Nature Biotechnology 20; 497-500 (2002)); enhanced U6 promoter (e.g.; Xia et al.; Nucleic Acids Res. 2003 Sep. 1; 31 (17)); human H1 promoter (H1); and 7SK.

### Example 31, Addition of Origins of Replication

In any of Examples 29 to 30, origins of replication are one or more selected from the following:
f1 replication origin; SV40 origin of replication; pMB1 origin of replication; adeno origin of replication; AAV origin of replication; and BBV origin of replication.

### Example 32, Vector Type

In any of Examples 27 to 31, the vector includes a viral vector, a non-viral vector, or any combination thereof.

### Example 33, Viral Vectors

In Example 32, the vector includes one or more selected from the following:
retrovirus; lentivirus; adenovirus; adeno-associated virus; vaccinia virus; poxvirus; Herpes simplex virus; and any combination thereof.

### Example 34, Non-Viral Vectors

In Example 32, the vector includes non-viral vectors selected from the following:
plasmid; phage; naked DNA; DNA complex; PCR amplicon; mRNA; and any combination thereof.

### Example 35, Plasmid

In Example 34, the plasmid includes any one selected from the following:
pcDNA series; pS456; p326; pACYC177; ColE1; pKT230; pME290; pBR322; pUC8/9; pUC6; pBD9; pHC79; plJ61; pLAFR1; pHV14; pGEX series; pET series; pUC19; and any combination thereof.

### Example 36, Phage

In Example 34, the phage includes any one selected from the following:
λgt4λB; λ-Charon; λΔz1; M13; and any combination thereof.

### Example 37, Single Vectors

In any of Examples 27 to 36, the vector is single vector of one molecule.

### Example 38, Multiple Vectors

In any of Examples 27 to 37, the vector is a plurality of vectors of two or more molecules.

### Example 39, Each Component Contained in Different Vector

In Example 38, nucleic acids encoding the Cas12a proteins and nucleic acids encoding the programmable guide RNAs are contained in different molecular vectors.

### Example 40, DNA and RNA Specification

In any of Examples 27 to 39, the nucleic acids encoding the Cas12a proteins and the nucleic acids encoding the programmable guide RNAs are each independently DNA or RNA.

### Example 41, Cas12a Protein and Programmable Guide RNA Combinations

In any of Examples 27 to 40, the Cas12a protein and the scaffold of the programmable guide RNA are selected from the following combinations:
a protein having an amino acid sequence selected from SEQ ID NO: 1 and SEQ ID NOS: 264 to 274 or a protein having the amino acid sequence and with nuclear localization signals linked among Cas12a proteins, and a scaffold having a direct repeat sequence of SEQ ID NO: 33;
a protein having an amino acid sequence selected from SEQ ID NO: 3 and SEQ ID NOS: 275 to 279 or a protein having the amino acid sequence and with nuclear localization signals linked among Cas12a proteins, and a scaffold having a direct repeat sequence of SEQ ID NO: 35; or
a protein having an amino acid of SEQ ID NO: 2 or a protein having the amino acid sequence and with a nuclear localization signal linked among Cas12a proteins, and a scaffold having a direct repeat sequence of SEQ ID NO: 34.

### CRISPR/Cas12a Compositions

### Example 42, CRISPR/Cas12a Compositions

A CRISPR CRISPR/Cas12a Composition includes the following:
a Cas12a protein from Example 1, a variant of the Cas12a protein from any of Examples 2 to 5, or a Cas12a protein with a nuclear localization signal linked from any of Examples 9 to 12 (hereinafter, collectively referred to as Cas12a protein), or a nucleic acid encoding any of the Cas12a proteins; and
a programmable guide RNA from any of Examples 13 to 24 or a nucleic acid encoding the guide RNA,
wherein, the programmable guide RNA binds with a Cas12a protein to form a CRISPR/Cas12a complex, and
the CRISPR/Cas12a complex has the function of cleaving a target nucleic acid targeted by a guide domain of the programmable guide RNA, or a nucleic acid adjacent thereto.

### Example 43, CRISPR/Cas12a Complexes Included

In Example 42, the CRISPR/Cas12a composition includes the CRISPR/Cas12a complexes from any of Examples 25 to 26.

### Example 44, CRISPR/Cas12a Vectors Included

In Example 42, the CRISPR/Cas12a composition includes expression vectors for CRISPR/Cas12a system components from any of Examples 27 to 41.

### Example 45, Cas Encoding mRNAs Included

In Example 42, the CRISPR/Cas12a composition includes mRNAs encoding the Cas12a proteins, and programmable guide RNAs.

### Example 46, Cas12a Protein and Programmable Guide RNA Combinations

In any of Examples 42 to 45, the Cas12a protein and the scaffold of the programmable guide RNA are selected from the following combinations:
a protein having an amino acid sequence selected from SEQ ID NO: 1 and SEQ ID NOS: 264 to 274 or a protein having the amino acid sequence and with nuclear localization signals linked among Cas12a proteins, and a scaffold having a direct repeat sequence of SEQ ID NO: 33;
a protein having an amino acid sequence selected from SEQ ID NO: 3 and SEQ ID NOS: 275 to 279 or a protein having the amino acid sequence and with nuclear localization signals linked among Cas12a proteins, and a scaffold having a direct repeat sequence of SEQ ID NO: 35; or
a protein having an amino acid of SEQ ID NO: 2 or a protein having the amino acid sequence and with a nuclear localization signal linked among Cas12a proteins, and a scaffold including a direct repeat sequence of SEQ ID NO: 34.

### Compositions for Editing Eukaryotic Genomes

### Example 47, Compositions for Editing Eukaryotic Genomes

A composition for editing eukaryotic genome includes the following:
a Cas12a protein from Example 1, a variant of the Cas12a protein from any of Examples 2 to 5, or a Cas12a protein with a nuclear localization signal linked from any of Examples 9 to 12 (hereinafter, collectively referred to as Cas12a protein), or a nucleic acid encoding any of the Cas12a proteins; and
a programmable guide RNA from any one of Examples 13 to 24, or a nucleic acid encoding the guide RNA,
wherein, the programmable guide RNA binds with a Cas12a protein to form a CRISPR/Cas12a complex, and
the CRISPR/Cas12a complex has the function of cleaving a target nucleic acid in the eukaryotic genome, targeted by a guide domain of the programmable guide RNA, or a nucleic acid adjacent thereto.

### Example 48, CRISPR/Cas12a Complexes Included

In Example 47, the CRISPR/Cas12a composition includes the CRISPR/Cas12a complexes from any of Examples 25 to 26.

### Example 49, CRISPR/Cas12a Vectors Included

In Example 47, the CRISPR/Cas12a composition includes an expression vector for any CRISPR/Cas12a system component from any of Examples 27 to 41.

### Example 50, Cas Encoding mRNAs Included

In Example 47, the CRISPR/Cas12a compositions includes mRNAs encoding the Cas12a proteins, and programmable guide RNAs.

### Example 51, Cas12a Protein and Programmable Guide RNA Combinations

In any of Examples 47 to 50, the Cas12a protein and the scaffold of the programmable guide RNA are selected from the following combinations:
a protein having an amino acid sequence selected from SEQ ID NO: 1 and SEQ ID NOS: 264 to 274 or a protein having the amino acid sequence and with nuclear localization signals linked among Cas12a proteins, and a scaffold having a direct repeat sequence of SEQ ID NO: 33;
a protein having an amino acid sequence selected from SEQ ID NO: 3 and SEQ ID NOS: 275 to 279 or a protein having the amino acid sequence and with nuclear localization signals linked among Cas12a proteins, and a scaffold having a direct repeat sequence of SEQ ID NO: 35; or
a protein having an amino acid of SEQ ID NO: 2 or a protein having the amino acid sequence and with a nuclear localization signal linked among Cas12a proteins, and a scaffold having a direct repeat sequence of SEQ ID NO: 34.

### Method for Editing Eukaryotic Genomes

### Example 52, Gene Editing Method #1—Composition Delivery

A method for editing eukaryotic genomes includes the following:
delivering a CRISPR/Cas12a complex from any of Examples 25 to 26, a CRISPR/Cas12a component expression vector from any of Examples 27 to 41, a CRISPR/Cas12a composition from any of Examples 42 to 46, or a composition for editing eukaryotic genes from any of Examples 47 to 51 into the eukaryotic cell,
wherein, guide domains of the programmable guide RNAs are designed to target a target nucleic acid in the eukaryotic genome.

### Example 53, Alternative Expressions for Delivery

In Example 52, “delivery to eukaryotic cells” is replaced by the following expressions:
introduction into eukaryotic cells; administration into eukaryotic cells; injection into eukaryotic cells; transfection into eukaryotic cells; transduction into eukaryotic cells; or a combination of the expressions.

### ####Example 54, Gene Editing Method #2—Complex Contact

A method for editing eukaryotic genomes includes the following:
a process of making contact or inducing contact between a CRISPR/Cas12a complex from any of Examples 25 to 26 and a genome of the eukaryotic cell,
wherein, the guide domains of the programmable guide RNAs are designed to target their target nucleic acids in the eukaryotic genome.

### Example 55, Delivery Method Limited

In any of Examples 52 to 54, the delivery of the eukaryotic gene editing compositions to the eukaryotic cells is performed by a method selected from the following:
electroporation; gene gun; ultrasonic perforation; magnetofection; temporary cell compression or squeezing; cationic liposome method; Lithium acetate-DMSO; lipid-mediated transfection; Calcium phosphate precipitation; lipofection; Polyethyleneimine (PEI)-mediated transfection; DEAE-dextran mediated transfection; nanoparticle-mediated nucleic acid delivery (See Panyam et., al Adv Drug Deliv Rev. 2012 Sep. 13. pii: S0169-409X (12) 00283-9. doi: 10.1016/j.addr.2012.09.023); or a combination of the methods.

### Example 56, Relationships to PAM Sequences

In any of Examples 52 to 55,
the target nucleic acids are double-stranded DNAs, and
the double-stranded DNAs include the following: a protospacer adjacent motif (PAM) sequence, a first strand (non-target strand) having a protospacer sequence adjacent to the PAM, and a second strand (target strand) having a nucleic acid sequence complementary to the first strand, and
guide domains of the programmable guide RNAs have nucleic acid sequences that are about 80% or more, about 81% or more, about 82% or more, about 83% or more, about 84% or more, about 85% or more, about 86% or more, about 87% or more, about 88% or more, about 89% or more, about 90% or more, about 91% or more, about 92% or more, about 93% or more, about 94% or more, about 95% or more, about 96% or more, about 97% or more, about 98% or more, about 99% or more, or 100% matched, identical, equivalent, and/or homologous to the protospacer sequence.

### Example 57, PAM Sequences

In Example 56,
the PAM sequences are 5′-TTTV-3′ or 5′-TTTN-3′, wherein V is A, C, or G, and N is A, C, G, or T.

### Example 58, Eukaryotic Cell Type

In a method from any of Examples 52 to 57, the eukaryotic cells are selected from plant cells, non-human animal cells, and human cells.

### Example 59, Human Cells

In Example 58, the eukaryotic cells are human induced Pluripotent Stem (iPS) cells.

### Example 60, Isolated Cells

In any of Examples 52 to 59, the eukaryotic cells are cells that are isolated from an organism.

### Example 61, Method Performance Environment

In any of Examples 52 to 60, the method is performed in vivo, in vitro, and/or ex vivo.

### Method for Cleaving Nucleic Acids in Cytoplasm

### Example 62, Method for Cleaving Nucleic Acids in Eukaryotic Cell Cytoplasm

A method for cleaving nucleic acids in the eukaryotic cell cytoplasm includes the following:
delivering a CRISPR/Cas12a composition to the eukaryotic cell,
wherein, the CRISPR/Cas12a composition includes the following:
a Cas12a protein from Example 1, a variant of the Cas12a protein from any of Examples 2 to 5 (hereinafter, collectively referred to as Cas12a protein), or a nucleic acid encoding any of the Cas12a proteins; and
a programmable guide RNA from any of Examples 13 to 24 or a nucleic acid encoding the programmable guide RNA,
wherein, the Cas12a protein does not include a nuclear localization signal,
the programmable guide RNA binds with a Cas12a protein to form a CRISPR/Cas12a complex, and
the CRISPR/Cas12a complex bound with the guide domain of the programmable guide RNA targets a nucleic acid in the eukaryotic cell cytoplasm.

### Example 63, Cas12a Protein and Guide RNA Combinations

In Example 62, the Cas12a protein and the scaffold of the programmable guide RNA are selected from the following combinations:
a protein having an amino acid sequence selected from SEQ ID NO: 1 and SEQ ID NOS: 264 to 274 or a protein having the amino acid sequence and with nuclear localization signals linked among Cas12a proteins, and a scaffold having a direct repeat sequence of SEQ ID NO: 33;
a protein having an amino acid sequence selected from SEQ ID NO: 3 and SEQ ID NOS: 275 to 279 or a protein having the amino acid sequence and with nuclear localization signals linked among Cas12a proteins, and a scaffold having a direct repeat sequence of SEQ ID NO: 35; or
a protein having an amino acid of SEQ ID NO: 2 or a protein having the amino acid sequence and with a nuclear localization signal linked among Cas12a proteins, and a scaffold having a direct repeat sequence of SEQ ID NO: 34.

### Example 64, Expansion of Delivery Expressions

In any of Examples 62 to 63, the “delivery to eukaryotic cells” is replaced by the following expressions:
introduction into eukaryotic cells; administration into eukaryotic cells; injection into eukaryotic cells; transfection into eukaryotic cells; transduction into eukaryotic cells; or a combination of the expressions.

### Multiplex Vector Construct

### Example 65, Vector Constructs

A multiplex vector construct includes the following:
a promoter;
a DNA encoding a first guide RNA; and
a DNA encoding a second guide RNA,
wherein, the first guide RNA is any one selected from Examples 13 to 24,
the second guide RNA is any one selected from Examples 13 to 24,
the DNA encoding the first guide RNA is operably linked to a promoter,
the 5′ end of the DNA encoding the second guide RNA is directly connected to the 3′ end of
the first guide RNA, and
the first guide domain and the second guide domain have different nucleic acid sequences.

### Example 66, 3 or More Guide RNAs Included

Each of the multiplex vector constructs contains the following structure:

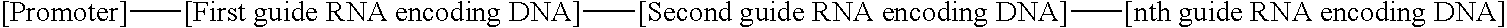

wherein, n is an integer of 3 or more,
the first guide RNA-encoding DNA, the second guide RNA-encoding DNA, . . . , the nth guide RNA encoding DNA are each independently a DNA encoding any one programmable guide RNA selected from Examples 13 to 24,
the first guide RNA encoding DNA is operably linked to the promoter, and
the first guide RNA-encoding DNA, the second guide RNA-encoding DNA, . . . , the nth guide RNA encoding DNA are directly connected.

### Method for Cleaving Multiple Target Nucleic Acids

### Example 67, Method for Cleaving Two or More Target Nucleic Acids

A method for cleaving two or more target nucleic acids in the eukaryotic genome includes the following:
delivering a multiplex editing composition to the eukaryotic cell,
wherein, the multiplex editing composition includes the following:
a vector construct from Example 65; and
a Cas12a protein from Example 1, a variant of the Cas12a protein from any of Examples 2 to 5, a Cas12a protein with a nuclear localization signal linked from any of Examples 9 to 12 (hereinafter, collectively referred to as Cas12a protein), or a nucleic acid encoding the Cas12a protein,
wherein, the first guide RNA and the second guide RNA contained in the vector construct each bind with a Cas12a protein to form a CRISPR/Cas12a complex,
as a result of performing the method, target nucleic acids, targeted by the first guide RNA and the second guide RNA, respectively, are edited.

### Example 68, Method for Cleaving Three or More Target Nucleic Acids

A method for cleaving three or more target nucleic acids in the eukaryotic genome includes the following:
delivering a multiplex editing composition to the eukaryotic cell,
wherein, the multiplex editing composition includes the following:
a vector construct from Example 66; and
a Cas12a protein from Example 1, a variant of the Cas12a protein from any of Examples 2 to 5, a Cas12a protein with a nuclear localization signal linked from any of Examples 9 to 12 (hereinafter, collectively referred to as Cas12a protein), or a nucleic acid encoding the Cas12a protein,
wherein, each guide RNA contained in the vector construct binds with a Cas12a protein to form a CRISPR/Cas12a complex, and
as a result of performing the method, more target nucleic acids than the number of types of guide RNA (i.e., n) contained in the vector construct are edited.

### Example 69, Delivery Order Irrelevant

In any of Examples 67 and 68, the vector construct and the Cas12a protein or the nucleic acid encoding the Cas12a protein are delivered to the eukaryotic cell simultaneously or in random order.

### Example 70, Cas12a Protein and Guide Combinations

In any of Examples 67 to 69, the Cas12a protein and the scaffold for the guide RNA are selected from the following combinations:
a protein having an amino acid sequence selected from SEQ ID NO: 1 and SEQ ID NOS: 264 to 274 or a protein having the amino acid sequence and with nuclear localization signals linked among Cas12a proteins, and a scaffold having a direct repeat sequence of SEQ ID NO: 33;
a protein having an amino acid sequence selected from SEQ ID NO: 3 and SEQ ID NOS: 275 to 279 or a protein having the amino acid sequence and with nuclear localization signals linked among Cas12a proteins, and a scaffold having a direct repeat sequence of SEQ ID NO: 35; or
a protein having an amino acid of SEQ ID NO: 2 or a protein having the amino acid sequence and with a nuclear localization signal linked among Cas12a proteins, and a scaffold having a direct repeat sequence of SEQ ID NO: 34.

### Example 71, Expansion of Delivery Expressions

In any of Examples 67 to 70, the “delivery to eukaryotic cells” is replaced by the following expressions:
introduction into eukaryotic cells; administration into eukaryotic cells; injection into eukaryotic cells; transfection into eukaryotic cells; transduction into eukaryotic cells; or a combination of the expressions.

### EXPERIMENTAL EXAMPLES

Hereinafter, the present disclosure provided by this specification will be described in more detail through experimental examples and examples. These examples are intended solely to illustrate the content disclosed by this specification. It will be apparent to those skilled in the art that the scope of the content disclosed by this specification is not to be construed as limited by these examples.

### Experimental Example 1. Experimental Methods and Materials

### Experimental Example 1.1. CRISPR Enzyme Expression Vector Preparation #1

Coding sequences for Cas12a proteins were codon-optimized for E. coli using a Twist Bioscience algorithm, and the gene was cloned into the pET21 (+) or pET28 vector at the BamHI and XhoI sites. Nucleic acids that might express a 6-His-tag and NLS were added to the PET21 vectors. Through this, NLSs were finally linked to the N-terminus and/or C-terminus of the Cas12a proteins. Vectors that might express CRISPR enzymes with a 6-His-tag attached to their C-terminus were prepared.

### Experimental Example 1.2. Guide RNA Preparation

Synthesis of sgRNAs was performed by in vitro transcription using a MEGAscript™ T7 transcription kit (ThermoFisher Scientific). Synthetic fragments were PCR-amplified (CloneAmp HiFi PCR Premix, Takara) to generate templates for sgRNA transcription. Then, the DNA templates were removed with DNaseI (ThermoFisher Scientific), and the transcribed RNA products were washed with a PCR purification kit (MEGAclear™ Transcription Clean-Up Kit) and eluted in nuclease-free water. The concentration and purity of the RNAs were determined by NanoDrop. RNA integrity was visualized by Ethidium Bromide staining of reaction products separated on a 2% agarose gel with 1× Tris-acetate EDTA (TAE) buffer.

### Experimental Example 1.3. CRISPR Enzyme Preparation and Purification

The vectors prepared according to Experimental Example 1.1 were expressed in E. coli strain (NiCo21 (DE3), T7 Express lysY/lq Competent E. coli; or BL21 (DE3), Competent cells from ThermoFisher Scientific, #EC0114). Briefly, E. coli cells were transformed with plasmid vectors according to the manufacturer's instructions. Single colonies were grown overnight in Luria-Bertani medium containing 100 μg ml-1 ampicillin at 37_. The cells were diluted 1:200 with 250 ml of the same medium and grown at 37_ until OD600=0.70-0.75. The cultures were incubated for 1 hour at room temperature and then incubated at 23_ for 16 to 18 hours with shaking (220 rpm). Thereby, protein expression was induced with 0.5 mM isopropyl-b-D-1-thiogalactopyranoside. Cell pellets were analyzed for protein expression using 10% SDS-PAGE. Protein purification was performed using the Ni-NTA Fast start kit (Qiagen; 30600) and then purified in HiTrap HP SP (GE Healthcare, 17115101). The amount of protein produced as a result of the incubation was measured by SDS-PAGE analysis. Purified Cas12a proteins were stored at −80° C. in a mixture of 20 mM sodium acetate (pH 6.0), 500 mM NaCl, 0.1 mM EDTA, 0.1 mM TCEP, and 50% (v/v) glycerol.

### Experimental Example 1.4. Verification of Prepared CRISPR Enzymes

The CRISPR enzyme prepared according to Experimental Example 1.3 was verified by Restriction Digest Analysis. Briefly, 1 μg of plasmid sample (NiCo21 (DE3), T7 Express lysY/lq Competent E. coli; or BL21 (DE3) Competent cells) was mixed with 1 μl of restriction enzyme, 2 μl of digestion buffer and nuclease-free water, making the final volume of the mixture set to 20 μl. Then, the mixture was incubated at 37_ for 30 minutes. The reaction products separated from there on a 1% agarose gel using 1×TAE (Tris-acetate EDTA) buffer were visualized by Ethidium Bromide staining.

### Experimental Example 1.5. CRISPR/Cas12a Complex Preparation

In NEB r2.1 buffer containing 50 mM NaCl, 10 mM Tris-HCl, 10 mM MgCl2, 100 μg/mL BSA (pH=7.9), 0.5 μM (or 20 ng) of CRISPR enzymes were incubated with 1 μM (or 5 ng) guide RNAs for 15 minutes at room temperature, individually pre-assembling RNP complexes.

### Experimental Example 1.6. Cell Culture

HEK293T (ATCC, CRL-11268) was used to evaluate in vivo cleavage activity for the CRISPR/Cas12a systems. The HEK cells were cultured at 37° C. and 5% CO₂ concentration in high-glucose Dulbecco's modified Eagle's medium (Hyclone, SH30243.01) supplemented with 10% fetal bovine serum (Gibco, 16000-044) and 1% penicillin-streptomycin (ThermoFisher, 15070063).

### Experimental Example 1.7. Measurement of In Vivo Gene Editing Activity #1

The CRISPR/Cas12a complexes prepared according to Experimental Example 1.5 were transfected into HEK293T cells cultured according to Experimental Example 1.6 using the Lipofectamine 2000 system (Thermo, 11668019). After culturing the transfected cells for 3 days, the cells were harvested and their genomic DNA was extracted with the TIANamp genomic DNA kit (Tiagen; 4992254) for T7E1 analysis (NEB; M0302S). For the genomic DNA of the extracted cells, gene editing activity was measured through T7E1 analysis (NEB; M0302S). The experimental results were confirmed through NSG analysis.

### Experimental Example 1.8. Measurement of In Vivo Gene Editing Activity #2

The CRISPR/Cas12a complexes prepared according to Experimental Example 1.5 were transfected into HEK293T cells cultured according to Experimental Example 1.6 using the Neon® Transfection System (ThermoFisher Scientific, MPK1096). After thawing the cells, the cells were subcultured five times, and 1×106 cells were treated at a ratio of (CRISPR enzyme:guide RNA=4:1) and used for cell transfection according to the manufacturer's protocol under the following conditions: Pulse voltage 1500V; Pulse Width: 30 ms; Number of pulses: 1. Afterward, the cells were cultured for 3 days at 37° C. under 5% CO₂ environment.

The cells cultured in 48-well plates were harvested with a mixture containing 5 μl Proteinase K and 100 μl of lysis buffer containing 1 M Tris-HCl (pH 7.5), 0.5 M EDTA (pH=8.0) and 10% SDS. The cell lysis solutions were then incubated at 50° C. for 1 hour, 80° C. for 20 minutes, and stored at 4° C. overnight. PCR reactions were performed in PrimeSTAR GXL DNA polymerase buffer (Takara, #R050A), and NSG analysis was performed on an iSeq100 (Illumnia). NSG results were verified through a web tool (http://www.rgenome.net/).

### Experimental Example 1.9. Plasmid Construction for Mammalian Cell Experiments

pCMV-dLbCpf1-BE-YE1 (Addgene, #154145) was modified to express a new Cas12a protein using Gibson assembly (NEBuilder HiFi DNA Assembly Cloning Kit, NEB #E5520). Novel Cas12a sequences were codon-optimized for humans using Integrated DNA Technologies (IDT)'s Codon Optimization Tool and ordered as a gBlock from IDT. Novel Cas12a variants were constructed using site directed mutagenesis (Q5-site directed mutagenesis kit, NEB #E0554S). crRNA-encoding plasmids were constructed by inserting PCR-amplified oligonucleotides into pLb-Cpf1-pGL3-U6-sgRNA (Addgene, #107682) using Gibson assembly (NEBuilder HiFi DNA Assembly Cloning Kit, NEB #E5520).

### Experimental Example 1.10. Protein Purification

The novel Cas12a proteins and plasmids encoding the novel Cas12a proteins were prepared according to Experimental Example 1.9. Each protein was constructed to include His6 at the N terminus. Rosetta-expressing cells (EMD Millipore) were transformed with the plasmids. Selected transformants were cultured overnight in Luria-Bertani (LB) medium containing 100 μg/ml kanamycin at 37° C. Ten ml of overnight cultured cells were inoculated into 400 ml LB medium containing 100 μg/ml kanamycin and incubated at 30° C. until the OD600 reached 0.5 to 0.6. The cell cultures were cooled to 16° C. for 1 hour, supplemented with 0.5 mM IPTG, and incubated for 14 to 18 hours. For protein purification, the cells were harvested by centrifugation at 5000 g for 10 min at 4° C. The cells were lysed by sonication in 5 ml of lysis buffer (50 mM NaH₂PO₄, 300 mM NaCl, 1 mM dithiothreitol (DTT), 10 mM imidazole, pH 8.0) supplemented with lysozyme (Sigma) and proteolysis inhibitors (Roche complete, EDTA-free). The soluble lysate obtained after centrifugation at 18,000 g for 30 minutes at 4° C. was incubated with Ni-NTA agarose resin (Qiagen) for 1 hour at 4° C. The lysate/Ni-NTA mixture was applied to the column and washed with buffer (50 mM NaH₂PO₄, 300 mM NaCl, 20 mM imidazole, pH 8.0). The novel Cas12a proteins (and their variants) were eluted with elution buffer (50 mM NaH₂PO₄, 300 mM NaCl, 250 mM imidazole, pH 8.0). The buffers for the eluted protein solutions were exchanged for storage buffer (20 mM HEPES-KOH (pH 7.5), 150 mM KCl, 1 mM DTT, 20% glycerol), and then the proteins were concentrated using a centrifugal filtration device (Millipore).

### Experimental Example 1.11. In Vitro Plasmid Cleavage and Analysis

To verify the nuclease function of the novel Cas12a, 1 μg of PCR amplification products containing a target sequence were incubated with ribonucleoprotein complexes at 37° C. To generate ribonucleoprotein complexes, the purified novel Cas12a (300 mM) was incubated with in vitro transcribed crRNA (300 mM) for 15 minutes. Thereafter, these ribonucleoprotein complexes were incubated with PCR amplification products for 8 hours. After incubation, the cleaved amplification products were purified using a PCR Product Purification Kit (MG Med, MK12020) and then subjected to agarose gel electrophoresis. Band intensity was measured using ImageJ software.

### Experimental Example 1.12. Targeted Deep Sequencing

To analyze editing frequency, the target regions were amplified by nested first and second PCRs, followed by a third PCR using Tru-Seq HT Dual index-containing primers and PrimeSTAR® GXL DNA Polymerase (TAKARA), to generate deep sequencing libraries. The library was sequenced using an Illumina iSeq paired-end sequencing system. Base edit and unwanted indel frequencies were expressed as a percentage of sequencing reads containing correct edits or indels out of total sequencing reads. The computer program used to analyze editing frequency was available on the prime_editor_analysis site on GitHub.

### Experimental Example 2. Confirmation of In Vivo Gene Editing Activity of CRISPR/Cas12a Systems

### Experimental Example 2.1. CRISPR/Cas12a Compositions Used in Experiments and Gene Editing Activity Confirmation Method

To determine whether the CRISPR/Cas12a systems disclosed herein may edit genes in eukaryotic cells, in vivo gene editing activity experiments were performed targeting an EMX1 gene of immortalized HEK cells.

A target nucleic acid sequence within the EMX1 gene is as follows:

(SEQ ID NO: 136)
CTCATCTGTGCCCCTCCCTCCCTG

The composition of each CRISPR/Cas12a complexes used in the experiments are shown in the following table:

TABLE 1
Composition of Each of CRISPR/Cas12a Complexes Used in Experiments:

| | | CRISPR | | |
|---|---|---|---|---|
| | Cas12a | enzyme | | |
| | protein | (Cas12a + NLS) | | |
| | (SEQ | (SEQ ID | guide RNA | Guide Domain of |
| Label | ID NO) | NO) | Direct repeat of | guide RNA |
| ID204 | 4 | 125 | UAAUUUCUACUAUUCGUAG | CUCAUCUGUGCCCCUCCCUCCCU |
| | | | AU (SEQ ID NO: 36) | G (SEQ ID NO: 137) |
| ID206 | 5 | 126 | UUAAUUUCUACUUUCGUAG | SEQ ID NO: 137 |
| | | | AU (SEQ ID NO: 37) | |
| ID210 | 6 | 127 | CAAUUUCUACUUUCAGUAG | SEQ ID NO: 137 |
| | | | AU (SEQ ID NO: 38) | |
| ID211 | 7 | 128 | UUAAUUUCUACUUGUGUAG | SEQ ID NO: 137 |
| | | | AU (SEQ ID NO: 39) | |
| ID218 | 1 | 120 | UAAUUUCUACUAAGUGUAG | SEQ ID NO: 137 |
| | | | AU (SEQ ID NO: 33) | |
| ID219 | 9 | 129 | UAAAUUUCUACUAUUGUAG | SEQ ID NO: 137 |
| | | | AU (SEQ ID NO: 41) | |
| ID223 | 10 | 130 | AAAAUUUCUACUAUUGUAG | SEQ ID NO: 137 |
| | | | AU (SEQ ID NO: 42) | |
| ID224 | 11 | 131 | AAAAUUUCUACUAUUGUAG | SEQ ID NO: 137 |
| | | | AU (SEQ ID NO: 43) | |
| ID230 | 12 | 132 | UAAAUUUCUACUAUUGUAG | SEQ ID NO: 137 |
| | | | AU (SEQ ID NO: 44) | |
| ID238 | 13 | 133 | AUAAUUUCUACUGUUGUAG | SEQ ID NO: 137 |
| | | | AU (SEQ ID NO: 45) | |
| ID245 | 15 | 134 | AUAAUUUCUACUGUUGUAG | SEQ ID NO: 137 |
| | | | AU (SEQ ID NO: 47) | |

Among the compositions of the CRISPR/Cas12a complexes, the sequences of Cas12a proteins were obtained from KR 2023-0111399 A, and the compositions of the guide RNAs were determined through additional research by the inventors of the present disclosure.

The specific experimental method was as follows:
1) CRISPR enzyme expression vectors were prepared according to Experimental Example 1.1, and using these, CRISPR enzymes were prepared and purified according to Experimental Example 1.3.
2) Guide RNAs were prepared according to Experimental Example 1.2.
3) Using the CRISPR enzymes and guide RNAs prepared above, CRISPR/Cas12a complexes were prepared according to Experimental Example 1.5.
4) The in vivo gene editing activity was measured according to Experimental Example 1.7, using the CRISPR/Cas12a complexes described above and the Immortalized HEK cells prepared according to Experimental Example 1.6.

### Experimental Example 2.2. CRISPR/Cas12a Preparation Results Used in Experiments

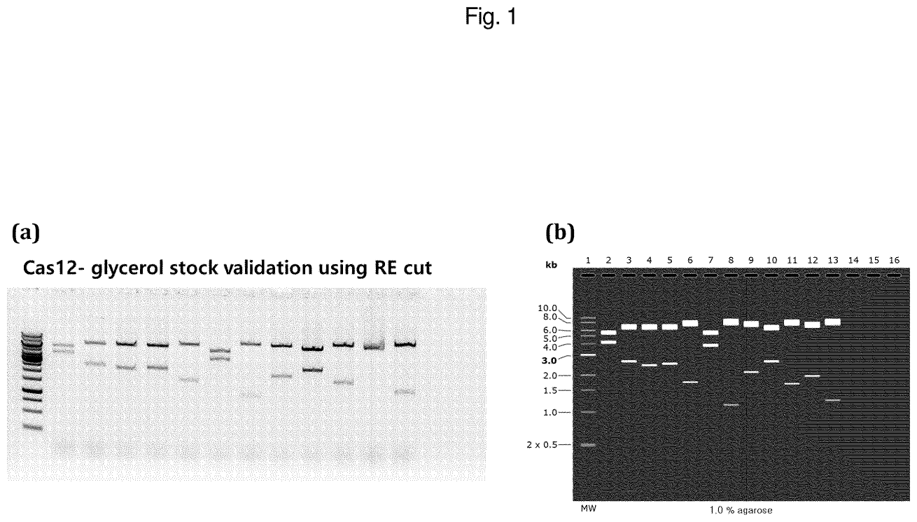

The results of expressing the CRISPR enzyme according to Experimental Example 2.1 are shown in FIG. 1. In FIG. 1, the Label, Restriction enzyme, and fragment bp for each defense are shown in the following table.

TABLE 2

| | | Restriction | Fragment 1 | Fragment 2 | Fragment 3 |
|---|---|---|---|---|---|
| No | Label | Enzyme | (bp) | (bp) | (bp) |
| 1 | 1 kb DNA | — | — | — | — |
| | Ladder | | | | |
| 2 | ID204 | EcoRV | 1.5447 | 2.4083 | — |
| 3 | ID206 | EcoRV | 1.6587 | 2.2625 | 3.102 |
| 4 | ID210 | SphI | 1.6672 | 2.2438 | — |
| 5 | ID211 | EcoRV | 1.6587 | 2.2523 | — |
| 6 | ID218 | HindIII | 1.7342 | 2.1738 | — |
| 7 | ID219 | EcoRV | 1.5444 | 2.3786 | — |
| 8 | ID223 | KpnI | 1.8008 | 2.1138 | — |
| 9 | ID224 | EcoRV | 1.7437 | 2.2087 | — |
| 10 | ID230 | SphI | 1.6519 | 2.2648 | — |
| 11 | ID233 | HindIII | 1.7688 | 2.1695 | — |
| 12 | ID238 | MluI | 1.7181 | 2.1932 | — |
| 13 | ID245 | HindIII | 1.799 | 2.1249 | — |

Looking at the results, it was confirmed that each Cas12a protein was well expressed according to Experimental Example 2.1.

### Experimental Example 2.3. Results of In Vivo Gene Editing Activity Experiments

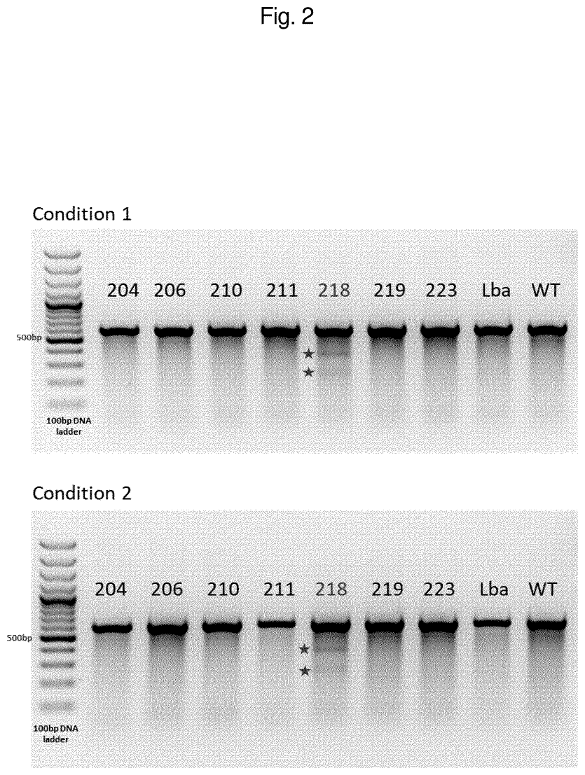

The results of the T7E1 assay performed according to Experimental Example 2.1 are shown in FIG. 2.

As a result of the experiments, it was confirmed that only the CRISPR/Cas12a system including the Cas12a protein of ID218 edited the EMX1 target in the HEK cell, and a related band appeared.

To quantitatively confirm the results, the results of NGS sequence analysis are shown in the following table:

TABLE 3
NGS Results for HEK Cell Endogenous
EMX1 Gene Editing Activity:

| ID | Total Read | Wild type | Insertion | Deletion | In-del (%) | |
|---|---|---|---|---|---|---|
| 204 | 5017 | 5017 | 0 | 0 | 0.0 | (0.0%) |
| 206 | 4478 | 4478 | 0 | 0 | 0.0 | (0.0%) |
| 210 | 4192 | 4192 | 0 | 0 | 0.0 | (0.0%) |
| 211 | 5144 | 5144 | 0 | 2 | 2 | (0.0%) |
| 218 | 3636 | 3244 | 13 | 379 | 392 | (10.8%) |
| 219 | 4652 | 4646 | 0 | 6 | 6 | (0.0%) |
| 223 | 5248 | 5248 | 0 | 0 | 0.0 | (0.0%) |
| 224 | 3912 | 3912 | 0 | 0 | 0.0 | (0.0%) |
| 230 | 4101 | 4101 | 0 | 0 | 0.0 | (0.0%) |
| 238 | 5485 | 5485 | 0 | 0 | 0.0 | (0.0%) |
| 245 | 6172 | 6172 | 0 | 0 | 0.0 | (0.0%) |
| LB | 5147 | 5147 | 0 | 64 | 64 | (1.2%) |

As a result of the experiments, as confirmed in FIG. 2, it was confirmed that only the CRISPR/Cas12a system including the Cas12a protein of ID218 induced the generation of indels in the endogenous EMX1 gene of the HEK cells. Therefore, it could be concluded that the CRISPR/Cas12a system including the Cas12a protein of ID218 had eukaryotic gene editing activity.

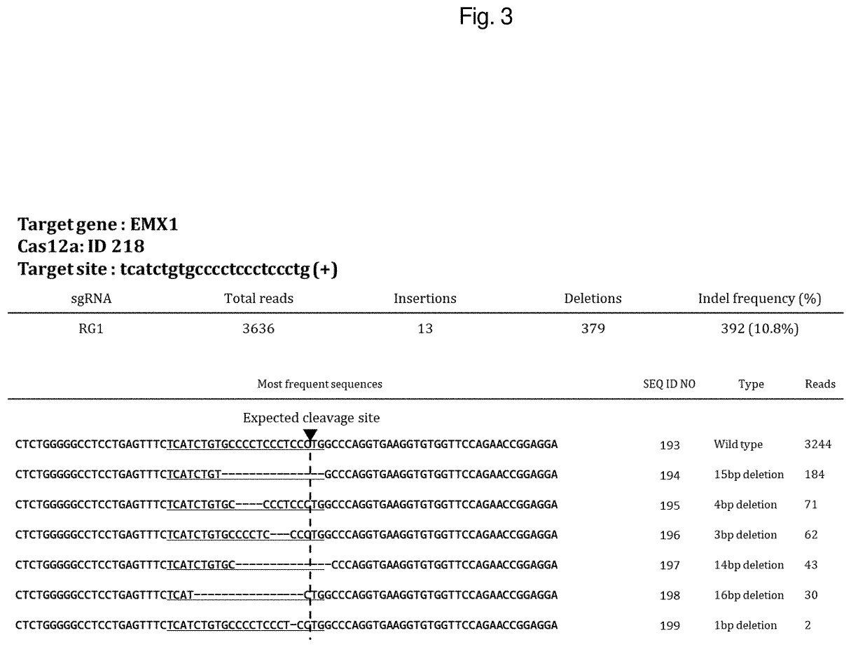

Furthermore, mutation analysis was performed on the indel introduction results and is shown in FIG. 3.

It was confirmed that the CRISPR/Cas12a system including the Cas12a protein of ID218 caused various types of mutations in the endogenous EMX1 gene of HEK cells.

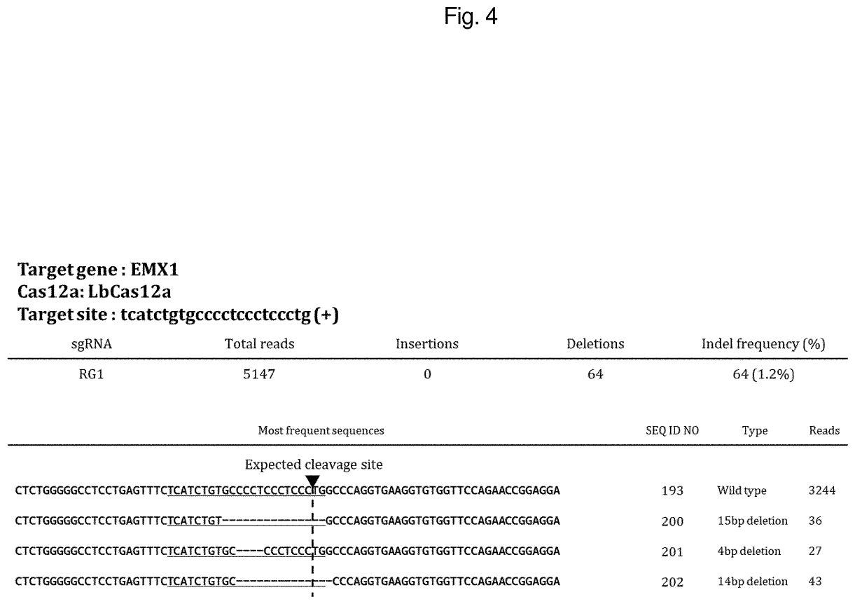

For reference, the results of Mutation Analysis for LbCas12a used as a control are shown in FIG. 4.

### Experimental Example 3. Confirmation of Gene Editing Activity According to Nuclear Localization Signal Compositions

### Experimental Example 3.1. CRISPR/Cas12a Compositions Used in Experiments and Gene Editing Activity Confirmation Method

As confirmed in Experimental Example 2, the CRISPR/Cas12a system including the Cas12a protein of ID218 exhibited eukaryotic cell editing activity. Furthermore, to examine the influence of nuclear localization signals on eukaryotic cell editing activity, the in vivo gene editing activity of the CRISPR/Cas12a systems including the Cas12a protein of ID218 and various combinations of NLSs was measured.

As in Experimental Example 2, in vivo gene editing activity experiments were performed targeting the EMX1 gene of immortalized HEK cells.

A target nucleic acid sequence within the EMX1 gene is as follows:

(SEQ ID NO: 136)
CTCATCTGTGCCCCTCCCTCCCTG

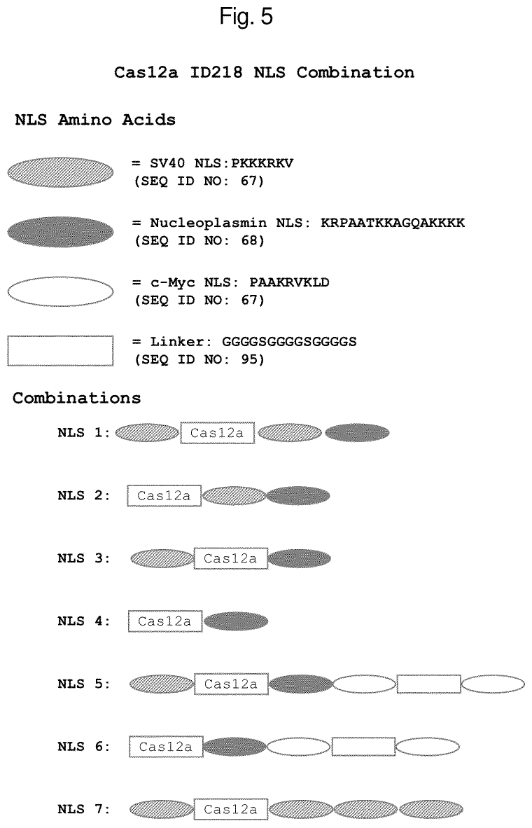

The composition of each CRISPR/Cas12a system used in the experiments is schematically shown in FIG. 5, and the specific sequence composition is shown in the following table:

TABLE 4
Composition of Each of CRISPR/Cas12a Systems Used in Experimental Example 3:

| | | Cas12a | | CRISPR | | |
|---|---|---|---|---|---|---|
| | | protein | | enzyme | Direct | Guide |
| | NLS at N- | (SEQ ID | NLS at C- | (SEQ ID | repeat of | domain of |
| Label | terminal | NO) | terminal | NO) | guide RNA | guide RNA |
| NLS1 | PKKKRKV | 1 | PKKKRKVKRP | 118 | UAAUUUC | CUCAUCU |
| (#1) | (SEQ ID | | AATKKAGQAK | | UACUAAG | GUGCCC |
| | NO: 67) | | KKK (SEQ ID | | UGUAGAU | CUCCCU |
| | | | NO: 69) | | (SEQ ID | CCCUG |
| | | | | | NO: 33) | (SEQ ID |
| | | | | | | NO: 137) |
| NLS2 | | 1 | SEQ ID NO: 69 | 119 | SEQ ID NO: | SEQ ID |
| (#2) | | | | | 33 | NO: 137 |
| NLS3 | SEQ ID | | KRPAATKKAG | 120 | SEQ ID NO: | SEQ ID |
| (#3) | NO: 67 | 1 | QAKKKK (SEQ | | 33 | NO: 137 |
| | | | ID NO: 68) | | | |
| NLS4 | | 1 | SEQ ID NO: 68 | 121 | SEQ ID NO: | SEQ ID |
| (#4) | | | | | 33 | NO: 137 |
| NLS5 | SEQ ID | 1 | KRPAATKKAG | 122 | SEQ ID NO: | SEQ ID |
| (#5) | NO: 67 | | QAKKKKPAAK | | 33 | NO: 137 |
| | | | RVKLDGGGG | | | |
| | | | SGGGGSGGG | | | |
| | | | GSPAAKRVKL | | | |
| | | | D (SEQ ID NO: | | | |
| | | | 70) | | | |
| NLS6 | — | 1 | SEQ ID NO: 70 | 123 | SEQ ID NO: | SEQ ID |
| (#6) | | | PKKKRKVPKK | | 33 | NO: 137 |
| NLS7 | SEQ ID | 1 | KRKVPKKKRK | 124 | SEQ ID NO: | SEQ ID |
| (#7) | NO: 67 | | V (SEQ ID NO: | | 33 | NO: 137 |
| | | | 71) | | | |

The specific experimental method was as follows:
1) CRISPR enzyme expression vectors were prepared according to Experimental Example 1.1, and using these, CRISPR enzymes were prepared and purified according to Experimental Example 1.3. Herein, BL21 competent cells (ThermoFisher) were used as competent cells, and were grown until OD600=0.5 to 0.7. The cultures were incubated for 1 hour at room temperature and then incubated at 23° C. for 18 hours with shaking (180 rpm). Thereby, protein expression was induced with 0.5 mM isopropyl-b-D-1-thiogalactopyranoside.
2) Guide RNAs were prepared according to Experimental Example 1.2.
3) Using the CRISPR enzymes and guide RNAs prepared above, CRISPR/Cas12a complexes were prepared according to Experimental Example 1.5.
4) The in vivo gene editing activity was measured according to Experimental Example 1.7, using the CRISPR/Cas12a complexes described above and the Immortalized HEK cells prepared according to Experimental Example 1.6.

### Experimental Example 3.2. CRISPR/Cas12a Preparation Results Used in Experiments

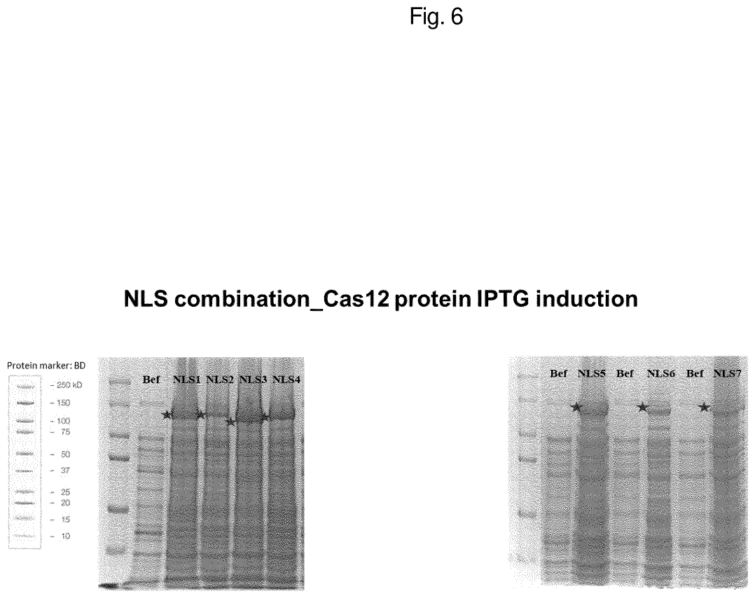

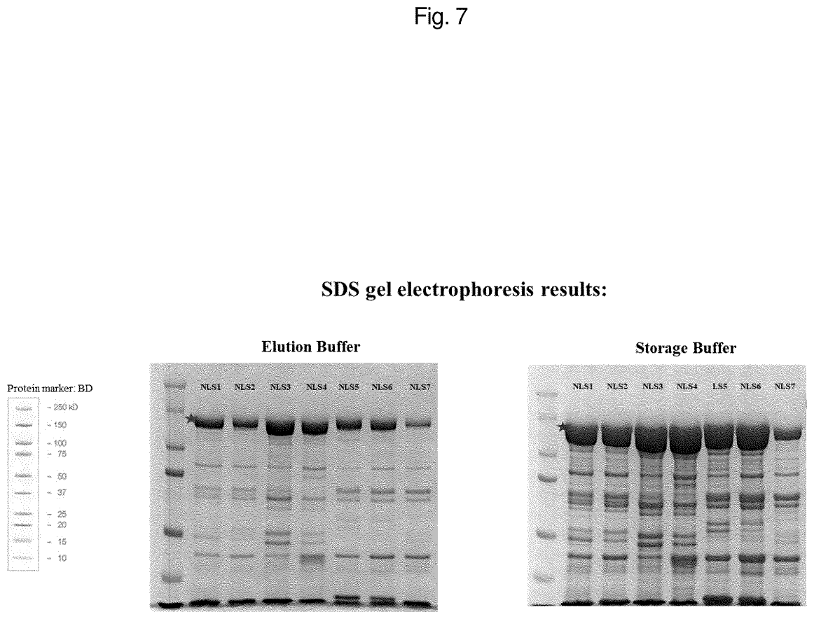

The evaluation results of the CRISPR enzyme, expressed according to Experimental Example 3.1, as described in Experimental Example 1.4 are shown in FIGS. 6 and 7.

Looking at the results, it was confirmed that each CRISPR enzyme was well expressed according to Experimental Example 3.1.

The molecular weight and concentration of each expression result are shown in the following table:

TABLE 5
Molecular Weight and Concentration of Each of CRISPR Enzymes
Expressed According to Experimental Example 3.1:

| Label | MW(Kda) | Conc(mg/ml) |
|---|---|---|
| NLS1 | 149 | 8.565 |
| NLS2 | 148 | 8.757 |
| NLS3 | 148 | 13.363 |
| NLS4 | 147 | 11.594 |
| NLS5 | 151 | 11.22 |
| NLS6 | 150 | 10.15 |
| NLS7 | 149 | 7.491 |

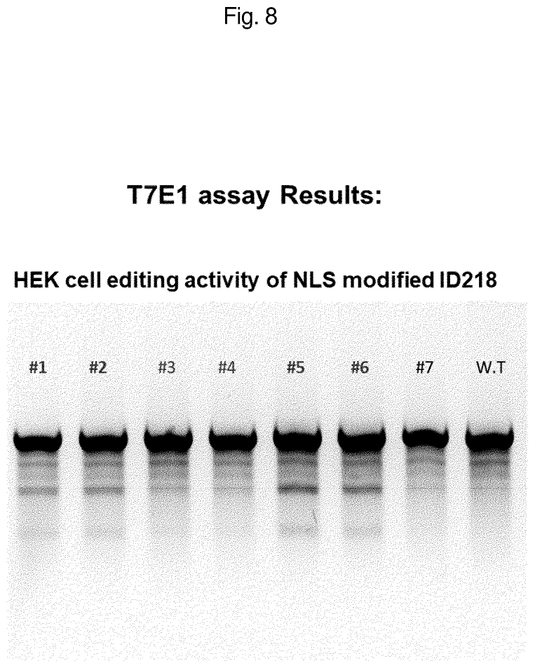

Experimental Example 3.3. Results of In Vivo Gene Editing Activity Experiments The results of the gene editing activity experiments for CRISPR/Cas12a systems targeting the endogenous EMX1 gene in HEK cells according to Experimental Example 3.1 are shown in FIG. 8.

As a result of the experiments, it was confirmed that bands indicating that the EMX1 gene was edited were clearly visible in NLS1 to NLS6, and that the editing activity appeared differently depending on the combination of NLSs used.

### Experimental Example 3.4. Results of In Vivo Gene Editing Activity Experiments #2

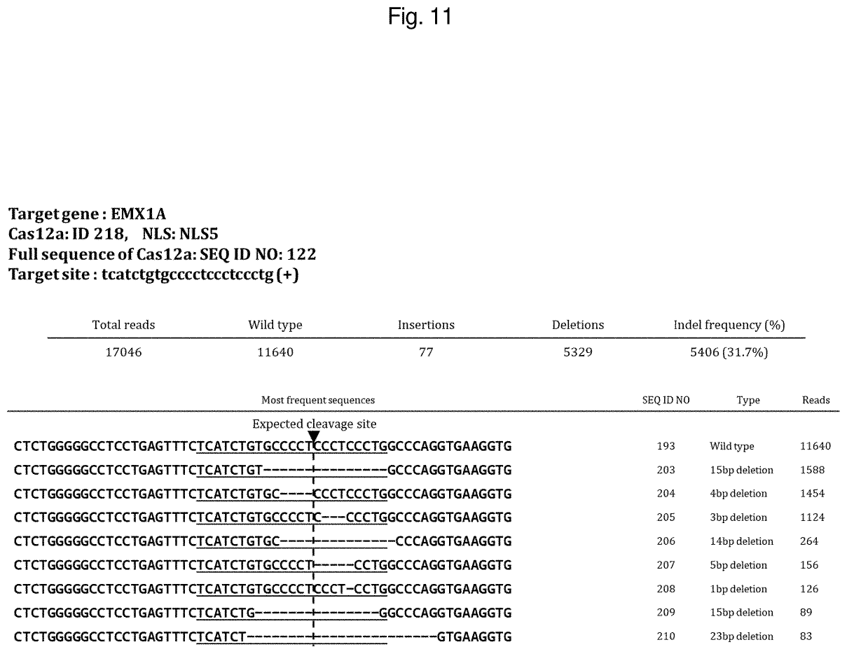

Among the CRISPR/Cas12a systems in Table 5, the results of confirming the in vivo gene editing activity for the CRISPR/Cas12a systems specific to NLS5 by Mutation Analysis are shown in FIG. 11.

As a result of the experiments, it was confirmed that the CRISPR/Cas12a system of ID218, which contained an NLS construct specific to NLS5, exhibited significantly high in vivo gene editing activity.

### Experimental Example 4. Confirmation of In Vivo Gene Editing Activity of CRISPR/Cas12a Systems #2

### Experimental Example 4.1. CRISPR/Cas12a Compositions Used in Experiments and Gene Editing Activity Confirmation Method

To determine whether the CRISPR/Cas12a systems disclosed herein enable to edit genes in eukaryotic cells, in vivo gene editing activity experiments were performed by targeting several genes (EMX1, MTAP, DYRK1A, and DNMT1A) in the HEK293T cell genome.

The gene sequences and protospacer sequences used in the experiments are shown in the following table:

TABLE 6
Genes and Protospacer Sequences Targeted for Gene Editing:

| Gene | | |
|---|---|---|
| name | Target sequence | Protospacer sequence |
| MTAP | CCATCTGTACCCCCAAAAACTATTGAAATTAAAAA | GCCCCAATAATCCCCACATG |
| | AAAATTGTGATTGATGTGGTTTCGCTGCATCCTC | TCA (SEQ ID NO: 139) |
| | GCCCAAATCTCATTTGTATTTTAGCCCCAATAATC | |
| | CCCACATGTCATGGAAGGGACCTGGTGGCAGGT | |
| | AATTGAATCATGGAGGTGGGTCTTTCTCGTGCTG | |
| | TTCTCGTGATAGTGAATAAATCTCACAAGATCTGA | |
| | TGGTTTTATAAAGGGGAGTTCCCCTGCACATGC | |
| | (SEQ ID NO: 138) | |
| DYRK1 | TCTTAAAACCTTGTCACACACAATGAAACTTTGCT | GAAGCACATCAAGGACATTC |
| A | GTTCACTGTCAGTTATAACTTACATGAGGTGACC | TAA (SEQ ID NO: 142) |
| | CATTTCCATTCAAGGGTITTAGAAGCACATCAAG | |
| | GACATTCTAAGGATGATTGACTTACACAATGATCT | |
| | CTGAACATGCCTCCTGCCTTCTCCTCACTCTTGA | |
| | GTATTTGCTTAGGGGAGC (SEQ ID NO: 141) | |

The composition for each of the CRISPR/Cas12a complexes used in the experiments is shown in the following table:

TABLE 7
Composition of Each of CRISPR/Cas12a
Complexes Used in Experiments:

| | Cas12a | CRISPR | Direct repeat | Guide domain |
|---|---|---|---|---|
| | protein | enzyme | of guide RNA | of guide RNA |
| Label | (SEQ ID NO) | (SEQ ID NO) | (SEQ ID NO) | (SEQ ID NO) |
| 204 | 4 | 256 | 36 | MTAP: 140 |
| | | | | DYRK1A: 143 |
| 206 | 5 | 257 | 37 | MTAP: 140 |
| | | | | DYRK1A: 143 |
| 210 | 6 | 258 | 38 | MTAP: 140 |
| | | | | DYRK1A: 143 |
| 211 | 7 | 259 | 39 | MTAP: 140 |
| | | | | DYRK1A: 143 |
| 217 | 8 | 260 | 40 | MTAP: 140 |
| | | | | DYRK1A: 143 |
| 218 | 1 | 253 | 33 | MTAP: 140 |
| | | | | DYRK1A: 143 |
| 230 | 12 | 261 | 44 | MTAP: 140 |
| | | | | DYRK1A: 143 |
| 233 | 2 | 254 | 34 | MTAP: 140 |
| | | | | DYRK1A: 143 |
| 236 | 3 | 255 | 35 | MTAP: 140 |
| | | | | DYRK1A: 143 |
| 239 | 14 | 262 | 46 | MTAP: 140 |
| | | | | DYRK1A: 143 |
| 250 | 16 | 263 | 48 | MTAP: 140 |
| | | | | DYRK1A: 143 |

In the table above, the CRISPR enzyme sequences represent sequences including those for Cas12a and NLSs.

Among the compositions of the CRISPR/Cas12a complexes, the sequences of Cas12a proteins were obtained from KR 2023-0111399 A, and the compositions of the guide RNAs were determined through additional research by the inventors of the present disclosure.

The specific experimental method was as follows:
1) CRISPR enzyme expression vectors were prepared according to Experimental Example 1.1, and using these, CRISPR enzymes were prepared and purified according to Experimental Example 1.3.
2) Guide RNAs were prepared according to Experimental Example 1.2.
3) Using the CRISPR enzymes and guide RNAs prepared above, CRISPR/Cas12a complexes were prepared according to Experimental Example 1.5.
4) The in vivo gene editing activity was measured according to Experimental Example 1.8, using the CRISPR/Cas12a complexes described above and the Immortalized HEK cells prepared according to Experimental Example 1.6.

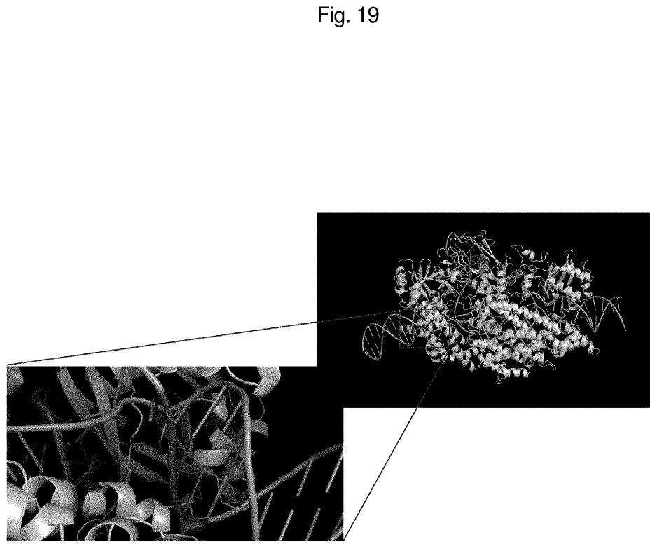

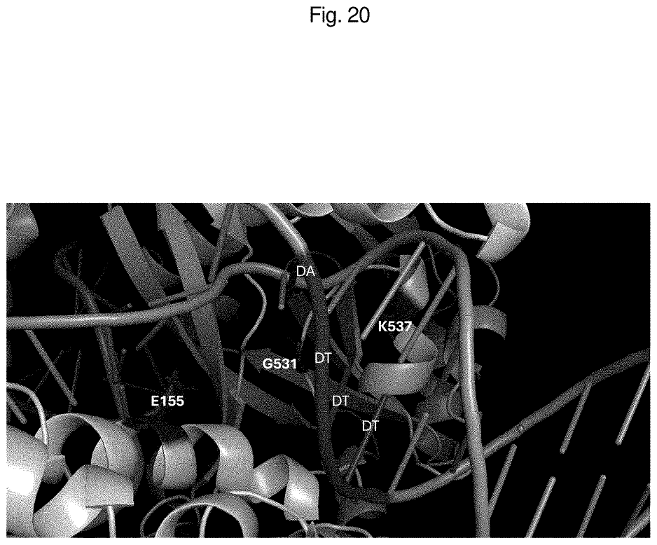

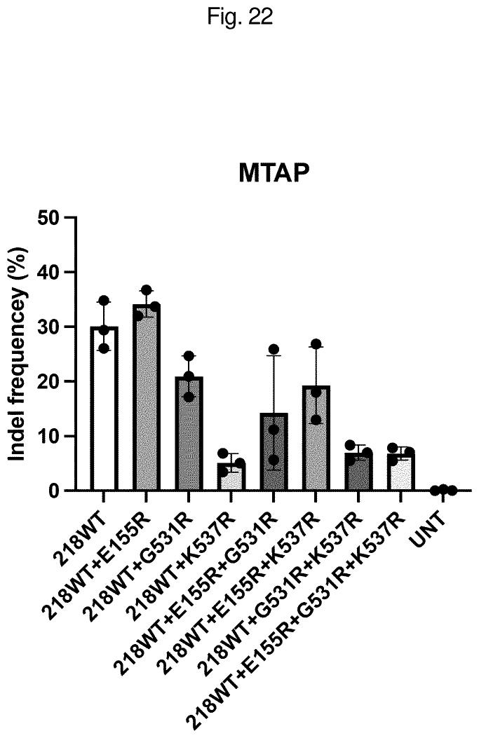

Information on each primer used in the experiments is shown in FIGS. 19 to 22:

### Experimental Example 4.2. CRISPR/Cas12a Preparation Results Used in Experiments

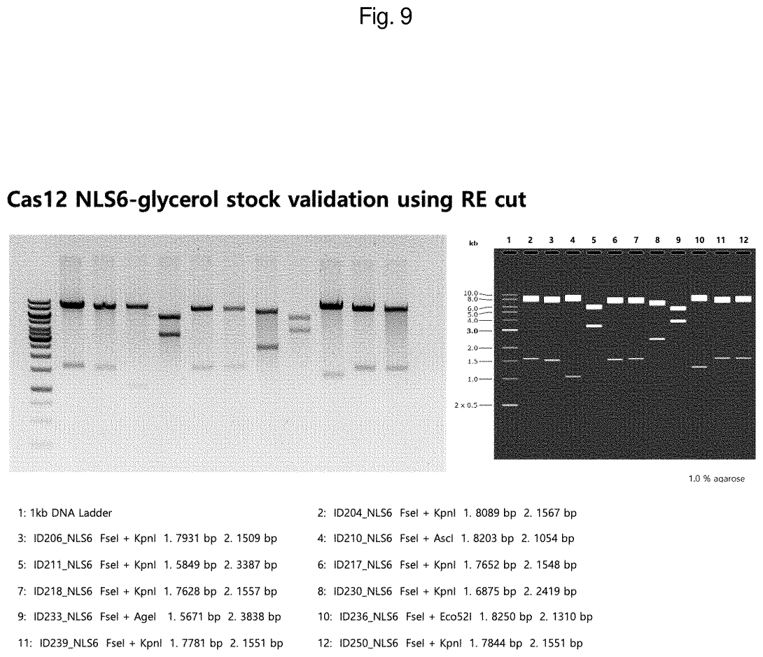

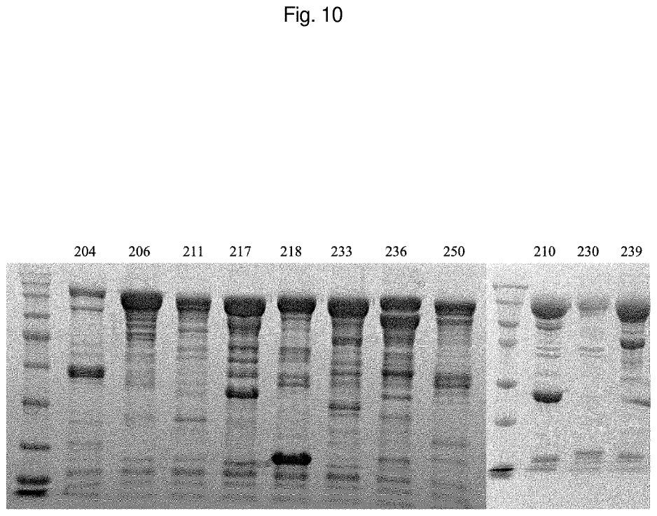

The evaluation results of the CRISPR enzymes, prepared according to Experimental Example 4.1, as described in Experimental Example 1.4 are shown in FIG. 9. The results of SDS-PAGE analysis according to Experimental Example 1.3 are shown in FIG. 10.

Looking at the results, it was confirmed that each CRISPR enzyme was well expressed according to Experimental Example 4.1.

### Experimental Example 4.3. Results of In Vitro Gene Editing Activity Experiments

To evaluate in vitro cleavage activity of the CRISPR enzymes prepared according to Experimental Example 4.1, the following experiments were performed:
1) CRISPR/Cas12a complexes were assembled according to Experimental Example 1.5 using prepared CRISPR enzymes and guide RNAs. Herein, to determine activity depending on pH conditions, the CRISPR/Cas12a complexes were assembled in buffers of pH=7.9 (NEB buffer 2.1), pH=6.5 (50 mM NaCl, 10 mM Tris-HCl, 10 mM MgCl2, 100 μg/mL BSA) and pH=4.5 (50 mM NaCl, 10 mM sodium acetate, 10 mM MgCl2, 0.1 mM DTT), respectively.
2) The CRISPR/Cas12a complexes assembled in 1) were added to 1 μg of the DNA templates of a SCN1A gene to initiate a cleavage reaction. Herein, to determine activity depending on reaction temperature, each reaction mixture was incubated at 25° C., 37° C., and 60° C. for 1 hour.
3) The cleavage activity of the CRISPR/Cas12a complexes was visualized by Ethidium Bromide staining of the reaction products separated on a 1% agarose gel using 1×TAE (Tris-acetate EDTA) buffer.

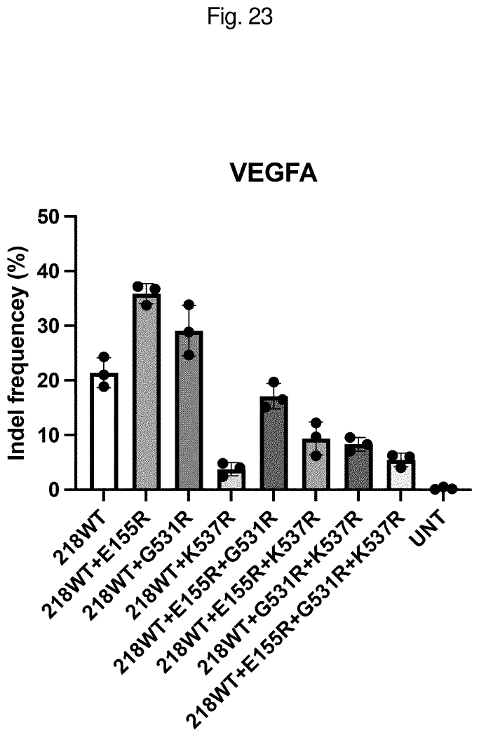

The results of the in vitro cleavage activity experiments for each of the CRISPR/Cas12a complexes are shown in FIG. 23.

As a result of the experiments, most CRISPR/Cas12a complexes showed in vitro nucleic acid cleavage activity.

### Experimental Example 4.4. In Vivo Gene Editing Activity Experiments #1—Experimental Method

To confirm the in vivo gene editing activity of the CRISPR enzymes according to Experimental Example 4.1, the following experiments were performed:
1) CRISPR/Cas12a complexes were assembled according to Experimental Example 1.5 using CRISPR enzymes prepared according to Experimental Example 4.1 and guide RNAs.
2) The CRISPR/Cas12a complexes assembled in 1) were transfected into HEK293T cells according to Experimental Example 1.8, and gene editing activity was measured.

### Experimental Example 4.5. In Vivo Gene Editing Activity Experiments #2—MTAP Gene

The results of measuring in vivo gene editing activity of each of the CRISPR/Cas12a complexes targeting an MTAP gene are shown in the following table:

TABLE 8
MTAP Gene Editing Activity:

| Label | Total Reads | Wild Type | Insertion | Deletion | Indel (%) | |
|---|---|---|---|---|---|---|
| 204 | 14275 | 14270 | 0 | 5 | 5 | (0.0%) |
| 206 | 15000 | 14998 | 0 | 2 | 2 | (0.0%) |
| 210 | 3402 | 3402 | 0 | 0 | 0 | (0.0%) |
| 211 | 16722 | 16718 | 0 | 4 | 4 | (0.0%) |
| 217 | 13953 | 13928 | 2 | 23 | 25 | (0.2%) |
| 218 | 3894 | 372 | 136 | 3386 | 3522 | (90.4%) |
| 230 | 3769 | 3769 | 0 | 0 | 0 | (0.0%) |
| 233 | 14303 | 12183 | 95 | 2025 | 2120 | (14.8%) |
| 236 | 4346 | 3607 | 52 | 686 | 739 | (17.0%) |
| 239 | 3311 | 3311 | 0 | 0 | 0 | (0.0%) |
| 250 | 12860 | 12860 | 0 | 0 | 0 | (0.0%) |

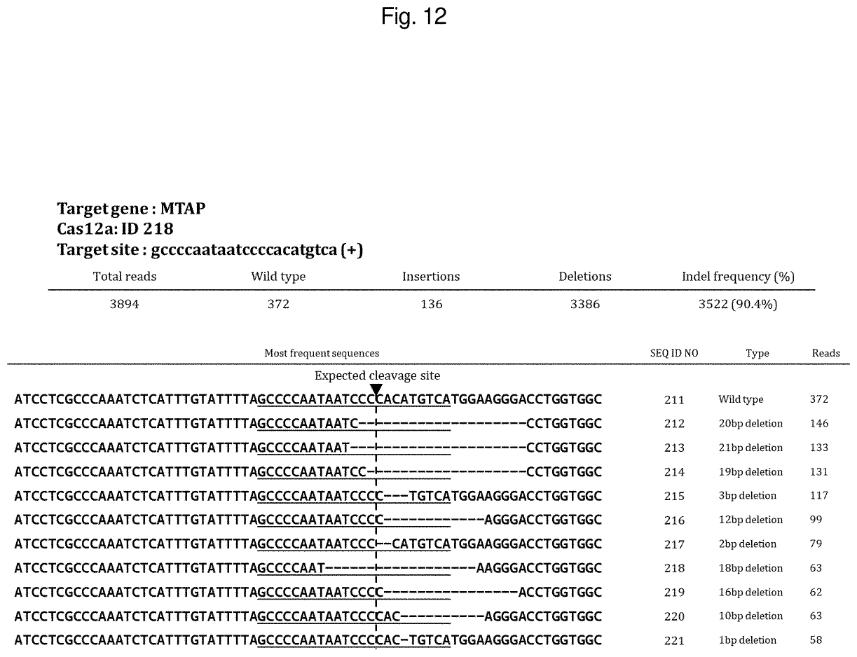

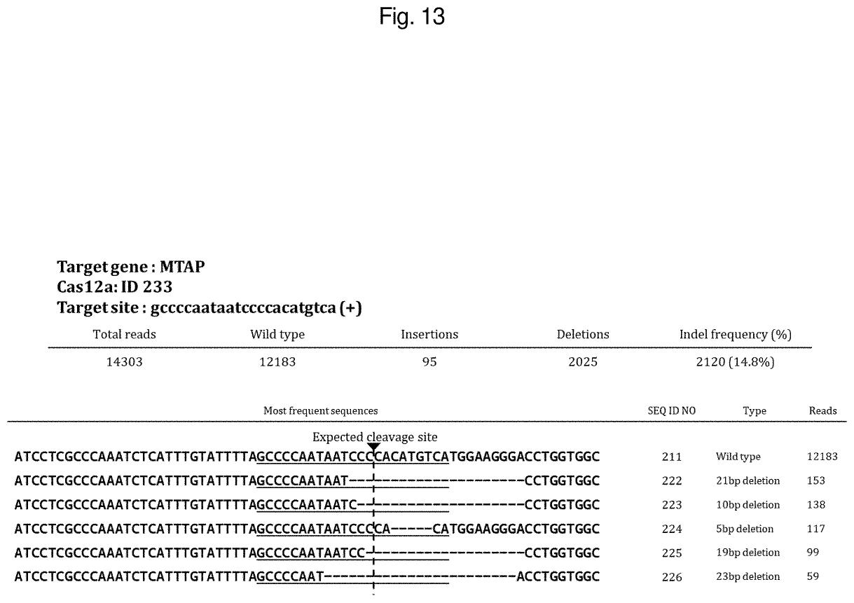

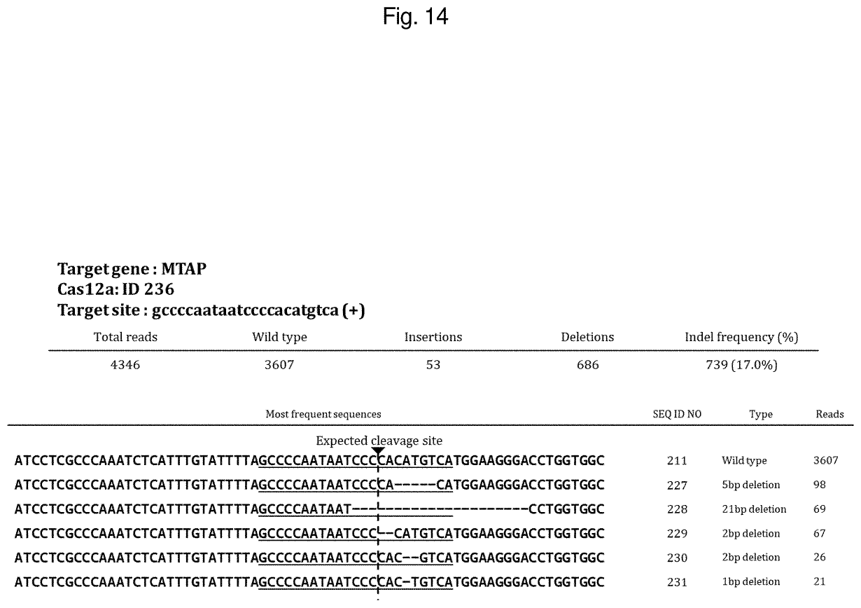

The NGS sequencing results of MTAP gene editing by ID 218, ID 233, and ID 236 are shown in FIGS. 12 to 14.

As a result of the experiments, it was confirmed that the CRISPR/Cas12a complexes including Cas12a proteins for ID 218, ID 233, and ID 236 introduced a significant proportion of indels into the MTAP gene.

### Experimental Example 4.6. In Vivo Gene Editing Activity Experiments #3—DYRK1A Gene

The results of measuring in vivo gene editing activity of each CRISPR/Cas12a complexes targeting a DYRK1A gene are shown in the following table:

TABLE 9
DYRK1A Gene Editing Activity:

| Label | Total Reads | Wild Type | Insertion | Deletion | Indel (%) | |
|---|---|---|---|---|---|---|
| 204 | 15584 | 15582 | 0 | 2 | 2 | (0.0%) |
| 206 | 16591 | 16588 | 0 | 3 | 3 | (0.0%) |
| 210 | 4161 | 4159 | 0 | 2 | 2 | (0.0%) |
| 211 | 18417 | 18386 | 0 | 31 | 31 | (0.2%) |
| 217 | 13894 | 13894 | 0 | 0 | 0 | (0.0%) |
| 218 | 4199 | 1219 | 121 | 2859 | 2980 | (71.0%) |
| 230 | 3531 | 3531 | 0 | 0 | 0 | (0.0%) |
| 233 | 14386 | 12628 | 56 | 1702 | 1758 | (12.2%) |
| 236 | 4779 | 4531 | 4 | 244 | 248 | (5.2%) |
| 239 | 3697 | 3695 | 0 | 2 | 2 | (0.1%) |
| 250 | 14234 | 14229 | 0 | 5 | 5 | (0.0%) |

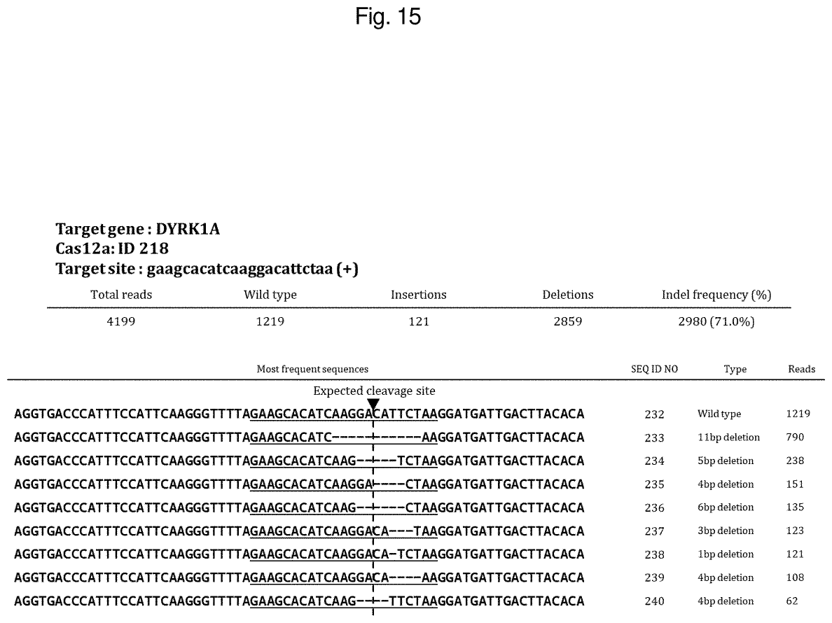

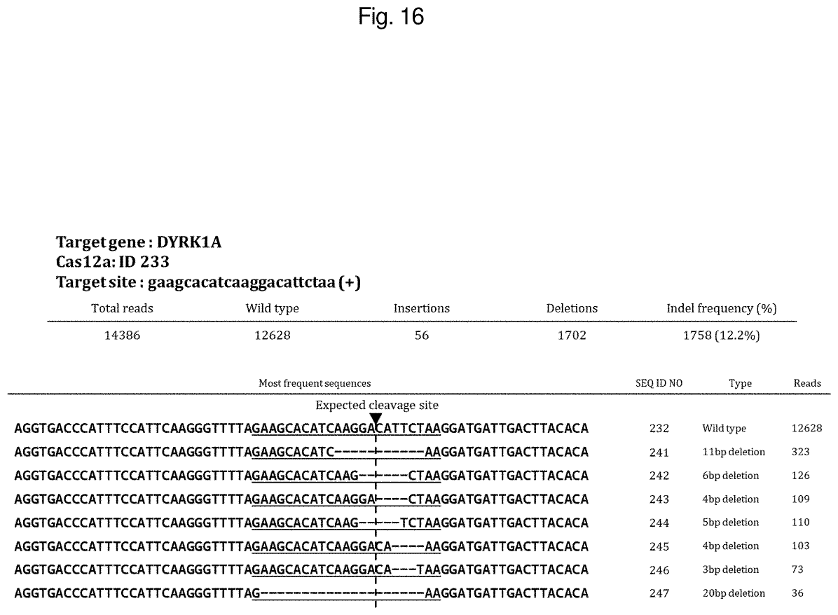

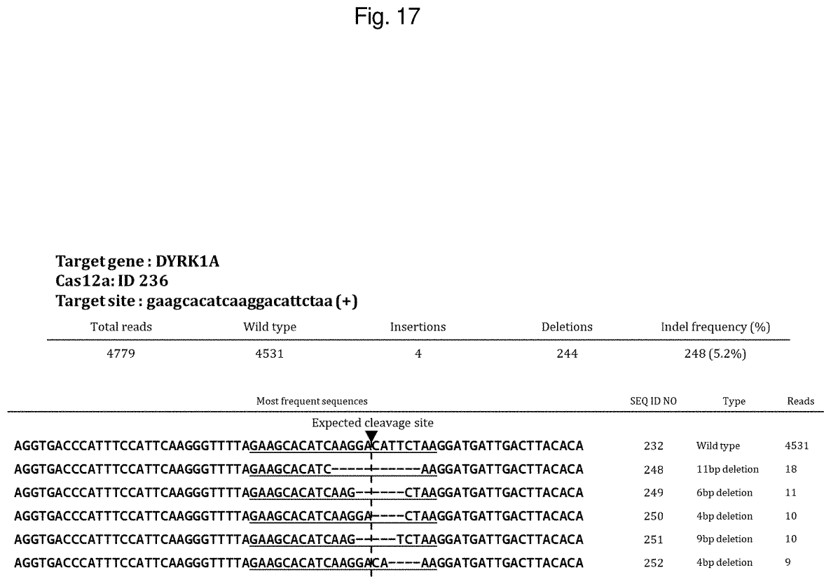

The NGS sequencing results of DYRK1A gene editing by ID 218, ID 233, and ID 236 are shown in FIGS. 15 to 17.

As a result of the experiments, it was confirmed that the CRISPR/Cas12a complexes including Cas12a proteins for ID 218, ID 233, and ID 236 introduced a significant proportion of indels into the DYRK1A gene.

### Experimental Example 4.7. In Vivo Gene Editing Activity Experiments #4—Interpretation of Experiment Results

As a result of the experiments, not all CRISPR enzymes produced had in vivo gene editing activity. Only the novel Cas12a proteins having the amino acid sequences of SEQ ID NO: 1, SEQ ID NO: 2, and SEQ ID NO: 3, respectively were confirmed to have gene editing activity. When each of the novel Cas12a proteins having the sequences constituted a CRISPR/Cas12a system with an appropriate NLS and an appropriate guide RNA, there was some difference in editing efficiency for each gene sequence, but the novel Cas12a proteins having the sequences could sufficiently edit the cell genome in vivo.

### Experimental Example 5. Eukaryotic Genome Editing Activity Experiments of CRISPR/Cas12a Systems Including Cas12a Protein Variants #1

### Experimental Example 5.1. Search for PAM Recognition Site Mutations

By referring to the conventional literature (Kleinstiver B P, Sousa A A, Walton R T, Tak Y E, Hsu J Y, Clement K, Welch M M, Horng J E, Malagon-Lopez J, Scarfo I, Maus M V, Pinello L, Aryee M J, Joung J K. Engineered CRISPR-Cas12a variants with increased activities and improved targeting ranges for gene, epigenetic and base editing. Nat Biotechnol. 2019 March; 37 (3): 276-282. doi: 10.1038/s41587-018-0011-0. Epub 2019 Feb. 11. Erratum in: Nat Biotechnol. 2020 July; 38 (7): 901. doi: 10.1038/s41587-020-0587-z. PMID: 30742127; PMCID: PMC6401248.), an attempt was made to find mutation positions in the PAM recognition sites of the Cas12a proteins discovered above.

Specifically, AlphaFold was used to analyze the structure of the CRISPR/Cas12a system including the Cas12a protein of SEQ ID NO: 1 bound to a target nucleic acid.

The analysis results are shown in FIGS. 19 to 21.

As a result of the analysis, it was revealed that positions E155, G531, and K537 in the amino acid sequence of SEQ ID NO: 1 were positions that significantly interacted with the PAM sequence.

Herein below, gene editing efficiency was measured by functionally introducing mutations at the positions identified above.

### Experimental Example 5.2. Experimental Methods and Compositions Used in Experiments

In Experimental Example 5.1, variants were prepared in which the amino acid at a specific position was replaced with arginine, and used in the experiments. All possible combinations of proteins were prepared, replacing each position with arginine. Specifically, according to Experimental Example 1.9, a plasmid encoding each variant Cas12a protein was constructed, and a guide RNA plasmid capable of operating with its corresponding Cas12a protein was also prepared. The corresponding plasmid vector was transfected into cells according to Experimental Example 1.6 using the Lipofectamine 2000 system (Thermo, 11668019). Afterward, the cell genome was extracted and gene editing activity was measured. The extracted cell genome was analyzed according to Experimental Example 1.12.

The composition for each of the CRISPR/Cas12a complexes used in Experimental Example 5 is shown in the following table:

TABLE 10

| | | Cas12a | | Direct | Guide domain |
|---|---|---|---|---|---|
| | Chart | protein | | repeat of | of guide RNA |
| Label | Label | (SEQ ID NO) | C-terminal NLS | guide RNA | (SEQ ID NO) |
| Example | 218WT | 1 | KRPAATKKAGQ | UAAUUUCUA | DYRK1A: 143 |
| 5.1 | | | AKKKKPAAKRV | CUAAGUGUA | MTAP: 140 |
| | | | KLDGGGGSGG | GAU | VEGFA: 419 |
| | | | GGSGGGGSPA | (SEQ ID | FANCF: 420 |
| | | | AKRVKLD (SEQ | NO: 33) | CFTR: 421 |
| | | | ID NO: 70) | | |
| Example | 218WT + | 264 | (Same as above) | (Same as | (Same as |
| 5.2 | E155R | | | above) | above) |
| Example | 218WT + | 265 | (Same as above) | (Same as | (Same as |
| 5.3 | G531R | | | above) | above) |
| Example | 218WT + | 266 | (Same as above) | (Same as | (Same as |
| 5.4 | E155R + | | | above) | above) |
| | G531R | | | | |
| Example | 218WT + | 267 | (Same as above) | (Same as | (Same as |
| 5.5 | E155R + | | | above) | above) |
| | K537R | | | | |
| Example | 218WT + | 299 | (Same as above) | (Same as | (Same as |
| 5.6 | K537R | | | above) | above) |
| Example | 218WT + | 300 | (Same as above) | (Same as | (Same as |
| 5.7 | G531R + | | | above) | above) |
| | K537R | | | | |
| Example | 218WT + | 301 | (Same as above) | (Same as | (Same as |
| 5.8 | E155R + | | | above) | above) |
| | G531R + | | | | |
| | K537R | | | | |
| Control | UNT | — | — | — | — |

### Experimental Example 5.3. Experiment Results

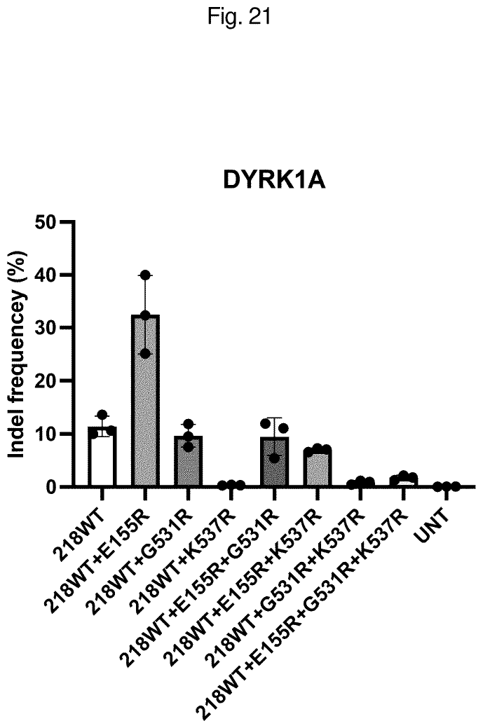

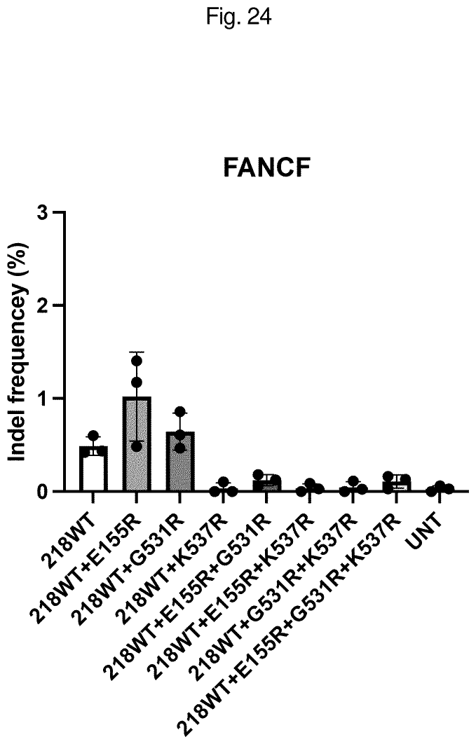

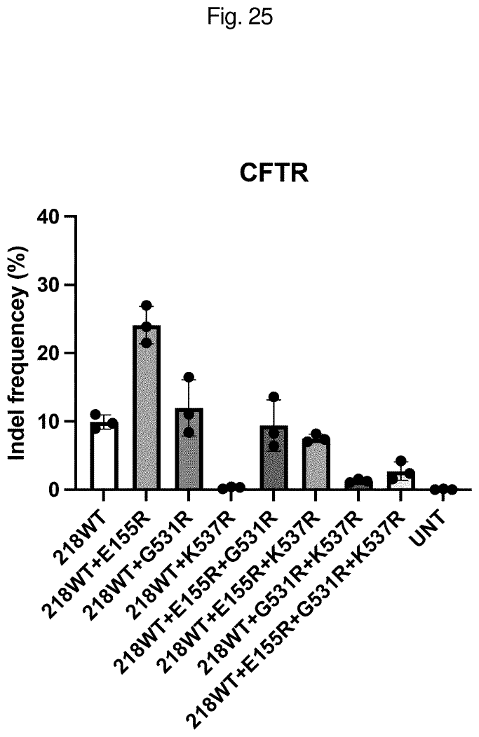

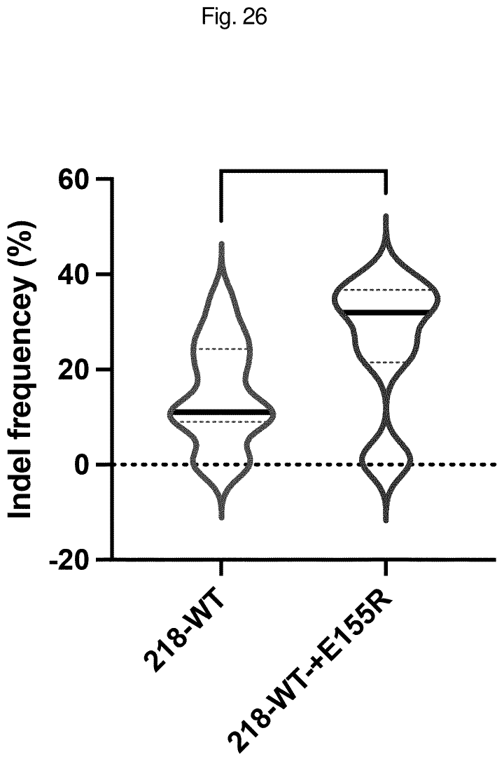

The experimental results are shown in FIGS. 21 to 26. Quantitative data for the experimental results are shown in the following table:

TABLE 11

| Label | MTAP | DYRK1A | VEGFA | FANCF | CFTR | site3 |
|---|---|---|---|---|---|---|
| 218WT | 26.08 | 10.60 | 21.02 | 0.42 | 8.95 | 13.07 |
| (Example 5.1) | 34.82 | 9.99 | 18.85 | 0.60 | 11.02 | 24.67 |
| | 29.40 | 13.61 | 24.30 | 0.44 | 9.73 | 18.36 |
| 218WT + | 33.68 | 25.11 | 37.17 | 1.40 | 26.97 | 13.10 |
| E155R | 31.99 | 39.97 | 36.71 | 1.17 | 23.85 | 19.89 |
| (Example 5.2) | 36.75 | 32.30 | 33.73 | 0.48 | 21.47 | 15.26 |
| 218WT + | 17.16 | 9.53 | 28.85 | 0.47 | 16.48 | 6.38 |
| G531R | 20.94 | 7.53 | 33.87 | 0.61 | 11.06 | 12.38 |
| (Example 5.3) | 24.67 | 11.85 | 24.64 | 0.86 | 8.39 | 17.55 |
| 218WT + | 5.65 | 5.40 | 15.10 | 0.06 | 13.58 | 11.67 |
| E155R + | 11.20 | 11.06 | 16.52 | 0.18 | 6.37 | 15.23 |
| G531R | 25.90 | 11.92 | 19.69 | 0.13 | 8.26 | 7.21 |
| (Example 5.4) | | | | | | |
| 218WT + | 12.98 | 7.05 | 6.20 | 0.00 | 8.20 | 4.32 |
| E155R + | 18.01 | 7.26 | 12.23 | 0.03 | 7.03 | 8.53 |
| K537R | 26.85 | 6.56 | 9.65 | 0.09 | 7.35 | 7.36 |
| (Example 5.5) | | | | | | |
| 218WT + | 3.43 | 0.41 | 2.39 | 0.00 | 0.36 | 0.84 |
| K537R | 5.05 | 0.27 | 4.82 | 0.10 | 0.13 | 1.02 |
| (Example 5.6) | 6.85 | 0.32 | 4.02 | 0.00 | 0.43 | 1.25 |
| 218WT + | 5.57 | 0.45 | 8.32 | 0.00 | 1.06 | 6.66 |
| G531R + | 7.00 | 0.95 | 9.56 | 0.03 | 1.25 | 5.81 |
| K537R | 8.34 | 1.13 | 7.07 | 0.11 | 1.58 | 5.74 |
| (Example 5.7) | | | | | | |
| 218WT + | 5.49 | 1.26 | 4.02 | 0.03 | 1.54 | 1.77 |
| E155R + | 7.87 | 2.15 | 6.03 | 0.16 | 4.23 | 2.62 |
| G531R + | 7.05 | 1.79 | 6.29 | 0.13 | 2.39 | 2.53 |
| K537R | | | | | | |
| (Example 5.8) | | | | | | |
| UNT | 0.26 | 0.04 | 0.51 | 0.03 | 0.10 | 0.07 |
| (Control) | 0.03 | 0.02 | 0.13 | 0.06 | 0.00 | 0.22 |
| | 0.06 | 0.08 | 0.07 | 0.00 | 0.00 | 0.27 |

All figures express the indel introduction rate as a percentage. The experiments were repeated 3 times for each experimental example. As a result of the experiments, those in Example 5.2 and Example 5.3 showed higher eukaryotic genome editing efficiency than the novel Cas12a protein (Example 5.3) in which no mutation was introduced. In Example 5.4 and Example 5.5, excellent editing efficiency was not shown, but editing efficiency was shown at a usable level.

### Experimental Example 6. Eukaryotic Genome Editing Activity Experiments of CRISPR/Cas12a Systems Including Cas12a Protein Variants #2

### Experimental Example 6.1. Search for Mutation Positions at PAM Recognition Sites

According to Experimental Example 5.1, positions that significantly interacted with PAM were found in the amino acid sequence of SEQ ID NO: 3 through AlphaFold structural analysis.

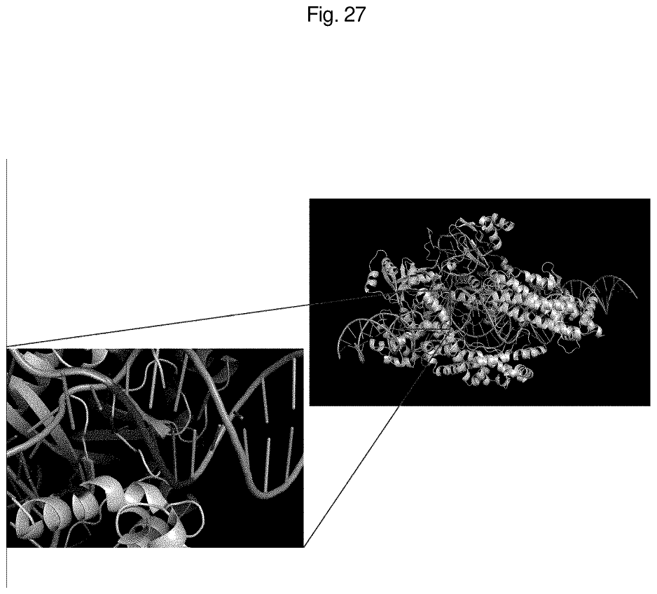

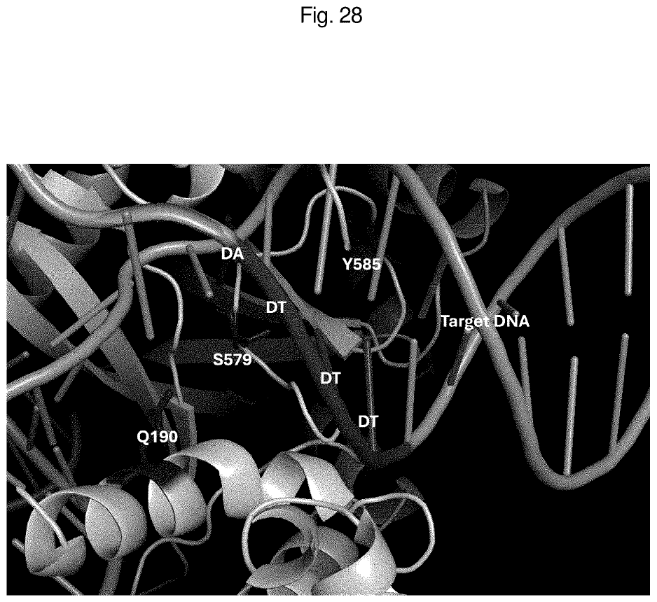

The analysis results are shown in FIGS. 27 to 28.

As a result of the analysis, it was revealed that positions Q190, S579, and Y585 in the amino acid sequence of SEQ ID NO: 3 were positions that significantly interacted with the PAM sequence.

Herein below, gene editing efficiency was measured by functionally introducing mutations at the positions identified above.

### Experimental Example 6.2. Experimental Methods and Compositions Used in Experiments

In Experimental Example 6.1, variants were prepared in which the amino acid at a specific position was replaced with arginine, and used in the experiments. All possible combinations of proteins were prepared, replacing each position with arginine. Specifically, according to Experimental Example 1.9, a plasmid encoding each variant Cas12a protein was constructed, and a guide RNA plasmid capable of operating with its corresponding Cas12a protein was also prepared. The corresponding plasmid vector was transfected into cells according to Experimental Example 1.6 using the Lipofectamine 2000 system (Thermo, 11668019). Afterward, the cell genome was extracted and gene editing activity was measured. The extracted cell genome was analyzed according to Experimental Example 1.12.

The composition for each of the CRISPR/Cas12a complexes used in Experimental Example 6 are shown in the following table:

TABLE 12

| | | Cas12a | | | |
|---|---|---|---|---|---|
| | | protein | | | Guide domain |
| | Chart | (SEQ ID | | Direct repeat | of guide RNA |
| Label | Label | NO) | C-terminal NLS | of guide RNA | (SEQ ID NO) |
| Example | 236-WT-NLS6 | 3 | KRPAATKKAGQ | UAAAUUUCU | DYRK1A: 143 |
| 6.1 | | | AKKKKPAAKRV | ACUGUUGUA | VEGFA: ? |
| | | | KLDGGGGSGG | GAU (SEQ ID | CFTR: ? |
| | | | GGSGGGGSPA | NO: 36) | |
| | | | AKRVKLD (SEQ | | |
| | | | ID NO: 70) | | |
| Example | 236-WT | 276 | (Same as above) | (Same as | (Same as |
| 6.2 | (Q190R)-NLS6 | | | above) | above) |
| Example | 236-WT | 277 | (Same as above) | (Same as | (Same as |
| 6.3 | (S579R)-NLS6 | | | above) | above) |
| Example | 236-WT | 275 | (Same as above) | (Same as | (Same as |
| 6.4 | (Q190R + | | | above) | above) |
| | S579R)-NLS6 | | | | |
| Example | 236-WT | 278 | (Same as above) | (Same as | (Same as |
| 6.5 | (Q190R + | | | | |
| | Y585R)-NLS6 | | | above) | above) |
| Example | 236-WT | 279 | (Same as above) | (Same as | (Same as |
| 6.6 | (Q190R + | | | above) | above) |
| | S579R + | | | | |
| | Y585R)-NLS6 | | | | |
| Example | 236-WT | 305 | (Same as above) | (Same as | (Same as |
| 6.7 | (Y585R)-NLS6 | | | above) | above) |
| Example | 236-WT | 306 | (Same as above) | (Same as | (Same as |
| 6.8 | (S579R + | | | above) | above) |
| | Y585R)-NLS6 | | | | |
| Control | UNT | — | — | — | — |

### Experimental Example 6.3. Experiment Results

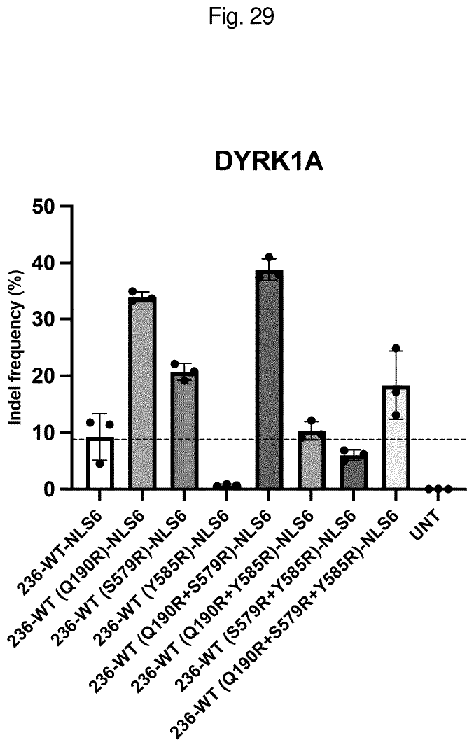

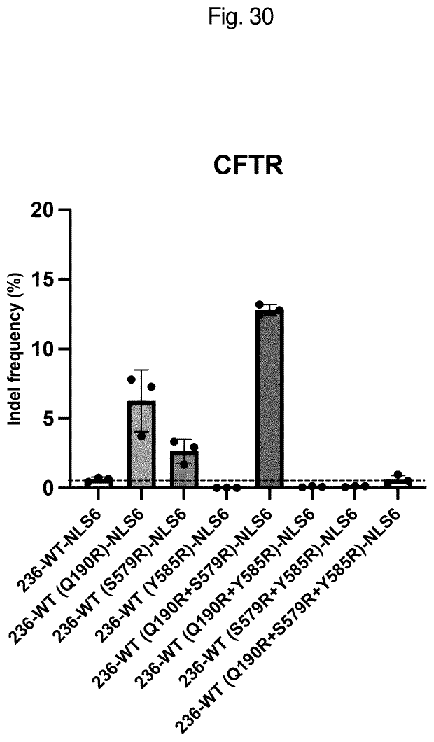

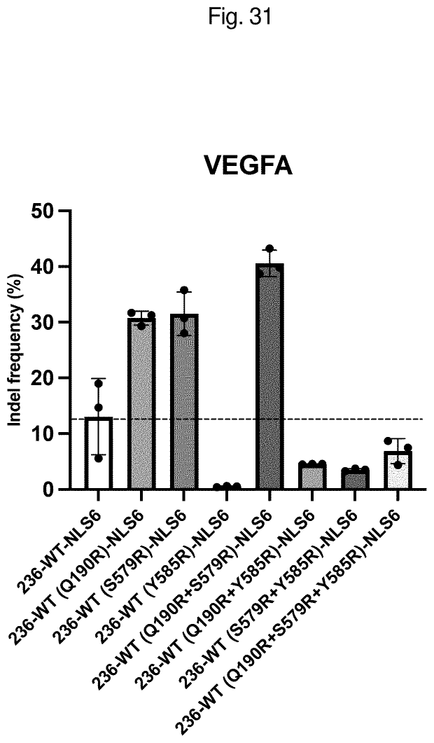

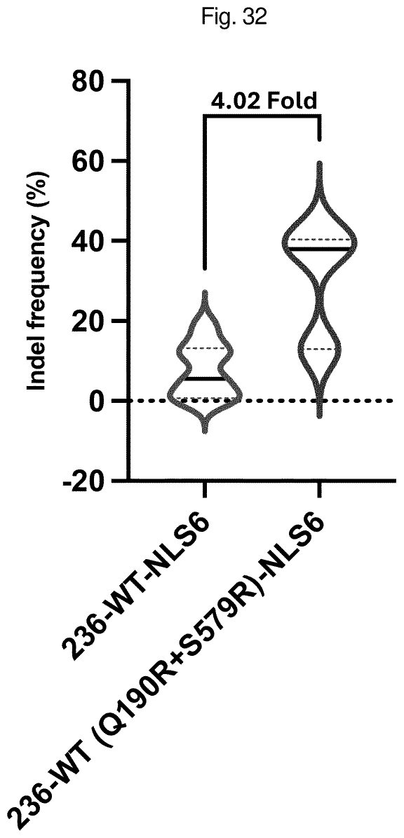

The experimental results are shown in FIGS. 29 to 32. Quantitative data for the experimental results are shown in the following table:

TABLE 13

| Label | DYRK1A | CFTR | VEGFA |
|---|---|---|---|
| 236-WT-NLS6 | 4.50 | 0.47 | 14.67 |
| (Example 6.1) | 11.76 | 0.76 | 5.57 |
| | 11.38 | 0.68 | 18.96 |
| 236-WT (Q190R)-NLS6 | 33.69 | 3.73 | 31.69 |
| (Example 6.2) | 34.95 | 7.29 | 31.28 |
| | 33.27 | 7.82 | 29.31 |
| 236-WT (S579R)-NLS6 | 20.81 | 1.69 | 30.74 |
| (Example 6.3) | 22.11 | 3.34 | 35.76 |
| | 19.14 | 2.93 | 28.03 |
| 236-WT (Q190R + S579R)-NLS6 | 37.40 | 12.43 | 38.69 |
| (Example 6.4) | 40.98 | 13.19 | 39.77 |
| | 37.94 | 12.80 | 43.22 |
| 236-WT (Q190R + Y585R)-NLS6 | 9.09 | 0.09 | 4.48 |
| (Example 6.5) | 12.15 | 0.06 | 4.56 |
| | 9.71 | 0.14 | 4.55 |
| 236-WT (Q190R + Y585R)-NLS6 | 9.09 | 0.09 | 4.48 |
| (Example 6.6) | 12.15 | 0.06 | 4.56 |
| | 9.71 | 0.14 | 4.55 |
| 236-WT (Q190R + S579R + Y585R)-NLS6 | 13.05 | 0.40 | 4.35 |
| (Example 6.7) | 17.15 | 0.53 | 8.70 |
| | 24.85 | 0.97 | 7.48 |
| 236-WT (Y585R)-NLS6 | 0.68 | 0.05 | 0.54 |
| (Example 6.8) | 0.89 | 0.03 | 0.59 |
| | 0.51 | 0.02 | 0.41 |
| 236-WT (S579R + Y585R)-NLS6 | 4.97 | 0.07 | 3.26 |
| (Example 6.9) | 6.20 | 0.16 | 3.52 |
| | 6.86 | 0.15 | 3.74 |
| UNT | 0.04 | — | — |
| (Control) | 0.03 | — | — |
| | 0.03 | — | — |

All figures express the indel introduction rate as a percentage. The experiments were repeated 3 times for each experimental example. As a result of the experiments, those in Examples 6.2 to 6.4 showed higher eukaryotic genome editing efficiency than the novel Cas12a protein (Example 6.1) in which no mutation was introduced. In Example 6.5 and Example 6.6, excellent editing efficiency was not shown, but editing efficiency was shown at a usable level.

### Experimental Example 7. Eukaryotic Genome Editing Activity Experiments of CRISPR/Cas12a Systems Including Cas12a Protein Variants #3

### Experimental Example 7.1. Search for Ultra Nuclease Mutation Positions

By referring to the conventional literature (Zhang, L., Zuris, J. A., Viswanathan, R. et al. AsCas12a ultra nuclease facilitates the rapid generation of therapeutic cell medicines. Nat Commun 12, 3908 (2021). https://doi.org/10.1038/s41467-021-24017-8; Hsiung, C. C S., Wilson, C. M., Sambold, N. A. et al. Engineered CRISPR-Cas12a for higher-order combinatorial chromatin perturbations. Nat Biotechnol (2024). Https://doi.org/10.1038/s41587-024-02224-0), Ultra Nuclease mutation positions were found in the Cas12a protein having the amino acid sequence of SEQ ID NO: 1. In particular, an attempt was made to find positions corresponding to M537 and F870 of the AsCas12a protein disclosed in the conventional literature.

Additionally, by referring to the conventional literature (Naqvi, M. M., Lee, L., Montaguth, O. E. T. et al. CRISPR-Cas12a-mediated DNA clamping triggers target-strand cleavage. Nat Chem Biol 18, 1014-1022 (2022). Https://doi.org/10.1038/s41589-022-01082-8), an attempt was made to find mutation positions known to increase nucleic acid cleavage efficiency by increasing an R-loop dwell time.

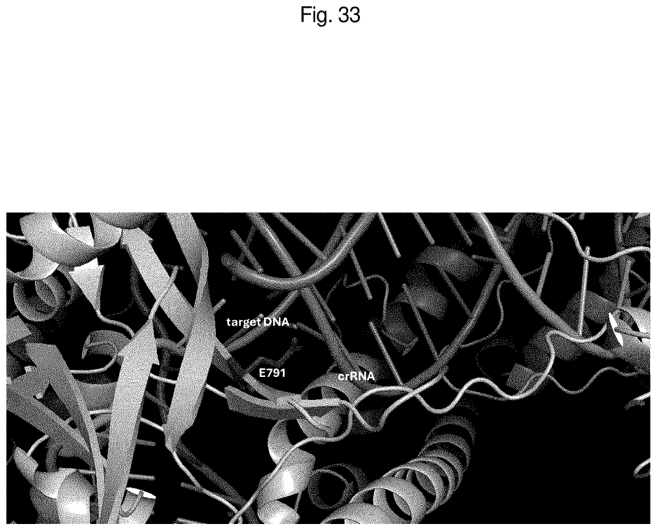

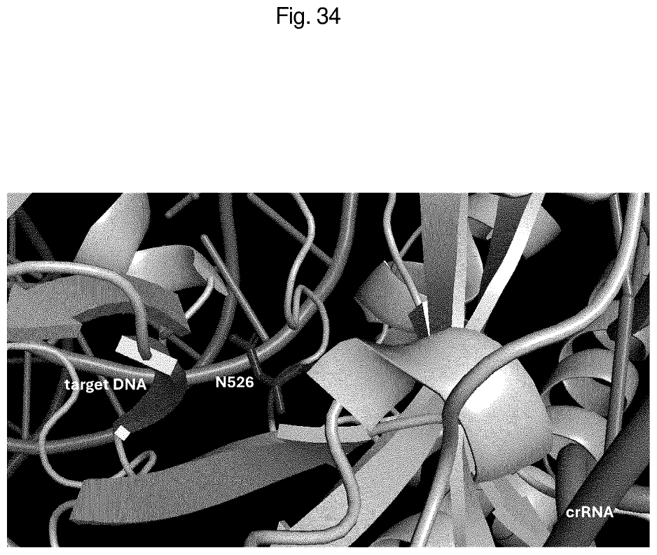

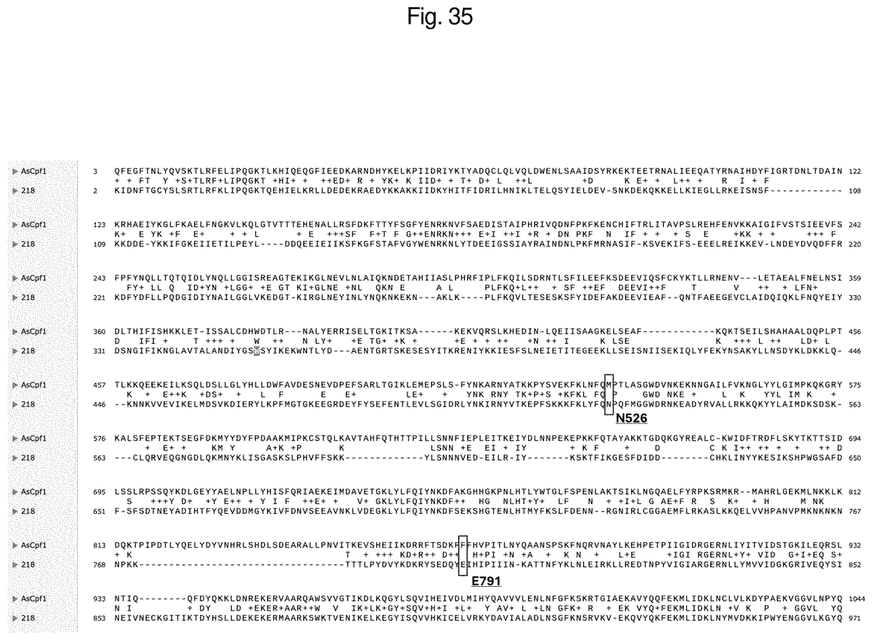

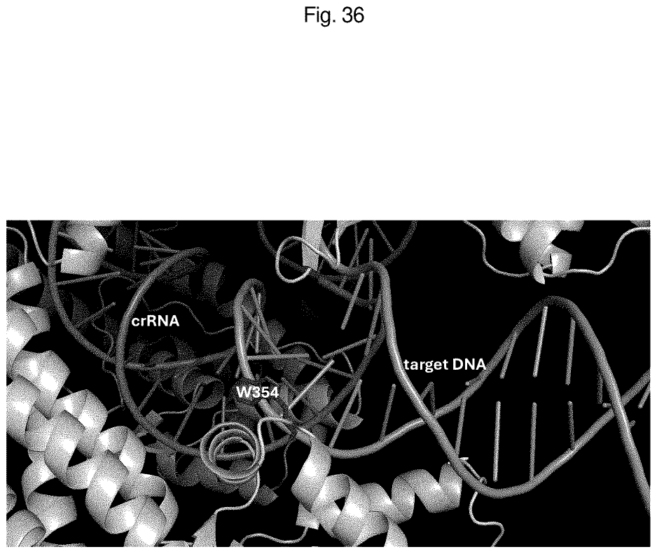

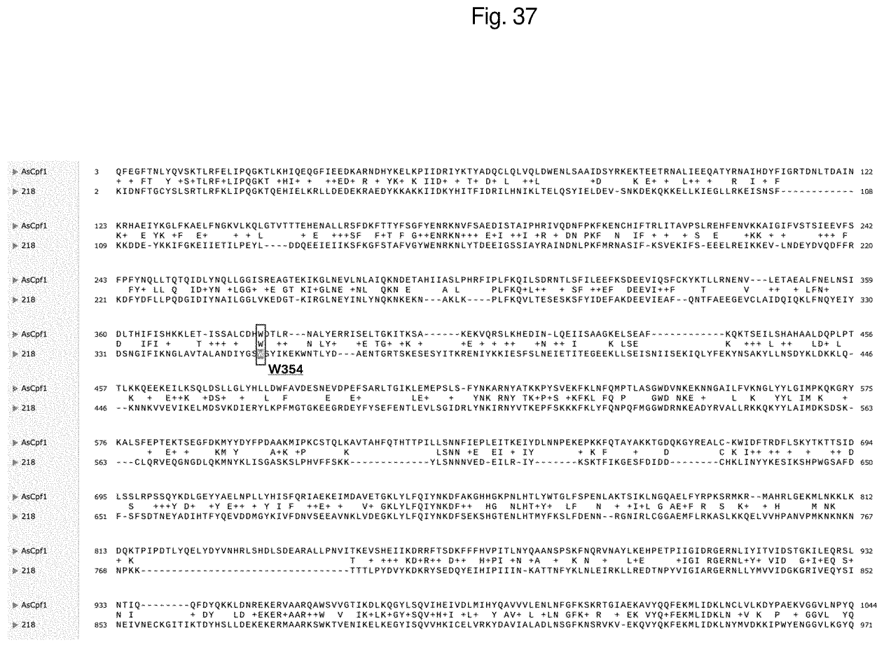

The analysis results are shown in FIGS. 33 to 37.

As a result of the analysis, the positions corresponding to the Ultra Nuclease mutation were found to be N526 and E791 in the amino acid sequence of SEQ ID NO: 1.

In addition, the position corresponding to the R-loop mutation was found to be W354 in the amino acid sequence of SEQ ID NO: 1.

Hereinafter, Cas12a protein variants were prepared by combining the mutations identified at the positions above with the PAM recognition site mutation described in Experimental Example 5, and the gene editing efficiency was measured.

### Experimental Example 7.2. Experimental Methods and Compositions Used in Experiments

In Experimental Example 7.1, variants were prepared in which the amino acid at a specific position was replaced with arginine and leucine, respectively, and used in the experiments. All possible combinations of proteins were prepared, replacing each mutation position with an appropriate amino acid. Specifically, according to Experimental Example 1.9, a plasmid encoding each variant Cas12a protein was constructed, and a guide RNA plasmid capable of operating with its corresponding Cas12a protein was also prepared. The corresponding plasmid vector was transfected into cells according to Experimental Example 1.6 using the Lipofectamine 2000 system (Thermo, 11668019). Afterward, the cell genome was extracted and gene editing activity was measured. The extracted cell genome was analyzed according to Experimental Example 1.12.

The composition for each of the CRISPR/Cas12a complexes used in Experimental Example 7 is shown in the following table:

TABLE 14

| | | Cas12a | | | |
|---|---|---|---|---|---|
| | | protein | | | Guide domain |
| | Chart | (SEQ ID | | Direct repeat | of guide RNA |
| Label | Label | NO) | C-terminal NLS | of guide RNA | (SEQ ID NO) |
| Example | 218-WT | 1 | KRPAATKKAGQ | UAAUUUCUA | DYRK1A: 143 |
| 7.1 | | | AKKKKPAAKRV | | MTAP: 140 |
| | | | KLDGGGGSGG | CUAAGUGUA | VEGFA: 419 |
| | | | GGSGGGGSPA | GAU (SEQ ID | FANCF: 420 |
| | | | AKRVKLD (SEQ | NO: 33) | CFTR: 421 |
| | | | ID NO: 70) | | Site3: 422 |
| Example | 218-WT | 264 | (Same as above) | (Same as | (Same as |
| 7.2 | (E155R) | | | above) | above) |
| Example | 218-WT (E791L) | 269 | (Same as above) | (Same as | (Same as |
| 7.3 | | | | above) | above) |
| Example | 218-WT (E155R | 270 | (Same as above) | (Same as | (Same as |
| 7.4 | + E791L) | | | above) | above) |
| Example | 218-WT (E155R | 271 | (Same as above) | (Same as | (Same as |
| 7.5 | + N526R + | | | above) | above) |
| | E791L) | | | | |
| Example | 218-WT | 272 | (Same as above) | (Same as | (Same as |
| 7.6 | (N526R) | | | above) | above) |
| Example | 218-WT (N526R | 273 | (Same as above) | (Same as | (Same as |
| 7.7 | + E791L) | | | above) | above) |
| Example | 218-WT (E155R | 274 | (Same as above) | (Same as | (Same as |
| 7.8 | + N526R) | | | above) | above) |
| Example | 218-WT | 302 | (Same as above) | (Same as | (Same as |
| 7.9 | (W354A) | | | above) | above) |
| Example | 218-WT (E155A | 303 | (Same as above) | (Same as | (Same as |
| 7.10 | + W354A) | | | above) | above) |
| Control | UNT | — | — | — | — |

### Experimental Example 7.3. Experiment Results

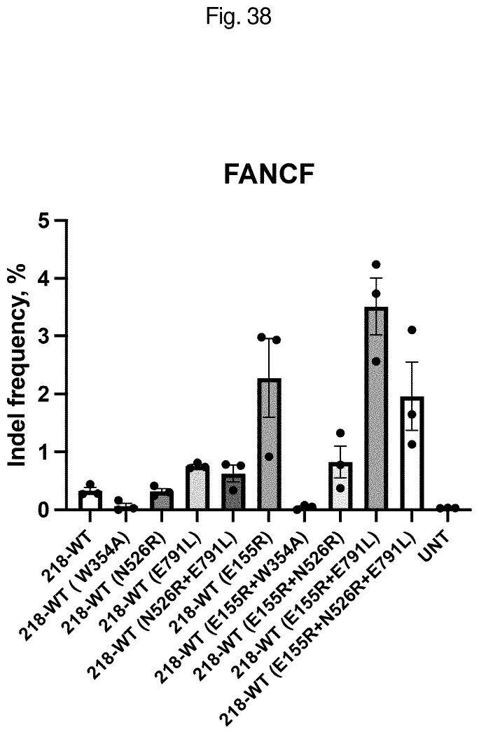

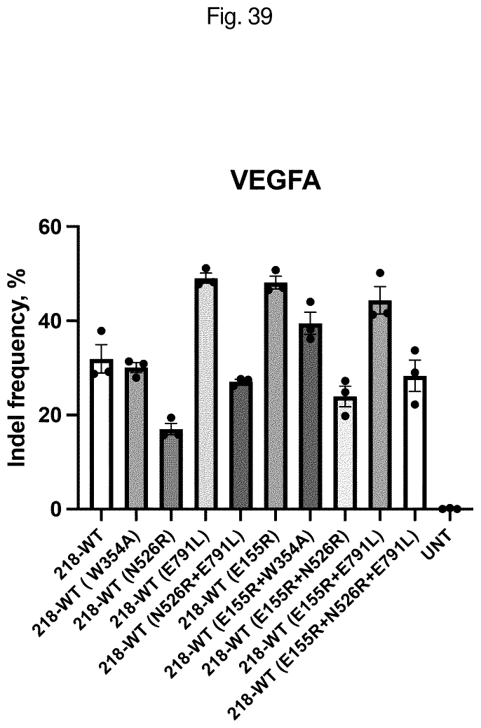

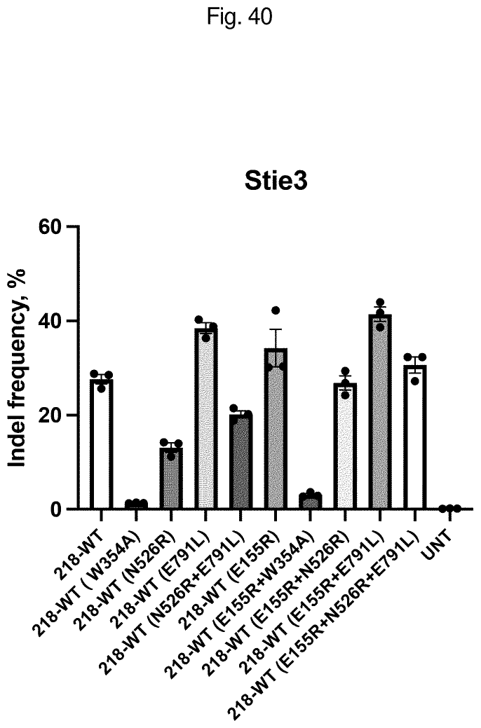

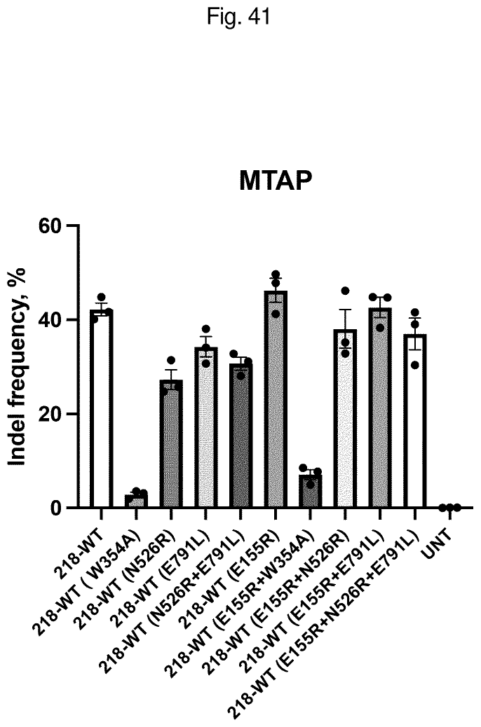

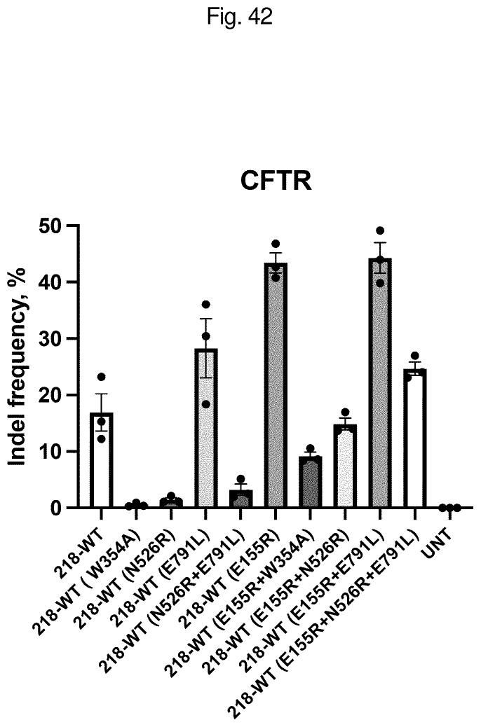

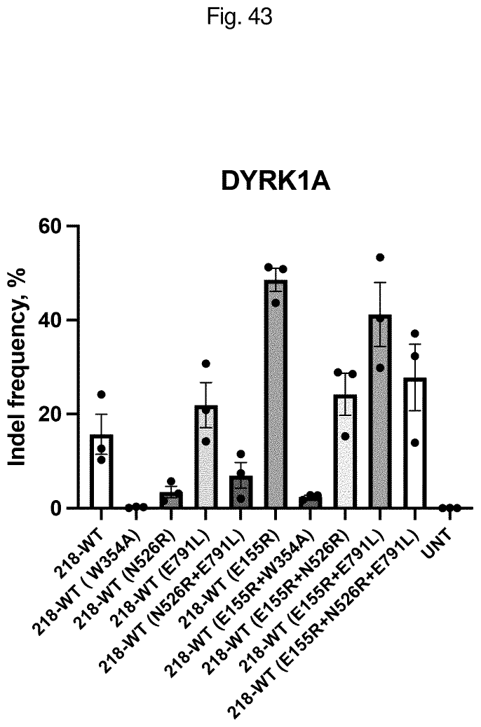

The experimental results are shown in FIGS. 38 to 43. Quantitative data for the experimental results are shown in the following table:

TABLE 5

| Label | FANCF | VEGFA | site3 | MTAP | CFTR | DYRK1A |
|---|---|---|---|---|---|---|
| 218-WT | 0.28 | 29.18 | 25.60 | 40.14 | 15.32 | 10.27 |
| (Example 7.1) | 0.27 | 28.71 | 28.52 | 41.68 | 23.24 | 12.67 |
| | 0.44 | 37.87 | 28.75 | 44.82 | 12.22 | 24.18 |
| 218-WT | 0.92 | 47.02 | 30.13 | 41.22 | 42.70 | 43.61 |
| (E155R) | 2.93 | 50.79 | 30.34 | 49.66 | 46.79 | 51.24 |
| (Example 7.2) | 2.98 | 46.53 | 42.21 | 47.83 | 40.79 | 50.85 |
| 218-WT | 0.72 | 48.20 | 36.32 | 38.08 | 30.42 | 14.16 |
| (E791L) | 0.74 | 51.16 | 38.81 | 30.68 | 18.34 | 20.89 |
| (Example 7.3) | 0.81 | 47.78 | 40.27 | 34.06 | 36.06 | 30.72 |
| 218-WT | 3.73 | 41.61 | 38.63 | 38.30 | 43.97 | 29.82 |
| (E155R + | 4.24 | 50.16 | 41.73 | 44.73 | 39.83 | 40.37 |
| E791L) | 2.56 | 41.27 | 43.96 | 44.82 | 49.14 | 53.31 |
| (Example 7.4) | | | | | | |
| 218-WT | 1.65 | 22.20 | 27.22 | 30.38 | 26.97 | 32.34 |
| (E155R + | 1.13 | 33.75 | 32.34 | 41.60 | 23.10 | 37.15 |
| N526R + | 3.10 | 29.02 | 32.34 | 39.03 | 24.01 | 13.90 |
| E791L) | | | | | | |
| (Example 7.5) | | | | | | |
| 218-WT | 0.30 | 15.75 | 11.15 | 24.70 | 1.08 | 1.55 |
| (N526R) | 0.41 | 19.42 | 13.97 | 31.44 | 2.13 | 3.11 |
| (Example 7.6) | 0.24 | 15.82 | 14.24 | 25.70 | 1.16 | 5.72 |
| 218-WT | 0.33 | 27.46 | 20.20 | 32.76 | 2.54 | 2.02 |
| (N526R + | 0.76 | 26.16 | 18.85 | 28.05 | 1.99 | 7.42 |
| E791L) | 0.79 | 27.64 | 21.47 | 31.14 | 5.16 | 11.51 |
| (Example 7.7) | | | | | | |
| 218-WT | 0.37 | 19.77 | 24.23 | 32.84 | 14.03 | 15.26 |
| (E155R + | 0.78 | 24.85 | 29.39 | 35.19 | 13.69 | 28.89 |
| N526R) | 1.33 | 27.20 | 26.81 | 46.20 | 16.98 | 28.53 |
| (Example 7.8) | | | | | | |
| 218-WT | 0.03 | 27.91 | 1.26 | 1.96 | 0.41 | 0.07 |
| (W354A) | 0.16 | 30.89 | 1.32 | 3.11 | 0.93 | 0.34 |
| (Example 7.9) | 0.00 | 31.36 | 1.45 | 3.57 | 0.33 | 0.23 |
| 218-WT | 0.05 | 38.20 | 2.89 | 4.92 | 8.37 | 1.79 |
| (E155R + | 0.08 | 36.18 | 3.68 | 7.74 | 8.61 | 2.73 |
| W354A) | 0.00 | 44.00 | 2.73 | 8.50 | 10.57 | 2.77 |
| (Example 7.10) | | | | | | |
| UNT | 0.03 | 0.21 | 0.20 | 0.03 | 0.00 | 0.03 |
| (Control) | 0.03 | 0.03 | 0.17 | 0.07 | 0.00 | 0.00 |
| | 0.03 | 0.00 | 0.13 | 0.07 | 0.04 | 0.05 |

All figures express the indel introduction rate as a percentage. The experiments were repeated 3 times for each experimental example. As a result of the experiments, those in Examples 7.2 to 7.5 showed higher eukaryotic genome editing efficiency than the novel Cas12a protein (Example 7.1) in which no mutation was introduced. In Example 7.6 and Example 7.8, excellent editing efficiency was not shown, but editing efficiency was shown at a usable level.

### Experimental Example 8. Eukaryotic Genome Editing Activity Experiments of CRISPR/Cas12a Systems Including Cas12a Protein Variants #4

Experimental Example 8.1. Experimental Methods and Compositions Used in Experiments Variants, in which the corresponding amino acid at a specific position in Experimental Example 5.1 was replaced with arginine, and variants combining additional mutations identified based on the conventional literature (Yamano T, Nishimasu H, Zetsche B, Hirano H, Slaymaker I M, Li Y, Fedorova I, Nakane T, Makarova K S, Koonin E V, Ishitani R, Zhang F, Nureki O. Crystal Structure of Cpf1 in Complex with Guide RNA and Target DNA. Cell. 2016 May 5; 165 (4): 949-62. doi: 10.1016/j.cell.2016.04.003. Epub 2016 Apr. 21. PMID: 27114038; PMCID: PMC4899970.), and wild-type Cas12a proteins with a modified nuclear localization signal were used in the experiments.

Specifically, according to Experimental Example 1.9, a plasmid encoding each variant Cas12a protein was constructed, and a guide RNA plasmid capable of operating with its corresponding Cas12a protein was also prepared. The corresponding plasmid vector was transfected into cells according to Experimental Example 1.6 using the Lipofectamine 2000 system (Thermo, 11668019). Afterward, the cell genome was extracted and gene editing activity was measured. The extracted cell genome was analyzed according to Experimental Example 1.12.

The composition for each of the CRISPR/Cas12a complexes used in Experimental Example 8 is shown in the following table:

TABLE 16

| | | | | | | Guide |
|---|---|---|---|---|---|---|
| | | | | | | domain of |
| | | | Cas12a | | | guide RNA |
| | | | protein | | Direct repeat | (SEQ ID |
| Label | Chart | N-terminal | (SEQ ID | | of guide | NO) |
| Example | Label | NLS | NO) | C-terminal NLS | RNA | DYRK1A: |
| 8.1 | 218-WT | PKKKRKV | 1 | SEQ ID NO: 67 | UAAUUUCU | 143 |
| | | (SEQ ID | | | ACUAAGU | MTAP: 140 |
| | | NO: 67) | | | GUAGAU | VEGFA: ? |
| | | | | | (SEQ ID NO: | FANCF: ? |
| | | | | | 33) | CFTR: ? |
| Example | 218-WT- | — | 1 | KRPAATKKA | (Same as | (Same as |
| 8.2 | NLS6 | | | GQAKKKKPA | above) | above) |
| | | | | AKRVKLDGG | | |
| | | | | GGSGGGGS | | |
| | | | | GGGGSPAAK | | |
| | | | | RVKLD (SEQ | | |
| | | | | ID NO: 70) | | |
| Example | 218-WT- | — | 286 | SEQ ID NO: 70 | (Same as | (Same as |
| 8.3 | S1137A- | | | | above) | above) |
| | NLS6 | | | | | |
| Example | 218-WT- | | 300 | SEQ ID NO: 70 | (Same as | (Same as |
| 8.4 | RR-NLS6 | | | | above) | above) |
| Example | 218-WT- | SEQ ID | 301 | SEQ ID NO: 67 | (Same as | (Same as |
| 8.5 | RRR | NO: 67 | | | above) | above) |
| Example | 218-WT- | | 301 | SEQ ID NO: 70 | (Same as | (Same as |
| 8.6 | RRR- | | | | above) | above) |
| | NLS6 | | | | | |
| | 218-WT- | | | | | |
| Example | S1137A- | | 304 | SEQ ID NO: 70 | (Same as | (Same as |
| 8.7 | RRR- | | | | above) | above) |
| | NLS6 | | | | | |
| Control | UNT | — | — | — | — | — |

Site3: ?

### Experimental Example 8.2. Experiment Results

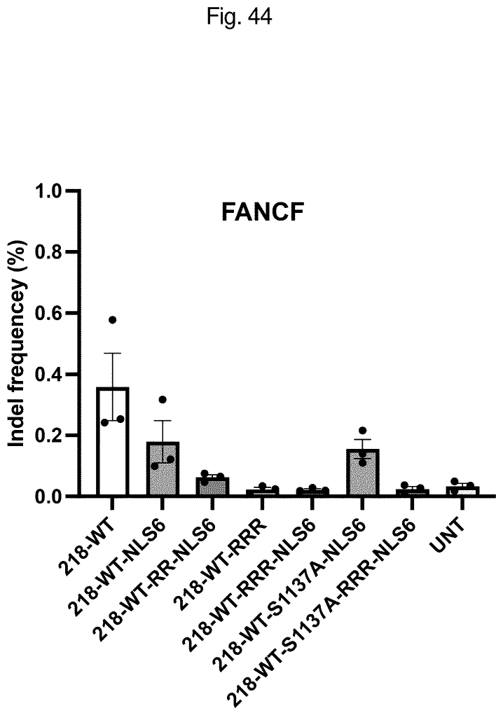

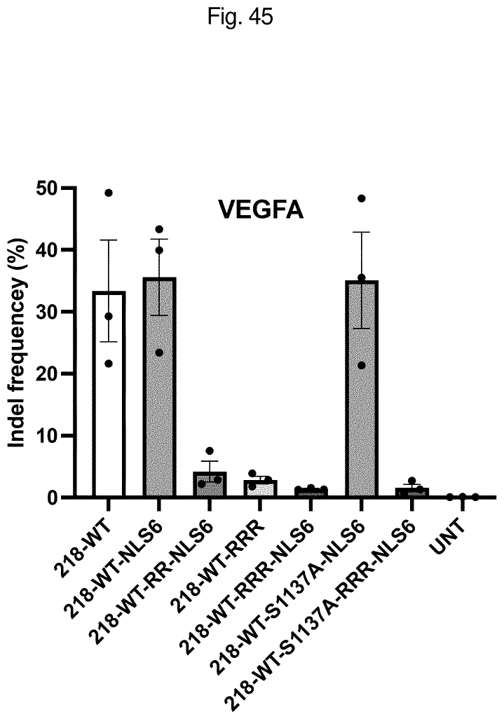

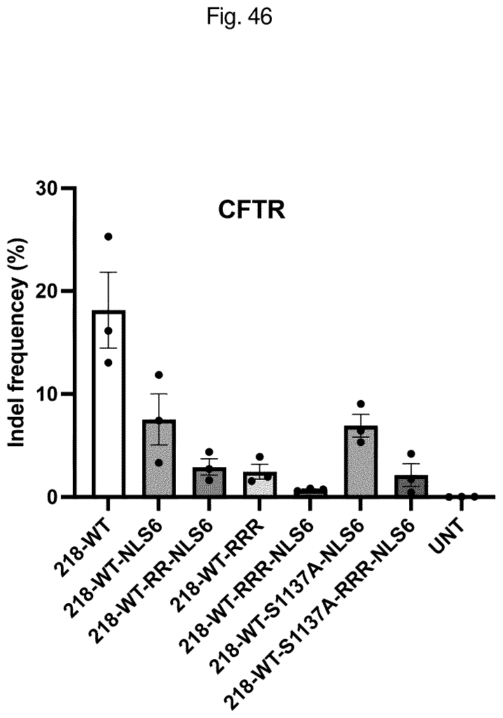

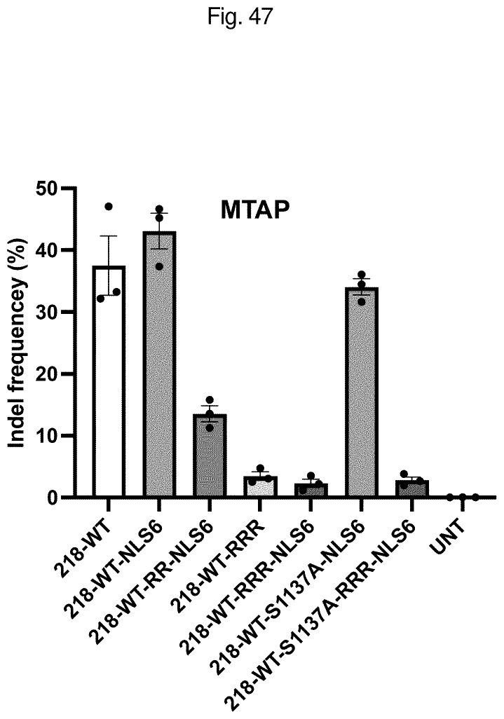

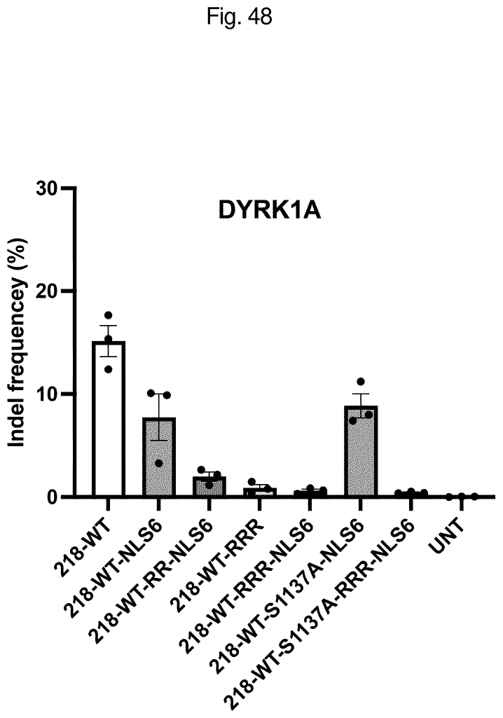

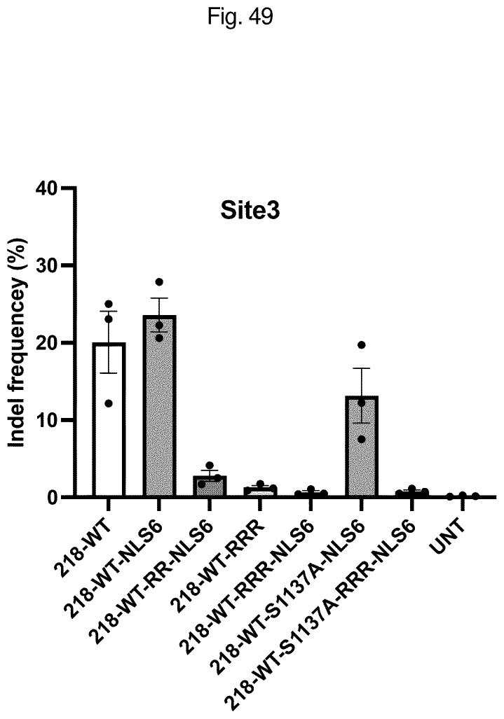

The experimental results are shown in FIGS. 44 to 49.

As a result of the experiments, Examples 8.1 to 8.2 showed high eukaryotic genome editing efficiency. In Example 8.3, excellent editing efficiency was not shown, but editing efficiency was shown at a usable level.

### Experimental Example 9. Confirmation of Multiple Target Nucleic Acid Cleavage Activity Using Multiplex Vector Load

### Experimental Example 9.1. Experiment Overview and Examples Used in Experiments

When the novel CRISPR/Cas12a systems disclosed herein have the characteristics of the known CRISPR/Cas12a, a nucleic acid encoding a guide RNA will be capable of self-maturation even when it does not contain a terminator or other components.

Furthermore, even when transcription is induced by linking DNA encoding multiple guide RNAs without any spacers or terminators, each guide RNA will mature without any problems.

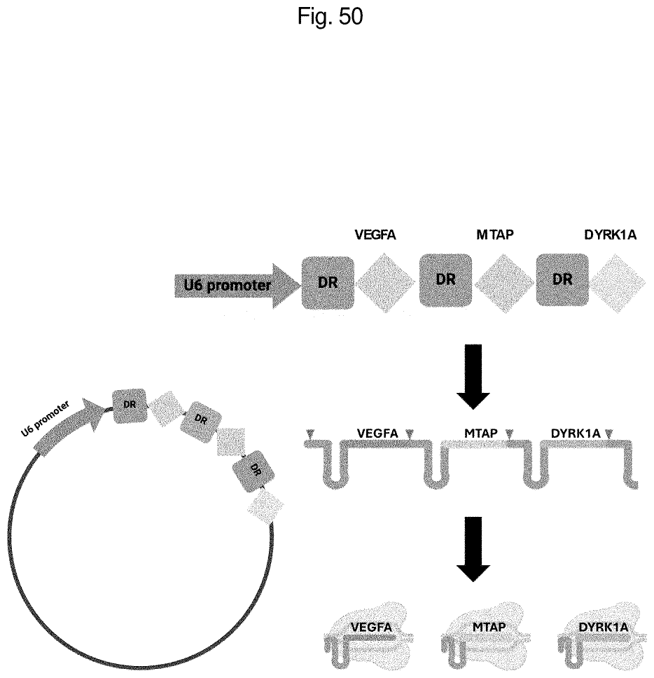

The process of self-maturation of multiple guide RNA sequences linked to one promoter is schematically shown in FIG. 50.

Applying these features, it is very easy to design a vector construct that induces simultaneous editing of multiple target nucleic acids.

Referring to Experimental Example 1, vector constructs containing the encoding nucleic acids in the following table were prepared:

TABLE 17

| | Direct | Guide | Direct | Guide | Direct | Guide |
|---|---|---|---|---|---|---|
| Promoter | Repeat #1 | Domain #1 | Repeat #2 | Domain #2 | Repeat #3 | Domain #3 |
| GAGGGCCTAT | TAATTTCT | CTCTCAAG | SEQ ID | GCCCCAA | SEQ | GAAGCAC |
| TTCCCATGATT | ACTAAGTG | ACCCACAA | NO: 50 | TAATCCCC | ID | ATCAAGGA |
| CCTTCATATTT | TAGAT | TCCAGGC | | ACATGTCA | NO: 50 | CATTCTAA |
| GCATATACGA | (SEQ ID | (SEQ ID | | (SEQ ID | | (SEQ ID |
| TACAAGGCTG | NO: 50) | NO: 415) | | NO: 139) | | NO: 142) |
| TTAGAGAGAT | | | | | | |
| AATTGGAATTA | | | | | | |
| ATTTGACTGTA | | | | | | |
| AACACAAAGA | | | | | | |
| TATTAGTACAA | | | | | | |
| AATACGTGAC | | | | | | |
| GTAGAAAGTA | | | | | | |
| ATAATTTCTTG | | | | | | |
| GGTAGTTTGC | | | | | | |
| AGTITTAAAAT | | | | | | |
| TATGTTTTAAA | | | | | | |
| ATGGACTATC | | | | | | |
| ATATGCTTAC | | | | | | |
| CGTAACTTGA | | | | | | |
| AAGTATTTCG | | | | | | |
| ATTTCTTGGCT | | | | | | |
| TTATATATCTT | | | | | | |
| GTGGAAAGGA | | | | | | |
| C (SEQ ID NO: | | | | | | |
| 423) | | | | | | |

Vector constructs were prepared containing DNA sequences listed in the table above and linked in order from the 5′ end to the 3′ end. The vector constructs prepared above were delivered to cells together with the nucleic acids encoding the Cas12a protein having the amino acid of SEQ ID NO: 1 prepared according to Experimental Example 1.9, and it was confirmed whether a target sequence gene of each guide RNA was edited.

### Experimental Example 9.2. Experiment Results

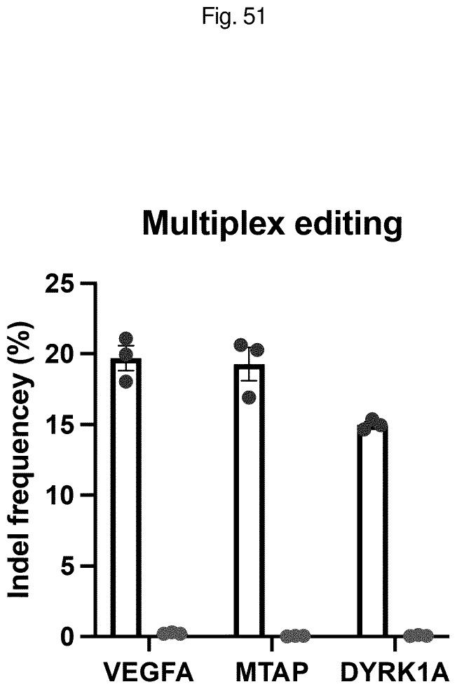

The experimental results of Experimental Example 9.1 are shown in FIG. 51.

As a result of the experiments, it was confirmed that when the vector constructs of Experimental Example 9.1 and the Cas12a proteins of SEQ ID NO: 1 were delivered together to cells, indels were introduced in all of the multiple targets as intended.

This means that by using the vector constructs containing a plurality of guide RNAs and the Cas12a protein disclosed in this specification, a plurality of target nucleic acids in the eukaryotic genome could be edited.

### Experimental Example 10. Primer Sequences Used in Experimental Examples 2 to 10

Primer sequences used in Experimental Examples 2 to 8 are shown in the following table:

TABLE 18

| | | SEQ ID |
|---|---|---|
| PRIMER NAME | PRIMER SEQUENCE | NO |
| ID_204_FWD | GAAATTAATACGACTCACTATAGGTAATTTCTACTA | 147 |
| | TTCGTAGAT | |
| ID_204_RG1_REV | CAGGGAGGGAGGGGCACAGATGAATCTACGAATA | 148 |
| | GTAGAAATTA | |
| ID_206_FWD | GAAATTAATACGACTCACTATAGGTTAATTTCTACT | 149 |
| | TTCGTAGAT | |
| ID_206_RG1_REV | CAGGGAGGGAGGGGCACAGATGAATCTACGAAA | 150 |
| | GTAGAAATTAA | |
| ID_210_FWD | GAAATTAATACGACTCACTATAGGCAATTTCTACTT | 151 |
| | TCAGTAGAT | |
| ID_210_RG1_REV | CAGGGAGGGAGGGGCACAGATGAATCTACTGAAA | 152 |
| | GTAGAAATTG | |
| ID_211_FWD | GAAATTAATACGACTCACTATAGGTTAATTTCTACT | 153 |
| | TGTGTAGAT | |
| ID_211_RG1_REV | CAGGGAGGGAGGGGCACAGATGAATCTACACAA | 154 |
| | GTAGAAATTAA | |
| ID_218_FWD | GAAATTAATACGACTCACTATAGGTAATTTCTACTA | 155 |
| | AGTGTAGAT | |
| ID_218_RG1_REV | CAGGGAGGGAGGGGCACAGATGAATCTACACTTA | 156 |
| | GTAGAAATTA | |
| ID_219_230_FWD | GAAATTAATACGACTCACTATAGGTAAATTTCTACT | 157 |
| | ATTGTAGAT | |
| ID_219_230_ | CAGGGAGGGAGGGGCACAGATGAATCTACAATAG | 158 |
| RG1_REV | TAGAAATTTA | |
| ID_223_FWD | GAAATTAATACGACTCACTATAGGAAAATTTCTACT | 159 |
| | ATTGTAGAT | |
| ID_223_RG1_REV | CAGGGAGGGAGGGGCACAGATGAATCTACAATAG | 160 |
| | TAGAAATTTT | |
| ID_238_FWD | GAAATTAATACGACTCACTATAGGATAATTTCTACT | 161 |
| | GTTGTAGAT | |
| ID_238_RG1_REV | CAGGGAGGGAGGGGCACAGATGAATCTACAACA | 162 |
| | GTAGAAATTAT | |

TABLE 19

| PRIMER NAME | PRIMER SEQUENCE | SEQ ID NO |
|---|---|---|
| ID_204_250_FWD | GAAATTAATACGACTCACTATAGGTAATTTCTA | 163 |
| | CTATTCGTAGAT | |
| ID_204_250_MTAP_ | TGACATGTGGGGATTATTGGGGCATCTACGA | 164 |
| REV | ATAGTAGAAATTA | |
| ID_204_250_DYRK1A | TTAGAATGTCCTTGATGTGCTTCATCTACGAA | 165 |
| REV | TAGTAGAAATTA | |
| ID_206_FWD | GAAATTAATACGACTCACTATAGGTTAATTTCT | 166 |
| | ACTTTCGTAGAT | |
| ID_206_MTAP_REV | TGACATGTGGGGATTATTGGGGCATCTACGA | 167 |
| | AAGTAGAAATTAA | |
| ID_206_DYRK1A_REV | TTAGAATGTCCTTGATGTGCTTCATCTACGAA | 168 |
| | AGTAGAAATTAA | |
| ID_210_FWD | GAAATTAATACGACTCACTATAGGCAATTTCT | 169 |
| | ACTTTCAGTAGAT | |
| ID_210_MTAP_REV | TGACATGTGGGGATTATTGGGGCATCTACTG | 170 |
| | AAAGTAGAAATTG | |
| ID_210_DYRK1A_REV | TTAGAATGTCCTTGATGTGCTTCATCTACTGA | 171 |
| | AAGTAGAAATTG | |
| ID_211_FWD | GAAATTAATACGACTCACTATAGGTTAATTTCT | 172 |
| | ACTTGTGTAGAT | |
| ID_211_MTAP_REV | TGACATGTGGGGATTATTGGGGCATCTACAC | 173 |
| | AAGTAGAAATTAA | |
| ID_211_DYRK1A_ | TTAGAATGTCCTTGATGTGCTTCATCTACACA | 174 |
| REV | AGTAGAAATTAA | |
| ID_217_FWD | GAAATTAATACGACTCACTATAGGAATTTCTA | 175 |
| | CTACTATGTAGAT | |
| ID_217_MTAP_REV | TGACATGTGGGGATTATTGGGGCATCTACAT | 176 |
| | AGTAGTAGAAATT | |
| ID_217_DYRK1A_ | TTAGAATGTCCTTGATGTGCTTCATCTACATA | 177 |
| REV | GTAGTAGAAATT | |
| ID_218_FWD | GAAATTAATACGACTCACTATAGGTAATTTCTA | 178 |
| | CTAAGTGTAGAT | |
| ID_218_MTAP_REV | TGACATGTGGGGATTATTGGGGCATCTACAC | 179 |
| | TTAGTAGAAATTA | |
| ID_218_DYRK1A_ | TTAGAATGTCCTTGATGTGCTTCATCTACACT | 180 |
| REV | TAGTAGAAATTA | |
| ID_230_FWD | GAAATTAATACGACTCACTATAGGTAAATTTCT | 181 |
| | ACTATTGTAGAT | |
| ID_230_MTAP_REV | TGACATGTGGGGATTATTGGGGCATCTACAA | 182 |
| | TAGTAGAAATTTA | |
| ID_230_DYRK1A_ | TTAGAATGTCCTTGATGTGCTTCATCTACAAT | 183 |
| REV | AGTAGAAATTTA | |
| ID_233_FWD | GAAATTAATACGACTCACTATAGGAAAATTTC | 184 |
| | TACTCTTGTAGAT | |
| ID_233_MTAP_REV | TGACATGTGGGGATTATTGGGGCATCTACAA | 185 |
| | GAGTAGAAATTTT | |
| ID_233_DYRK1A_ | TTAGAATGTCCTTGATGTGCTTCATCTACAAG | 186 |
| REV | AGTAGAAATTTT | |
| ID_236_FWD | GAAATTAATACGACTCACTATAGGTAAATTTCT | 187 |
| | ACTGTTGTAGAT | |
| ID_236_MTAP_REV | TGACATGTGGGGATTATTGGGGCATCTACAA | 188 |
| | CAGTAGAAATTTA | |
| ID_236_DYRK1A_ | TTAGAATGTCCTTGATGTGCTTCATCTACAAC | 189 |
| REV | AGTAGAAATTTA | |
| ID_239_FWD | GAAATTAATACGACTCACTATAGGATAATTTCT | 190 |
| | ACTATTGTAGAT | |
| ID_239_MTAP_REV | TGACATGTGGGGATTATTGGGGCATCTACAA | 191 |
| | TAGTAGAAATTAT | |
| ID_239_DYRK1A_ | TTAGAATGTCCTTGATGTGCTTCATCTACAAT | 192 |
| REV | AGTAGAAATTAT | |

TABLE 20

| | | SEQ ID |
|---|---|---|
| PRIMER NAME | PRIMER SEQUENCE | NO |
| ID_218_DNMT1_ | CCTTTATTTTAGCTGAAGGGAAAATCTACACTTAGTAGA | 387 |
| REV | AATTA | |
| ID_218_DNMT3B_ | GAGGTATCCAGCAGAGGGGAGAAATCTACACTTAGTA | 388 |
| REV | GAAATTA | |
| ID_218_DYRK1A_ | TTAGAATGTCCTTGATGTGCTTCATCTACACTTAGTAGA | 389 |
| REV | AATTA | |
| ID_218_EMX1_ | CAGGGAGGGAGGGGCACAGATGAATCTACACTTAGTA | 390 |
| REV | GAAATTA | |
| ID_230_FWD | GAAATTAATACGACTCACTATAGGTAAATTTCTACTATT | 391 |
| | GTAGAT | |
| ID_230_MTAP_ | TGACATGTGGGGATTATTGGGGCATCTACAATAGTAGA | 392 |
| REV | AATTTA | |
| ID_230_DNMT1_ | CCTTTATTTTAGCTGAAGGGAAAATCTACAATAGTAGAA | 393 |
| REV | ATTTA | |
| ID_230_DNMT3B_ | GAGGTATCCAGCAGAGGGGAGAAATCTACAATAGTAG | 394 |
| REV | AAATTTA | |
| ID_230_DYRK1A_ | TTAGAATGTCCTTGATGTGCTTCATCTACAATAGTAGAA | 395 |
| REV | ATTTA | |
| ID_230_EMX1_ | CAGGGAGGGAGGGGCACAGATGAATCTACAATAGTAG | 396 |
| REV | AAATTTA | |
| ID_233_FWD | GAAATTAATACGACTCACTATAGGAAAATTTCTACTCTT | 397 |
| | GTAGAT | |
| ID_233_MTAP_ | TGACATGTGGGGATTATTGGGGCATCTACAAGAGTAG | 398 |
| REV | AAATTTT | |
| ID_233_DNMT1_ | CCTTTATTTTAGCTGAAGGGAAAATCTACAAGAGTAGA | 399 |
| REV | AATTTT | |
| ID_233_DNMT3B_ | GAGGTATCCAGCAGAGGGGAGAAATCTACAAGAGTAG | 400 |
| REV | AAATTTT | |
| ID_233_DYRK1A_ | TTAGAATGTCCTTGATGTGCTTCATCTACAAGAGTAGA | 401 |
| REV | AATTTT | |
| ID_233_EMX1_ | CAGGGAGGGAGGGGCACAGATGAATCTACAAGAGTA | 402 |
| REV | GAAATTTT | |
| ID_236_FWD | GAAATTAATACGACTCACTATAGGTAAATTTCTACTGTT | 403 |
| | GTAGAT | |
| ID_236_MTAP_ | TGACATGTGGGGATTATTGGGGCATCTACAACAGTAGA | 404 |
| REV | AATTTA | |
| ID_236_DNMT1_ | CCTTTATTTTAGCTGAAGGGAAAATCTACAACAGTAGA | 405 |
| REV | AATTTA | |
| ID_236_DNMT3B_ | GAGGTATCCAGCAGAGGGGAGAAATCTACAACAGTAG | 406 |
| REV | AAATTTA | |
| ID_236_DYRK1A_ | TTAGAATGTCCTTGATGTGCTTCATCTACAACAGTAGAA | 407 |
| REV | ATTTA | |
| ID_236_EMX1_ | CAGGGAGGGAGGGGCACAGATGAATCTACAACAGTAG | 408 |
| REV | AAATTTA | |
| ID_239_FWD | GAAATTAATACGACTCACTATAGGATAATTTCTACTATT | 409 |
| | GTAGAT | |
| ID_239_MTAP_ | TGACATGTGGGGATTATTGGGGCATCTACAATAGTAGA | 410 |
| REV | AATTAT | |
| ID_239_DNMT1_ | CCTTTATTTTAGCTGAAGGGAAAATCTACAATAGTAGAA | 411 |
| REV | ATTAT | |
| ID_239_DNMT3B_ | GAGGTATCCAGCAGAGGGGAGAAATCTACAATAGTAG | 412 |
| REV | AAATTAT | |
| ID_239_DYRK1A_ | TTAGAATGTCCTTGATGTGCTTCATCTACAATAGTAGAA | 413 |
| REV | ATTAT | |
| ID_239_EMX1_ | CAGGGAGGGAGGGGCACAGATGAATCTACAATAGTAG | 414 |
| REV | AAATTAT | |

TABLE 21
NGS 1st PCR Primer

| Label | Sub Label | Sequence | SEQ ID NO |
|---|---|---|---|
| FANCF | FANCF_T7_FWD | ACCGTTACAAAGCTAGTCCC | 307 |
| | FANCF_T7_REV | CCAAAGTTAATGCACCGAGG | 308 |
| PD1 | PD1_T7_FWD | ACGTCGTAAAGCCAAGGTTA | 309 |
| | PD1_T7_REV | CTCCCTTCAACCTGACCTG | 310 |
| VEGFA | VEGFA_T7_FWD | AAGCGACTCAACCTGGTAAA | 311 |
| | VEGFA_T7_REV | AGTCAAGCCATGCCTTTTTC | 312 |
| Site4 | Site4_T7_FWD | CCAAACAAGGAAGCCAAAGT | 313 |
| | Site4_T7_REV | AAAGAGCAAACCTGAACTGG | 314 |
| CFTR | CFTR_T7_FWD | GCTTGGACTTCATGTGTGTC | 315 |
| | CFTR_T7_REV | TAACTGGAAACTTCAGCGGT | 316 |
| Site6 | Site6_T7_FWD | CCTGTATGTAGCGCAAGAGA | 317 |
| | Site6_T7_REV | CCCGAGCCCTGTCTATAAAT | 318 |
| IFTAP | IFTAP_T7_FWD | AACCCACCCACGATTGTAAA | 319 |
| | IFTAP_T7_REV | GCTGACAGTATTGTGCAGTG | 320 |
| Site6-T | Site6-T_T7_FWD | GTCCATAGGCGAGAAGAACA | 321 |
| | Site6-T_T7_REV | GAGTTGAGAGGATGGGATGG | 322 |
| VEGF-T | VEGF-T_T7_FWD | CCAGAGTCCCCCAGAAATAC | 323 |
| | VEGF-T_T7_REV | GAGCCCGATACACACACTTA | 324 |
| DNMT1 | DNMT1_T7E1_FWD | GAACGTTCCCTTAGCACTCT | 325 |
| | DNMT1_T7E1_REV | GCAAATCACGAATACCCACC | 326 |
| DYRK1A | DYRK1A_T7E1_FWD | AGTACACCTTCTTTCGCAGT | 327 |
| | DYRK1A_T7E1_REV | GAGACGTTATGCATTCACCA | 328 |
| EMX1 | EMX1_T7E1_FWD | TGGGTCATAGGCTCTCTCAT | 329 |
| | EMX1_T7E1_REV | GAGATTGGAGACACGGAGAG | 330 |
| DNMT3B | DNMT3B_T7E1_FWD | AAGTTTCTTCAGCGGTCTCT | 331 |
| | DNMT3B_T7E1_REV | TTCGCTGACTCTCTTTTGGA | 332 |
| MTAP | MTAP_T7E1_FWD | GAGGTTTGGAGGGGTTAAGG | 333 |
| | MTAP_T7E1_REV | TAAAGACACCCCCAAGACTG | 334 |
| RUNX1 | RUNX1_T7E1_FWD | GGCCTCATAAACAACCACAG | 335 |
| | RUNX1_T7E1_REV | AAGACCAGCATGTACTCACC | 336 |

TABLE 22
NGS 2nd PCR Primer (Adapt)

| | | | SEQ ID |
|---|---|---|---|
| Label | Sub Label | Sequence | NO |
| FANCF | FANCF_Adapt_FW | ACACTCTTTCCCTACACGACGCTCTTCCGATC | 337 |
| | D | TCACTTTTATAACGAAGAACTCTTTGTG | 338 |
| | FANCF_Adapt_ | GTGACTGGAGTTCAGACGTGTGCTCTTCCGAT | |
| | REV | CTCATCATTAGAAGCTTGGATGCTC | |
| PD1 | PD1_Adap_FWD | ACACTCTTTCCCTACACGACGCTCTTCCGATC | 339 |
| | | TAGAGAGGAACCCAGGAGTTC | |
| | PD1_Adap_REV | GTGACTGGAGTTCAGACGTGTGCTCTTCCGAT | 340 |
| | | CTGTTCTTAGGTAGGTGGGGTC | |
| VEGFA | VEGFA_Adap_FW | ACACTCTTTCCCTACACGACGCTCTTCCGATC | 341 |
| | D | TGCCAGGCATTGAAGAAGG | |
| | VEGFA_Adap_RE | GTGACTGGAGTTCAGACGTGTGCTCTTCCGAT | 342 |
| | V | CTGAACCATGCCATGTCCCTAG | |
| Site4 | Site4_Adap_FWD | ACACTCTTTCCCTACACGACGCTCTTCCGATC | 343 |
| | | TGTGGAGTTTGTATTTTGCATACTCAAG | |
| | Site4_Adap_REV | GTGACTGGAGTTCAGACGTGTGCTCTTCCGAT | 344 |
| | | CTCAAGAGTTCTCAGCCCTATATTGATAG | |
| CFTR | CFTR_Adap_FWD | ACACTCTTTCCCTACACGACGCTCTTCCGATC | 345 |
| | | TCAGCCCTTTCCCAGGTAAG | |
| | CFTR_Adap_REV | GTGACTGGAGTTCAGACGTGTGCTCTTCCGAT | 346 |
| | | CTCTCTCCTCTCCCCATGATG | |
| Site6 | Site6_Adap_FWD | ACACTCTTTCCCTACACGACGCTCTTCCGATC | 347 |
| | | TCTGAGGGGCTCTGCATTG | |
| | Site6_Adap_REV | GTGACTGGAGTTCAGACGTGTGCTCTTCCGAT | 348 |
| | | CTAGGGAGGAAACCCTGACTTC | |
| IFTAP | IFTAP_Adap_FWD | ACACTCTTTCCCTACACGACGCTCTTCCGATC | 349 |
| | | TGGCAGGCAGACTCGATAC | |
| | IFTAP_Adap_REV | GTGACTGGAGTTCAGACGTGTGCTCTTCCGAT | 350 |
| | | CTCTCTAACCTATTTGCCAAGGTAGG | |
| Site6-T | Site6- | ACACTCTTTCCCTACACGACGCTCTTCCGATC | 351 |
| | T_Adap_FWD | TTACCCAGGCATGGTGGTG | |
| | Site6- | GTGACTGGAGTTCAGACGTGTGCTCTTCCGAT | 352 |
| | T_Adap_REV | CTGTTGAGAGGATGGGATGGAAC | |
| VEGF-T | VEGF- | ACACTCTTTCCCTACACGACGCTCTTCCGATC | 353 |
| | T_Adap_FWD | TCTTGGCTAGGGGGCAATG | |
| | VEGF- | GTGACTGGAGTTCAGACGTGTGCTCTTCCGAT | 354 |
| | T_Adap_REV | CTCTTGGGCACAATGCTTATATGTG | |
| DNMT1 | DNMT1_ADAPT_F | ACACTCTTTCCCTACACGACGCTCTTCCGATC | 355 |
| | WD | TCCACTTATTGGGTCAGCTGT | |
| | DNMT1_ADAPT | GTGACTGGAGTTCAGACGTGTGCTCTTCCGAT | 356 |
| | REV | CTATCAGTGCATGTTGGGGATT | |
| DYRK1A | DYRK1A_ADAPT | ACACTCTTTCCCTACACGACGCTCTTCCGATC | 357 |
| | FWD | TTCTTAAAACCTTGTCACACACA | |
| | DYRK1A_ADAPT | GTGACTGGAGTTCAGACGTGTGCTCTTCCGAT | 358 |
| | FWD | CTGCTCCCCTAAGCAAATACTC | |
| EMX1 | EMX1_ADAPT_F | ACACTCTTTCCCTACACGACGCTCTTCCGATC | 359 |
| | WD | TGTAGCCTCAGTCTTCCCATC | |
| | EMX1_ADAPT_RE | GTGACTGGAGTTCAGACGTGTGCTCTTCCGAT | 360 |
| | V | CTTTCTTCTTCTGCTCGGACTC | |
| DNMT3B | DNMT3B_Adapt_F | ACACTCTTTCCCTACACGACGCTCTTCCGATC | 361 |
| | WD | TGGTAACAGAGCGACAACAGT | |
| | DNMT3B_Adapt_R | GTGACTGGAGTTCAGACGTGTGCTCTTCCGAT | 362 |
| | EV | CTTGTTTGGTGGTCTCTTCACA | |
| MTAP | MTAP_ADAPT_F | ACACTCTTTCCCTACACGACGCTCTTCCGATC | 363 |
| | WD | TCCATCTGTACCCCCAAAAAC | |
| | MTAP_ADAPT_R | GTGACTGGAGTTCAGACGTGTGCTCTTCCGAT | 364 |
| | EV | CTGCATGTGCAGGGGAACTC | |
| RUNX1 | RUNX1_ADAPT_F | ACACTCTTTCCCTACACGACGCTCTTCCGATC | 365 |
| | WD | TAGCATCACCAACCCACAG | |
| | RUNX1_ADAPT_R | GTGACTGGAGTTCAGACGTGTGCTCTTCCGAT | 366 |
| | EV | CTCGGGGGACTCAATGATTTCT | |

TABLE 23
3rd Index Primer

| | Sub | | SEQ ID |
|---|---|---|---|
| Label | Label | Sequence | NO |
| D501~ | D501 | AATGATACGGCGACCACCGAGATCTACACtatagcctAC | 367 |
| D508 | | ACTCTTTCCCTACACGAC | |
| | D502 | AATGATACGGCGACCACCGAGATCTACACatagaggcA | 368 |
| | | CACTCTTTCCCTACACGAC | |
| | D503 | AATGATACGGCGACCACCGAGATCTACACcctatcctAC | 369 |
| | | ACTCTTTCCCTACACGAC | |
| | D504 | AATGATACGGCGACCACCGAGATCTACACggctctgaAC | 370 |
| | | ACTCTTTCCCTACACGAC | |
| | D505 | AATGATACGGCGACCACCGAGATCTACACaggcgaagA | 371 |
| | | CACTCTTTCCCTACACGAC | |
| | D506 | AATGATACGGCGACCACCGAGATCTACACtaatcttaAC | 372 |
| | | ACTCTTTCCCTACACGAC | |
| | D507 | AATGATACGGCGACCACCGAGATCTACACcaggacgtA | 373 |
| | | CACTCTTTCCCTACACGAC | |
| | D508 | AATGATACGGCGACCACCGAGATCTACACgtactgacAC | 374 |
| | | ACTCTTTCCCTACACGAC | |
| D701~ | D701 | CAAGCAGAAGACGGCATACGAGATcgagtaatGTGACT | 375 |
| D702 | | CAAGCAGAAGACGGCATACGAGATtctccggaGTGACT | 376 |
| | | GGAGTTCAGACGTGT | |
| D703 | | CAAGCAGAAGACGGCATACGAGATaatgagcgGTGACT | 377 |
| | | GGAGTTCAGACGTGT | |
| D704 | | CAAGCAGAAGACGGCATACGAGATggaatctcGTGACT | 378 |
| | | GGAGTTCAGACGTGT | |
| D705 | | CAAGCAGAAGACGGCATACGAGATttctgaatGTGACTG | 379 |
| | | GAGTTCAGACGTGT | |
| D706 | | CAAGCAGAAGACGGCATACGAGATacgaattcGTGACT | 380 |
| | | GGAGTTCAGACGTGT | |
| D707 | | CAAGCAGAAGACGGCATACGAGATagcttcagGTGACT | 381 |
| | | GGAGTTCAGACGTGT | |
| D708 | | CAAGCAGAAGACGGCATACGAGATgcgcattaGTGACT | 382 |
| | | GGAGTTCAGACGTGT | |
| D709 | | CAAGCAGAAGACGGCATACGAGATcatagccgGTGACT | 383 |
| | | GGAGTTCAGACGTGT | |
| D710 | | CAAGCAGAAGACGGCATACGAGATttcgcggaGTGACT | 384 |
| | | GGAGTTCAGACGTGT | |
| D711 | | CAAGCAGAAGACGGCATACGAGATgcgcgagaGTGAC | 385 |
| | | TGGAGTTCAGACGTGT | |
| D712 | | CAAGCAGAAGACGGCATACGAGATctatcgctGTGACTG | 386 |
| | | GAGTTCAGACGTGT | |

### INDUSTRIAL APPLICABILITY

Using the new CRISPR/Cas12a system disclosed in the present disclosure and the method for editing eukaryotic genomes, a target nucleic acid in the eukaryotic genome can be edited. Therefore, the new CRISPR/Cas12a system can be used in various contexts that require eukaryotic genome editing.

1. A method for editing a target nucleic acid in a genome of a eukaryotic cell genome, comprising:
delivering a composition for editing the genome of the eukaryotic cell into the eukaryotic cell,
wherein the composition for editing the genome of the eukaryotic cell comprises:
a Cas12a protein linked to one or more nuclear localization signals (NLS), or a nucleic acid encoding the Cas12a protein linked to one or more NLSs; and
a guide RNA or a nucleic acid encoding the guide RNA,
wherein the guide RNA comprises a guide domain and a scaffold,
wherein the guide domain targets the target nucleic acid,
the Cas12a protein and the scaffold are selected from following combinations:
Cas12a protein comprises an amino acid sequence selected from SEQ ID NOS: 1, 264 to 274, and the scaffold comprises a nucleic acid sequence of SEQ ID NO: 33; or
Cas12a protein comprises an amino acid sequence selected from SEQ ID NOS: 3, 275 to 279, and the scaffold comprises a nucleic acid sequence of SEQ ID NO: 35.

2. The method for editing a target nucleic acid in the genome of the eukaryotic cell of claim 1, wherein the Cas12a protein linked to one or more nuclear localization signals has a structure as follows: 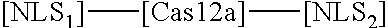
wherein the NLS₁ comprises an amino acid sequence selected from SEQ ID NOs: 67 to 94, or is absent,
wherein the Cas12a is the Cas12a protein described in claim 1,
wherein the NLS₂ comprises an amino acid sequence selected from SEQ ID NOs: 67 to 94, or is absent,
wherein at least one of the NLS₁ and NLS₂ is present.

3. The method for editing a target nucleic acid in the genome of the eukaryotic cell of claim 2, wherein the structure of the Cas12a protein linked to one or more nuclear localization signals is as follows:
the NLS₁ is SEQ ID NO: 67, and the NLS₂ is SEQ ID NO: 67; or
the NLS₁ is absent, and the NLS₂ is SEQ ID NO: 70.

4. The method for editing a target nucleic acid in the genome of the eukaryotic cell of claim 1, wherein the composition for editing the genome of the eukaryotic cell comprises a CRISPR/Cas12a complex in which the Cas12a protein linked to one or more nuclear localization signals is combined with the guide RNA.

5. The method for editing a target nucleic acid in the genome of the eukaryotic cell of claim 1, wherein the composition for editing the genome of the eukaryotic cell comprises the nucleic acid encoding the Cas12a protein linked to one or more nuclear localization signals, and the nucleic acid encoding the guide RNA.

6. A composition for editing a genome of a eukaryotic cell comprising:
a Cas12a protein linked to one or more nuclear localization signals (NLS), or a nucleic acid encoding the Cas12a protein linked to one or more nuclear localization signals; and
a guide RNA or a nucleic acid encoding the guide RNA,
wherein the guide RNA comprises a guide domain and a scaffold,
wherein the 3′end of the scaffold are linked to the 5′end of the guide domain;
wherein the guide RNA binds with the Cas12a protein to form a complex,
wherein the guide domain targets a predetermined target nucleic acid,
the Cas12a protein and the scaffold are selected from following combinations:
Cas12a protein comprises an amino acid sequence of SEQ ID NO: 1, and the scaffold comprises a nucleic acid sequence of SEQ ID NO: 33;
Cas12a protein comprises an amino acid sequence of SEQ ID NO: 3, and the scaffold comprises a nucleic acid sequence of SEQ ID NO: 35; or
Cas12a protein comprises an amino acid sequence of SEQ ID NO: 2, and the scaffold comprises a nucleic acid sequence of SEQ ID NO: 34.

7. The composition for editing the genome of the eukaryotic cell of claim 6, wherein the composition comprises a complex formed by the binding of the Cas12a protein and the guide RNA.

8. The composition for editing the genome of the eukaryotic cell of claim 6, wherein the composition comprises the nucleic acid encoding the Cas12a protein, and the nucleic acid encoding the guide RNA.

9. The composition for editing the genome of the eukaryotic cell of any one of claim 6, wherein the Cas12a protein linked to one or more nuclear localization signals has a structure as follows: 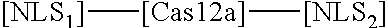
wherein the NLS₁ comprises an amino acid sequence selected from SEQ ID NOs: 67 to 94, or is absent,
wherein the Cas12a is the Cas12a protein described in claim 1,
wherein the NLS₂ comprises an amino acid sequence selected from SEQ ID NOs: 67 to 94, or is absent,
wherein at least one of the NLS₁ and NLS₂ is present.

10. A CRISPR/Cas12a composition comprising:
a Cas12a protein, or a nucleic acid encoding the Cas12a protein; and
a guide RNA or a nucleic acid encoding the guide RNA,
wherein the guide RNA comprises a guide domain and a scaffold,
wherein the 3′end of the scaffold are linked to the 5′end of the guide domain;
wherein the guide RNA binds with the Cas12a protein to form a complex,
wherein the guide domain targets a predetermined target nucleic acid,
the Cas12a protein and the scaffold are selected from following combinations:
Cas12a protein comprises an amino acid sequence selected from SEQ ID NOS: 264 to 274, and the scaffold comprises a nucleic acid sequence of SEQ ID NO: 33; or
Cas12a protein comprises an amino acid sequence selected from SEQ ID NOS: 275 to 279, and the scaffold comprises a nucleic acid sequence of SEQ ID NO: 35.

11. The CRISPR/Cas12a composition of claim 10, wherein the composition comprises a complex formed by the binding of the Cas12a protein and the guide RNA.

12. The CRISPR/Cas12a composition of claim 10, wherein the composition comprises the nucleic acid encoding the Cas12a protein, and the nucleic acid encoding the guide RNA.

13. The CRISPR/Cas12a composition of claim 10, wherein the Cas12a protein further comprises one or more nuclear localization signals,
the Cas12a protein has a structure as follows: 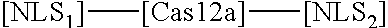
wherein the NLS₁ comprises an amino acid sequence selected from SEQ ID NOs: 67 to 94, or is absent,
wherein the Cas12a is the Cas12a protein described in claim 1,
wherein the NLS₂ comprises an amino acid sequence selected from SEQ ID NOs: 67 to 94, or is absent,
wherein at least one of the NLS₁ and NLS₂ is present.

14. A method for editing two or more target nucleic acids in a genome of a eukaryotic cell, comprising:
(a) delivering a Cas12a protein linked to one or more nuclear localization signals, or a nucleic acid encoding the Cas12a protein linked to one or more nuclear localization signals into the eukaryotic cell,
(b) delivering a vector structure into the eukaryotic cell,
wherein the vector structure comprises a nucleic acid encoding a first guide RNA, and a nucleic acid encoding a second guide RNA,
wherein the first guide RNA comprises a first guide domain and a first scaffold,
wherein the first guide domain targets a first target nucleic acid within the genome of the eukaryotic cell,
wherein the second guide RNA comprises a second guide domain and a second scaffold,
wherein the second guide domain targets a second target nucleic acid within the genome of the eukaryotic cell,
wherein the nucleic acid encoding the first guide RNA is operably linked to a promoter,
wherein the nucleic acid encoding the second guide RNA is directly linked to the 3′ end of the first guide RNA;
wherein the (a) and the (b) are performed simultaneously or in any order,
the Cas12a protein, the first scaffold, and the second scaffold are selected from the following combinations:
Cas12a protein comprises an amino acid sequence selected from SEQ ID NOS: 1, 264 to 274, the first scaffold comprises a nucleic acid sequence of SEQ ID NO: 33, and the second scaffold comprises a nucleic acid sequence of SEQ ID NO: 33; or
Cas12a protein comprises an amino acid sequence selected from SEQ ID NOS: 3, 275 to 279, the first scaffold comprises a nucleic acid sequence of SEQ ID NO: 35, and the second scaffold comprises a nucleic acid sequence of SEQ ID NO: 35.


SEQUENCE LISTING (423 sequences)

SEQ ID NO: 1 - protein, 1221 aa, synthetic construct
  source 1..1221: note = New Cas12a protein, AA sequence, id218
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQK

SEQ ID NO: 2 - protein, 1322 aa, synthetic construct
  source 1..1322: note = New Cas12a protein, AA sequence, id233
TFYNIILKKYKNIEGKMNQNTSISQFTGLYPVSKTLRFELKPMGKTLEKIKETGIIENDKRRHNDYFDAKKIIDTYHKYFIDAALSKFSRIDWNPLKEAIEGSLDKSDASKKKLEKIQTEFRKKIAKALTTHDHYKELTASTPKDLFLKVFPDHFGKQPAIDTFDGFSSYFTGFQENRQNIYSDEAISTAIPYRLVHDNFPKFLSNIEVYKTLKDNAPSVLSDAENELKDFLNGKSLANIFELNAYNDVLTQSGIDFFNQVIGGISGEGGEKKTRGINEFSNLYRQQHPEFAQKRLATKMIPLYKQILSDRETKSFILESYSTDSQVQESVKEFFESQILNCDIAGRKVNVLKELSSLIKRITEFDLGSIYVNQEELSNISLELFKSWNTINAVLFKDAENRIGSAEKAANKKKIDAWMKSNEFSIATLNLAIAESDSEEISRVKIESYWNDFEAKVQSILCGDNRRNLDEFLSATFNENNALREDSEIIGKLKAFLDALIEIMHSIKPLISDAENRDLSFYNELMPLYDQLSLVVPLYNKIRNYATQKLTESEKFKLNFDCPTLADGWDQNKEKDNKSILLRKDGLYYLGVMNTNDMPKIEEINALGNEDCYEKMIYKQFDCLKQIPKCTTQTKAAKAHFEAGKTEDFVIKDKSFNGPFVISEYIWKLNNYVWNGEKFVLKFGDADKRPKQFQMGYYKETNDLLGYKKALADWIDFCKKFVKTYISASGYNYDFLDSDKYNSLDEFFSYLKTICYKITFSKIPTSQIDEWVNEGKLFLFQIYNKDFAPGAKGSPNLHTLYWKSVFSPENLKDVVVKLNGEAELFYRPSSVKKPYSHKVGEKLVNRIGKDGLPLPESVFGELFRYFNGKLDGELSDEAKKYLDVAVVKDVKHEIVKDRRYTQDKFEFHVPLTLNFKADSKNEYMNERVRHFLKDNPDVNIIGIDRGERHLLYMTLINQKGEILKQKSFNIVESVNYQAKLIQREKERDAARRSWSSVGKIKDLKEGFLSQVIHEITTTMIENNAIVVLEDLNFGFKRGRFCVERQVYQKFEKMLIDKLNYLVFKNKPEGDVGGVLKGYQLAEKFDSFQKLGKQSGFLFYIPAAYTSKIDPTTGFANLFNMTELTSAEKKKEFLSHFEDITYDGKNDRFLFSFDYKNFKCFQTDYIKKWTVYSQGKRIVYDKESKSAKEISPVEIIKAALAKQNIALTDQLDVLSAINSAEASPKSASFFGDICYAFEKTLQMRNSIPNTDEDYLVSPVLNKKGEFYDSRSCGDTLPKNADANGAYHIALKGLYLIKNVFDAGGKDLKISHEDWFKFAQSRNS

SEQ ID NO: 3 - protein, 1339 aa, synthetic construct
  source 1..1339: note = New Cas12a protein, AA sequence, id236
AFVICYNTYSRTYLEKEIISMKTIESFCGQKKGYSRSITLRNRLIPIGKTEENIRKLKLLDKDIDRSKAYVEVKALIDDFHRVFIEDVLSKTELPWEPLYDQFELYQNEKDKQKKNKIKKDLEELQKGLRKSIVKQFKADDRFDKLFKKELLTEFVPSVIKNDDSGTISDKQAALDIFKGFATYFTGFHQNRQNMYSEEAQSTAISNRIVNENFPKFYANIQTFKYLEENFPQIISDTEQSLSDFLGDKKLKDIFSIDGFNKVLSQSGIDFYNTVIGGISEEAGTQKVQGLNEKINLASQQLSSEEKHKLKKKMTVLYKQILSDRSTASFIPVGFEKSEEVYDSVKEFKELSLDKNIAAINTIFSRDDYDLTRIFVPAKEITEFSLKLFGHWSILQDGLFLLGKDNAKKDLSEKQITELIKEIAKKDYSLAELQNAYERWTKENDIPVEKTVKNYFRLAELRTDEKTKEKNFTDIQKELEKAFVQIDFDKKENLIKEKEAATPIKNFLDEVQNLFHYLKLVDYRGEEEKDSDFYAKYDQVLQALAEIIPLYNKVRNFVTKKPNEVKKVKLNFECSSFLSGWGTDYGTKEAHIFIDDGKYYLGIVNEKLSKEDIKFLEEKSPRMIKKVVYDFQKPDNKNTPRLFIRSKGTSYAPSVSQYDLPIESVIEIYDKGLFKTEYRKVNPSVYKESLVKMIDYFKLGFTRHESYKHYNFSWKDSKDYNDISEFYADVMNSCYQLKFEYINYDNLLSLVDAGKLFLFQIYNKDFSTGKDGANGSTGKKNLHTLYWENLFSEENLKDICLKLNGEAELFWRDVNPNIKNICHKKGSILVNRTTSDGKVIPEDIYQEIYKFKNPDKQEKDFKISDEAKALLENGKVVCKEAKFDITKDRHFTQQTYLFHCPITMSFKAPEITGRKFNEKVQSILKANPDVKIIGLDRGERHLIYLSLINQKGEIELQKTLNLVDQVRNDKTVSVNYQEKLVHKEGERDKARKNWQSISNIKELKEGYLSNVVHEIAKLMVENNAIVVMEDLNFGFKRGRFAVERQVYQKFENMLIEKLNYLVFKDKAVTEPGGVLNAYQLTDKSANVSDVGKQCGWIFYVPAAYTSKIDPKTGFANLFYTAGLTNVEKKKEFFDKFEAIRYDSKTDSFVLSFDYKDFSDNADYKKKWSLYSRGERLVFSKAEKNVISVNPTENLKALFDKQGINWKSEENFIDQINAVQAERENVSFFDGLYRSFTAILQMRNSVPNSSKMEDDYLISPVMAEDGTFYDSRKEAAKGKDEQGKWISKLPVDADANGAYHIALKGLYLLQNDFNVNDKGYIDNISNADWFEFAQEKNYAK

SEQ ID NO: 4 - protein, 1371 aa, synthetic construct
  source 1..1371: note = New Cas12a protein, AA sequence, id204
QNIESFTNLYSLTKVVKFNLDPIPETKKWLKAFGKVFTHNHSENLEDNFFKLDLKIEDAKRIIQDVLNSVHEAIITQALESEEAKSINISSYYELSNQRNHDLSDLEQRLREQIKVCFIKQLKDFCSEVSPKKKNSDDENGLPDLLKMLGSSNMLDYLSKSASKYSSPIHDKKSIEDACKDLKGFWGVLDKYIKNRENYYVFDKEQDTAVATRIVSDLLPMFCKNCKQYLTNKALYDSIFTSLKSSGIIMQMINPKTNEMRSIESFDSSIYNISGFYKYISQSGIETYNAIISRNNILVNLYNQQQKKANRINGFVELHKQIGCRESESTERIVICKDWDSTESKELGISMESMIKRIICMVNETIVPTEPNILHPRVYNLISWLENAVDWDGIYVSDKALSIISSHYLEDWYSLQQLLLNKESVGTYNQRKESNEQFKLNSAVELMPVFRALDCKDFDSVFKKSIYNDYSDILNKNLPPSRNLINMLCHDVKQIINNELPIYTKCITQLLTTKISKVQREGFFKQEEIIDIIRDYLDCVLSIARFVYFFSVRQNKIKGAPYNMELLQIVSSLLSTELGNRRLWYDEVRNYLLKSPQDEVKENKLKLNFNSSSLLKGWSIGEERTKLSLLLKNEGCYYLCILKNDKTCKKLFDINTQNNIYIEHGNAYRMMLRNLSFKTLVGKGYVSMFGTKYSEEDDEVAIQHAKQLIRQNYLKNYPELSAVITKPHSTKSAFFADVDSILKDYAQCYYNPINWNLIEKFANEGNIYVFRIHAKDYKDTSKGKKDLQTIYWEDVLSGHSSHSLSAYGEVFRRDAISTDKTKPFIHKKGSVLINKRDIDGNSIPNSIYIIIYALLNKVPLPKTAEEKDIERAKSLIEKNIIISKESWMNIIKDARFYGEEKYFLHNPICFNYKSIGFKSEDFSKDIPYHVINPIIQELMHNSCAPTSFIGIDRGEKNLIYACKINSEGSILFCNHYNIVHGINYLEKLEERADSRLKAKRSWKQQESIKNLKEGYISHVVHQLLEDVISVGNENLTPSYIVLEKLSREMKRGRQKIEKQVYQKFELALASKLGFYVNKDIEEEKPGSIQMPLQFVPPVNTFDQIDKKDSFGIMLYTRANYTSVTDPLTGWRKTIYITNGNNEDILKELTEAFDDIFFDGVDYVFSYVEKNSGQRWNIYSGNHGKSLDRFEYNKDTKCYESYNIVEVLDVLFADFNKSSSLLEQMRRGVKLKKAEENRTAYDSLRKAINMIQQIRNTGNEIKDNNFLQSPIRKDGKHFDTRNANDFRDLNLHFIKDADANGAYNIARKGLIMDAHYKYWMEQGKPNVKKKNKKEKEVSALSLYVSDREWDMWLLNREMWEKHLSEFSLRKQD

SEQ ID NO: 5 - protein, 1299 aa, synthetic construct
  source 1..1299: note = New Cas12a protein, AA sequence, id206
SRDFSQFTNQYKLSQTLQFSLIPQGRTREFMDSFLIHDQSRSDAYPEVKKILDNAHKALIERTLGSISQRIHSLSDKEKKKFASILNGDDINWLQLDKAVENEAENGKDATDAVRLEYCRLLAMLFSYDDAFESLVKNATPDEYFKNELKKMALSDDDTARFLEKFKGFACYFKGFQENRRNIYSQEAQSTAAPHRAIYDNFPKMKRCIKIFNHIKDAYPEILTTATTELAELLNGQSIDSVFSIQEYSNYLSQKGIDKFNSILGGYTTKDGQKVKGINECINLYRQQHEDARKDRSFSTMPLLFKQILSDRSSLSFIPQMFQNDQELFNTLKEFIQQLDHVTTADGIDRPLIPSFRDCLDNISIDDGILVSASSLTEISMRASGSWNAIPDAMENWAAVNISTKALREKWLKKNSYSLTELDALHLEKETDEKRISCDILDYWKGKKALDCYDRYDALLKDITSLMSAPVNDIPLAERKDDVALIKDYLDSLMDILHLGKPLYCQDILDRNEQFYSLFDGLYEQLQIVIPLYNRIRNYLTQKADTTKKVKLMFDSPTLADGWDQNKEQTNNAIILLKDGKYYLGIMNPAKKPVMSKMVVQKAESEVEVYQKMVYKYLPGPNKMLPKVFFSTKGQKDFNPPKSLLEKYEAKMHIKSSPDFSLAFLHELIDYFKASIAKHPDWSKFGFKFSPTASYQGIDEFYREISNQGYRLSFEIIPKEKIDDLVNNEQLLLFQIYNKDFAPGATGTPDKFTIYWKMLFSPENLSNVILKLNGEAELFWRKQVIAKPFSHKLNEKMVNRTYVDGTDSTGRPIVKHMPEKVHAVIFDYVNGKNDDIHKKEYQDALALYDSGKVIVKDVTHPIIKDRRYTQESFSFHVPITINFTAPDKPSKFNDSIIAFLKNNPDVKIIGIDRGERNLIYFSLIDQQGNILKQGSLNTVNPRTEARIEQVNYHELLNQKEKERDEARKSWSSIGKIKDLKAGYLSQVIHQIAPLMVENNAIVILEDLNFGFKRGRFAIEKQVYQNFEKALITKLNYLVFKEIQDYNTAGSVLIGYQLTDKFVSFEKMGKQTGALFYVPAAYTSKIDPTTGFANLFNTRKCTNVESRKVFLMTFDAIKYNAVRKSFAFSFDYAKFQTSAKSFQNKWTVYTSQKRLVFNPLTRQDTAVNPTQMIVDALNKRGVSLSDEFDLKAYLQNTDAIKANAEFFKTIFIALEYTLQMRNSNSSSGEDYIESPVLNKSGEFFRSSEDNPALPIDADANGAYHIAMKGLFLLLNVFNQGKKDLKIEHQAWFEFMQKRNM

SEQ ID NO: 6 - protein, 1231 aa, synthetic construct
  source 1..1231: note = New Cas12a protein, AA sequence, id210
AYSDFIGMYPLQKTLRFELKPQGRTMETLKEKDYLKNDLYRASQYAGMKKLIDDYHRSFIDESLMTSEIDWQPLADALDARKNNDEKSKKQLEILQDQYRRQVSKLFTGKEVFKSLFSKEVLDLVREKCVKERDDSKIQICDAFNRYSVYFTGFHENRRNFYVSDKQSTSISKRLVDDNFPKFLENIRIYEKIEVECPEIIEKVKNELKLTDLKKYFSVSEFNRFVRQCGIDDYNWILGGHVGEEAHVKGFNGFLNEAYQQGLLKTRVLMRPLYKMILSISESKSVLPYQFKDEKEMMDAVVSFIQKANEGIIAQAGDLLKSVESTDLSRVFIPDKQLTELSQILFGDWGALGTMMREYKAYDLGDPTMSKTMKKVDNWMKSSEFSLEDVKKAVLHADQEFTLENYEVRVDQVCSKLIETNAVSELKSVKIDGTDGETRNRSAECLRCILEGYQDLLHCMNMFKVSDELDRDEAFYSEFDLILGELQEIIPLFNKVRNYCTKKAYNETKVKLKLSTPTLADGWDLDKEKDNKAVILRSGGQYYLGVNCDKGFLDFSKYETDDEECFEKLNYKLLPGPNKMLPKVFFSKSWKSEHTIPERIQDGYDRGIFKKGPEFDLMFCHELIDFYKESISIHPVWSKFDFRFAETSDYQDISQFYADVQRQGFKMSFSKVSKSEVYKLVDEGKLMLFKIHSKDFSENSRGMPNMHTLYWRALFSEENLANIRIKLNGEAELFFREKSIDTPVVHKSGSVLVNKTFNGKHVPDKIYYELFRYRNGIITALSPEAKEYEGAIVSSVANYDIVKDRRYTVDKLFFHVPLTFNFGVPDRSKDINRMVLEHAIKSKDTKIIGIDRGERNLIYISMIDKEGKILLQKSLNVIDGVDYHGNLDVREHENKEARRNWTKVAGIKDLKEGYLSAAVSQIARMVVENDAIVVMENLNYGFKHGRFKIEKQVYQKFESMLIDKLNYLVFKDKEDFTSDGRAMRGYQLTKSIDSVKEIGTQTGVIFYVNAAYTSKIDPTTGFINQIQFSTKSTLSAKRDLLCNIDSIRYDPSEECYIFRIDYTKVGDRTKEFKNVWDIYTRGDRIVYSTKERCDKHLHPTEIMTKALESAGIGKEGELKEKICSADSKVIEAVVYALDLTLRLRNEDRETDYILSPVRNADGKFFCTLDGDLSLPLDSDANGAYNIALKGLLMLKMTEESNGPDADKYDISLVTNSDWLTFCQTGHRTWKS

SEQ ID NO: 7 - protein, 1231 aa, synthetic construct
  source 1..1231: note = New Cas12a protein, AA sequence, id211
TNFYSSFTNLRSVSKTLRFSLSPVGKTRENIRSANIIPQDEKRMQDYLVLKNVFDAYHKAYIDQHLCRIQGDWSELANALDERQSAPDADALNKMQETYRKEISNLLSGKGTGKNQYLFPADGDGGSLLEMLDPADMIKQTVKEKKDAEKGNRPPQYLIPEFGQNIKEVEQFNRFAGYFEDYKKTRENIYASEGSTSVAHRIVHENFPKFMSNVHTFRRLPESLRQKFEHDLSELLDGASLETMFDVDYFNVVLAQAGIERYNTLLGGKKEESGVKLQGLNELCNLEFQQGNLPKKVVFVPLYKQILSDREKASFLPEKFKSVEEMLESIADYRQSLSNLMASQHCLLDENMSTGYCDAGHVYIRKSQLASLSMLCFNEWSTFRTAVEAKDVYTLSELLQAAGKDILEDVRRELNFSLDSIDEAGEALLPVLEKQTLTEDDYAIIKEYLDAVIAAQHILKVFEAEDDAEKDPVFYGVADILNDALRQGVRLYDRVRNYATGKPYAEDKFRLTFFAPTLLNGWDKNKERDNCCMLFRKDGLYYLGILNAKNKPLLDEEAEIEGASYEKMVYKYLPEPNKMLPKVFNKSQKWIAAHGLDDDIRDGYEKGLHKVGQSFDKAFMHKLIDYYKACISEYEDWATFRFRFSATDTYQTMADFYNEIAAQSYKVFFTKVSEQQINAAVEAGQLYLFQIYNKDFSPYSHGAPNLHTLYWRELFSAENLQNPLVKLNGEAEIFYRHPSIANPFRHQKGDVLINKTDAAGRTVPEDRYRAAATDAANGMPVQALRASYPELIFKVADREIVKDKRYTKESFAFHVPLTLNNDNNAGFKLNMAVCEKLQSDGEYYIMGIDRGERHLLYISVIDRNGRIVEQRSLNVVGKNRVDYQAKLDALERQRQEARKNWRAITAIKELKEGYLSMAIHEITELMLQYPCILIMEDLNFGFKRGRFRVEKQVYQKFEKMLIEKLGFWMQKDKAADAAGGIRHAYQLTESFQSFQKLGKQSGVIFYVPANYTSKIDPKTGFVNLFGSRQLTYTSVSAAKAFFGTMREIGYDAQRDCYRFAFRYSDYDLYQQDHRDSWTVFTAGKGRIAHTVKNGRHTFEEIDVTERMKELFQRFGLDPYAADLRGAIDAADKKDFFSELLWLFKVTVQLRYEDSEQDFILSPIEKDGRFFDSRQAKKDEPQDGDANGAYHIALQGLRLARERIKDGRVQKDPKGKQTLNWLRFAQRKDYLE

SEQ ID NO: 8 - protein, 1219 aa, synthetic construct
  source 1..1219: note = New Cas12a protein, AA sequence, id217
KKIDHFTGCYSVSKTLRFKLIPVGKTQENIVAKRLLEEDENRAEDYKAAKKIIDRYHKSFIDDVLKSVKLSHLNEYASLFLKGNRSSDENETLEKLEETLRNEISKAFTENEGFALLFKKELIEDELPERLEGDELRIINSFKGFTTAFSGFHENRRNMYAADKKSTAIAFRCVNENLQRFLQNIACFEKIKCILDADMCKTIESDILSGDYSIRDFFVVDFYNLVLTQQGIDLYNALIGGVVKEDGRKIQGLNECINLHNQQNKDRLPHLKPLYKQVLSDRTGESFYGEGLTSDEELYDAMRNTVGFESMIYKNYRKAEELFASLDLGAFDGIYVKNGPAVTNISNDAYGHWGNFTEKWNEQYDLANEKSNAKDLEKFEEKRRKDFKKIESFSLGEIIKLMEDNEKLPTAVVKGIKDRIAAVEMAYANAKTVLTEGFKTEKQLKKDEVTVGRIKALLDSLKELESFLKQFKGTGKESGRNELFYGDYTLYCDMLSEVDELYNSVRNYVTQKPYSTDKFKLYFQNPQFLNGWDRNKETDYQSVILRNNDQYYLGIMDSKSRDIFRRVPESDEKGYEKMVYKQIPNASKYLSSKQINPQNPPEEIVRILEKKKTDNRSLTADEIKTFISYIQDDFLKNYSFLRDEKGKEYFDFKFKKPEAYRRLNDFFDDVDKQGYSVKFSDVPKTYVDSLVEKGELYLFRIYNKDFSEASHGTPNLHTMYFRSLFDGANQGVIRLAGEAEMFMRRASIKKDELIVHPANTAINNKNPLNPRKTRTLPYDVIKDRRFSEDQYELHMAISINKIPENGIKINHDVRKFLREDENPYVIGIDRGERNLLYVVVIDGKGKIVEQHSLNEIINEIHDVKIVTDYHFLLDQKEKDRLAARQDWTTIENIKELKEGYISQVVHKICELVEKYDAMIALEDLNSGFKNSRVKVEKQVYQKFEKMLIDKLNYLVNKKTPIEEKGSVIHGYQLTNKFESFKTMGTQNGIMFYIPAWLTSKIDPTTGFSDLLKPKYTNMESARSFVGTFDFIRYNGDEGYYEFGIDYSKFGCDADHRKKWTLCSQGSRIRTFRNPLKNNEWDNEEVNLTEMFDTLFEKYKVNGKSGDLRDKICGVTEADFYKEFYGLLRLMLQMRNSITGRTDVDYLISPVKNRHNDFFDTRNADKTQPMDADANGAYNIARKVLWAIEVFKKTEEDKLDKTKIAISNKEWLKFAQTMVD

SEQ ID NO: 9 - protein, 1271 aa, synthetic construct
  source 1..1271: note = New Cas12a protein, AA sequence, id219
IIYNCYIGGSFMKKIDSFTNCYSLSKTLRFKLIPIGATQSNFDLNKMLDEDKKRAENYSKAKSIIDKYHRFFIEKVLSSVTENKAFDSFLEDVRAYAELYYRSNKDDSDKASMKTLESKMRKFIALALQSDEGFKDLFGQNLIKKTLPEFLESDTDKEIIAEFDGFSTYFTGFFNNRKNMYSADDQSTAISYRCINDNLPKFLDNVRTFKNSDVANTLNNNLKILNEDFDGIYGTSAEDVFNVDYFPFVLSQKGIEAYNSILGGYTNSDGSKIKGLNEYINLYNQKNGNIHRIPKMKQLFKQILSERESVSFIPEKFDSDDDVLSSINDYYLERDGGKVLSIEKTVEKIEKLFSAVTDYSTDGIFVKNAAELTAVCSGAFGYWGTVQNAWNNEYDALNGYKETEKYIDKRKKAYKSVESFSLADIQKYADVSESSEANAEVTEWLRNEIKEKCNLAVQGYESSKDLISKPYTESKKLFNNDNAVELIKNALDSVKELENVLRLLLGTGKEESKDEYFYGEFLPCYERICEVDSLYDKVRNYMTQKLYKTDKIKINFSNSHFLSGWAQTYSTKGALIVKKENNYYLVIVDKKLSNDDIVFLGTNTQLSPAERIVYDFQKPDNKNTPRLFIRSKGTSYAPAVKEYDLPISDIIEIYDNEYFKTEYRKINPKGYKEALIKLIDYFKLGFSRHESYRCFNFKWKESEQYSDISEFYNDVVKSCYQLKSESINFDSLLKLVDEGKLYLFQLYNKDFSEHSKGTPNLHTLYFKMLFDERNLENVVFKLNGEAEMFYREASISKDDMIVHPKNQPIKNKNEQNSRKQSTFKYDIVKDRRYTVDQFMLHIPITLNFTANGGTNINNEVRKALKDCDKNYVIGIDRGERNLLYICVVDSEGRIIEQYSLNEIINEYNGNTYSTDYHALLDKKEKERLESRKAWKTVENIKELKEGYISQVVHKICELVEKYDAVIVMEDLNFGFKQGRSGKFEKSVYQKFEKMLIDKLNYFADKKKSPEEIGSVLNAYQLTNAFESFEKMGKQNGFIFYVPAYLTSKIDPTTGFADLLHPSSKQSKESMRDFVGRFDSITFNKTENYFEFELDYNKFPRCNTDYRKKWTVCTYGSRIKTFRNPEKNSEWDNKTVELTPAFMALFEKYSIDVNGDIKAQIMSVDKKDFFVELIGLLRLTLQMRNSETGKVDRDYLISPVKNSEGVFYNSDDYKGIENASLPKDADANGAYNIARKGLWIIEQIKACENDAELNKIRLAISNAEWLEYAQKK

SEQ ID NO: 10 - protein, 1243 aa, synthetic construct
  source 1..1243: note = New Cas12a protein, AA sequence, id223
MKSIKNFHNLYPVQKTLRFRLEPIGKTDDLIARNQILETDERRAAEYLKVKEYIDQYHRHFIENALSSPMLKVESMGKQDSLTDFADCYNNDNTEKRSEHLEKIQEKLRSQIVKRFSQLPAFARITKKELIKEDLPQFLKDKREQEIVSHFDEFTTYFTGFHQNRMNMYTADTKSTSIAFRLINQNLVKFVDNCNILEKVASSLSNEIIAKLDKDFEPFLNEHSALDLFKIENYNELLTQSQIEVYNSIIGGRVDEGNKIEIKGLNQYINEFNQTHEKSQRIPKLKPLFKQILSEDVGVSFRMEQFTDASQVQATIKEEYIKLESGVFDKLKEMIKSLSTFNLNGIYLANDLGLTDICQRHYGAWDILNNALITAFDAHIPRKKKQSQEKHDEQARKYLKGIKSISLGKIDSLLAEVTGKSIVDYFTSLGAINNETTQRENLFALIQNRYNAIKNVLTCPTASDELLRKNTEGIKDLLDAIKDLQHFIKPLCGYGEEADKDDIFYSDFSPLYETLDNTITPLYNKVRSYLTKKPYKLDKFKLNFDNAQLLDGWDINKENAYLSILLRKDGNFYLAIANKNDNRAITEINQAQADDGSDCYEKLNYKLIPSPFRMLPKVFFSRKGLETFNPSQEILDIYNNKEFQLGDKFDKEALYKLIDFYKTSIPNYESWQFFDFNFMPTQSYKSINEFYSDIEKQGYKIDFKQVPVSLINKLNDDGLLYLFKIVNKDFSPYSKGRPNLHTIYWKMLFDDNNLKDVIYKLNGKAEMFFRRNSITNPVIHPANKDIANKSTYNKQHKAVSKFDYDIIKDRRFTRNQYEFHVPITMNFKSASNGHFNRDVLAFIKQNGIKHIIGIDRGERHLLYLTMINLKGEIVKQLSLNSVACNPNNPDYKQDYNELLSIKEGDRLSARRNWATIENIKELKAGYLSQIVHLLSKMMIENDAILVLENLNTGFMRGRQKVEKSVYLKFEKMLIDKLNYVVDKTASINEPCGALKALQLTDTYDNFNKYQKGNVRQCGFIFYIPAWNTSKTDPATGFVNLFDTRLSTMGEIKSFFSKFDQIKYNSKNDSLEFTFDYNKFTSRAEGTRTCWTISSQGERIFTHRSKEQNNQFVSDTIYPTLIFKDVFSKNNIDINGNLKDEIASIDSLEALKQLLHAFKLVMQMRNSVTGTEVDFLLSPAIDAKGSNFDSRKGIKTLPENADANGAYNIARKGLMIIDQIKNTNDIDKIKYAVSNKDWLKFAQG

SEQ ID NO: 11 - protein, 1370 aa, synthetic construct
  source 1..1370: note = New Cas12a protein, AA sequence, id224
LKTKTITIMKEKEQYPDFTNFTNLYQLSKTLRFELKPIGKTEETFKQWLGAMNSAGNEDNNDNLFAQDKKIKDAYVVIKSIMDKLHEQFIEKSLTSEEAKNIDFSDFLDIYKYRGNDFPKDFEKKLREQIGNTYKVAGAYFSKEISKALNKEFKTKKPFECLTDAKMLNYLSANVKEIAEQNNIDQETLANHIKQFKGFWGYLSNYNKNRENYYTTDKEQSTAVATRIVHDNLPTFCDNIIRFEERNDEYCGICEYLKDKNIETKIKNNQGNEVDVEPITKDVFKIKHFNNCLAQSQIKEYNRIIGNYNYQINLYNQARSSENDFRKLDEFETLYKQIGCGEGRSLFETLHNYDDLNKLLEKIKEAGDSFFKECGYNEISTVPAFIKFLKECNNWDGIYMSSTAINRISNRYFANWHSIKDKLKDNKACVTYDKNREEPIQLRDAVELSDLFKVLDDKEQSEHFFKESLSNDINEKECPSKNLINLLCFDIEQNISKFLANSENIINLLNGNKDNQASEEDKIKQIKEWLDAAIDTMRIVRYFAVRKSKMKGNLPNTTMEKALNNLLYNDDAQWFKWYDLVRNYLTKKPQDDAKENKLKLNFGNPSLLGGWSDGQEQTKAATLLKYNNELYLCILKIRNIFDTSKADNPIYTKAPFCACRLILRNLKFQTLAGKGFKSKYGIKYSEMGNEDQSQAIKCLKEIIRERYAKKYPPLEQIINQEYSDKKSFDNEVKEALKECYVCEFVPINWDVVSKKQNSTDLFLFKIHCKDYLPKATGKKDLQTMYWEDVLSDSSNHQLCGGAEIFMREPVPKESPVKHGVGSKLVNKRDKDGNTIPEQIYKEIYLFANDKVKKENLSAEAKNYIESEKVTIKEAEYEIVKDNRFYGETKYMFHCPITINFKAKGYKYAYPEVNEKITDALQQSDNLQFIGIDRGEKHLVYSCTIDKEGKIVKCNHHDNINGTDYVQKLEAVANERIAAKKNWQQQNKIKDLKAGYISHVVHRLVDETVKDGDKITPHAYIVLEDLNSEMKRGRQKIEKQVYQNLELALAKKFNFVVDKEAKQGELGSVSKALQLTPPVNNYQDIEGKKQFGVMLYTRANYTSITDPTTGWRKTIYIKNGKDEEIRKQILEKFTDFGFAGKDYYFEYTEENAGHTWRMYSGKNGEPLPRFQNKKQMEQDKNIWVPEQINVKEILDKLFANFDKGRSFKEQVNEEVKLEKIEGRSETAWQSLRYALNMIQQIRNSGVEKTDDNFLYSPVRNENGEHFDTRNHENNGDLSPIVDADANGAFNIARKGLIMDAHIKHWINSGRPKIKKDGKETPDLDLFISDREWDLWLLDREEWKKYLPVFASKSKKDADKTTSTKSPKRKKK

SEQ ID NO: 12 - protein, 1250 aa, synthetic construct
  source 1..1250: note = New Cas12a protein, AA sequence, id230
KDLKQFIGIYPVSKTLRFELKPIGKTLDWIRERNVVEKDERKSQDYPVVKQHIDEYHKVCISESLKNVCFDWKPLKEAIEKHHKLKSDESKKQLETEQTNMRKQIVLAIKRFKHYPELTAATPKDLIQNVLPEITNDSSLNSFNRFASYFKGFQENRKNIYSLDAISTGVPFRLVHDNFPKFLSNIDIYENIKSTAPETLELAAKELQPFLEGVMLDDIFSLDFYNSLLTQDGIDFFNRVIGGVSEEGKQKFRGINEFTNLYRQQHPELSANKKAMTMIPLFKQILSERETLSYIPKQIENENELTETIKQFYSHITNFERNGKTINILNELVRLLGQVTSYNQDRIFVSSKYITDISQKLYGRWNAINDYLYERAVEMFGDVSVAKNKKLVDNYIDKDVYSASELGFDENNNLSTFYSELPQIVGSVNGNWIQFCEWCKIDKKQKFLNNAEGTEIIKKLLDAIMEVMHKCNVLVVSEEYDLDKDFYNNFFPLFTELENIVFVYNRVRNFLTKKPSDVKKFKLNFGVPTLCDGWDLNKEKDNKAILLFKDNRSYLGIMNPEDYPKIKESNDSGDGYFSKMVYKLLPGPNKMLPKVFFSKKGRETFSPSDRIIDIYESGAFKKGSSNFSIVCLHELIDFYKDAINRHEDWSKYNFKFSPTDSYKDIGAFYNEISKQAYKIRFTNISEQEINQLVDKGQLYLFQLYNKDYASGAHGRKNLHTLYWENLFKEENLNNLVLKLNGQAELFYRPQSINEPVAHKVGSIMLNRRDKSGMPIPETVYRSLYLYFNGKKKEEELDSVEKSYIDNVIVKKVTHEIVKDKRFTKPEFFFHVPITFNSNADGNEFINENVIDYITDNPDVNIIGIDRGERHLIYLTLINQQGEILKQKTFNTVGTYNYHAKLEQREQERQEARKSWQSIGKIKDLKEGFLSAVIHEIALMMAEYNAIVVLEDLNFGFKRGRFKVERQVYQKFEKMLIDKLNYLAFKDRKSDETGGILRGYQFAQKFVSFQRLGKQSGFLFYVPAAYTSKIDPVTGFVNHFNFNDITNAEKRKQFFCKMERIEMKNDNIEFEFDYRKFKTYQTDFQNVWTASTFGKRIVMKTDESGRKQMADYYPTNEILAAFKKYGYELTNGTDIKSLLQDIEADQKSASLFSNLFYAFQKTLQMRNSNHATEEDYILSPIAKHGYHFCSTEEANKGKDEFGNWLSKLPVDADANGAYHIALKGLYLLKNPQTKKIEHEKWFKFMVEKPYK

SEQ ID NO: 13 - protein, 1232 aa, synthetic construct
  source 1..1232: note = New Cas12a protein, AA sequence, id238
KEQFINQYPISKTLQFSLIPIGKTEENFNNKLLLEEDEKRAEEYQKVKEYIDRYHKFFIETALLNVSLNGLEEYVSLYFKPSKSDADVKAMSDIEAKLRKQISKAMTSHELYKDLFSKDMIDSILPKYLQKEEERSNVEMFRGFSTYFQGFYTNRKNMYSEEAQSTAISYRCINDNLPKFLDNVKSFEKIREAFNEEKITTINEEFFGILGKYATDIFNVDYFNFVLSQPGIDKYNKIIGGYVCSDGTKIQGLNELINLYNQQVAKSDKSKRLPLLKMLYKQILSDRESVSFIPEKYKDDNEIIASINEYFSETALETVAALKELFDKFSSFDLKGVYISSGLAVTSLSNDVFGSWNAFSSAWETRYSEQNPKKKNKSQEKYDEELKANFKKNKSFSLDEFQQLGSEMKEPDSIGSVLDFYAGAVSEKADNIRALYIAAKELLTPNYAEKYDKKLIKNHVAVEKIKSLLDAVKSLEKLVKPLLGTGKEEEKDNVFYGKFLPLYDSLTLIDRIYDKVRNYVTQKPYSKDKIKLNFENPQLLSGWDKNKESDCRGILLRRNGNYYLGIIDKSNSKVFQSYPVPEFSDDEYYEKIIYKQINGCSKYFSIKQIKPQNPPENIIKYLSKDFDKKTMTKGELVELIKYVVEDFIPNYSPLLDENGKCFFDFKYKPYDQYTSWNHFCKSLDPQAYTIRFSKVKKDYIDNLIEIGFLYLFQIYNKDFSEYSHGTPNLHTLYFKTLFDKKNFADVVFKLSGGAEMFFRKASIKENEKTVHPANQPIINKNPDNSKKQSEFSYDLVKDKRYTKRQFSIHIPITINFKANGREFINYEVRRALNSDYKPCVIGIDRGERNLIYITVINDTGEIVEQMSLNEIISDNGYKVDYQKLLDKKEKARDEARKSWGTIENIKELKEGYISQVIHKICELVIKYDAVIAMEDLNFGFKRGRFNVEKQVYQKFENMLISKLNYLCDKKMEANAEGGVLKAYQLTNKFDGVNKGKQNGIIFYVPAWLTSKIDPVTGFVDLLHPKYTSVEESHNLFGNFDDIRYNAVTDMFEFDLDYSKFPKSKADFKKKWTVCTNSDRIMTFRNPAKNSEWDNKRVILTDEFKKLFEEFGVDYKSNLREKILGISNPDFNKRLVKFLALTLQMRNSVTGSTNPEDDYLISPVRDKNGVFYDSRNFIGNDAVLPADADANGAYNIARKGLWAIDVIKSTSSDMLDKADLSISNAEWLEYVQK

SEQ ID NO: 14 - protein, 1263 aa, synthetic construct
  source 1..1263: note = New Cas12a protein, AA sequence, id239
KEFTNLYSLSKTLRFELQPIGKTLENINANGFLTNDAHRAESYKKVKKLIDEYHKDFIDKVLGDFKLNAEHLQLYYDLYSQTTKDKQFKDVQDKLRKAIAGALKADDRYKTIDKKELIRQDMKKFLNNDTDKALLDEFYEFTTYFTGFHENRKNMYSDEAKSTAIAYRLIHDNLPKFIDNMAVFKKIANTSVADNFSAIYKAFEECLNVNSIDEMFSLDYYNDILTQTQIEVYNSIIGGRTLEDGTKIQGINEYVNLYNQQPANKKERLPKLKPLFKQILSDRVQLSWLQEEFNTGADVINAVKEYCVSYFDNVEESIKVLLTSISDYDLSKIYITNDLAITDVSQRIFGEWSIIPNAIDFRIRKDNPQRASEKEEKYSERISKIKKQSKSFSLAYINECLSELKDTDIADYYTTLGAINTESKQEPSIPTSIQVHYNALKPILDADYPREKNLSQDKLTVMQLKDLLDDFKALQRFIKPLLGNGDEAEKDEKFYGELMQLWEMIDSITPLYNKVRNFCTRKPFSTEKIKVNFENAQLLDGWDENKENTNASIILRKDGMYYLGIMKKEHRNILAQTMPSNGDCYDKVVYKFFKDITTMVPKCTTQIKSVKEHFANSNEDYTLFDKDKFLTPVVITKEIFDLNNVLYDGVKKFQIGYLNNTGDNQGYNHAVEIWKSFCMQFLKAYKSTSIYNFSSIEQNIDKYNDLNSFYGAVNLLLYSLTYRKVSVDYIHQLVDDDKMYLFKIYNKDFSPFSKGTPNMHTLYWKMLFDECNLSDVVYKLNGQAEVFFRKKSINCQHPTHPANKPIDNKNANNPKKQSTFEYDLIKDKRYTVDKFMFHVPITLNFKGIGNGDINMQVREYIKASDNLHFIGIDRGERHLLYICVINSKGKIVEQYSLNEIINNYKGTEYSTDYHTLLSEREKKRKEERSSWQTIEGIKELKAGYLSQVIHKISQLMIKYNAIVLLEDLNPGFKQGRQKVESSVYQQFEKALIEKLNYLVDKNKDVNEIGGLLHAYQLTNDPKLPNKNSKQSGFLFYVPAWNTSKIDPVTGFVNLLDTRYENVAKAQTFFNKFDSIRYNKENDCFEFCFDYTNFTAKAEETRTKWTLCTYGTRIETFRNAEKNSNWDSKEIDLTTEWKSLFAKHNISLNANLKEAILLQANKNFYADILHLMKLTLQMRNSVTGTDIDYMVSPVANESGEFFDSRNAKEGLPVNADANGAYNIARKGLWLAQQIKDANDISDVKLAITNKEWLQFAQTKPYLKD

SEQ ID NO: 15 - protein, 1274 aa, synthetic construct
  source 1..1274: note = New Cas12a protein, AA sequence, id245
NKFVNLYSVIKTLRFELVPIGKTKVNIEKSNLLMDDAKRAEDCKIVKSLMDRYHKQFIKSRLCNLELPLEENGQKCSLEEYRVLYEMKKRNAEQEATFAEVKGNLRALIAKHLTAKGTAYDRIFKKELIREDLLNFLEDESEREIVEQFADFTTYFYGFHKNRANMYSAEEKSTAIAYRLINQNLPKFMDNMRTFMRIAQTSVAEHFGEIYTGWKKFLNVESLEEFFSLKNFTKTLTQEHIEVYNYVIGKRILEDGAEIKGINEYVNLYNQQQKDKRNRLPFMVPLYKQILSDREQLSWLAEEFDSDEKMQKAVLETYGNLYDKLMGDENESLRSLLKNIDDYDLTQIHISNDLSLTEISQHIFGKYDAFTKGIKERLSGIVARKKNDKEEQYEDRINEVFKSYKSFSIADLNQLLLPEVEDGRPKNIEEYFKSLGAIHTDTYQKENLFTQIENAYTEASPILRMKETGDHLSQNKGAVEKIKNLLDALKALQHFIKPLTGSGEESDKDGRFYGAFQMIWDELDAVTPLYNKVRNWLTKKPYSTEKFKLNFASPTLLSGWTKPQDNSCVILSDDECCAFYLGIIDSHHRTLFNDIDRIHQKFRPGGMKIMNYYQGGDMGKNVQNLMMENGRVKKVNGRKETSGPFAGENLRLEQAKNKYLPPEINRIRKMASYKPNNSNYNKKDLELFIEYYMTLVKDYYPKCSFSFYPPSHYQSFDAFLTHIQQQSYQIHFSPISKSFIDNMVDAGKLYLFRIWNKDFSEHSKGTPNLHTLYWKMLFDERNLRDVVYKLNGQAEVFFRKSSISHENRIIHPANKEIENKNTLTEKKTSLFTYDIVKDKRFTVDKFQFHVPITINFKAKGLKNINPLVIDVIREGGFTHIIGIDRGERHLLYLSLIDLKGRIVKQMSLNQIINDYEGHQHKTDYHALLMKREGERTNARKSWDTIEKIKELKEGYLSQVIHLITKMMVKYKAIVVLEDLNSSFVRGRQKIERQVYEKFEKMLIDKLNCYIDKKRDPECEGGMLHALQLANKFKSFSKMGKQCGCLFYIPAWNTSKIDPVTGFVNLLDTRYESVKASKEFFSHFDCITFNATMDWFEFSFDYDNFHKKAEGTQTQWTVFSRGSRIHTFRNPEKNNQWDDEEINLTDKFKELFAKYSINYRGNLKDAMQDSNEATFFKELMGLMKLLMQMRNSKANSEIDYMLSPVSDKNGNFYDSRVCGGSLPENADANGAYNIARKGLMLIRNIQAAPDDKKPSLTITNKDWLCFAQTKPYLED

SEQ ID NO: 16 - protein, 1284 aa, synthetic construct
  source 1..1284: note = New Cas12a protein, AA sequence, id250
KDLKQFSNLYKVSKTLRFELQPIGKTKENIEKTGILARDEQMAKDYMLVKGFIDEYHKQFIKDRLWNFKIPLKSEGHLDSLEEYQMFYELSKRDDAQEAAFTEVKDNLRSIIAKRLTKSSSAFDRIFKKELIREDLINFLENEEDKEIVRQFADFTTYFTGFHENRRNMYDAEEKSTAIAYRLIHQNLPKFMDNLKAFAKIAETPVAEHFADIYEGWKERLNVGSIDEIFQLDYFSETLTQSHIEVYNYIIGKKILEDGTEIKGINEYVNLYNQQQKDKSKRLPFLVPLYKQILSDREKLSWMAEEFDSDDKMLSAINESYSHLHDLLMGEENESLRSLLMHIKDFNLEQINITNDISLTDISQHLFNRYDVFTNGIKDELRIIIPRKKKETDEVFEDRISKIFKNRKSFSIAFLDKLPQPVMEDGKPRSIEEYFLSLGAVNTGTTQKENLFARIENAYEDARTVLQMKDTNDTLSQNKPAVAKIKTLLDAIKDLQHFIKPLLGSGEENEKDELFYGVFQIIWDELDTITPLYNKVRNWLTRKPYSTEKIKLNFDNAQLLDGWDENKETANASIILCKNGQFYLGILNKDYRKLLGMPMPSEGECYDKVVYKFFKDITTMVPKCTTQKKDVVAHFAQSTENYILFDKKTFNAPVTITKEIYDLNNVLYNGIKKFQIEYLRSTGDKSGYEHAVFTWKSFCMQFLKAYKSTSIYNLALVEQNIDSYSDLSSFYNAVNLLLYNLSYRKVSVDYIHTLVEEGKLYLFRIWNKDFSEYSKGTPNLHTLYWKMLFDERNLCDVIYKLNGQAEVFYRKASIKPENRIIHPAHHPITNKNELNDKRTSTFEYDIIKDRRYTVDKFQFHVPITINFKAIGQSNINSIVQDTIRGGGFTHIIGIDRGERHLLYLSMIDLKGNIVKQMTLNDIINEYNGHKYKTNYHDLLTKREGDRTDARRSWDTIENIKELKEGYLSQVIHIISKMMVEYNAIVVLEDLNTGFMRGRQKIERQVYEKFEKMLIEKLNCYIDKHADPLEDGGVLHPLQLTCDAQKWKRSHQCGCLFYIPAWNTSKIDPVTGFVNLFDTRYDTREKARLFFSKFQRISYNSEKDWFEFTFDYNNFTSKAEGTRTEWTLCTYGERIHTFRNPKKNHQWDDEQIVLTENFKQLFDKYGIDIHGNLKEAISTQSDSQFFRDLLGLMKLLLQMRNSITNSEVDYLLSPVADETGHFFDSREEIESLPSNADANGAYNIARKGLWVIKKIQETAENEKFSLAISNKEWLQFAQTKPYLNE

SEQ ID NO: 17 - DNA, 3663 bp, synthetic construct
  source 1..3663: mol_type = other DNA; note = New Cas12a protein, codon sequence, id218
aagaagattgacaacttcacaggctgctattccctgtcacgcaccttgcgtttcaagctgatcccgcaagggaagacccaggagcacatcgagctgaagcgcctgttagacgaggacgagaagcgcgcggaagactataagaaggcgaagaaaattatcgataagtaccacatcaccttcatcgaccgcatcctgcacaacatcaagctgaccgagttacaatcgtatatcgagctggatgaggtgtccaacaaagatgagaagcaaaagaaggagctgctgaagatcgaagggcttctgcgcaaggagatcagtaactcgttcaagaaggacgacgagtataagaagatcttcggcaaggagattatcgaaaccattctcccggagtacctggacgatcaggaagagatcgaaatcatcaagagttttaaaggattcagcacggcgttcgtggggtactgggaaaaccgtaagaacctgtacacagacgaggagatcggtagcagcatcgcctatcgcgccatcaatgacaacctgccgaagtttatgcgtaacgcgtctatcttcaaaagtgtcgagaagatcttctcggaagaggagctgcgcgagattaagaaggaagtgttgaacgatgaatatgacgtccaagatttcttccgtaaggacttctacgacttcctgcttcctcaagatggcatcgacatctacaacgcaatcctgggcggactcgtgaaggaagacggcacgaaaatccgcggtttgaacgagtacattaacttatacaaccaaaagaacaaggagaagaacgccaaactcaagcccttgtttaagcaagtccttaccgaaagcgaaagtaagtcattctacatcgacgaattcgcgaaggacgaagaggttattgaagcgttccaaaacaccttcgccgaagagggcgaggtgtgcttggctatcgaccagattcaaaagctgttcaatcagtacgagatctacgattcgaacggcattttcatcaagaacggactggcagtgacggccctggccaacgacatctacggctcgtggtcatacattaaagagaagtggaacacactgtatgacgcggagaacaccggacgtaccagcaaggagagcgagagctacattacgaagcgtgagaacatctacaagaagatcgagtcattcagtctcaacgagatcgagacgatcaccgagggtgaggagaagctcttgtccgagatctctaacatcatcagcgaaaagattcaactgtacttcgagaagtacaactccgcgaaatacttgctcaacagcgactacaagctcgataagaaactgcaaaagaacaacaaggtggtcgaggtcatcaaagagttgatggacagcgtgaaggacatcgaacgttacctgaagcccttcatggggactgggaaggaagaaggacgtgacgagtacttctacagcgagtttgagaacacgcttgaggtgctgtcaggcatcgaccgtctctacaacaagattcgtaactatgtaaccaaggagccgttcagcaagaagaagttcaagctgtacttccaaaacccgcagttcatgggtggctgggaccgtaacaaagaggctgactatcgcgtggccctgcttcgcaagaagcagaagtactacctggcaatcatggacaagagcgactcgaagtgtctgcagcgtgttgagcaaggcaacggcgaccttcagaagatgaactacaagctgatcagtggtgcgtcgaagtcgcttccgcacgttttcttttcgaagaagtaccttagcaataataacgtcgaggacgagattctgcgcatctacaaatccaagactttcatcaaaggagaaagcttcgacattgacgactgtcacaagttaatcaattactacaaggagtcgattaagagccacccgtggggttctgcattcgacttcagcttttcggacactaacgagtacgccgatatccacacgttttaccaagaggtagacgacatgggttacaagattgttttcgataacgtcagcgaagaagccgtgaacaagctggtcgacgagggcaaactgtacctgtttcagatctacaacaaggacttcagcgagaagtcccacggtaccgagaaccttcacaccatgtacttcaagtctttattcgacgagaataaccgtggtaacatccgcttatgcggcggcgcggagatgttcctgcgcaaagcgagcctgaagaagcaagagcttgtggtacaccctgcgaacgtcccgatgaagaacaagaacaagaataacccgaagaagaccaccaccctgccctacgacgtttacaaggacaagcgttactcagaggaccaatacgaaattcacatccccatcatcatcaacaaggcaaccaccaatttctacaagctgaacctggagattcgcaagctgctgcgtgaggatactaacccgtacgtgatcggcatcgaccgcggcgagcgcaacctgctgtacatggttgtgatcgatggcaaggggcgtatcgtggagcagtacagcattaacgaaatcgttaacgagtgcaagggcatcacgatcaagaccgactaccactcccttctggacgagaaggagaaggagcgtatggcggcccgcaaatcatggaaaacggtagagaacatcaaggagctgaaagagggatacatctcgcaagttgtgcacaagatctgcgagttagtgcgcaagtacgacgcagtcattgccctcgaagacttgaacagcggcttcaagaactctcgtgtgaaggtcgagaagcaggtgtaccagaagttcgagaagatgctgatcgacaagttgaactacatggttgacaagaagatcccgtggtacgagaacggcggcgtgttgaaaggctaccagcttaccaacaagttcgagagctttaagaaaatggggacccagaacggtatgatgttttatatcccggcgtggctgactagcaaaatcgacccgagcacgggcttcgtgaacctgctgcgcacccgctacaaatcagtacaggatagtaagaatttcatcaacaaaatcagctccatcaaatacgacaaagacgaggagatgttttgcttcgccatcgactacagcaactttgaacacaccaacgctgactataagaagaagtggattctgtactccaacggggagcgtattaagcacttccgcaacccggaaaagaactctgagttcgactacaagatcatcaaccttactacggagttcaagaacctgttcgaggagtacaacatcgcctacgagctgggtgaggatattaaagagcagatcaacgttattaacgagaagaagttcttcgagaagttcatgtcaacggtgacgctgatgttacagatgcgtaactcaatcaccggtcgcaccgacattgactatttaatctctccggttaagaacgacaccggcaatttctacgacagtcgtaacttcgaagcgctggagaacgcggtgctgccgaaggacgcagacgccaacggcgcgtacaatattgctcgcaaggtgctgtgggcgatcgagcaattccgcgaatcggatgagagcaagcttgaaaaggtgagcattgcgatctcgaacaagaagtggctcgaatacgctcagaag

SEQ ID NO: 18 - DNA, 3966 bp, synthetic construct
  source 1..3966: mol_type = other DNA; note = New Cas12a protein, codon sequence, id233
accttctacaacatcattctgaagaaatacaagaacattgaaggtaagatgaaccagaacacgtctatcagccagttcacgggtctgtacccggttagcaagacactgcgttttgagctcaaaccgatgggcaagaccctcgaaaagattaaggaaaccggtattatcgagaacgacaagcgtcgccataatgattacttcgatgccaagaaaatcatcgacacttaccacaagtatttcatcgacgcggccttatcgaaattcagtcgtattgactggaatccgttaaaagaggccatcgagggctcactggacaagtccgatgctagcaagaagaagttagaaaagattcaaaccgagttccgtaagaagatcgcaaaggcgctcaccacccacgatcattacaaggagcttacagcctcaacgccgaaggacctgttcctcaaggttttccccgatcacttcggtaagcaacccgcaatcgacaccttcgacggcttctcttcctacttcaccggctttcaggagaaccgccagaatatctattctgatgaggcgatttcaaccgcaattccgtatcgccttgtgcacgataactttcctaagtttctgtcaaacatcgaggtgtacaagacactgaaggataacgcaccttctgtgttaagcgacgccgagaacgagcttaaggacttccttaatggtaagagtttagcaaatatctttgagctgaacgcctacaacgacgttctgacccagagcggtatcgacttcttcaaccaggttatcggcggcatttctggggaaggcggtgaaaagaaaacccgtggtattaacgagttttccaatctgtaccgtcagcaacacccggagttcgctcagaaacgcctggctaccaaaatgatcccgctgtataaacagatcctgtccgaccgtgagacgaagtccttcatcctggagtcatacagcaccgactcgcaggtccaggagtcggtaaaggagttctttgagagccagattttgaactgcgacatcgcaggccgcaaagtgaacgtgctcaaggagctgtcaagcctgatcaaacgcattaccgagttcgacctgggctctatttacgtaaaccaggaagagctgagtaatatcagtctggagttattcaagagctggaatactatcaacgccgtgctctttaaggacgcggagaaccgtatcggtagcgcggagaaggccgcgaacaagaagaagattgatgcgtggatgaaatcaaacgagttttctatcgcgaccctgaacctggcaatcgcggagagcgacagcgaggaaatcagccgcgtaaagatcgagagctactggaacgacttcgaagccaaagtgcagtcgatcctgtgtggggataatcgtcgtaacttagacgagttcttaagcgccaccttcaatgaaaacaatgccctgcgcgaggactcggagattatcggtaagcttaaagcgtttctggacgcgcttattgagattatgcactccattaaaccgctgatctcagacgcggagaaccgcgatctgtcattttataatgagctgatgccactttatgatcagctcagcctggtggtgccgctgtataacaaaatccgcaactacgccacgcaaaagttgaccgagtcagagaaattcaaactgaacttcgactgtcccaccttagcggacggttgggatcagaacaaagagaaagataataagagcatcctgctgcgtaaagatggactgtactacttaggggttatgaacaccaacgacatgccaaagatcgaagagatcaacgctttgggtaacgaggactgctacgagaaaatgatttacaagcagttcgactgcctgaagcagatcccgaagtgtaccacccagacgaaggcagccaaagcgcacttcgaggccggcaagacggaagacttcgttatcaaggacaagtcattcaacggaccgttcgtgattagtgagtacatctggaagttgaataactacgtgtggaacggcgagaaattcgtgctgaaattcggggacgcggacaaacgcccgaaacagtttcaaatggggtactacaaggagaccaacgacctgctgggctacaagaaagctcttgcagactggatcgacttttgcaagaagttcgttaagacctacatctctgccagcgggtacaactacgacttcttggacagcgataaatacaacagcctggacgagttcttctcttacctgaagacaatctgctataagatcacgttttctaagatcccgacgagccagatcgacgagtgggtaaacgagggtaagctgtttctgtttcaaatctataataaagactttgcaccaggtgccaagggtagtccgaacctgcataccctctactggaaatctgtctttagcccagagaatttaaaagacgtggtagtgaaacttaacggtgaggcggagttattttaccgcccatctagcgtgaagaagccgtactcacataaggttggggagaagttagtcaaccgcattggtaaggatggcttgccgctgccagagtctgttttcggtgagttgtttcgttactttaatggtaagctggacggtgagctgtccgacgaggcgaagaaatacctggacgttgccgtggtcaaagatgtaaagcacgagatcgttaaagaccgtcgctatacccaggataaattcgagtttcacgttcctctgaccttgaattttaaagcggactcgaagaacgagtatatgaacgagcgcgtgcgccacttcctgaaggataacccggacgtgaatattattgggatcgatcgcggtgagcgccacctgctgtacatgacgcttattaaccagaaaggcgagatcctgaaacaaaagagctttaacattgtggaatccgtgaattatcaggctaagttaatccagcgcgagaaagagcgcgatgcggcgcgtcgcagctggtctagcgtgggtaagattaaagatctgaaggaaggttttctgagtcaagtgatccacgagattacgaccaccatgatcgagaacaacgcaatcgtggtgctggaagaccttaatttcggattcaaacgtgggcgcttttgcgttgagcgccaagtgtaccagaaatttgagaagatgctgatcgataagcttaattacctggtgtttaagaacaaacccgagggtgacgttggcggtgtgctgaagggctaccagctggcagagaagttcgattcttttcaaaagctggggaaacaatctggcttcttgttttacatcccggccgcctacacgtcgaaaatcgaccctacgacgggctttgccaacttatttaacatgacggagctgacatcagcggagaagaagaaagagtttctcagccatttcgaggatatcacgtacgatggcaagaacgatcgtttcctgttttcgttcgactataagaattttaaatgctttcagaccgactatattaagaaatggaccgtatattcccagggcaagcgtatcgtttacgataaagagtcgaagtcggcgaaagaaatctcacccgtggagattattaaagcggccttggcgaagcaaaatattgcgctgacggatcagctggacgtcctcagcgcaattaacagcgcggaagcgtccccgaagtctgcgtcgttcttcggcgacatctgttacgccttcgagaagacgttgcagatgcgcaactcaatcccgaacacggacgaggattatttagtgagccctgttcttaataagaagggcgagttttacgacagccgctcgtgcggtgacactctgcccaagaacgcggacgcgaacggcgcatatcacatcgctctgaaaggcctgtacctgattaagaatgttttcgatgcgggcggtaaggacctgaagatcagccatgaggactggtttaaatttgcccagtcccgtaatagc

SEQ ID NO: 19 - DNA, 4017 bp, synthetic construct
  source 1..4017: mol_type = other DNA; note = New Cas12a protein, codon sequence, id236
gcgttcgtgatttgctacaacacctacagccgcacatacttggagaaagaaatcatcagtatgaagaccatcgagtcattctgtgggcaaaagaaaggctacagccgcagcatcaccttgcgcaaccgcttaatcccgatcggcaaaaccgaggagaacattcgcaaattgaagctgctggataaggacatcgaccgttcaaaggcgtacgtagaggtcaaggcgttgatcgatgacttccaccgcgtattcatcgaggacgtgctgagtaagaccgagttaccttgggagccgctgtacgaccagttcgagttgtatcaaaatgagaaggacaagcagaagaagaataaaatcaagaaagacttggaagagttgcagaagggcttgcgcaagtcgatcgtcaagcagttcaaagcggacgaccgcttcgataagcttttcaagaaagagttgttaaccgagtttgtacctagcgtaatcaagaacgacgactcgggcaccatcagcgacaagcaagcagccctggacattttcaaagggttcgcgacatacttcactgggtttcatcaaaatcgccaaaacatgtatagcgaagaggcccaaagcaccgcgattagcaaccgcatcgtcaacgagaatttccccaaattctacgcgaacatccaaacgttcaagtatcttgaggagaattttccgcaaattattagcgatactgagcagagcctgtcagacttccttggggataagaagcttaaggacatcttcagcatcgacggattcaacaaagtcctgtcgcagtctggcatcgacttctataacaccgtcattggcggcatctcggaagaggcaggcactcagaaggtgcaaggtctcaacgagaagatcaacttggcgagccaacaattatcaagtgaggagaaacacaagctgaagaagaaaatgaccgtactgtacaagcaaatcctgtccgatcgctctactgcgtcattcatcccagtgggcttcgagaagagcgaagaggtctacgattctgtaaaggaattcaaggagttgagcctggacaagaacatcgcggcgattaataccatcttttcacgcgatgactacgacctgacccgtatcttcgttccggcgaaggagattactgagttttcccttaagctgttcggccattggagcatcctgcaagacggactgtttctgttgggtaaggacaacgcgaagaaggatctgagcgagaagcaaatcacggagctgatcaaggagattgcgaagaaggattacagtctggcggaactgcaaaacgcttacgagcgttggactaaagagaatgacatccccgttgagaagacggttaagaactactttcgcctggccgagctgcgtactgacgagaagactaaagagaagaacttcactgacatccagaaggagttggagaaggcgttcgttcagatcgacttcgacaagaaggagaatctcatcaaggagaaggaagcagcgacgcccatcaagaactttctggacgaggtgcagaacttgttccattacctgaagttagttgattatcgtggcgaggaagagaaagactccgacttctacgcgaagtacgatcaagtcttgcaggcactggccgagatcatcccgctgtacaataaagtccgtaacttcgtgacgaagaaaccgaacgaggtgaagaaagtgaaactgaacttcgaatgctctagctttctgagtgggtggggaacggactatgggaccaaggaagcccacatcttcatcgacgacggcaagtactaccttggcatcgtcaacgagaagttaagcaaggaagacattaagttccttgaggagaagtcgccacgtatgatcaagaaagtggtctacgacttccaaaagccggacaacaagaacacccctcgtctgttcatccgttctaaaggcactagctacgcgccgtcggtaagccagtacgacctgcctattgagagcgtaatcgagatttacgacaagggcctgttcaagaccgagtatcgcaaggttaacccgtccgtctataaagagtccttagtgaagatgatcgactacttcaagttgggattcacccgccacgaatcttacaagcattacaacttcagctggaaagacagcaaggactataatgacatctctgagttctacgcggacgtgatgaacagttgttaccaactcaagttcgagtacatcaactacgacaacctgctgagccttgtagatgcggggaagctgtttctgtttcagatttataataaggattttagcacgggtaaggacggcgcgaacggcagcaccgggaagaagaatttgcacaccctgtactgggagaatttattctctgaggagaacttaaaggatatttgtctgaagttgaatggcgaggcggagttattttggcgcgacgttaacccgaatattaagaacatctgtcacaagaagggctcaatcctggtgaatcgtactacgagcgacggcaaggtgattccggaagatatctaccaagagatctacaaattcaagaacccggataaacaagaaaaggacttcaaaatcagcgacgaggcgaaggccctgctggagaacggtaaggtagtatgtaaggaagcgaaattcgacatcacaaaggatcgtcacttcacgcaacagacctacctgttccattgtccgatcaccatgtccttcaaggctccggagatcaccggccgcaaattcaacgagaaggtacaaagcatcttaaaagccaacccggacgttaagattattggcctggatcgcggcgagcgccacttaatttacctctcccttattaaccagaagggcgagatcgagctgcagaagaccctgaacctcgtagaccaggtgcgcaatgacaagaccgtatcggttaactaccaagagaagttagtccacaaggaaggcgagcgtgacaaagcccgcaagaattggcagagcattagcaatattaaagagttgaaggaaggctacctgagtaacgttgtgcacgagatcgctaagttaatggttgagaacaatgctattgtggtcatggaagatctgaacttcggcttcaagcgcgggcgcttcgcagtagagcgccaagtgtatcagaaatttgaaaacatgcttatcgagaagctgaactacttggtgttcaaggacaaagcggtgaccgaacctggtggtgtgttaaacgcgtaccaattgaccgacaagtccgccaacgtgtctgacgtgggcaagcaatgtggatggatcttttacgtcccagcggcatacacctctaagatcgacccaaagacgggcttcgctaacttgttttacaccgcggggcttaccaacgtggagaagaagaaagagttcttcgacaaattcgaggctattcgttacgattcgaagaccgacagcttcgtgctgtcgttcgattataaggacttttctgacaacgccgactacaagaagaagtggtccttgtacagtcgcggcgagcgcctggtcttctctaaggcggagaagaacgttatcagcgtgaacccgactgagaatctgaaggcgctgttcgacaagcaaggtatcaattggaaatctgaggagaatttcatcgaccagattaacgcggtccaagctgagcgtgagaatgtgtcgttcttcgatggcctgtatcgctcgtttactgcgatcctgcaaatgcgcaacagtgtaccgaatagctcgaagatggaagacgactacttaatcagcccagttatggccgaggatggtaccttttacgattcccgtaaagaggcggcgaagggtaaagatgagcagggcaaatggatctctaagctgcccgtggacgccgacgccaatggcgcgtatcatatcgcgctgaaaggcctgtacctcttgcaaaacgatttcaacgtgaacgataaggggtacattgacaacatctcaaacgctgactggttcgagttcgcgcaagaaaagaattacgccaag

SEQ ID NO: 20 - DNA, 4113 bp, synthetic construct
  source 1..4113: mol_type = other DNA; note = New Cas12a protein, codon sequence, id204
cagaatatcgagagcttcaccaatctctactcactgaccaaggtagtgaaattcaacctggacccgatcccggagacaaagaagtggctgaaagcgttcggtaaagtgttcacccataaccacagtgagaatctggaagataacttctttaagctggatcttaagatcgaggatgcaaagcgtatcattcaggacgtgttaaacagtgttcacgaagcgattattacgcaggcgctcgaaagcgaggaagccaaaagcattaatatttcgagctactacgagcttagcaaccagcgcaaccatgacctgtcagacttggaacagcgtctgcgcgaacaaattaaagtctgcttcattaagcaactgaaggacttttgctccgaagtctcacctaagaagaagaacagcgatgacgagaatggcttaccggacttgctcaagatgcttggttcgtccaacatgctcgactacctgagcaagagcgcgtcaaagtactcctcaccgattcatgacaagaagtcaatcgaggacgcctgcaaggacttgaaaggtttctggggtgtgctcgacaagtatattaagaaccgcgaaaactattacgttttcgataaagagcaagacacggccgtggcaacccgcatcgtgtcggaccttttgccgatgttctgcaagaactgtaaacagtacctgactaataaggcgctgtatgacagcatcttcacatcgcttaaaagctccggcattattatgcaaatgatcaacccgaagacgaacgaaatgcgttcgatcgagtcgtttgatagtagcatctataacatctcagggttctataagtatattagtcaaagcggcatcgagacgtacaacgccattatctcgcgcaataatattctggtgaatctctataatcaacaacagaagaaggcgaaccgtattaacggctttgtggagttgcataaacagattggctgtcgcgagtctgagagtacagaacgtattgtaatctgcaaggactgggattctactgagagtaaggaattgggcattagtatggaatctatgattaagcgtattatctgtatggtcaatgagacgattgtgccgacggagccgaatattctgcatccgcgtgtttataaccttattagctggcttgaaaacgcggtcgactgggacgggatctatgtcagtgacaaggcgctgagcatcatctcaagtcactacctggaagactggtacagcttgcaacagctcctgctgaacaaggagtccgtaggcacctataaccaacgtaaagagagcaatgagcagtttaagctgaattcagccgtcgagcttatgccggtcttccgtgcgctcgactgcaaggacttcgattcagttttcaagaagtccatttacaacgattattcagacatcctgaataagaacctgccaccgagccgtaacttaatcaacatgttgtgtcacgacgtcaaacaaatcattaataacgagttacccatctacacgaaatgtattacccagctcctcacaactaagatcagcaaggtgcagcgtgagggcttctttaagcaagaggaaatcatcgacattatccgcgactacttagattgcgtgctgagtatcgcccgctttgtgtacttcttcagcgtccgccaaaataaaatcaagggcgccccgtataacatggagctgctccagatcgtttcaagtttactgagcacggagcttggcaaccgccgtctgtggtacgacgaagtgcgtaactacctgttaaaatctccccaggacgaagtgaaagaaaataagcttaagctcaactttaattcgagctctctgctgaagggatggtctatcggcgaggagcgtactaaattaagcttacttctgaagaacgaagggtgttattatttgtgcatcctcaagaacgacaagacatgtaagaagttgttcgatattaacacccagaacaatatttacattgagcacggcaatgcgtaccgcatgatgctgcgtaacctgtcgttcaagacgttggtgggcaagggatatgtatctatgttcggcacgaagtattcggaagaagacgacgaggtcgccatccaacacgccaaacagctgatccgccagaattacctgaagaattacccggagttgtcagcggtgatcaccaaaccgcattcaaccaaatcagccttctttgccgacgttgactcgatcttgaaagactatgcgcagtgctattacaatcccatcaactggaatctcatcgaaaagttcgcgaacgagggcaacatctacgtgttccgtatccatgcgaaggattataaggatacgagtaagggcaagaaagacctgcaaactatttactgggaagacgtcttaagtggtcattcctcgcactccctttcggcgtatggtgaagttttccgccgcgacgcgatcagcactgacaagaccaaacccttcatccataagaaggggagcgtcctgatcaacaaacgcgacatcgacggcaattcaatccccaactctatttatattattatttacgcgcttctgaataaggtaccgctgcccaagaccgccgaggagaaggacattgagcgcgcaaagtccctgatcgaaaagaacatcatcatctcaaaggagagctggatgaatattatcaaggacgcacgtttctacggcgaggagaaatactttctgcataacccgatctgtttcaattacaaatctattggcttcaagtcggaagatttcagcaaggatatcccctaccacgtgatcaacccgattattcaagagctgatgcataactcatgtgccccgaccagcttcatcggtatcgatcgcggtgaaaagaaccttatctacgcttgcaaaatcaactccgagggctccatcctgttctgcaatcactacaacatcgtgcacgggattaactatttggagaagttagaggagcgcgcggacagccgcttgaaggcgaaacgttcctggaaacaacaggagagtatcaagaacttgaaagagggctacatttcccacgtagtgcatcaacttcttgaggacgttattagcgttggcaacgaaaacctcacaccaagttacattgtcctggagaagttaagccgcgagatgaaacgtggacgtcagaaaattgaaaagcaggtttaccaaaagtttgagctggccctggcctctaagcttggcttttacgttaataaggacatcgaggaagagaaacctggtagtatccagatgcccctgcagttcgtgccaccggttaataccttcgatcagattgataagaaggatagtttcggcatcatgctgtacacccgcgctaattatacatcagttaccgatccattgacgggctggcgcaaaacaatctacatcaccaacgggaataacgaggacatcttaaaggagctgacggaagccttcgatgacattttcttcgacggtgtggactacgtgttctcatacgttgagaagaacagcggccagcgctggaatatttactctggaaaccatggtaagtctctcgatcgcttcgagtataacaaggataccaagtgttacgaatcctataacatcgtcgaagtgctcgacgtgcttttcgcggattttaacaagagcagttcactcctggagcaaatgcgtcgcggcgtgaaactgaagaaagcagaggagaatcgcacagcctacgacagcttgcgtaaggcgattaacatgattcagcaaatccgcaacacgggtaacgagatcaaggataataacttcctgcagtcgcctattcgtaaggatggtaaacactttgatacgcgcaacgcgaacgacttccgcgacctgaacctgcattttatcaaggacgctgacgcgaacggtgcatataacattgcgcgcaagggcttaatcatggacgcacattacaagtactggatggagcagggcaagcctaacgttaagaagaagaacaagaaggagaaggaagttagcgcgctctcactttacgtctcggaccgcgagtgggatatgtggctcctgaaccgcgagatgtgggagaagcacctttcagagttttcacttcgtaaacaagat

SEQ ID NO: 21 - DNA, 3897 bp, synthetic construct
  source 1..3897: mol_type = other DNA; note = New Cas12a protein, codon sequence, id206
tctcgcgacttttctcagtttacgaaccaatataagcttagccaaactctgcaattcagcctgatcccgcagggccgtacccgcgagtttatggactcttttctgatccatgatcagtcacgctcggacgcataccccgaggtgaagaagattctggacaacgcgcataaggcgctgatcgaacgcaccctgggtagcatttctcagcgcattcactcgctgtctgataaggaaaagaagaagtttgcatcgatccttaacggagacgatattaactggcttcagttagacaaggctgtggagaacgaggcagagaacggaaaggatgccacggacgcagtacgcttagagtattgtcgcttactcgccatgttgttcagttacgatgacgccttcgagtcgttggtgaagaatgccaccccggacgaatactttaagaacgaattgaagaaaatggctctgtccgacgacgacaccgcccgcttccttgagaagtttaagggctttgcctgttacttcaagggtttccaggagaatcgtcgtaacatttattcacaggaagcacagtcaacagccgccccacaccgcgcaatttacgataactttcctaagatgaagcgctgtattaaaatcttcaaccacattaaggacgcataccccgagattctcactaccgccaccactgagttagctgaactgcttaatgggcagagcatcgacagtgtgttcagcattcaggagtacagcaactatctgagccagaagggaatcgacaagtttaattccattctgggcggctacaccaccaaggatggccagaaagttaagggcattaacgagtgtatcaatctttaccgtcagcaacacgaagacgcgcgcaaagaccgcagtttctcaaccatgccgctgctgttcaaacaaatcctgagcgaccgctcgtcattaagcttcatccctcaaatgtttcaaaacgaccaggagttatttaacaccctgaaagagtttattcagcagctggatcacgttaccacggcagatggaatcgatcgcccgttaatcccgtcgtttcgtgactgcttggataatatttctatcgatgacggaatcctggtatcagcgtcgtcccttactgagatctcgatgcgtgcgtctggcagctggaatgcaatcccggacgcgatggaaaactgggcagccgtgaacatctcgacaaaggccctgcgcgagaagtggcttaagaagaattcatactcgttaacagagcttgacgcgctgcacttggagaaagagaccgacgagaaacgcatttcttgcgatatcctggactactggaaggggaagaaggcacttgactgttacgaccgctacgacgccttactgaaagacatcaccagtctgatgagtgctccggtgaacgatatccctctggccgagcgtaaggacgacgtggcgttgattaaggattaccttgactctttaatggatattcttcacctcggtaagcccctgtactgccaggatattttagaccgtaatgaacaattttactcattgttcgacggcctgtacgagcaactgcagattgttattccgctgtataaccgtattcgcaactacttaacgcagaaagccgacacgaccaagaaagtaaaacttatgttcgatagcccgaccctggccgacggttgggaccagaataaagaacaaaccaacaacgcgattatcttactgaaggatggtaagtactatctggggattatgaacccggctaagaagccggtgatgagtaagatggtagtgcagaaagcggaatccgaagtagaggtgtaccaaaagatggtgtacaaatacctgccgggccccaataagatgttaccgaaagtattctttagtacgaaaggccaaaaggactttaacccgccgaagagcctgcttgagaaatacgaggctaagatgcacattaaaagctctccggacttttccttggcttttctgcacgagctgatcgattactttaaggcatctattgcaaaacacccggattggagtaagtttggatttaagttctcaccgacagcctcataccagggtatcgacgagttttaccgcgagatttctaaccagggttaccgcctctcgttcgaaatcatcccgaaggagaagattgacgatctcgtgaataacgagcagctgttgctgtttcagatttacaacaaggactttgcgccaggcgcgacagggacccctgataagttcactatttattggaaaatgctgtttagtccggagaacctgtctaatgtaatcctgaagttgaacggtgaggccgaactgttttggcgcaagcaggtgattgcaaaaccctttagccacaagctgaacgagaagatggttaatcgcacctacgttgatggtaccgattccaccgggcgccctatcgttaagcatatgcctgagaaggtgcacgcagttatcttcgactacgttaacggtaagaacgacgatattcacaagaaagagtatcaggatgcgttagccctgtacgacagcggtaaggtgatcgttaaggacgttacccacccgatcattaaagaccgtcgctatactcaggagtcgtttagctttcacgtgccgattaccattaactttaccgcgcctgacaagccatcaaagtttaacgactcgattattgcgtttctgaagaataacccggacgttaagattattggaattgatcgtggcgagcgcaacctgatctacttttcgctgatcgatcaacaggggaacattctgaagcagggctccttgaacaccgttaacccgcgcactgaggctcgcatcgagcaagtgaactaccacgagttactcaaccagaaagagaaggagcgtgacgaggctcgtaaatcatggagtagtattggcaagattaaggacctgaaagcaggttatcttagccaagtgattcaccagattgcgccgcttatggtagagaataacgcgattgtgatcctggaagaccttaacttcggcttcaagcgcggtcgctttgctatcgagaagcaagtctaccaaaatttcgagaaagcgctgattactaagctgaactacctggtgtttaaagagattcaggattacaatacggccgggagcgtcttaatcggctaccaacttaccgataagttcgtgtcatttgagaaaatgggcaaacaaacaggtgcactgttttatgtgccagcggcgtacaccagtaagatcgaccctactaccggcttcgctaacttgtttaacactcgtaagtgtacaaacgtcgagtcacgcaaggtcttcctgatgaccttcgacgctattaagtacaatgctgtccgcaagtcgtttgccttttcgttcgattacgcgaagtttcaaaccagtgctaagtcttttcagaataagtggactgtttacacctcgcagaaacgcctggtgtttaacccattaacccgccaggacaccgctgtaaacccgacacaaatgatcgttgacgcgttaaacaagcgcggtgtttcgttatccgacgagttcgatctgaaagcctacctgcaaaacacagatgcgattaaggcaaacgcagagttcttcaaaaccatctttatcgcactcgaatacactctgcagatgcgcaattcgaacagctcatcgggcgaggattacatcgagagccccgtgttaaataagtccggtgagttctttcgtagctccgaggataaccctgctcttccgattgacgccgacgcaaatggtgcgtaccacattgcgatgaagggattattcttgcttttgaacgtgtttaaccagggaaagaaggacctcaagattgaacaccaagcgtggtttgagtttatgcaaaagcgcaacatg

SEQ ID NO: 22 - DNA, 3693 bp, synthetic construct
  source 1..3693: mol_type = other DNA; note = New Cas12a protein, codon sequence, id210
gcctactcagacttcattggcatgtaccctctgcaaaagaccttacgctttgagttaaagccacaaggacgcaccatggaaactctgaaagaaaaggattaccttaagaatgatctgtatcgcgccagccagtacgcggggatgaagaagttaattgacgactaccaccgctcttttatcgacgaatcgctgatgacctcggaaatcgattggcaaccgcttgcagacgcgctcgacgcgcgcaagaataacgacgaaaagagtaagaagcagcttgagattcttcaagaccaataccgccgtcaggtttcgaaactgtttacaggcaaggaagtgtttaagtcgcttttctctaaagaggtgctcgatttagtccgcgaaaagtgcgtaaaggagcgtgatgattcgaaaattcaaatctgcgacgcatttaaccgctactcggtatactttaccgggtttcatgaaaatcgccgtaacttttatgtgtccgacaagcagtcaacgtctatttctaagcgtctggtagatgataattttccgaagtttctggaaaacatccgcatttacgaaaagatcgaagtggaatgcccagagatcattgagaaagtgaagaatgagttaaagctgactgacctcaagaaatattttagcgtgagcgagtttaaccgtttcgttcgccaatgtggcattgatgactacaactggattctcggtggtcacgtcggagaagaagcccacgtgaaagggtttaacggttttctcaacgaggcctaccaacagggtctgctgaagacacgcgtactgatgcgtccgctgtataaaatgatcctctcaattagcgagagcaagtctgtactgccttaccaatttaaagacgagaaggaaatgatggacgccgtggtctcatttattcaaaaggcgaacgaagggatcattgcgcaagcgggcgaccttctgaagagtgttgagtcaacggacctgtcccgtgtgtttatcccggacaaacaacttacggagctcagccaaattttattcggtgattggggcgctctcggcaccatgatgcgcgaatataaggcttacgatctcggggacccgactatgagcaaaacaatgaagaaggtagataactggatgaagagcagcgaattctcgttggaagacgtcaagaaggcagtcttgcacgccgaccaagagttcactctggaaaactacgaggtgcgtgtcgaccaagtgtgcagtaagttgatcgaaacaaacgctgtcagcgagcttaagagcgttaaaattgatggcaccgatggcgagacgcgcaaccgcagtgcagaatgccttcgttgtatccttgagggctaccaagacctgttgcattgtatgaacatgtttaaagtgtcggatgagctcgatcgcgacgaggctttctactcagagtttgacttaattctgggcgagttgcaagagattattccgctgtttaataaagtacgcaactactgtaccaagaaggcgtataacgagacaaaggtaaagctgaagttgtctaccccgactttagcggatggctgggatcttgacaaagaaaaggataataaggccgtgattctccgctcaggcggccaatactacttgggcgtaaactgtgataaaggcttcttagacttttccaaatacgagaccgacgacgaagagtgcttcgaaaagctcaactataagttactgccgggccctaataaaatgttaccgaaagtgttcttttcaaagtcgtggaagtcagaacataccatcccggaacgtattcaagatggttatgatcgtgggattttcaagaaaggcccggagtttgacctgatgttttgccacgaactcatcgacttctataaagaaagtatttccatccacccggtgtggtcgaaatttgactttcgtttcgcagagacgagtgattaccaagatatttcgcagttttacgccgacgtacaacgccaaggctttaagatgagctttagtaaagtttcaaagtccgaggtgtataagttagttgacgagggcaagcttatgttatttaaaatccacagtaaggacttttctgaaaacagtcgcggcatgcccaacatgcatacgctgtactggcgtgccctgttttcggaagaaaacctcgccaatattcgcattaagcttaatggtgaggcggaacttttctttcgcgagaaaagcatcgacactccggtcgtacacaaatctggtagcgtacttgttaacaagacctttaatggtaaacacgtaccggataaaatttactacgaattatttcgctaccgcaatggaattatcacggctctttcacctgaggcaaaggaatacgaaggtgccatcgtgagttctgtagcgaactacgacattgtaaaggaccgtcgctatacggttgacaagctgttctttcatgtcccactgacttttaacttcggcgtacccgatcgcagtaaggatattaatcgtatggtactggaacacgccattaagtctaaagacaccaaaattattggtattgaccgcggtgagcgtaacttgatctacatctcgatgatcgataaggaaggcaaaattttacttcagaaatcactcaatgtgattgacggtgttgactaccatgggaacttggatgtgcgtgaacacgaaaacaaagaggcgcgccgcaactggacaaaagtcgcgggcattaaagacctgaaggaaggctacctgtcagcagcagtctcgcaaatcgcgcgcatggtcgtcgaaaatgacgccatcgtcgtgatggaaaatttgaactatggttttaagcatggtcgctttaagattgagaaacaagtctaccagaaattcgagagtatgctcatcgacaagctgaactacctggtattcaaggataaggaagactttacctcagatggccgcgccatgcgcggctatcaactgactaagtctatcgattcagtgaaagaaatcggaacacaaacgggcgtgatcttttacgtcaacgccgcctacacgagcaaaattgacccgaccactggttttattaatcagatccagtttagcacgaaatcgactctctctgcaaaacgcgacctgctctgtaacattgactcaattcgctacgacccctcagaagagtgctacatctttcgcatcgactacacaaaagttggtgatcgtacgaaagagtttaagaacgtgtgggatatttacacccgtggcgaccgcatcgtgtattccactaaagagcgctgcgacaagcacttgcacccaacggagattatgactaaggcactggagagcgcaggcatcggcaaggaaggcgagttgaaggaaaagatctgctcggcggatagcaaagtgattgaagcggtagtttacgcgttagaccttacgctgcgtttacgtaatgaagaccgcgaaacggactatattttgtccccggtgcgtaatgcagatggtaaattcttttgcaccttggatggcgaccttagtcttccgctggacagcgacgctaatggtgcctacaatattgcgttgaaaggtttgttaatgctgaagatgaccgaggagtcaaatggcccagacgctgataagtatgatatttcattagtaacgaacagcgactggctgacgttctgccagaccggccatcgcacgtggaaatca

SEQ ID NO: 23 - DNA, 3693 bp, synthetic construct
  source 1..3693: mol_type = other DNA; note = New Cas12a protein, codon sequence, id211
acaaacttttacagttcattcactaacttgcgttcggtctcaaagacactgcgcttcagtttgtcacccgttggcaagacgcgtgaaaatattcgctcagcaaacattatccctcaagatgagaagcgtatgcaagactaccttgtactgaagaacgttttcgatgcgtaccataaggcatatattgaccaacacctttgtcgtattcaaggcgactggtcggagttagcgaatgcgttggatgagcgtcagagcgcacccgatgcggacgctctcaataagatgcaggaaacgtatcgtaaggaaatcagcaacttactgtctggtaagggtaccggtaagaatcagtacctgtttccggccgatggggatggcggttctctgctcgaaatgcttgatccggcggacatgattaagcaaactgtaaaggagaagaaagatgctgagaagggtaatcgcccaccacagtacttgatccctgagttcgggcagaacatcaaggaagtagagcaattcaatcgtttcgcgggctattttgaagattataagaaaacccgcgagaatatctatgcgtcagagggatcaacgagtgtggcgcatcgcattgttcacgagaattttccaaaatttatgagtaatgtgcacacttttcgccgcctgccggagagcttgcgtcaaaagttcgaacacgacctgagtgaattgctcgatggggcctcgttggagaccatgtttgatgtagattactttaatgtggtccttgcccaagctggcatcgagcgctataacacattgttaggcggcaagaaagaggagtcaggtgttaaactgcaaggtctgaatgaactctgtaacctggaatttcaacaaggtaacctgcctaagaaagtcgttttcgtgccactgtataagcagattctgtcggatcgtgagaaagccagttttctccccgaaaagttcaaatcggttgaggagatgttggaaagtattgctgactatcgtcagtcattaagcaatctcatggcaagccagcattgcttactggacgagaacatgtcaacgggatactgtgatgcggggcacgtgtacattcgtaagagccaactggcctctctcagcatgctgtgtttcaacgaatggtcaaccttccgcacagccgttgaggccaaggacgtctataccttaagcgagctgttacaagctgcgggcaaggatatcttagaggatgtgcgtcgtgaattaaatttcagtctggactccattgacgaggcaggcgaagcactgcttcctgtactggagaaacagactctcaccgaggatgactacgcgattattaaagagtatttggatgccgtgattgccgcccaacacatcttgaaggtattcgaggcagaagacgatgctgagaaagatcctgtattttatggagtggccgacattcttaacgacgctctccgccagggtgtgcgtttatacgatcgcgttcgtaattacgctacgggtaaaccatatgctgaagataaatttcgcttaaccttcttcgcacccactttactcaatggatgggacaagaacaaggagcgtgataattgttgtatgttgtttcgtaaagatggcctgtactaccttgggattctgaacgccaagaacaagcctttattggacgaggaagccgagattgagggtgcctcttatgagaagatggtgtacaagtacctgcccgaacccaataagatgctcccgaaagtatttaacaagagccagaagtggattgcggcgcacggtttagatgatgacattcgtgatggctatgagaaaggattgcacaaagtgggtcaatcgttcgataaggcctttatgcacaagctgattgactactacaaagcgtgtatttctgagtacgaggattgggccacgtttcgttttcgcttcagtgcgaccgatacttaccagaccatggcggatttctacaatgagattgcggcacaatcttacaaagtattcttcaccaaagtcagtgagcaacaaattaatgcggccgtggaagcggggcagctgtacctctttcaaatctacaataaggatttctcgccgtacagccatggtgctcctaacctgcacacgctttattggcgtgagctctttagcgccgagaatctgcagaatccccttgtaaaattgaacggtgaagccgaaatcttttatcgtcacccatctatcgcaaacccttttcgtcaccagaagggtgatgtactgattaataagaccgatgctgccggtcgtacggtcccagaggatcgttaccgtgccgccgcgaccgatgctgcaaatgggatgcctgttcaagcactgcgtgcatcatacccggaattaattttcaaggttgcggatcgcgagattgttaaagataaacgttacactaaggaatccttcgcgttccacgttccactcacactgaacaatgataataacgcgggtttcaaacttaacatggcagtgtgcgagaaattgcagtcggatggcgagtactatattatgggcattgatcgtggtgagcgccacctgttatacatttcggtaatcgatcgtaatggtcgtattgtcgaacaacgcagccttaacgtagtcggaaagaatcgtgtcgactaccaagcaaaactcgatgcgttggagcgccagcgccaagaagcgcgtaagaattggcgtgcgatcacggcaattaaagagctgaaagagggatacctgagtatggccattcatgagattacagagttaatgctgcagtacccgtgtatccttatcatggaagacctcaattttgggtttaagcgcggtcgttttcgtgttgagaaacaagtgtaccagaaattcgagaaaatgctgattgagaaattaggtttctggatgcaaaaggataaggcggcagacgccgccggcggtatccgtcacgcgtatcaattaactgaaagctttcaatcttttcagaaactcggaaagcaatcgggtgtaatcttttacgtgcctgctaattacacgagtaaaattgaccccaagacggggttcgttaacctgtttggcagtcgtcagctgacgtacacgtcggtgtccgccgcgaaggcgttctttggcactatgcgcgaaattggttacgatgcacaacgcgactgttatcgtttcgcgtttcgttattctgattacgatttgtatcagcaagaccaccgtgactcttggacggttttcactgccgggaagggtcgcattgcacacacagttaagaatggtcgccacacttttgaagagattgatgtaacggaacgcatgaaggagctctttcagcgtttcgggctggacccgtacgcagcagaccttcgtggtgcaattgatgccgccgacaagaaggacttctttagcgagttactgtggttgtttaaagtgaccgtacaactgcgctacgaggattccgagcaagactttattctgagcccgatcgagaaggacggtcgcttcttcgattcgcgtcaggccaagaaggatgagccacaagatggtgatgcgaatggtgcctaccacatcgcgttgcaaggccttcgcttagctcgtgagcgtattaaagatggacgtgtacagaaggacccaaagggtaaacaaacactgaattggttacgtttcgctcagcgcaaagattacctggag

SEQ ID NO: 24 - DNA, 3657 bp, synthetic construct
  source 1..3657: mol_type = other DNA; note = New Cas12a protein, codon sequence, id217
aagaagattgaccactttaccggctgctattcagtttccaagactctgcgcttcaaactgattcccgtaggcaagacccaagagaacatcgtggcgaagcgcctgctggaagaggacgaaaatcgtgccgaagactacaaggcagcgaagaaaattatcgatcgctatcacaaaagcttcatcgacgacgtcctgaagtcagtcaagttgtcccacttaaacgaatatgcgagtcttttccttaagggtaaccgctcatccgacgagaacgaaaccctggagaaattggaagagaccttacgtaatgagatttcgaaagcgttcacggagaatgagggattcgcgttattattcaagaaagagttaatcgaggatgagctcccggagcgcttagagggtgacgagctgcgtatcatcaacagtttcaagggtttcactaccgcattctccgggtttcacgaaaaccgtcgtaacatgtacgctgcggacaagaagtctaccgcaatcgccttccgctgcgttaacgaaaatctccagcgtttccttcagaacatcgcgtgtttcgagaaaattaagtgcattctcgacgccgacatgtgcaagaccatcgagtccgacattctgtctggtgactactcgattcgtgacttcttcgtggtggatttctacaacttagtgctgacccaacaaggcatcgatctgtataacgcgttaattggtggtgtagtaaaagaggacggccgcaaaattcaaggtttgaacgagtgcatcaacttgcacaatcagcaaaacaaagatcgcttgccgcacctgaagccactgtacaagcaagtcctgtcggaccgcaccggagaatcgttttacggagaggggcttacaagtgatgaggagctgtacgacgccatgcgtaacaccgtgggattcgaaagcatgatttacaagaattaccgtaaagcagaagagctgttcgccagcctggacttgggcgccttcgatggtatttacgttaagaacgggccagcagtgacaaatattagcaacgacgcgtacggccactggggtaacttcaccgagaagtggaatgagcagtacgatctggccaacgaaaagagcaacgctaaggaccttgagaagttcgaagagaagcgtcgcaaggactttaagaaaattgaatccttcagcctgggcgaaattattaagttaatggaagataacgaaaagctgcctaccgctgtcgtcaaaggcatcaaggatcgtattgcggcggtagagatggcatacgcaaacgctaaaaccgtgctgaccgagggattcaagacggagaagcaactgaagaaggacgaagttacggtcggccgcattaaggccctgttagactccctcaaagagttagagtccttcctgaagcaatttaaagggaccggaaaagagtcaggccgcaacgagctgttttatggcgactacaccttatattgcgacatgctgagtgaggtggacgaactgtacaacagcgtgcgcaactacgtcacgcagaagccatactcgactgacaagtttaagttatacttccaaaacccgcaatttcttaacggctgggaccgtaacaaggagacggactaccagagcgtaattctgcgtaataatgaccaatactacctgggtatcatggactccaaaagccgcgacatctttcgtcgtgtacctgaatccgacgagaaaggttacgaaaagatggtttacaaacaaattcccaacgccagcaagtatttgagttcgaaacaaatcaacccgcaaaacccgccggaagaaattgtacgcatcctggagaagaagaagacggacaatcgtagcttgacggctgacgaaattaaaaccttcatttcttacatccaggacgacttcttaaagaattacagcttccttcgtgacgagaagggcaaagaatacttcgacttcaagttcaagaaacccgaggcatatcgccgcctcaacgacttcttcgatgacgtagacaaacaaggctacagtgtgaaattcagtgacgtgccaaagacctacgtggactccctggtcgagaagggtgagttatacttattccgcatctacaacaaggacttcagcgaggcttcccacggtaccccgaacctgcacaccatgtacttccgtagcctctttgatggtgccaaccagggcgtgatccgtctcgcgggcgaggccgaaatgttcatgcgtcgtgccagtatcaagaaggacgaactgatcgtgcaccccgcgaacaccgccattaataacaagaaccctttgaaccctcgtaagacccgcacactgccatacgacgtgattaaggaccgccgtttcagcgaggatcaatacgaactgcacatggcgatcagtattaacaaaattccggagaatgggatcaaaattaaccacgacgtgcgtaagttcttacgcgaggatgaaaacccatacgtaattggcattgatcgcggcgagcgcaaccttctgtacgtggtggtcattgacggcaaaggcaagattgtcgagcaacatagccttaacgaaatcatcaacgagattcacgacgtgaagattgtgaccgattaccacttcctgttagatcagaaggagaaagatcgtctcgccgcgcgccaagactggacgaccattgaaaatattaaagagcttaaagagggctacatctcacaagtggtacataaaatttgcgagctggtcgagaagtatgacgcgatgattgctcttgaggatctcaactcggggtttaagaacagccgcgtaaaagtcgagaagcaagtgtaccaaaagttcgaaaagatgcttattgacaagctgaactacctcgttaataagaaaaccccaatcgaagagaagggttccgtgatccatggttaccaacttacaaataagttcgagtcgtttaaaaccatgggtacgcagaacggtattatgttttacattccggcgtggctgacctctaagatcgaccccactacgggcttcagcgatctgcttaagcccaagtatactaacatggagagcgcacgctcattcgtaggtactttcgacttcatccgctacaatggtgacgagggttattatgaatttggcattgactacagcaagttcggttgcgacgccgaccaccgcaagaagtggaccttgtgctcgcaaggaagccgcattcgtaccttccgcaacccgctgaagaataatgaatgggataacgaggaagttaacctgaccgaaatgttcgacacgctgtttgagaaatataaagttaacgggaagtctggggacttacgcgacaaaatttgcggcgttaccgaagccgacttctacaaggagttctacggactcctgcgcttgatgcttcaaatgcgtaatagtattactggccgtaccgacgttgactaccttatttccccggtcaagaaccgtcacaacgacttcttcgacacgcgtaatgccgacaagacccaacccatggacgccgacgctaatggagcttacaacatcgcgcgtaaagttttatgggcaatcgaggtattcaagaaaaccgaagaggacaagctcgacaaaactaaaatcgccatttcaaataaagagtggttgaaattcgcgcagacgatggttgac

SEQ ID NO: 25 - DNA, 3819 bp, synthetic construct
  source 1..3819: mol_type = other DNA; note = New Cas12a protein, codon sequence, id219
atgataatttataattgttatatcggaggcagttttatgaaaaaaatagatagctttactaactgttattctcttagcaaaaccttgagattcaagctgatacctattggcgctacgcaaagtaattttgatttaaacaaaatgcttgacgaagataaaaaaagggcagaaaactattctaaggcaaaaagcattattgataaatatcatcgcttttttattgaaaaagttttatcttcagttaccgagaataaggcttttgacagcttcctcgaagatgtcagagcatacgctgagctttattacagatcaaataaagatgacagcgacaaggcttcaatgaaaacacttgaaagcaaaatgcgtaagttcattgctttagctttacagtcggatgaaggttttaaagatttgttcggacagaatttaatcaaaaagactcttcccgaatttcttgaaagcgatacggacaaggagataattgcggaattcgatggtttctcaacatattttaccggtttcttcaataatcgcaaaaacatgtacagcgcagacgatcaatcaacagcaatttcctaccgttgcattaatgataaccttccaaagttccttgacaatgtcagaacatttaagaattctgatgttgccaacactctcaacaataaccttaaaattctcaatgaagattttgacggcatttacggaacctctgccgaagatgtattcaatgttgattatttcccgtttgtgctttcacagaaaggaattgaagcatataattctatactcggtggctatacaaactctgacggcagtaagattaaaggcttaaacgaatatatcaatctttacaaccaaaagaacgggaacatacatcgtattccaaaaatgaagcagttgtttaaacagattttaagcgaaagggaaagtgtttcattcatacccgaaaaatttgattcggatgatgatgtcctttcttcaattaatgattattatcttgaaagagacggaggaaaagttctttcaattgaaaaaacggttgaaaagattgaaaaactattcagcgccgttacggattacagcactgacggaatatttgttaagaatgccgcagaacttacagctgtctgctcgggagcattcggctattggggcactgttcaaaatgcctggaacaacgagtatgatgcccttaacggttataaagaaaccgaaaaatatatcgataaaagaaaaaaagcgtataaatcggttgaaagcttttctcttgctgatattcaaaagtatgccgatgtttcagaatcttccgaagcaaacgctgaagttacggaatggcttcggaatgaaataaaagaaaaatgcaatttggcggttcagggatatgaatcttccaaggacctgatttcaaaaccttatactgagtcaaaaaaattatttaataatgataatgcggtagaattgattaaaaatgccctcgattccgtgaaggaacttgaaaatgttcttcggcttttgctcggcacaggtaaagaagaatcaaaggatgaatatttctacggcgaatttcttccttgctatgagcgtatctgtgaagttgattcactttatgacaaggtccgtaattatatgacacagaagctgtataagacggataagattaagattaatttcagcaacagccattttttaagcgggtgggcgcagacttattcaaccaaaggtgctttaattgtaaaaaaagagaataattattatttagtgattgttgataaaaagctttcaaatgatgacatagtgttcctgggtacaaatactcaactaagtcctgcagaaaggattgtatatgattttcaaaagcctgataacaaaaacaccccaaggctgtttattcgttcaaaaggaacaagctatgctccggcagtaaaagagtatgatttgcctatatcggatattattgagatatatgataacgaatactttaaaactgaataccgaaaaattaatcctaagggatataaagaagccctcataaaacttatagattatttcaagcttggcttcagcaggcatgaatcatatcgttgttttaatttcaaatggaaagaaagcgaacaatatagcgatatttccgagttctacaatgatgttgtcaaatcctgttatcaattaaagagcgaatcgatcaattttgacagtttattaaaacttgtagatgagggtaaactctatctgtttcagctgtacaacaaggatttttccgaacacagtaagggcactcctaatctccatactctttatttcaaaatgctgtttgatgaaagaaaccttgaaaatgttgtattcaaactcaacggtgaagccgaaatgttctatcgtgaagcaagtatcagtaaggatgatatgattgttcacccaaaaaatcagcccatcaaaaacaagaatgagcaaaacagcagaaagcaaagcacatttaaatatgacattgttaaagacagacgctatactgttgaccagtttatgcttcatataccgataacgctcaattttaccgcaaatggcggcacaaatataaacaatgaagtccgcaaggctctcaaggactgtgataagaactatgttataggtattgaccgtggcgagagaaatcttctttatatctgtgtggttgattcggaaggcagaattattgaacagtattcattaaacgagattatcaatgaatataacggcaatacttattcaaccgactatcacgctcttctcgacaagaaggagaaagagcgtctggaatcccgcaaagcttggaaaaccgttgaaaatattaaggaactgaaagagggatatatcagtcaggttgttcataaaatttgcgagcttgttgaaaaatatgatgctgttatcgttatggaagatttgaactttggctttaaacagggccgtagcggaaagtttgaaaaatcagtttatcagaagtttgaaaaaatgcttattgataagctcaattactttgctgataagaaaaaatctcccgaagaaatcggaagcgttctgaacgcatatcagcttactaatgcttttgaaagctttgagaagatgggaaagcagaatgggtttatcttttatgttcctgcgtatcttacgagtaaaattgacccgacgacaggctttgcagacctgcttcatccgtcgtcaaagcaaagcaaggaatctatgcgtgattttgtaggccgctttgactcaatcacattcaacaaaacagaaaactactttgaatttgaacttgattataacaagttcccgagatgtaatacggattacagaaagaagtggaccgtctgtacttacggcagccgtataaaaaccttcaggaatcccgagaaaaacagtgaatgggacaataaaacggttgaattaacgcctgctttcatggctctttttgaaaaatattcaatagatgttaacggagatattaaggcgcagataatgtccgttgacaaaaaagatttctttgttgagcttattggccttctgaggcttactcttcaaatgagaaacagcgaaacaggcaaggtcgatagagattatcttatatcacctgttaaaaacagcgagggcgtattctataacagcgatgattacaagggtattgaaaacgcttcgttacccaaagacgcagatgcaaacggtgcatacaatattgcaagaaaaggcttgtggattattgagcagattaaagcttgtgaaaatgatgcggagcttaacaaaattcgccttgctatatctaacgccgaatggcttgaatacgcacagaaaaaatga

SEQ ID NO: 26 - DNA, 3735 bp, synthetic construct
  source 1..3735: mol_type = other DNA; note = New Cas12a protein, codon sequence, id223
atgatgaaaagtattaagaatttccacaacctttatccagtacaaaaaacattgcgttttaggctggaaccaattggaaagaccgatgatctcatcgcacgcaatcaaattttagagactgatgagcgacgagcagccgaatatctcaaagttaaggagtatatcgaccaatatcatcgtcatttcattgagaatgcattaagcagccccatgcttaaagttgaatcaatgggcaaacaagattcactaacggattttgccgactgctacaacaatgacaacactgaaaaacgaagcgagcacctcgaaaagattcaagaaaagttaaggtcacaaattgtcaaaagattctcccaattaccggcttttgcccgaataactaagaaggagctcattaaagaagatttaccacagttcctaaaagacaagagagagcaagagattgtatctcatttcgacgaatttactacctatttcactggattccatcagaatcgcatgaacatgtacaccgccgatacaaaatccacttctattgcctttagactcattaaccaaaacttagtcaaatttgttgacaactgcaatatcctcgaaaaagttgccagttcactcagtaacgagattattgcaaaacttgacaaagactttgaaccgttccttaacgaacactctgctctcgatttgttcaagattgagaactataatgaattgctcactcaatctcaaattgaggtatataattcgattattggcggaagagtggatgagggtaataaaatcgagattaaaggattaaaccagtatattaatgagttcaatcaaactcatgagaagtcgcaaagaatacctaaactcaaacccttgtttaaacaaatcctgagtgaagatgtgggcgtgtcatttagaatggagcaattcaccgatgcaagccaagtacaagctaccataaaagaggaatacattaaattggagtctggagtttttgacaagctcaaagaaatgattaagagtttgtcaacattcaacctaaatggaatttatctagccaatgatttaggtctgacagacatctgccaacgtcattatggcgcttgggacatattaaataatgcgctcatcaccgcatttgacgctcatatacctcgcaagaaaaagcagtcgcaggagaaacatgatgagcaagcaaggaagtacctaaagggcataaaaagtatctctttaggcaaaatagacagtctcttggccgaagtaacagggaagtctattgttgactatttcacaagcctgggtgctatcaacaatgaaaccacacagcgtgagaacctatttgctcttatccaaaaccgttacaatgcaattaaaaatgtacttacttgtcccacggcatctgacgaactattacgcaaaaacactgaaggtatcaaggatttactagatgctatcaaagacttacaacattttattaagcctttgtgtggctatggcgaagaggctgacaaagacgacatattttatagcgatttctctcctctttatgaaacgcttgacaacaccattactcctttatataataaggtaagaagttatctaacaaagaagccttacaaactagacaagtttaagcttaatttcgacaacgcccaattactagatggatgggatataaacaaagaaaatgcttatttgtctatcctgttacgcaaagacggcaacttctatttagcgatagcaaacaaaaatgacaatcgtgctattaccgaaattaatcaagcacaagctgatgacggtagcgattgttatgagaagctgaactacaaactaattccttcgccatttaggatgctccctaaagtgttcttctcaagaaaaggattagagacttttaatccatcgcaagaaatacttgacatttacaataataaggaattccagttaggtgataaatttgataaagaagctctttataaattaattgacttctacaagacttcaatacctaattatgaaagctggcaattttttgatttcaacttcatgccaacacaatcctataagtctatcaatgagttctatagcgatattgaaaagcagggttataagatagatttcaagcaagtgccagttagcttaatcaacaaactcaatgatgatggactgctttatcttttcaagatagtcaacaaagatttctctccttattctaaaggacgtccaaatttgcacaccatttactggaagatgctctttgatgacaacaacctcaaggatgtaatatacaaacttaacggcaaagccgagatgtttttccgcagaaactctatcactaatcctgttatccatcctgccaacaaggacattgcgaacaagagtacttacaacaaacaacacaaagctgtgagcaagtttgattatgacatcatcaaggatcgtagattcacacgcaaccagtatgagtttcatgtgccaataactatgaacttcaagtcggcaagcaatggacatttcaatcgagatgtcttggcttttatcaaacagaatggcatcaagcacatcattggcattgaccggggagagcgacatcttctatacctgacaatgataaatctcaagggggagattgtgaaacagttatcactgaacagcgtggcttgtaaccccaacaatcctgattacaaacaggactataatgaattgctttcaatcaaggaaggcgaccggctgagcgcacgtcgcaactgggcaacaatagagaacataaaagaactgaaagctggatatcttagccaaattgttcatctcctgtctaaaatgatgatagaaaacgacgccatcctggtgcttgagaaccttaatactggcttcatgagggggcggcaaaaagttgaaaaatcagtatatctcaagtttgagaaaatgcttatcgacaagctcaactatgtcgttgacaaaacagccagcattaatgagccatgcggagcattaaaagcactacagctcaccgacacctacgacaactttaacaagtatcaaaaaggcaatgttcgtcagtgcggatttatcttctacatcccggcttggaatacgagcaagaccgaccctgctaccggcttcgtaaatctctttgacacacggttatcaaccatgggtgaaattaaatctttcttcagcaaatttgaccaaatcaagtataattccaaaaacgattcattagaattcacttttgattacaacaagttcacttcaagagccgaaggcactcgcacatgctggactattagctcacaaggtgagcgcattttcactcatcgcagcaaggagcaaaacaatcagtttgtttctgatacaatttacccaacactaatttttaaagacgtattcagcaagaacaacattgacattaatggcaatctgaaagatgaaatcgcatctattgatagccttgaagcactcaagcaactattgcatgcctttaaacttgtgatgcaaatgagaaacagcgtgacaggcaccgaagttgacttcttactatcaccagctatagatgctaaaggctctaattttgactctcggaaaggaatcaaaacgcttcccgaaaatgctgatgctaatggtgcttacaacatagcacgcaaagggttgatgataattgaccaaattaaaaacaccaatgatatcgataagattaaatatgcagtaagcaataaggattggcttaagtttgcacaaggataa

SEQ ID NO: 27 - DNA, 4113 bp, synthetic construct
  source 1..4113: mol_type = other DNA; note = New Cas12a protein, codon sequence, id224
ttgaaaacgaaaactataactataatgaaagaaaaagaacagtacccagattttacaaattttacaaatttgtatcaactttcaaagaccttgcgttttgagttgaaaccgataggcaagacagaagagacctttaagcagtggcttggagctatgaatagtgcaggcaacgaggataacaacgacaacttgtttgcacaagataagaaaatcaaagacgcttatgttgtaataaagtcaataatggataaactccatgagcagtttattgaaaaatctttaacatcagaagaagctaagaatattgatttttcagattttcttgatatatataaatatagagggaacgattttccgaaagattttgaaaaaaaattgcgagaacagattgggaacacctataaagttgcgggtgcttatttttccaaagaaataagcaaggctcttaataaagaatttaaaacaaaaaaaccgtttgaatgtcttactgatgcaaagatgcttaattatttatctgctaatgtgaaagaaattgcagagcagaacaatatagaccaagaaacacttgccaatcatattaagcagttcaaaggtttttggggctacttatctaactataacaaaaatcgcgagaattattatacaacggacaaagagcaatccactgcagttgccacacgtattgttcacgataatttacctaccttttgcgacaatattattcgttttgaagaacgcaatgacgaatactgtggtatttgtgaatatttgaaagataaaaacattgaaacaaagataaaaaataatcaaggaaatgaagtagatgtagaacccataaccaaagatgtttttaaaataaagcacttcaacaactgtcttgctcagtcgcagattaaagagtacaatcgtattattggcaattacaattatcagataaacctttacaaccaagctcgcagcagcgaaaatgacttcaggaagttagatgagtttgaaacgctctacaaacagattggttgtggagaagggagatcgttgtttgaaaccttgcataattacgatgatttaaataaacttcttgaaaagattaaggaagcaggagatagtttctttaaagagtgtggatataatgaaattagcactgttccagcatttataaaattcttgaaagagtgcaacaactgggacggtatttatatgtcaagtacagccattaatagaatatctaaccgctattttgccaattggcacagtattaaagacaagttaaaagataacaaagcttgtgtcacttacgataaaaatagagaagaaccaatacaactgcgtgatgccgtggagctttctgacttgtttaaggtattagatgataaagaacaatcggaacatttcttcaaggaatccctttccaatgatattaacgaaaaagagtgtccaagtaagaatctaataaatcttttatgctttgatattgagcaaaacatcagtaaatttcttgctaattccgaaaatattatcaacttgctaaacggcaacaaagacaatcaggcaagcgaagaagacaaaataaagcagataaaggagtggcttgatgcagctatagatacaatgcgaatcgtacgctattttgccgtacgcaaaagcaaaatgaaaggaaatctacccaacacgacaatggaaaaagcattgaacaacttgttatacaatgacgatgctcaatggtttaagtggtatgatcttgtccgcaactatctaacaaagaaaccgcaagacgatgctaaagaaaataaactgaagctgaattttgggaatccatctttgttgggtggttggagtgatggtcaggagcaaacgaaagccgcaactttattaaaatataataatgagttgtatttgtgcatattaaaaataaggaatatttttgacacatcaaaagctgacaatccaatttatacaaaagcaccattttgtgcatgtagattgattttaagaaacctaaagtttcaaactcttgcaggcaaaggttttaaaagtaagtacggaatcaaatatagtgaaatgggtaatgaagaccaatcacaagcaataaaatgtttaaaagaaatcatccgagaacgatatgcaaaaaaatatcctccgttggaacaaattataaaccaagaatattctgataaaaaaagttttgataatgaagtcaaggaagctttaaaagaatgctatgtctgtgaatttgtaccaataaattgggatgtggtttctaaaaagcaaaacagtacagatttattcttattcaaaatacattgcaaagactatttgccaaaggccacaggtaaaaaagatttgcagacaatgtattgggaagatgtattgtctgacagtagcaaccatcaattgtgtggtggtgctgagatatttatgagagaacctgtacccaaagaatctccagtaaaacatggtgtcggctctaaacttgtaaataagagagataaagatggtaatacaattccagagcagatttataaagaaatatatttatttgcgaatgacaaggtaaaaaaagaaaacctttcagcagaagctaaaaattatattgaatcagagaaagtaacaatcaaagaagcggaatatgaaattgttaaagacaatcgtttttatggtgaaacaaaatacatgttccattgcccaattacaataaactttaaggcaaaaggatataaatatgcttatccagaagtcaatgagaaaataacagatgctcttcaacaatccgataacttacaatttattggtatagatcgaggagaaaagcatcttgtttatagttgcacaatagacaaagaaggtaagattgttaaatgcaaccaccatgacaatatcaatggcactgattatgtacaaaaattggaagctgttgctaatgaacgtatcgccgccaaaaagaattggcagcagcagaacaaaatcaaggatctgaaagcgggctacatttcgcatgtagtacatcgtttggtggatgaaaccgttaaagacggcgacaaaattaccccacatgcatatattgtcttggaagacttgaacagtgagatgaagcgtggtcgccagaagattgagaagcaggtgtatcagaatttggagttggctcttgctaagaagtttaattttgtagtggataaagaagcaaaacaaggagaattaggttctgtaagcaaggctttacaactaacaccgccagtcaataactatcaagacatagagggtaagaaacagtttggtgtgatgctttatactcgtgccaactatacttcaataaccgatccaacaacaggatggcgtaagaccatttacataaaaaatggtaaagatgaggaaataaggaaacaaattcttgagaaattcaccgactttggttttgctggaaaagattattactttgaatacaccgaagaaaatgctggacatacatggcgtatgtattcaggtaaaaatggtgaaccgctgccgcgttttcagaacaagaaacagatggagcaagacaaaaacatctgggttccggaacaaatcaatgtgaaggaaatcctcgataaactatttgcaaatttcgacaaagggagatcttttaaggaacaggtaaatgaagaagtcaaacttgaaaagattgaagggagaagtgagactgcttggcaatctctgcgttatgcgctgaatatgattcaacagattcgtaattcgggggtggaaaaaacggacgacaatttcttgtattcaccggtacgtaatgaaaatggagaacattttgatacaagaaatcatgagaacaacggtgatttgagtccgatagtggatgccgatgccaacggagcattcaatatcgcccgcaaaggtttgattatggatgcacatataaagcattggattaacagcggacggcccaaaataaaaaaagacggaaaagaaacacctgatttggatttgtttatttccgatagagagtgggatttgtggctattggacagagaagagtggaaaaaatacttgccagtattcgcttctaaaagtaaaaaggatgctgataaaactacctctacaaagtccccaaaaagaaagaagaaataa

SEQ ID NO: 28 - DNA, 3750 bp, synthetic construct
  source 1..3750: mol_type = other DNA; note = New Cas12a protein, codon sequence, id230
aaggacctgaagcagtttatcggcatctaccccgtatcgaagacactgcgcttcgagttgaagccaattggtaagaccctggactggattcgtgaacgcaacgtggtggagaaggatgaacgcaagtcgcaggactatccggtggtcaagcagcacatcgatgagtatcacaaagtctgtatttccgaaagcttaaagaacgtttgcttcgattggaaaccgcttaaggaagcaattgaaaagcaccacaagttgaagagtgacgagagcaagaaacaactggaaaccgagcagacgaatatgcgtaagcagatcgtgttagcgatcaagcgctttaagcattatcccgagcttactgccgccacgccgaaggatctgattcagaacgtactcccggaaatcaccaatgacagcagcctgaacagttttaatcgcttcgcgagttatttcaaaggttttcaggagaatcgcaagaatatctactcactggacgcgattagtaccggagtaccgtttcgcttagttcacgacaactttcccaaattcttatccaacatcgacatttacgagaacattaaaagcactgcgccggagacgcttgagttagccgccaaggaactgcaaccctttcttgagggagtgatgctggacgacattttcagcttagacttttataacagtctcctgacccaggatggcatcgacttcttcaaccgcgtgatcggcggggtctcggaagagggcaagcagaaattccgcggaatcaacgaattcacaaatctgtaccgtcagcagcacccggaattaagcgccaataagaaagctatgacgatgattcccctctttaaacaaattctgagtgaacgcgagaccttaagttatatcccgaaacaaattgagaacgagaacgagttaacggaaaccatcaaacagttttacagccatattaccaatttcgagcgcaatggtaaaacgatcaacattctgaacgagttagtgcgtctcctgggccaggtgacgtcttataaccaggatcgcattttcgtgtctagcaaatacattacagatatctcgcagaagctgtacggtcgctggaacgcgattaatgactacttgtacgagcgcgcggtagagatgtttggtgacgtgtcagtggcgaagaataagaagttagtagataattatatcgacaaggacgtctatagcgcttctgaactgggattcgatgagaataacaacctgagtacgttttactccgaactccctcagatcgtgggatcggtcaacggtaattggattcagttttgtgagtggtgtaagattgacaagaagcaaaagttcttaaataacgcagagggtaccgagattattaagaagcttctggacgcaattatggaagtcatgcacaagtgtaacgttctggtagtaagcgaggagtacgacctggacaaggacttttataacaacttctttccactgtttactgagctggaaaatatcgtgttcgtttacaatcgcgtccgtaacttcttaactaagaagccgagtgacgttaagaaattcaaacttaatttcggcgtcccgacgctgtgtgatggctgggacctcaacaaggagaaggataataaagcgatcttactgtttaaggataatcgcagctatctggggattatgaatcccgaggattaccctaagattaaagagtcgaacgactcgggcgacggctactttagtaagatggtgtataagctgctgccaggcccaaataaaatgctccctaaggtgttctttagcaagaagggacgcgagactttcagtccctctgaccgtatcatcgacatctacgagtctggcgcctttaagaagggatcaagcaatttctctatcgtatgtctgcatgagctcatcgatttctataaggatgccattaatcgccacgaggattggagcaagtataatttcaaattctcccctacggattcctacaaagacatcggtgcgttttacaatgaaatcagcaaacaggcctacaagattcgcttcacgaatattagcgagcaggagattaatcagttggtcgataaaggccagctgtatctgtttcagctgtacaacaaagactacgcgtcgggcgcccacggccgcaagaatctgcacaccctctactgggaaaatcttttcaaagaggagaatttaaacaacctggttctgaagctgaatggacaagcggagttattttatcgcccccagagtattaatgaaccggtggcccacaaagttggctctatcatgttaaaccgtcgcgataaatctggcatgcctatcccggaaaccgtgtaccgcagtctctatctgtacttcaacggaaagaagaaggaagaggagcttgactcagtggaaaagagctacatcgataacgtgatcgtgaagaaggtgacccacgaaatcgttaaggataaacgctttaccaaacccgagttcttctttcacgtaccaatcacgtttaactcgaacgccgatggtaatgagttcattaatgaaaatgtgattgactacatcacggataatccggacgtcaacatcattggcatcgaccgtggagagcgccatctgatttatctgacgctgattaaccaacagggtgagatcttaaaacagaagacgtttaatacagtgggcacctacaactatcacgccaagctggagcaacgcgaacaggagcgtcaggaagcgcgtaaatcgtggcaatcaatcgggaaaatcaaggatttaaaggaaggtttcttgtcagctgtcatccacgaaatcgcactgatgatggcggaatataatgccatcgtagtgctggaagatctgaattttggttttaagcgcggccgctttaaggtagaacgccaggtgtaccaaaagttcgagaaaatgctgatcgataagctgaattaccttgcctttaaggatcgtaaaagcgacgaaactggtggtatcctgcgcggctaccaattcgcacagaaattcgtgagctttcaacgcctgggcaagcagtcaggctttctgttctacgtaccggcggcctacaccagcaaaatcgatccggtcacgggattcgtcaaccactttaatttcaatgatatcaccaacgccgagaaacgtaagcaattcttttgtaaaatggagcgcatcgagatgaagaacgataacatcgaattcgaattcgactaccgcaagtttaaaacctaccaaactgactttcaaaatgtatggacggcatccaccttcggtaagcgtattgttatgaagaccgatgagagtggccgtaaacagatggcagattattacccgaccaacgagattctggcggcctttaagaagtacggttacgagctgactaacgggaccgatattaaaagccttcttcaggatattgaagccgatcaaaagagcgccagcctgtttagtaacctgttttacgcgtttcagaaaactcttcagatgcgtaattcaaatcacgcgaccgaagaggattatattctgtcgccaatcgcgaagcatggatatcacttttgttcgacagaggaagcgaacaaaggtaaggatgagttcggtaactggctctccaagcttccggttgacgcggacgcgaatggtgcatatcatatcgctcttaagggcctgtacctgctcaagaacccgcagaccaagaagattgagcatgagaagtggttcaaattcatggtggagaagccgtacaaa

SEQ ID NO: 29 - DNA, 3702 bp, synthetic construct
  source 1..3702: mol_type = other DNA; note = New Cas12a protein, codon sequence, id238
atgaaagaacagtttatcaatcaatatccaataagcaaaactcttcagttttctttgattccaatcggaaaaacagaagaaaatttcaacaacaaacttcttcttgaagaagatgaaaaaagagcggaagaatatcagaaagttaaagaatatattgaccgttatcacaaattctttattgaaacagcattactgaatgtcagcctcaacggacttgaagaatatgtttccctgtatttcaaaccgtccaaaagtgatgccgatgtgaaagctatgtctgacattgaggcaaagcttcgcaaacagatttcaaaagctatgacttctcatgagctttataaagatttattcagtaaggatatgattgatagtattctgccgaaatatctgcagaaggaagaagaaagatcgaatgttgaaatgttccgcggattttcaacatattttcaaggcttttatactaacagaaagaatatgtattcggaagaagctcagtccactgctatatcataccgctgtattaatgataatctgccgaaatttcttgataatgtaaaatcttttgaaaaaataagagaagctttcaatgaagaaaaaataacgaccattaatgaagaattttttggaattcttggaaaatacgcaaccgatattttcaatgttgactacttcaactttgttctttctcagcccggtattgataaatacaacaaaataatcggcggttatgtgtgttctgacggaactaaaattcaaggattaaacgaactgatcaacctttataatcagcaggtagccaaatcagataaaagtaaacgcttgcctcttttgaaaatgctttataagcagattctcagcgacagagaatcggtatcttttattcccgagaaatataaagatgataatgagattattgcttcaataaacgaatatttttcagaaacagcattggaaacagttgctgcgctgaaagaattattcgacaaattttcatcttttgacctgaaaggcgtttatatttcatccggccttgccgtaacaagtctttcaaatgatgtttttggcagttggaatgctttttcttcagcttgggaaacgcgatattccgaacagaatcctaaaaagaaaaacaaaagtcaggaaaaatatgatgaagaattaaaagcaaatttcaagaaaaataagagcttcagtcttgatgagtttcagcaactcggttcagagatgaaagagcccgattcaatcggctctgttttagatttctatgcaggggctgtctctgaaaaagcggataatatccgtgcactttatattgcagccaaagaactgctaactcctaattatgctgagaagtatgataaaaagcttattaaaaaccatgttgctgttgagaagataaagagcctgcttgatgctgtaaaatcacttgaaaaacttgttaaaccgcttttgggaaccggaaaagaagaagaaaaggacaatgtgttctatggaaaatttcttccactatatgattcgttaactctcattgaccgtatttatgataaagtcagaaactatgtgactcagaaaccttattcaaaagataaaattaaactgaattttgaaaatccgcaattacttagtggctgggacaagaataaagagagtgattgtaggggaattcttttaagaaggaatggcaactattatctgggtataatagacaaaagtaattcaaaagttttccaaagttatccggtgccggaatttagcgatgatgaatactacgaaaagataatttataaacaaattaacggatgttcaaaatatttcagcattaagcaaattaaaccacagaacccgccggaaaacattattaaatacctcagcaaagattttgataagaaaacaatgacaaaaggtgaactggttgaacttataaaatatgtggttgaagattttattcccaattatagtccgcttttggacgaaaacgggaaatgcttttttgattttaagtataaaccatacgatcaatatacatcatggaatcacttctgtaaaagtcttgatccgcaagcgtacacaatcagattttcaaaagttaaaaaagactatattgataatctcatcgaaatcggttttctttatcttttccaaatctataataaagatttttccgaatacagccatggcacaccaaatctccatactctttactttaaaactcttttcgacaaaaagaattttgccgatgttgtctttaagttgagtggtggagcggaaatgtttttccgtaaagcaagcattaaagaaaacgaaaaaacagttcatcctgccaatcaaccgatcatcaataaaaatcccgataacagcaaaaaacagagcgagttttcatatgatcttgttaaagataaaaggtatacaaagaggcagttttcaattcatattcccattacgatcaatttcaaagccaacggtcgagaatttatcaattatgaggtcagacgcgctttgaattctgattacaaaccctgtgttatcggtattgacagaggcgaaaggaatctgatttatataactgtcattaatgacacgggtgaaattgttgaacagatgtcgcttaatgagattatcagtgataacggctataaagtcgattatcagaaattacttgacaaaaaagaaaaagcgagggatgaagcccgtaaaagttggggaactattgaaaatatcaaggaacttaaagagggctatatcagtcaggttattcataaaatctgtgaacttgtcattaaatatgatgctgttatcgcaatggaagatttgaatttcggcttcaagcgtgggcgttttaatgttgaaaaacaggtttatcagaaatttgagaatatgcttatctcaaagctgaattatctctgtgataaaaagatggaggcgaacgctgaaggcggtgttttaaaagcatatcaactgacaaataagtttgatggtgtaaacaaaggcaaacaaaacggaataattttttatgttcccgcttggctgaccagtaaaattgacccggtaaccggttttgttgatttgcttcatccgaaatatacaagtgttgaagaatcacataatttgttcggtaattttgatgatattcgatataacgccgttaccgacatgttcgagtttgaccttgactattcaaagtttccgaaaagcaaagcggatttcaaaaagaaatggactgtgtgcaccaattcggacaggattatgacattcagaaatccggcaaagaacagtgaatgggataataaacgagtcattcttaccgatgaattcaaaaaactgtttgaggaattcggcgttgactacaaaagcaatctcagggagaaaatcctcggtatttcaaacccggatttcaacaaacgtcttgtcaaatttttagctctgaccctccagatgagaaacagcgtaacaggcagcactaatcccgaagatgactatctgatttcccctgtcagagacaaaaacggcgtattttatgacagccgtaattttatcgggaatgacgctgtgcttcccgccgacgctgacgcgaacggcgcgtataatattgcaagaaagggcctttgggcaatagatgtgataaaatcaacttcgtctgatatgcttgataaagctgacctttctatttccaatgctgaatggcttgaatatgttcaaaaatga

SEQ ID NO: 30 - DNA, 3789 bp, synthetic construct
  source 1..3789: mol_type = other DNA; note = New Cas12a protein, codon sequence, id239
aaggagttcaccaatctgtactcgcttagcaaaactctgcgtttcgaattacaaccgattggtaaaacgttagaaaacattaacgccaatggcttcctcactaatgacgcgcaccgtgccgaaagctacaagaaagtgaagaagctgatcgacgaataccataaagatttcattgacaaggttctcggcgattttaaattgaacgcggagcacctgcaattatactacgatctgtacagtcaaacgaccaaggacaaacaattcaaagatgtgcaagataagttacgtaaagcgatcgctggcgccctgaaggccgacgaccgttacaaaactattgacaagaaagaactgatccgccaggacatgaagaagtttctcaacaacgacactgataaagccctcttagacgagttttacgaattcactacatacttcacggggtttcacgaaaatcgcaagaatatgtactcagatgaggccaagtcgaccgcgatcgcctaccgccttatccacgataatcttccgaaattcatcgacaatatggcggttttcaagaagattgcgaacacctcggtggcggacaacttttccgccatctataaagccttcgaggaatgtttgaacgttaactcgatcgacgaaatgtttagcctggattattacaatgacatcctgacgcagacgcaaatcgaagtttacaactcgattatcggcggccgcacattagaggacggaacgaaaatccaaggtattaacgaatacgtgaacctttataaccagcagccggcgaataagaaggagcgcctgccgaagcttaagcccttattcaaacagatcctgagtgatcgtgttcaactgtcttggctgcaagaagaatttaacaccggcgcagacgtaatcaacgcggtgaaggagtactgtgtgagctactttgacaacgtggaagagagcattaaggtgctgctgacctcgatttccgattacgacctgagcaagatctatattacgaatgatttagcaattaccgatgtgagccaacgtatcttcggagagtggagtattattccaaatgcgatcgacttccgtatccgcaaggacaacccacagcgcgcctcggagaaggaagaaaagtacagcgaacgtatttcaaagatcaagaagcaaagtaaatccttcagcctggcgtatattaatgagtgcctctcagagttgaaggacaccgacatcgcggattactatacgaccctgggtgccattaacaccgagtcaaagcaagaacccagtattccgactagcatccaggttcactataacgcactgaagccgatccttgacgcggactacccgcgcgagaagaatttatcgcaagacaaactgactgtaatgcaactgaaagacctcttggatgactttaaagccctgcagcgtttcattaaaccgctgctcggcaacggtgacgaagcggaaaaggatgaaaagttttacggcgagttaatgcagctttgggagatgattgatagcattaccccactgtacaataaagtccgtaacttttgcacccgtaaaccgttttcaacggagaaaatcaaagtcaactttgaaaacgcccagttgttagatggctgggatgaaaacaaggagaatacgaatgcgagtatcatcctgcgcaaagatgggatgtactacctgggaattatgaagaaggagcaccgtaacattttggcccaaactatgccgagcaacggagactgttatgacaaggtcgtgtacaagttcttcaaagatattacaaccatggttccgaagtgcacgacccaaattaagtctgttaaggaacacttcgccaactccaacgaagattacaccctgttcgataaagacaagttcctgacaccggtggttattacaaaggaaatcttcgacctgaacaacgttctgtacgatggcgtgaagaagtttcagatcggctatttaaacaacaccggggataaccagggctataaccacgccgttgagatttggaagtcgttctgtatgcagtttctgaaagcgtataaaagtaccagcatctataatttctcgtcaatcgagcaaaacattgacaagtacaatgacctgaatagcttttacggtgcagtgaacttgctgctttactcgctgacataccgcaaagtctcagttgactacatccaccaattagtcgacgatgataagatgtatctgtttaaaatttataacaaagatttcagcccgttttccaaaggtaccccgaacatgcacactctctactggaagatgctgttcgacgagtgcaacctttctgacgtagtgtacaagttgaacggacaagccgaagtcttctttcgtaagaaaagcattaattgccaacacccaacgcacccagcaaacaaaccgatcgacaataagaacgcaaataatccgaagaagcaatcaaccttcgaatacgatctgattaaagacaaacgttacaccgtagataagtttatgtttcatgtgcctatcacgcttaattttaaggggatcgggaacggcgatattaatatgcaagtgcgcgagtatatcaaagcgagcgacaatctgcatttcattgggatcgatcgtggcgaacgccatttgctgtacatttgtgtaatcaattcgaaagggaagattgtcgaacagtatagcttaaacgagattattaataattataagggcacggagtacagcaccgattaccatacccttttaagcgaacgtgaaaagaagcgcaaggaagagcgtagctcatggcagaccattgaagggattaaggaattaaaggcaggttacctgtcgcaagtaatccataaaatctcgcagcttatgattaagtacaacgcgatcgtactgttagaggatctgaaccccgggtttaagcagggacgccaaaaggttgaatcgagtgtataccaacaattcgagaaagccctgatcgaaaagttaaactacctcgtagataagaataaagatgtgaatgaaatcggcggtctgttgcacgcctaccaactgaccaacgatccgaagttaccaaataagaactcaaagcagagcggcttcttattttatgtgcccgcgtggaacacgtcgaaaattgacccagtaaccggttttgtgaatttattagacacgcgctacgagaatgtcgcgaaggcccaaaccttcttcaacaaattcgacagtatccgttataataaggaaaatgactgttttgaattttgtttcgactacacgaattttactgcgaaggcagaggagacccgcaccaaatggaccttgtgcacctacggtacgcgcatcgaaacctttcgtaacgctgagaagaattcgaactgggactcgaaggaaatcgacctgactacggaatggaagtcgctgttcgcgaaacataatattagccttaacgcgaaccttaaggaagccatcttgctgcaagctaacaagaacttttacgcagatatcctgcatttgatgaagttgactctgcagatgcgcaactcagtgacgggaaccgatattgattatatggtgagcccggttgcaaatgagtctggcgagttcttcgactcacgcaatgccaaagagggtttacccgttaacgcggacgcgaatggtgcgtataatatcgcccgcaagggcctgtggctcgcgcaacagattaaggatgctaacgatattagtgacgttaagttagcgattacgaataaggaatggctccagttcgcgcagacgaagccgtacttaaaagac

SEQ ID NO: 31 - DNA, 3828 bp, synthetic construct
  source 1..3828: mol_type = other DNA; note = New Cas12a protein, codon sequence, id245
atgaacaaatttgttaatctttactcggtcatcaaaacgttacgttttgaattggtacccataggaaaaaccaaggtgaacattgagaaaagcaatcttctcatggatgatgctaagcgggcagaagactgtaaaatcgtcaaaagccttatggacagatatcataagcagtttattaaatctcgtttgtgtaatctagaattgcctttagaggagaatggccaaaaatgttcgttggaagaataccgcgtcctctatgaaatgaaaaaacgcaatgcagagcaagaagctacattcgctgaagtaaaggggaatcttagagcattgattgctaagcacttgacggctaagggcactgcctatgaccgaatttttaaaaaagaactcatccgtgaagatttactgaacttcttggaagatgagagtgaaagggaaattgtggagcaatttgctgatttcacaacctatttttatggctttcataaaaatcgggctaatatgtattcagccgaagagaagtctactgcaattgcatataggcttattaatcagaatttgccgaaattcatggataacatgagaacctttatgagaatagcgcagacttctgtggcagaacattttggtgaaatctatacagggtggaagaaatttctgaatgtcgaaagcttggaagagttttttagcctgaagaattttacaaagacgcttactcaagagcatatcgaggtttacaattatgtcattggaaagcgcatacttgaagacggtgcagaaataaagggaatcaatgagtatgtaaacctttataatcagcaacagaaggacaagagaaaccgtctgccgtttatggttcctttgtataagcagattctcagcgatagagaacaattgtcatggctggcggaggagtttgattctgatgagaaaatgcaaaaagccgtgttggagacctatggcaatctgtatgataaattgatgggagacgagaatgagtcgctgcggtcgctgcttaagaatatcgatgactatgacctcacccaaatccatatatccaatgatttgtcactaacggaaatctcacagcatatctttggcaaatatgatgcatttactaagggaataaaagaaagactttctggtattgtggcaaggaagaaaaatgataaagaagaacaatatgaggataggataaatgaagtcttcaagtcgtataagagttttagcatcgccgaccttaatcaattattacttccagaagttgaagatggccgcccaaagaatatagaagaatattttaagtctctaggtgctatccatacggatacctatcagaaagagaatctatttactcaaatcgagaatgcatatacagaggcaagccctattctccggatgaaagagactggtgaccatctctcccaaaacaagggagctgttgagaagataaagaatctgcttgatgccctgaaagctctccaacattttataaaaccactaacaggtagtggggaggaaagtgataaggacggtcgcttctatggtgctttccagatgatatgggatgaattggatgccgtcactcccttgtataacaaggtgaggaactggctgactaaaaagccgtatagcacagaaaaattcaaattgaattttgcatctcctactttgttgtcgggatggactaagccacaagataattcatgtgtaatattgagtgatgatgagtgttgtgctttttatttaggtattattgattctcatcatagaacactctttaacgatatcgaccgtatccatcagaagtttcgtcctggaggaatgaaaatcatgaattattatcaagggggtgatatgggaaagaatgttcagaatcttatgatggaaaatggtagagttaaaaaggtaaatggtcgtaaagaaaccagtggaccgtttgctggtgaaaatttgcgtttagagcaggcaaaaaataaatatcttcctccagaaatcaacaggataagaaaaatggcatcgtataaaccaaataattctaattataataagaaagatttggaattgttcattgaatattatatgactttagtgaaggattattatccaaaatgttctttttctttttacccgccatctcactatcagtcatttgatgcatttttaacgcacatccaacagcagtcctatcaaatacatttttctcctatatcaaagtctttcattgataatatggtagatgcagggaaattgtatcttttccgtatatggaacaaagatttttctgaacacagcaaaggcactcctaatcttcacactctttattggaagatgctttttgacgagcgaaatcttcgtgatgttgtctataagttgaatggtcaggcagaggtgtttttccgcaagtccagcatctcccatgagaatagaatcattcatcctgctaataaggaaatcgaaaataaaaacactttgacggagaagaagacgagcttgtttacttatgatattgtcaaagataaaagatttacagtggacaagtttcaattccatgtgcctatcactatcaatttcaaggcaaaaggtttgaaaaacattaatcctcttgtcattgatgttatccgtgagggaggatttacgcatattataggtatcgaccgcggagagagacatttattatatttgtctctgattgatttgaaaggacgcattgttaagcagatgtcactcaatcaaattattaatgattatgaggggcatcagcacaagacggattatcatgctttgttgatgaaaagagaaggtgagcgaacaaatgctcgtaaaagttgggacacgattgaaaaaatcaaagaattgaaagagggatatcttagtcaagttattcatttgataacaaagatgatggttaagtataaagccatcgtggtgcttgaggatctcaatagtagttttgtccgtggaaggcagaaaattgagcggcaggtatatgagaaatttgagaagatgcttatcgacaagctgaattgctatattgacaagaagagagatccagagtgtgaaggtggtatgcttcatgctctgcaattagccaataagtttaagagcttttccaaaatggggaaacaatgtggctgtcttttctatattcctgcatggaacacaagcaagatagatcctgtaactggctttgtcaatttattggacacaaggtatgagtcggtgaaagcttcgaaggagtttttctctcattttgactgtatcacatttaatgctacgatggattggttcgaattttcgttcgattatgacaatttccacaagaaggctgagggaacacaaacgcaatggaccgttttcagcagaggatcccgtatccacactttccgcaatccggagaaaaataatcagtgggacgatgaggagataaacctcacagataagtttaaggaattatttgctaagtatagcatcaactatagaggtaatctcaaagatgcgatgcaagattcaaatgaagctactttctttaaggagttgatggggcttatgaaacttttgatgcagatgcgtaacagtaaggcaaattccgaaattgactatatgttgtcgcctgtttctgataagaacggcaatttctatgatagtcgtgtttgcggaggctctttacctgaaaatgcagatgctaatggtgcatacaatatagcacgtaaggggctgatgctcatcagaaatatacaggcagctcccgatgacaaaaagccatcgcttaccatcactaataaggattggttatgctttgcacaaaccaaaccatatcttgaggactga

SEQ ID NO: 32 - DNA, 3852 bp, synthetic construct
  source 1..3852: mol_type = other DNA; note = New Cas12a protein, codon sequence, id250
aaggatctgaagcagttctcgaatctctataaggtctctaaaacgctccgcttcgagctgcagccgattggcaaaacgaaggagaatatcgagaaaaccgggattcttgcgcgcgacgagcagatggccaaggactatatgctggtgaagggctttatcgacgaataccacaaacaatttattaaggatcgcttatggaacttcaagatcccgttaaagagcgagggacacctcgacagtttagaggagtaccaaatgttttacgagttatccaaacgcgacgacgcgcaggaagcggcattcacggaagtaaaagataacttgcgcagcattattgctaagcgtctgaccaagagtagcagtgcgttcgaccgtattttcaagaaagagctgattcgcgaagacctgatcaatttcctggagaacgaggaagacaaagagattgtccgccaatttgccgactttaccacgtatttcactggttttcatgaaaaccgtcgtaacatgtacgacgctgaagaaaagtccacggccattgcctaccgcctgatccaccaaaatctgccgaaattcatggacaatctgaaagcattcgccaaaatcgcggaaacccccgtggccgagcacttcgcggatatctacgaaggttggaaggagcgtctgaatgtggggtccatcgacgagatctttcagctggactactttagcgagaccttgacgcagtcacacatcgaagtgtacaactacattatcggcaagaagattttagaggacggtacggagatcaaaggcattaacgagtacgtgaatttgtacaaccaacaacagaaggacaaaagtaaacgccttccctttctggtcccactgtacaaacaaatcttatcggaccgtgaaaagctgtcttggatggcggaagagttcgactccgacgacaagatgctgtcagcgatcaatgaatcgtatagtcacctgcacgatctgctgatgggcgaggaaaacgaatctttacgctcgcttttaatgcacatcaaggactttaaccttgagcagattaacattacaaacgatattagcctgactgatatttctcaacacctgttcaatcgttacgacgtatttacgaacggtatcaaggacgagcttcgtattattatcccgcgcaagaagaaagagaccgacgaggttttcgaagaccgcatcagcaaaatctttaagaatcgtaaaagtttctcaatcgccttcttggataaacttccgcaacctgtgatggaagacgggaaaccccgttctatcgaggagtatttcctgtcgctgggcgcagtcaatacgggcacgacacagaaagagaatcttttcgcgcgtatcgagaatgcgtatgaagacgcgcgtacggtgctgcagatgaaggacactaacgacaccctgagccaaaataagccggccgttgcgaaaatcaaaaccctgctggatgccattaaggatttacaacatttcatcaaaccgctcttagggtcgggcgaagagaatgagaaggatgagctgttctatggggtatttcagatcatttgggatgagttagataccatcaccccgctgtacaacaaagtgcgtaattggctgacacgcaagccatacagcacagaaaagatcaagctgaactttgacaacgcacagcttttggacggttgggatgaaaacaaggagaccgccaacgcaagtatcatcctctgtaagaatggccaattttacctgggaattctgaacaaggactaccgcaaactgttaggaatgcccatgccgagtgagggtgagtgttacgacaaagttgtctacaagttctttaaagatattacaactatggtccccaagtgcaccacgcagaagaaggacgtggtggcgcacttcgcccagtctacggagaattacattctctttgataagaagaccttcaacgccccggtgacgattaccaaggagatttacgatttgaataatgtgctgtacaacggtattaagaagtttcagattgagtacttacgcagtactggggataagagcggctacgagcacgcggtctttacttggaagagtttctgcatgcagtttctgaaggcctataagagcacttcaatctataacctggccctggtggagcagaacattgatagctactccgatctgtctagcttttataatgccgtgaaccttctgctgtacaacctgtcatatcgcaaggtatcagtagactacattcataccttagtggaagaggggaagctgtatttatttcgtatctggaataaggacttttcggagtacagcaagggtaccccgaatctgcacactctgtactggaagatgctgtttgacgaacgtaatctttgtgacgtaatctataaattaaatgggcaagctgaggtattttaccgtaaagcttcaattaagccggagaatcgcattattcaccccgcccaccatccgatcaccaacaagaatgaattgaatgataagcgcacgtcaacctttgagtatgacatcatcaaggatcgccgttataccgtggacaaatttcaatttcacgtcccgatcactattaattttaaagcaattggccagtcgaatattaacagtattgtgcaagacaccatccgcggcggtggctttacccatattatcggcattgatcgcggcgaacgccacttactgtaccttagtatgatcgatttaaagggcaacattgtgaaacagatgacgctcaatgacattatcaacgagtacaacgggcacaaatacaaaaccaactaccatgatctgcttactaaacgtgagggcgatcgcaccgacgcacgccgcagctgggataccatcgaaaacattaaagagctgaaggaaggttacctttcgcaggttatccatattatctctaaaatgatggttgagtataacgccattgtcgtgcttgaggatctgaatactggcttcatgcgcggtcgccaaaagatcgaacgccaagtatacgaaaagttcgaaaagatgctgatcgagaagttgaactgctacatcgataaacacgccgaccctctggaagatggcggcgttctgcatccgctgcaactgacttgcgacgcacaaaagtggaagcgcagccaccagtgtggctgccttttctacatccctgcgtggaatactagtaaaattgatccggtgaccggattcgttaacttgtttgacacccgttatgatacccgtgaaaaggcccgcctgttctttagcaagtttcaacgtatctcttataactcagaaaaggattggttcgaatttaccttcgactataataattttactagcaaggccgagggtacgcgtacggaatggaccctgtgtacctatggtgaacgcattcacacttttcgtaacccaaagaagaaccaccagtgggacgatgaacagatcgttttgacggagaattttaagcagctgttcgataaatacggtattgatatccacggtaatctgaaggaagcgattagcactcagtcagacagccagttctttcgtgacctgctcggcttaatgaagctgttactgcaaatgcgcaacagtattactaattcggaagtcgattacttactgagcccggtcgcagatgagacgggtcacttcttcgacagtcgtgaggagatcgaaagcttgccgtcaaacgcggacgcgaatggtgcatacaatattgctcgtaagggactttgggtaatcaagaaaatccaggagaccgcggaaaatgagaaattctcacttgcaattagtaataaagagtggctgcaattcgctcaaacgaaaccatacctgaacgag

SEQ ID NO: 33 - RNA, 21 nt, synthetic construct
  source 1..21: mol_type = other RNA; note = Direct repeat sequence of crRNA for id218
taatttctactaagtgtagat

SEQ ID NO: 34 - RNA, 21 nt, synthetic construct
  source 1..21: mol_type = other RNA; note = Direct repeat sequence of crRNA for id233
aaaatttctactcttgtagat

SEQ ID NO: 35 - RNA, 21 nt, synthetic construct
  source 1..21: mol_type = other RNA; note = Direct repeat sequence of crRNA for id236
taaatttctactgttgtagat

SEQ ID NO: 36 - RNA, 21 nt, synthetic construct
  source 1..21: mol_type = other RNA; note = Direct repeat sequence of crRNA for id204
taatttctactattcgtagat

SEQ ID NO: 37 - RNA, 21 nt, synthetic construct
  source 1..21: mol_type = other RNA; note = Direct repeat sequence of crRNA for id206
ttaatttctactttcgtagat

SEQ ID NO: 38 - RNA, 21 nt, synthetic construct
  source 1..21: mol_type = other RNA; note = Direct repeat sequence of crRNA for id210
caatttctactttcagtagat

SEQ ID NO: 39 - RNA, 21 nt, synthetic construct
  source 1..21: mol_type = other RNA; note = Direct repeat sequence of crRNA for id211
ttaatttctacttgtgtagat

SEQ ID NO: 40 - RNA, 21 nt, synthetic construct
  source 1..21: mol_type = other RNA; note = Direct repeat sequence of crRNA for id217
aatttctactactatgtagat

SEQ ID NO: 41 - RNA, 21 nt, synthetic construct
  source 1..21: mol_type = other RNA; note = Direct repeat sequence of crRNA for id219
taaatttctactattgtagat

SEQ ID NO: 42 - RNA, 21 nt, synthetic construct
  source 1..21: mol_type = other RNA; note = Direct repeat sequence of crRNA for id223
aaaatttctactattgtagat

SEQ ID NO: 43 - RNA, 21 nt, synthetic construct
  source 1..21: mol_type = other RNA; note = Direct repeat sequence of crRNA for id224
aaaatttctactattgtagat

SEQ ID NO: 44 - RNA, 21 nt, synthetic construct
  source 1..21: mol_type = other RNA; note = Direct repeat sequence of crRNA for id230
taaatttctactattgtagat

SEQ ID NO: 45 - RNA, 21 nt, synthetic construct
  source 1..21: mol_type = other RNA; note = Direct repeat sequence of crRNA for id238
ataatttctactgttgtagat

SEQ ID NO: 46 - RNA, 21 nt, synthetic construct
  source 1..21: mol_type = other RNA; note = Direct repeat sequence of crRNA for id239
ataatttctactattgtagat

SEQ ID NO: 47 - RNA, 21 nt, synthetic construct
  source 1..21: mol_type = other RNA; note = Direct repeat sequence of crRNA for id245
ataatttctactgttgtagat

SEQ ID NO: 48 - RNA, 21 nt, synthetic construct
  source 1..21: mol_type = other RNA; note = Direct repeat sequence of crRNA for id250
taatttctactattcgtagat

SEQ ID NO: 49 - RNA, 16 nt, synthetic construct
  source 1..16: mol_type = other RNA; note = Stem-loop sequence for guide RNA
gtttcaaagattaaat

SEQ ID NO: 50 - DNA, 21 bp, synthetic construct
  source 1..21: mol_type = other DNA; note = Direct repeat encoding DNA sequence of crRNA for id218
taatttctactaagtgtagat

SEQ ID NO: 51 - DNA, 21 bp, synthetic construct
  source 1..21: mol_type = other DNA; note = Direct repeat encoding DNA sequence of crRNA for id233
aaaatttctactcttgtagat

SEQ ID NO: 52 - DNA, 21 bp, synthetic construct
  source 1..21: mol_type = other DNA; note = Direct repeat encoding DNA sequence of crRNA for id236
taaatttctactgttgtagat

SEQ ID NO: 53 - DNA, 21 bp, synthetic construct
  source 1..21: mol_type = other DNA; note = Direct repeat encoding DNA sequence of crRNA for id204
taatttctactattcgtagat

SEQ ID NO: 54 - DNA, 21 bp, synthetic construct
  source 1..21: mol_type = other DNA; note = Direct repeat encoding DNA sequence of crRNA for id206
ttaatttctactttcgtagat

SEQ ID NO: 55 - DNA, 21 bp, synthetic construct
  source 1..21: mol_type = other DNA; note = Direct repeat encoding DNA sequence of crRNA for id210
caatttctactttcagtagat

SEQ ID NO: 56 - DNA, 21 bp, synthetic construct
  source 1..21: mol_type = other DNA; note = Direct repeat encoding DNA sequence of crRNA for id211
ttaatttctacttgtgtagat

SEQ ID NO: 57 - DNA, 21 bp, synthetic construct
  source 1..21: mol_type = other DNA; note = Direct repeat encoding DNA sequence of crRNA for id217
aatttctactactatgtagat

SEQ ID NO: 58 - DNA, 21 bp, synthetic construct
  source 1..21: mol_type = other DNA; note = Direct repeat encoding DNA sequence of crRNA for id219
taaatttctactattgtagat

SEQ ID NO: 59 - DNA, 21 bp, synthetic construct
  source 1..21: mol_type = other DNA; note = Direct repeat encoding DNA sequence of crRNA for id223
aaaatttctactattgtagat

SEQ ID NO: 60 - DNA, 21 bp, synthetic construct
  source 1..21: mol_type = other DNA; note = Direct repeat encoding DNA sequence of crRNA for id224
aaaatttctactattgtagat

SEQ ID NO: 61 - DNA, 21 bp, synthetic construct
  source 1..21: mol_type = other DNA; note = Direct repeat encoding DNA sequence of crRNA for id230
taaatttctactattgtagat

SEQ ID NO: 62 - DNA, 21 bp, synthetic construct
  source 1..21: mol_type = other DNA; note = Direct repeat encoding DNA sequence of crRNA for id238
ataatttctactgttgtagat

SEQ ID NO: 63 - DNA, 21 bp, synthetic construct
  source 1..21: mol_type = other DNA; note = Direct repeat encoding DNA sequence of crRNA for id239
ataatttctactattgtagat

SEQ ID NO: 64 - DNA, 21 bp, synthetic construct
  source 1..21: mol_type = other DNA; note = Direct repeat encoding DNA sequence of crRNA for id245
ataatttctactgttgtagat

SEQ ID NO: 65 - DNA, 21 bp, synthetic construct
  source 1..21: mol_type = other DNA; note = Direct repeat encoding DNA sequence of crRNA for id250
taatttctactattcgtagat

SEQ ID NO: 66 - DNA, 16 bp, synthetic construct
  source 1..16: mol_type = other DNA; note = Stem-loop sequence for guide RNA encoding DNA
gtttcaaagattaaat

SEQ ID NO: 67 - protein, 7 aa, synthetic construct
  source 1..7: note = NLS, SV40
PKKKRKV

SEQ ID NO: 68 - protein, 16 aa, synthetic construct
  source 1..16: note = NLS, Nucleoplasmin
KRPAATKKAGQAKKKK

SEQ ID NO: 69 - protein, 23 aa, synthetic construct
  source 1..23: note = NLS, SV40-Nucleoplasmin
PKKKRKVKRPAATKKAGQAKKKK

SEQ ID NO: 70 - protein, 49 aa, synthetic construct
  source 1..49: note = NLS, Nucleoplasmin-c-Myc-Linker-c-Myc
KRPAATKKAGQAKKKKPAAKRVKLDGGGGSGGGGSGGGGSPAAKRVKLD

SEQ ID NO: 71 - protein, 21 aa, synthetic construct
  source 1..21: note = NLS, SV40-SV40-SV40
PKKKRKVPKKKRKVPKKKRKV

SEQ ID NO: 72 - protein, 9 aa, synthetic construct
  source 1..9: note = NLS, c-myc
PAAKRVKLD

SEQ ID NO: 73 - protein, 11 aa, synthetic construct
  source 1..11: note = NLS, c-myc
RQRRNELKRSP

SEQ ID NO: 74 - protein, 38 aa, synthetic construct
  source 1..38: note = NLS, hRNPA1 M9
NQSSNFGPMKGGNFGGRSSGPYGGGGQYFAKPRNQGGY

SEQ ID NO: 75 - protein, 42 aa, synthetic construct
  source 1..42: note = NLS, impotein alpha
RMRIZFKNKGKDTAELRRRRVEVSVELRKAKKDEQILKRRNV

SEQ ID NO: 76 - protein, 8 aa, synthetic construct
  source 1..8: note = NLS, myoma T protein
VSRKRPRP

SEQ ID NO: 77 - protein, 8 aa, synthetic construct
  source 1..8: note = NLS, myoma T protein
PPKKARED

SEQ ID NO: 78 - protein, 8 aa, synthetic construct
  source 1..8: note = NLS, human p53
PQPKKKPL

SEQ ID NO: 79 - protein, 12 aa, synthetic construct
  source 1..12: note = NLS, mouce c-able IV
SALIKKKKKMAP

SEQ ID NO: 80 - protein, 5 aa, synthetic construct
  source 1..5: note = NLS, influenza
DRLRR

SEQ ID NO: 81 - protein, 7 aa, synthetic construct
  source 1..7: note = NLS, influenza
PKQKKRK

SEQ ID NO: 82 - protein, 10 aa, synthetic construct
  source 1..10: note = NLS, herpes virus delta
RKLKKKIKKL

SEQ ID NO: 83 - protein, 10 aa, synthetic construct
  source 1..10: note = NLS, mouse Mx1 protein
REKKKFLKRR

SEQ ID NO: 84 - protein, 20 aa, synthetic construct
  source 1..20: note = NLS, human poly-ribose
KRKGDEVDGVDEVAKKKSKK

SEQ ID NO: 85 - protein, 17 aa, synthetic construct
  source 1..17: note = NLS, human glucocorticoid
RKCLQAGMNLEARKTKK

SEQ ID NO: 86 - protein, 7 aa, synthetic construct
  source 1..7: note = NLS, SV40 derivatives
PKTKRKV

SEQ ID NO: 87 - protein, 11 aa, synthetic construct
  source 1..11: note = NLS, SV40 derivatives
PAAKTKRKVLD

SEQ ID NO: 88 - protein, 9 aa, synthetic construct
  source 1..9: note = NLS, SV40 derivatives
PAAKTKRLD

SEQ ID NO: 89 - protein, 18 aa, synthetic construct
  source 1..18: note = NLS, SV40 derivatives
  VARIANT 3..11: note = X can be any amino acid
KRXXXXXXXXXPKTKRKV

SEQ ID NO: 90 - protein, 18 aa, synthetic construct
  source 1..18: note = NLS, nucleoplasmin derivatives
  VARIANT 3..12: note = X can be any amino acid
KRXXXXXXXXXXKKKKLD

SEQ ID NO: 91 - protein, 8 aa, synthetic construct
  source 1..8: note = NLS, nucleoplasmin derivatives
AAAKKKLD

SEQ ID NO: 92 - protein, 9 aa, synthetic construct
  source 1..9: note = NLS, nucleoplasmin derivatives
PAAKKKKLD

SEQ ID NO: 93 - protein, 7 aa, synthetic construct
  source 1..7: note = NLS, nucleoplasmin derivatives
PAAKKKK

SEQ ID NO: 94 - protein, 9 aa, synthetic construct
  source 1..9: note = c-Myc
PAAKRVKLD

SEQ ID NO: 95 - protein, 15 aa, synthetic construct
  source 1..15: note = AA Linker
GGGGSGGGGSGGGGS

SEQ ID NO: 96 - protein, 10 aa, synthetic construct
  source 1..10: note = AA Linker
GGGGSGGGGS

SEQ ID NO: 97 - protein, 5 aa, synthetic construct
  source 1..5: note = AA Linker
GGGGS

SEQ ID NO: 98 - protein, 6 aa, synthetic construct
  source 1..6: note = AA Linker
GGGGGG

SEQ ID NO: 99 - protein, 8 aa, synthetic construct
  source 1..8: note = AA Linker
GGGGGGGG

SEQ ID NO: 100 - protein, 5 aa, synthetic construct
  source 1..5: note = AA Linker
EAAAK

SEQ ID NO: 101 - protein, 10 aa, synthetic construct
  source 1..10: note = AA Linker
EAAAKEAAAK

SEQ ID NO: 102 - protein, 15 aa, synthetic construct
  source 1..15: note = AA Linker
EAAAKEAAAKEAAAK

SEQ ID NO: 103 - protein, 46 aa, synthetic construct
  source 1..46: note = AA Linker
AEAAAKEAAAKEAAAKEAAAKALEAEAAAKEAAAKEAAAKEAAAKA

SEQ ID NO: 104 - protein, 5 aa, synthetic construct
  source 1..5: note = AA Linker
PAPAP

SEQ ID NO: 105 - protein, 12 aa, synthetic construct
  source 1..12: note = AA Linker
AEAAAKEAAAKA

SEQ ID NO: 106 - protein, 20 aa, synthetic construct
  source 1..20: note = AA Linker
GGGGSGGGGSGGGGSGGGGS

SEQ ID NO: 107 - protein, 17 aa, synthetic construct
  source 1..17: note = AA Linker
VSQTSKLTRAETVFPDV

SEQ ID NO: 108 - protein, 6 aa, synthetic construct
  source 1..6: note = AA Linker
PLGLWA

SEQ ID NO: 109 - protein, 6 aa, synthetic construct
  source 1..6: note = AA Linker
RVLAEA

SEQ ID NO: 110 - protein, 10 aa, synthetic construct
  source 1..10: note = AA Linker
EDVVCCSMSY

SEQ ID NO: 111 - protein, 8 aa, synthetic construct
  source 1..8: note = AA Linker
GGIEGRGS

SEQ ID NO: 112 - protein, 10 aa, synthetic construct
  source 1..10: note = AA Linker
TRHRQPRQWE

SEQ ID NO: 113 - protein, 10 aa, synthetic construct
  source 1..10: note = AA Linker
AGNRVRRSVG

SEQ ID NO: 114 - protein, 9 aa, synthetic construct
  source 1..9: note = AA Linker
RRRRRRRRR

SEQ ID NO: 115 - protein, 4 aa, synthetic construct
  source 1..4: note = AA Linker
GFLG

SEQ ID NO: 116 - protein, 5 aa, synthetic construct
  source 1..5: note = N-terminal tag
MKIEE

SEQ ID NO: 117 - protein, 8 aa, synthetic construct
  source 1..8: note = C-terminal tag
LEHHHHHH

SEQ ID NO: 118 - protein, 1251 aa, synthetic construct
  source 1..1251: note = CRISPR enzyme comprising NLS and ID218 Cas12a protein
PKKKRKVKKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQKPKKKRKVKRPAATKKAGQAKKKK

SEQ ID NO: 119 - protein, 1244 aa, synthetic construct
  source 1..1244: note = CRISPR enzyme comprising NLS and ID218 Cas12a protein
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQKPKKKRKVKRPAATKKAGQAKKKK

SEQ ID NO: 120 - protein, 1244 aa, synthetic construct
  source 1..1244: note = CRISPR enzyme comprising NLS and ID218 Cas12a protein
PKKKRKVKKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQKKRPAATKKAGQAKKKK

SEQ ID NO: 121 - protein, 1237 aa, synthetic construct
  source 1..1237: note = CRISPR enzyme comprising NLS and ID218 Cas12a protein
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQKKRPAATKKAGQAKKKK

SEQ ID NO: 122 - protein, 1277 aa, synthetic construct
  source 1..1277: note = CRISPR enzyme comprising NLS and ID218 Cas12a protein
PKKKRKVKKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQKKRPAATKKAGQAKKKKPAAKRVKLDGGGGSGGGGSGGGGSPAAKRVKLD

SEQ ID NO: 123 - protein, 1270 aa, synthetic construct
  source 1..1270: note = CRISPR enzyme comprising NLS and ID218 Cas12a protein
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQKKRPAATKKAGQAKKKKPAAKRVKLDGGGGSGGGGSGGGGSPAAKRVKLD

SEQ ID NO: 124 - protein, 1249 aa, synthetic construct
  source 1..1249: note = CRISPR enzyme comprising NLS and ID218 Cas12a protein
PKKKRKVKKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQKPKKKRKVPKKKRKVPKKKRKV

SEQ ID NO: 125 - protein, 1394 aa, synthetic construct
  source 1..1394: note = CRISPR enzyme comprising NLS and ID204 Cas12a protein
PKKKRKVQNIESFTNLYSLTKVVKFNLDPIPETKKWLKAFGKVFTHNHSENLEDNFFKLDLKIEDAKRIIQDVLNSVHEAIITQALESEEAKSINISSYYELSNQRNHDLSDLEQRLREQIKVCFIKQLKDFCSEVSPKKKNSDDENGLPDLLKMLGSSNMLDYLSKSASKYSSPIHDKKSIEDACKDLKGFWGVLDKYIKNRENYYVFDKEQDTAVATRIVSDLLPMFCKNCKQYLTNKALYDSIFTSLKSSGIIMQMINPKTNEMRSIESFDSSIYNISGFYKYISQSGIETYNAIISRNNILVNLYNQQQKKANRINGFVELHKQIGCRESESTERIVICKDWDSTESKELGISMESMIKRIICMVNETIVPTEPNILHPRVYNLISWLENAVDWDGIYVSDKALSIISSHYLEDWYSLQQLLLNKESVGTYNQRKESNEQFKLNSAVELMPVFRALDCKDFDSVFKKSIYNDYSDILNKNLPPSRNLINMLCHDVKQIINNELPIYTKCITQLLTTKISKVQREGFFKQEEIIDIIRDYLDCVLSIARFVYFFSVRQNKIKGAPYNMELLQIVSSLLSTELGNRRLWYDEVRNYLLKSPQDEVKENKLKLNFNSSSLLKGWSIGEERTKLSLLLKNEGCYYLCILKNDKTCKKLFDINTQNNIYIEHGNAYRMMLRNLSFKTLVGKGYVSMFGTKYSEEDDEVAIQHAKQLIRQNYLKNYPELSAVITKPHSTKSAFFADVDSILKDYAQCYYNPINWNLIEKFANEGNIYVFRIHAKDYKDTSKGKKDLQTIYWEDVLSGHSSHSLSAYGEVFRRDAISTDKTKPFIHKKGSVLINKRDIDGNSIPNSIYIIIYALLNKVPLPKTAEEKDIERAKSLIEKNIIISKESWMNIIKDARFYGEEKYFLHNPICFNYKSIGFKSEDFSKDIPYHVINPIIQELMHNSCAPTSFIGIDRGEKNLIYACKINSEGSILFCNHYNIVHGINYLEKLEERADSRLKAKRSWKQQESIKNLKEGYISHVVHQLLEDVISVGNENLTPSYIVLEKLSREMKRGRQKIEKQVYQKFELALASKLGFYVNKDIEEEKPGSIQMPLQFVPPVNTFDQIDKKDSFGIMLYTRANYTSVTDPLTGWRKTIYITNGNNEDILKELTEAFDDIFFDGVDYVFSYVEKNSGQRWNIYSGNHGKSLDRFEYNKDTKCYESYNIVEVLDVLFADFNKSSSLLEQMRRGVKLKKAEENRTAYDSLRKAINMIQQIRNTGNEIKDNNFLQSPIRKDGKHFDTRNANDFRDLNLHFIKDADANGAYNIARKGLIMDAHYKYWMEQGKPNVKKKNKKEKEVSALSLYVSDREWDMWLLNREMWEKHLSEFSLRKQDKRPAATKKAGQAKKKK

SEQ ID NO: 126 - protein, 1322 aa, synthetic construct
  source 1..1322: note = CRISPR enzyme comprising NLS and ID206 Cas12a protein
PKKKRKVSRDFSQFTNQYKLSQTLQFSLIPQGRTREFMDSFLIHDQSRSDAYPEVKKILDNAHKALIERTLGSISQRIHSLSDKEKKKFASILNGDDINWLQLDKAVENEAENGKDATDAVRLEYCRLLAMLFSYDDAFESLVKNATPDEYFKNELKKMALSDDDTARFLEKFKGFACYFKGFQENRRNIYSQEAQSTAAPHRAIYDNFPKMKRCIKIFNHIKDAYPEILTTATTELAELLNGQSIDSVFSIQEYSNYLSQKGIDKFNSILGGYTTKDGQKVKGINECINLYRQQHEDARKDRSFSTMPLLFKQILSDRSSLSFIPQMFQNDQELFNTLKEFIQQLDHVTTADGIDRPLIPSFRDCLDNISIDDGILVSASSLTEISMRASGSWNAIPDAMENWAAVNISTKALREKWLKKNSYSLTELDALHLEKETDEKRISCDILDYWKGKKALDCYDRYDALLKDITSLMSAPVNDIPLAERKDDVALIKDYLDSLMDILHLGKPLYCQDILDRNEQFYSLFDGLYEQLQIVIPLYNRIRNYLTQKADTTKKVKLMFDSPTLADGWDQNKEQTNNAIILLKDGKYYLGIMNPAKKPVMSKMVVQKAESEVEVYQKMVYKYLPGPNKMLPKVFFSTKGQKDFNPPKSLLEKYEAKMHIKSSPDFSLAFLHELIDYFKASIAKHPDWSKFGFKFSPTASYQGIDEFYREISNQGYRLSFEIIPKEKIDDLVNNEQLLLFQIYNKDFAPGATGTPDKFTIYWKMLFSPENLSNVILKLNGEAELFWRKQVIAKPFSHKLNEKMVNRTYVDGTDSTGRPIVKHMPEKVHAVIFDYVNGKNDDIHKKEYQDALALYDSGKVIVKDVTHPIIKDRRYTQESFSFHVPITINFTAPDKPSKFNDSIIAFLKNNPDVKIIGIDRGERNLIYFSLIDQQGNILKQGSLNTVNPRTEARIEQVNYHELLNQKEKERDEARKSWSSIGKIKDLKAGYLSQVIHQIAPLMVENNAIVILEDLNFGFKRGRFAIEKQVYQNFEKALITKLNYLVFKEIQDYNTAGSVLIGYQLTDKFVSFEKMGKQTGALFYVPAAYTSKIDPTTGFANLFNTRKCTNVESRKVFLMTFDAIKYNAVRKSFAFSFDYAKFQTSAKSFQNKWTVYTSQKRLVFNPLTRQDTAVNPTQMIVDALNKRGVSLSDEFDLKAYLQNTDAIKANAEFFKTIFIALEYTLQMRNSNSSSGEDYIESPVLNKSGEFFRSSEDNPALPIDADANGAYHIAMKGLFLLLNVFNQGKKDLKIEHQAWFEFMQKRNMKRPAATKKAGQAKKKK

SEQ ID NO: 127 - protein, 1254 aa, synthetic construct
  source 1..1254: note = CRISPR enzyme comprising NLS and ID210 Cas12a protein
PKKKRKVAYSDFIGMYPLQKTLRFELKPQGRTMETLKEKDYLKNDLYRASQYAGMKKLIDDYHRSFIDESLMTSEIDWQPLADALDARKNNDEKSKKQLEILQDQYRRQVSKLFTGKEVFKSLFSKEVLDLVREKCVKERDDSKIQICDAFNRYSVYFTGFHENRRNFYVSDKQSTSISKRLVDDNFPKFLENIRIYEKIEVECPEIIEKVKNELKLTDLKKYFSVSEFNRFVRQCGIDDYNWILGGHVGEEAHVKGFNGFLNEAYQQGLLKTRVLMRPLYKMILSISESKSVLPYQFKDEKEMMDAVVSFIQKANEGIIAQAGDLLKSVESTDLSRVFIPDKQLTELSQILFGDWGALGTMMREYKAYDLGDPTMSKTMKKVDNWMKSSEFSLEDVKKAVLHADQEFTLENYEVRVDQVCSKLIETNAVSELKSVKIDGTDGETRNRSAECLRCILEGYQDLLHCMNMFKVSDELDRDEAFYSEFDLILGELQEIIPLFNKVRNYCTKKAYNETKVKLKLSTPTLADGWDLDKEKDNKAVILRSGGQYYLGVNCDKGFLDFSKYETDDEECFEKLNYKLLPGPNKMLPKVFFSKSWKSEHTIPERIQDGYDRGIFKKGPEFDLMFCHELIDFYKESISIHPVWSKFDFRFAETSDYQDISQFYADVQRQGFKMSFSKVSKSEVYKLVDEGKLMLFKIHSKDFSENSRGMPNMHTLYWRALFSEENLANIRIKLNGEAELFFREKSIDTPVVHKSGSVLVNKTFNGKHVPDKIYYELFRYRNGIITALSPEAKEYEGAIVSSVANYDIVKDRRYTVDKLFFHVPLTFNFGVPDRSKDINRMVLEHAIKSKDTKIIGIDRGERNLIYISMIDKEGKILLQKSLNVIDGVDYHGNLDVREHENKEARRNWTKVAGIKDLKEGYLSAAVSQIARMVVENDAIVVMENLNYGFKHGRFKIEKQVYQKFESMLIDKLNYLVFKDKEDFTSDGRAMRGYQLTKSIDSVKEIGTQTGVIFYVNAAYTSKIDPTTGFINQIQFSTKSTLSAKRDLLCNIDSIRYDPSEECYIFRIDYTKVGDRTKEFKNVWDIYTRGDRIVYSTKERCDKHLHPTEIMTKALESAGIGKEGELKEKICSADSKVIEAVVYALDLTLRLRNEDRETDYILSPVRNADGKFFCTLDGDLSLPLDSDANGAYNIALKGLLMLKMTEESNGPDADKYDISLVTNSDWLTFCQTGHRTWKSKRPAATKKAGQAKKKK

SEQ ID NO: 128 - protein, 1254 aa, synthetic construct
  source 1..1254: note = CRISPR enzyme comprising NLS and ID211 Cas12a protein
PKKKRKVTNFYSSFTNLRSVSKTLRFSLSPVGKTRENIRSANIIPQDEKRMQDYLVLKNVFDAYHKAYIDQHLCRIQGDWSELANALDERQSAPDADALNKMQETYRKEISNLLSGKGTGKNQYLFPADGDGGSLLEMLDPADMIKQTVKEKKDAEKGNRPPQYLIPEFGQNIKEVEQFNRFAGYFEDYKKTRENIYASEGSTSVAHRIVHENFPKFMSNVHTFRRLPESLRQKFEHDLSELLDGASLETMFDVDYFNVVLAQAGIERYNTLLGGKKEESGVKLQGLNELCNLEFQQGNLPKKVVFVPLYKQILSDREKASFLPEKFKSVEEMLESIADYRQSLSNLMASQHCLLDENMSTGYCDAGHVYIRKSQLASLSMLCFNEWSTFRTAVEAKDVYTLSELLQAAGKDILEDVRRELNFSLDSIDEAGEALLPVLEKQTLTEDDYAIIKEYLDAVIAAQHILKVFEAEDDAEKDPVFYGVADILNDALRQGVRLYDRVRNYATGKPYAEDKFRLTFFAPTLLNGWDKNKERDNCCMLFRKDGLYYLGILNAKNKPLLDEEAEIEGASYEKMVYKYLPEPNKMLPKVFNKSQKWIAAHGLDDDIRDGYEKGLHKVGQSFDKAFMHKLIDYYKACISEYEDWATFRFRFSATDTYQTMADFYNEIAAQSYKVFFTKVSEQQINAAVEAGQLYLFQIYNKDFSPYSHGAPNLHTLYWRELFSAENLQNPLVKLNGEAEIFYRHPSIANPFRHQKGDVLINKTDAAGRTVPEDRYRAAATDAANGMPVQALRASYPELIFKVADREIVKDKRYTKESFAFHVPLTLNNDNNAGFKLNMAVCEKLQSDGEYYIMGIDRGERHLLYISVIDRNGRIVEQRSLNVVGKNRVDYQAKLDALERQRQEARKNWRAITAIKELKEGYLSMAIHEITELMLQYPCILIMEDLNFGFKRGRFRVEKQVYQKFEKMLIEKLGFWMQKDKAADAAGGIRHAYQLTESFQSFQKLGKQSGVIFYVPANYTSKIDPKTGFVNLFGSRQLTYTSVSAAKAFFGTMREIGYDAQRDCYRFAFRYSDYDLYQQDHRDSWTVFTAGKGRIAHTVKNGRHTFEEIDVTERMKELFQRFGLDPYAADLRGAIDAADKKDFFSELLWLFKVTVQLRYEDSEQDFILSPIEKDGRFFDSRQAKKDEPQDGDANGAYHIALQGLRLARERIKDGRVQKDPKGKQTLNWLRFAQRKDYLEKRPAATKKAGQAKKKK

SEQ ID NO: 129 - protein, 1294 aa, synthetic construct
  source 1..1294: note = CRISPR enzyme comprising NLS and ID219 Cas12a protein
PKKKRKVIIYNCYIGGSFMKKIDSFTNCYSLSKTLRFKLIPIGATQSNFDLNKMLDEDKKRAENYSKAKSIIDKYHRFFIEKVLSSVTENKAFDSFLEDVRAYAELYYRSNKDDSDKASMKTLESKMRKFIALALQSDEGFKDLFGQNLIKKTLPEFLESDTDKEIIAEFDGFSTYFTGFFNNRKNMYSADDQSTAISYRCINDNLPKFLDNVRTFKNSDVANTLNNNLKILNEDFDGIYGTSAEDVFNVDYFPFVLSQKGIEAYNSILGGYTNSDGSKIKGLNEYINLYNQKNGNIHRIPKMKQLFKQILSERESVSFIPEKFDSDDDVLSSINDYYLERDGGKVLSIEKTVEKIEKLFSAVTDYSTDGIFVKNAAELTAVCSGAFGYWGTVQNAWNNEYDALNGYKETEKYIDKRKKAYKSVESFSLADIQKYADVSESSEANAEVTEWLRNEIKEKCNLAVQGYESSKDLISKPYTESKKLFNNDNAVELIKNALDSVKELENVLRLLLGTGKEESKDEYFYGEFLPCYERICEVDSLYDKVRNYMTQKLYKTDKIKINFSNSHFLSGWAQTYSTKGALIVKKENNYYLVIVDKKLSNDDIVFLGTNTQLSPAERIVYDFQKPDNKNTPRLFIRSKGTSYAPAVKEYDLPISDIIEIYDNEYFKTEYRKINPKGYKEALIKLIDYFKLGFSRHESYRCFNFKWKESEQYSDISEFYNDVVKSCYQLKSESINFDSLLKLVDEGKLYLFQLYNKDFSEHSKGTPNLHTLYFKMLFDERNLENVVFKLNGEAEMFYREASISKDDMIVHPKNQPIKNKNEQNSRKQSTFKYDIVKDRRYTVDQFMLHIPITLNFTANGGTNINNEVRKALKDCDKNYVIGIDRGERNLLYICVVDSEGRIIEQYSLNEIINEYNGNTYSTDYHALLDKKEKERLESRKAWKTVENIKELKEGYISQVVHKICELVEKYDAVIVMEDLNFGFKQGRSGKFEKSVYQKFEKMLIDKLNYFADKKKSPEEIGSVLNAYQLTNAFESFEKMGKQNGFIFYVPAYLTSKIDPTTGFADLLHPSSKQSKESMRDFVGRFDSITFNKTENYFEFELDYNKFPRCNTDYRKKWTVCTYGSRIKTFRNPEKNSEWDNKTVELTPAFMALFEKYSIDVNGDIKAQIMSVDKKDFFVELIGLLRLTLQMRNSETGKVDRDYLISPVKNSEGVFYNSDDYKGIENASLPKDADANGAYNIARKGLWIIEQIKACENDAELNKIRLAISNAEWLEYAQKKKRPAATKKAGQAKKKK

SEQ ID NO: 130 - protein, 1266 aa, synthetic construct
  source 1..1266: note = CRISPR enzyme comprising NLS and ID223 Cas12a protein
PKKKRKVMKSIKNFHNLYPVQKTLRFRLEPIGKTDDLIARNQILETDERRAAEYLKVKEYIDQYHRHFIENALSSPMLKVESMGKQDSLTDFADCYNNDNTEKRSEHLEKIQEKLRSQIVKRFSQLPAFARITKKELIKEDLPQFLKDKREQEIVSHFDEFTTYFTGFHQNRMNMYTADTKSTSIAFRLINQNLVKFVDNCNILEKVASSLSNEIIAKLDKDFEPFLNEHSALDLFKIENYNELLTQSQIEVYNSIIGGRVDEGNKIEIKGLNQYINEFNQTHEKSQRIPKLKPLFKQILSEDVGVSFRMEQFTDASQVQATIKEEYIKLESGVFDKLKEMIKSLSTFNLNGIYLANDLGLTDICQRHYGAWDILNNALITAFDAHIPRKKKQSQEKHDEQARKYLKGIKSISLGKIDSLLAEVTGKSIVDYFTSLGAINNETTQRENLFALIQNRYNAIKNVLTCPTASDELLRKNTEGIKDLLDAIKDLQHFIKPLCGYGEEADKDDIFYSDFSPLYETLDNTITPLYNKVRSYLTKKPYKLDKFKLNFDNAQLLDGWDINKENAYLSILLRKDGNFYLAIANKNDNRAITEINQAQADDGSDCYEKLNYKLIPSPFRMLPKVFFSRKGLETFNPSQEILDIYNNKEFQLGDKFDKEALYKLIDFYKTSIPNYESWQFFDFNFMPTQSYKSINEFYSDIEKQGYKIDFKQVPVSLINKLNDDGLLYLFKIVNKDFSPYSKGRPNLHTIYWKMLFDDNNLKDVIYKLNGKAEMFFRRNSITNPVIHPANKDIANKSTYNKQHKAVSKFDYDIIKDRRFTRNQYEFHVPITMNFKSASNGHFNRDVLAFIKQNGIKHIIGIDRGERHLLYLTMINLKGEIVKQLSLNSVACNPNNPDYKQDYNELLSIKEGDRLSARRNWATIENIKELKAGYLSQIVHLLSKMMIENDAILVLENLNTGFMRGRQKVEKSVYLKFEKMLIDKLNYVVDKTASINEPCGALKALQLTDTYDNFNKYQKGNVRQCGFIFYIPAWNTSKTDPATGFVNLFDTRLSTMGEIKSFFSKFDQIKYNSKNDSLEFTFDYNKFTSRAEGTRTCWTISSQGERIFTHRSKEQNNQFVSDTIYPTLIFKDVFSKNNIDINGNLKDEIASIDSLEALKQLLHAFKLVMQMRNSVTGTEVDFLLSPAIDAKGSNFDSRKGIKTLPENADANGAYNIARKGLMIIDQIKNTNDIDKIKYAVSNKDWLKFAQGKRPAATKKAGQAKKKK

SEQ ID NO: 131 - protein, 1393 aa, synthetic construct
  source 1..1393: note = CRISPR enzyme comprising NLS and ID224 Cas12a protein
PKKKRKVLKTKTITIMKEKEQYPDFTNFTNLYQLSKTLRFELKPIGKTEETFKQWLGAMNSAGNEDNNDNLFAQDKKIKDAYVVIKSIMDKLHEQFIEKSLTSEEAKNIDFSDFLDIYKYRGNDFPKDFEKKLREQIGNTYKVAGAYFSKEISKALNKEFKTKKPFECLTDAKMLNYLSANVKEIAEQNNIDQETLANHIKQFKGFWGYLSNYNKNRENYYTTDKEQSTAVATRIVHDNLPTFCDNIIRFEERNDEYCGICEYLKDKNIETKIKNNQGNEVDVEPITKDVFKIKHFNNCLAQSQIKEYNRIIGNYNYQINLYNQARSSENDFRKLDEFETLYKQIGCGEGRSLFETLHNYDDLNKLLEKIKEAGDSFFKECGYNEISTVPAFIKFLKECNNWDGIYMSSTAINRISNRYFANWHSIKDKLKDNKACVTYDKNREEPIQLRDAVELSDLFKVLDDKEQSEHFFKESLSNDINEKECPSKNLINLLCFDIEQNISKFLANSENIINLLNGNKDNQASEEDKIKQIKEWLDAAIDTMRIVRYFAVRKSKMKGNLPNTTMEKALNNLLYNDDAQWFKWYDLVRNYLTKKPQDDAKENKLKLNFGNPSLLGGWSDGQEQTKAATLLKYNNELYLCILKIRNIFDTSKADNPIYTKAPFCACRLILRNLKFQTLAGKGFKSKYGIKYSEMGNEDQSQAIKCLKEIIRERYAKKYPPLEQIINQEYSDKKSFDNEVKEALKECYVCEFVPINWDVVSKKQNSTDLFLFKIHCKDYLPKATGKKDLQTMYWEDVLSDSSNHQLCGGAEIFMREPVPKESPVKHGVGSKLVNKRDKDGNTIPEQIYKEIYLFANDKVKKENLSAEAKNYIESEKVTIKEAEYEIVKDNRFYGETKYMFHCPITINFKAKGYKYAYPEVNEKITDALQQSDNLQFIGIDRGEKHLVYSCTIDKEGKIVKCNHHDNINGTDYVQKLEAVANERIAAKKNWQQQNKIKDLKAGYISHVVHRLVDETVKDGDKITPHAYIVLEDLNSEMKRGRQKIEKQVYQNLELALAKKFNFVVDKEAKQGELGSVSKALQLTPPVNNYQDIEGKKQFGVMLYTRANYTSITDPTTGWRKTIYIKNGKDEEIRKQILEKFTDFGFAGKDYYFEYTEENAGHTWRMYSGKNGEPLPRFQNKKQMEQDKNIWVPEQINVKEILDKLFANFDKGRSFKEQVNEEVKLEKIEGRSETAWQSLRYALNMIQQIRNSGVEKTDDNFLYSPVRNENGEHFDTRNHENNGDLSPIVDADANGAFNIARKGLIMDAHIKHWINSGRPKIKKDGKETPDLDLFISDREWDLWLLDREEWKKYLPVFASKSKKDADKTTSTKSPKRKKKKRPAATKKAGQAKKKK

SEQ ID NO: 132 - protein, 1273 aa, synthetic construct
  source 1..1273: note = CRISPR enzyme comprising NLS and ID230 Cas12a protein
PKKKRKVKDLKQFIGIYPVSKTLRFELKPIGKTLDWIRERNVVEKDERKSQDYPVVKQHIDEYHKVCISESLKNVCFDWKPLKEAIEKHHKLKSDESKKQLETEQTNMRKQIVLAIKRFKHYPELTAATPKDLIQNVLPEITNDSSLNSFNRFASYFKGFQENRKNIYSLDAISTGVPFRLVHDNFPKFLSNIDIYENIKSTAPETLELAAKELQPFLEGVMLDDIFSLDFYNSLLTQDGIDFFNRVIGGVSEEGKQKFRGINEFTNLYRQQHPELSANKKAMTMIPLFKQILSERETLSYIPKQIENENELTETIKQFYSHITNFERNGKTINILNELVRLLGQVTSYNQDRIFVSSKYITDISQKLYGRWNAINDYLYERAVEMFGDVSVAKNKKLVDNYIDKDVYSASELGFDENNNLSTFYSELPQIVGSVNGNWIQFCEWCKIDKKQKFLNNAEGTEIIKKLLDAIMEVMHKCNVLVVSEEYDLDKDFYNNFFPLFTELENIVFVYNRVRNFLTKKPSDVKKFKLNFGVPTLCDGWDLNKEKDNKAILLFKDNRSYLGIMNPEDYPKIKESNDSGDGYFSKMVYKLLPGPNKMLPKVFFSKKGRETFSPSDRIIDIYESGAFKKGSSNFSIVCLHELIDFYKDAINRHEDWSKYNFKFSPTDSYKDIGAFYNEISKQAYKIRFTNISEQEINQLVDKGQLYLFQLYNKDYASGAHGRKNLHTLYWENLFKEENLNNLVLKLNGQAELFYRPQSINEPVAHKVGSIMLNRRDKSGMPIPETVYRSLYLYFNGKKKEEELDSVEKSYIDNVIVKKVTHEIVKDKRFTKPEFFFHVPITFNSNADGNEFINENVIDYITDNPDVNIIGIDRGERHLIYLTLINQQGEILKQKTFNTVGTYNYHAKLEQREQERQEARKSWQSIGKIKDLKEGFLSAVIHEIALMMAEYNAIVVLEDLNFGFKRGRFKVERQVYQKFEKMLIDKLNYLAFKDRKSDETGGILRGYQFAQKFVSFQRLGKQSGFLFYVPAAYTSKIDPVTGFVNHFNFNDITNAEKRKQFFCKMERIEMKNDNIEFEFDYRKFKTYQTDFQNVWTASTFGKRIVMKTDESGRKQMADYYPTNEILAAFKKYGYELTNGTDIKSLLQDIEADQKSASLFSNLFYAFQKTLQMRNSNHATEEDYILSPIAKHGYHFCSTEEANKGKDEFGNWLSKLPVDADANGAYHIALKGLYLLKNPQTKKIEHEKWFKFMVEKPYKKRPAATKKAGQAKKKK

SEQ ID NO: 133 - protein, 1255 aa, synthetic construct
  source 1..1255: note = CRISPR enzyme comprising NLS and ID238 Cas12a protein
PKKKRKVKEQFINQYPISKTLQFSLIPIGKTEENFNNKLLLEEDEKRAEEYQKVKEYIDRYHKFFIETALLNVSLNGLEEYVSLYFKPSKSDADVKAMSDIEAKLRKQISKAMTSHELYKDLFSKDMIDSILPKYLQKEEERSNVEMFRGFSTYFQGFYTNRKNMYSEEAQSTAISYRCINDNLPKFLDNVKSFEKIREAFNEEKITTINEEFFGILGKYATDIFNVDYFNFVLSQPGIDKYNKIIGGYVCSDGTKIQGLNELINLYNQQVAKSDKSKRLPLLKMLYKQILSDRESVSFIPEKYKDDNEIIASINEYFSETALETVAALKELFDKFSSFDLKGVYISSGLAVTSLSNDVFGSWNAFSSAWETRYSEQNPKKKNKSQEKYDEELKANFKKNKSFSLDEFQQLGSEMKEPDSIGSVLDFYAGAVSEKADNIRALYIAAKELLTPNYAEKYDKKLIKNHVAVEKIKSLLDAVKSLEKLVKPLLGTGKEEEKDNVFYGKFLPLYDSLTLIDRIYDKVRNYVTQKPYSKDKIKLNFENPQLLSGWDKNKESDCRGILLRRNGNYYLGIIDKSNSKVFQSYPVPEFSDDEYYEKIIYKQINGCSKYFSIKQIKPQNPPENIIKYLSKDFDKKTMTKGELVELIKYVVEDFIPNYSPLLDENGKCFFDFKYKPYDQYTSWNHFCKSLDPQAYTIRFSKVKKDYIDNLIEIGFLYLFQIYNKDFSEYSHGTPNLHTLYFKTLFDKKNFADVVFKLSGGAEMFFRKASIKENEKTVHPANQPIINKNPDNSKKQSEFSYDLVKDKRYTKRQFSIHIPITINFKANGREFINYEVRRALNSDYKPCVIGIDRGERNLIYITVINDTGEIVEQMSLNEIISDNGYKVDYQKLLDKKEKARDEARKSWGTIENIKELKEGYISQVIHKICELVIKYDAVIAMEDLNFGFKRGRFNVEKQVYQKFENMLISKLNYLCDKKMEANAEGGVLKAYQLTNKFDGVNKGKQNGIIFYVPAWLTSKIDPVTGFVDLLHPKYTSVEESHNLFGNFDDIRYNAVTDMFEFDLDYSKFPKSKADFKKKWTVCTNSDRIMTFRNPAKNSEWDNKRVILTDEFKKLFEEFGVDYKSNLREKILGISNPDFNKRLVKFLALTLQMRNSVTGSTNPEDDYLISPVRDKNGVFYDSRNFIGNDAVLPADADANGAYNIARKGLWAIDVIKSTSSDMLDKADLSISNAEWLEYVQKKRPAATKKAGQAKKKK

SEQ ID NO: 134 - protein, 1297 aa, synthetic construct
  source 1..1297: note = CRISPR enzyme comprising NLS and ID245 Cas12a protein
PKKKRKVNKFVNLYSVIKTLRFELVPIGKTKVNIEKSNLLMDDAKRAEDCKIVKSLMDRYHKQFIKSRLCNLELPLEENGQKCSLEEYRVLYEMKKRNAEQEATFAEVKGNLRALIAKHLTAKGTAYDRIFKKELIREDLLNFLEDESEREIVEQFADFTTYFYGFHKNRANMYSAEEKSTAIAYRLINQNLPKFMDNMRTFMRIAQTSVAEHFGEIYTGWKKFLNVESLEEFFSLKNFTKTLTQEHIEVYNYVIGKRILEDGAEIKGINEYVNLYNQQQKDKRNRLPFMVPLYKQILSDREQLSWLAEEFDSDEKMQKAVLETYGNLYDKLMGDENESLRSLLKNIDDYDLTQIHISNDLSLTEISQHIFGKYDAFTKGIKERLSGIVARKKNDKEEQYEDRINEVFKSYKSFSIADLNQLLLPEVEDGRPKNIEEYFKSLGAIHTDTYQKENLFTQIENAYTEASPILRMKETGDHLSQNKGAVEKIKNLLDALKALQHFIKPLTGSGEESDKDGRFYGAFQMIWDELDAVTPLYNKVRNWLTKKPYSTEKFKLNFASPTLLSGWTKPQDNSCVILSDDECCAFYLGIIDSHHRTLFNDIDRIHQKFRPGGMKIMNYYQGGDMGKNVQNLMMENGRVKKVNGRKETSGPFAGENLRLEQAKNKYLPPEINRIRKMASYKPNNSNYNKKDLELFIEYYMTLVKDYYPKCSFSFYPPSHYQSFDAFLTHIQQQSYQIHFSPISKSFIDNMVDAGKLYLFRIWNKDFSEHSKGTPNLHTLYWKMLFDERNLRDVVYKLNGQAEVFFRKSSISHENRIIHPANKEIENKNTLTEKKTSLFTYDIVKDKRFTVDKFQFHVPITINFKAKGLKNINPLVIDVIREGGFTHIIGIDRGERHLLYLSLIDLKGRIVKQMSLNQIINDYEGHQHKTDYHALLMKREGERTNARKSWDTIEKIKELKEGYLSQVIHLITKMMVKYKAIVVLEDLNSSFVRGRQKIERQVYEKFEKMLIDKLNCYIDKKRDPECEGGMLHALQLANKFKSFSKMGKQCGCLFYIPAWNTSKIDPVTGFVNLLDTRYESVKASKEFFSHFDCITFNATMDWFEFSFDYDNFHKKAEGTQTQWTVFSRGSRIHTFRNPEKNNQWDDEEINLTDKFKELFAKYSINYRGNLKDAMQDSNEATFFKELMGLMKLLMQMRNSKANSEIDYMLSPVSDKNGNFYDSRVCGGSLPENADANGAYNIARKGLMLIRNIQAAPDDKKPSLTITNKDWLCFAQTKPYLEDKRPAATKKAGQAKKKK

SEQ ID NO: 135 - DNA, 198 bp, synthetic construct
  source 1..198: mol_type = other DNA; note = Sequence of EMX1A gene
gtagcctcagtcttcccatcaggctctcagctcagcctgagtgttgaggccccagtggctgctctgggggcctcctgagtttctcatctgtgcccctccctccctggcccaggtgaaggtgtggttccagaaccggaggacaaagtacaaacggcagaagctggaggaggaagggcctgagtccgagcagaagaagaa

SEQ ID NO: 136 - DNA, 24 bp, synthetic construct
  source 1..24: mol_type = other DNA; note = Protospacer sequence of EMX1A gene
ctcatctgtgcccctccctccctg

SEQ ID NO: 137 - RNA, 24 nt, synthetic construct
  source 1..24: mol_type = other RNA; note = Guide domain sequence targeting EMX1A gene
ctcatctgtgcccctccctccctg

SEQ ID NO: 138 - DNA, 239 bp, synthetic construct
  source 1..239: mol_type = other DNA; note = Sequence of MTAP gene
ccatctgtacccccaaaaactattgaaattaaaaaaaaattgtgattgatgtggtttcgctgcatcctcgcccaaatctcatttgtattttagccccaataatccccacatgtcatggaagggacctggtggcaggtaattgaatcatggaggtgggtctttctcgtgctgttctcgtgatagtgaataaatctcacaagatctgatggttttataaaggggagttcccctgcacatgc

SEQ ID NO: 139 - DNA, 23 bp, synthetic construct
  source 1..23: mol_type = other DNA; note = Protospacer sequence of MTAP gene
gccccaataatccccacatgtca

SEQ ID NO: 140 - RNA, 23 nt, synthetic construct
  source 1..23: mol_type = other RNA; note = Guide domain sequence targeting MTAP gene
gccccaataatccccacatgtca

SEQ ID NO: 141 - DNA, 190 bp, synthetic construct
  source 1..190: mol_type = other DNA; note = Sequence of DRYK1A gene
tcttaaaaccttgtcacacacaatgaaactttgctgttcactgtcagttataacttacatgaggtgacccatttccattcaagggttttagaagcacatcaaggacattctaaggatgattgacttacacaatgatctctgaacatgcctcctgccttctcctcactcttgagtatttgcttaggggagc

SEQ ID NO: 142 - DNA, 23 bp, synthetic construct
  source 1..23: mol_type = other DNA; note = Protospacer sequence of DRYK1A gene
gaagcacatcaaggacattctaa

SEQ ID NO: 143 - RNA, 23 nt, synthetic construct
  source 1..23: mol_type = other RNA; note = Guide domain sequence targeting DRYK1A gene
gaagcacatcaaggacattctaa

SEQ ID NO: 144 - DNA, 222 bp, synthetic construct
  source 1..222: mol_type = other DNA; note = Sequence of DNMT1A gene
ccacttattgggtcagctgttaacatcagtacgttaatgtttcctgatggtccatgtctgttactcgcctgtcaagtggcgtgacaccgggcgtgttccccagagtgacttttccttttatttcccttcagctaaaataaaggaggaggaagctgctaaggactagttctgccctcccgtcacccctgtttctggcaccaggaatccccaacatgcactgat

SEQ ID NO: 145 - DNA, 23 bp, synthetic construct
  source 1..23: mol_type = other DNA; note = Protospacer sequence of DNMT1A gene
tttcccttcagctaaaataaagg

SEQ ID NO: 146 - RNA, 23 nt, synthetic construct
  source 1..23: mol_type = other RNA; note = Guide domain sequence targeting DNMT1A gene
tttcccttcagctaaaataaagg

SEQ ID NO: 147 - DNA, 45 bp, synthetic construct
  source 1..45: mol_type = other DNA; note = primer sequence for experimental example
gaaattaatacgactcactataggtaatttctactattcgtagat

SEQ ID NO: 148 - DNA, 44 bp, synthetic construct
  source 1..44: mol_type = other DNA; note = primer sequence for experimental example
cagggagggaggggcacagatgaatctacgaatagtagaaatta

SEQ ID NO: 149 - DNA, 45 bp, synthetic construct
  source 1..45: mol_type = other DNA; note = primer sequence for experimental example
gaaattaatacgactcactataggttaatttctactttcgtagat

SEQ ID NO: 150 - DNA, 44 bp, synthetic construct
  source 1..44: mol_type = other DNA; note = primer sequence for experimental example
cagggagggaggggcacagatgaatctacgaaagtagaaattaa

SEQ ID NO: 151 - DNA, 45 bp, synthetic construct
  source 1..45: mol_type = other DNA; note = primer sequence for experimental example
gaaattaatacgactcactataggcaatttctactttcagtagat

SEQ ID NO: 152 - DNA, 44 bp, synthetic construct
  source 1..44: mol_type = other DNA; note = primer sequence for experimental example
cagggagggaggggcacagatgaatctactgaaagtagaaattg

SEQ ID NO: 153 - DNA, 45 bp, synthetic construct
  source 1..45: mol_type = other DNA; note = primer sequence for experimental example
gaaattaatacgactcactataggttaatttctacttgtgtagat

SEQ ID NO: 154 - DNA, 44 bp, synthetic construct
  source 1..44: mol_type = other DNA; note = primer sequence for experimental example
cagggagggaggggcacagatgaatctacacaagtagaaattaa

SEQ ID NO: 155 - DNA, 45 bp, synthetic construct
  source 1..45: mol_type = other DNA; note = primer sequence for experimental example
gaaattaatacgactcactataggtaatttctactaagtgtagat

SEQ ID NO: 156 - DNA, 44 bp, synthetic construct
  source 1..44: mol_type = other DNA; note = primer sequence for experimental example
cagggagggaggggcacagatgaatctacacttagtagaaatta

SEQ ID NO: 157 - DNA, 45 bp, synthetic construct
  source 1..45: mol_type = other DNA; note = primer sequence for experimental example
gaaattaatacgactcactataggtaaatttctactattgtagat

SEQ ID NO: 158 - DNA, 44 bp, synthetic construct
  source 1..44: mol_type = other DNA; note = primer sequence for experimental example
cagggagggaggggcacagatgaatctacaatagtagaaattta

SEQ ID NO: 159 - DNA, 45 bp, synthetic construct
  source 1..45: mol_type = other DNA; note = primer sequence for experimental example
gaaattaatacgactcactataggaaaatttctactattgtagat

SEQ ID NO: 160 - DNA, 44 bp, synthetic construct
  source 1..44: mol_type = other DNA; note = primer sequence for experimental example
cagggagggaggggcacagatgaatctacaatagtagaaatttt

SEQ ID NO: 161 - DNA, 45 bp, synthetic construct
  source 1..45: mol_type = other DNA; note = primer sequence for experimental example
gaaattaatacgactcactataggataatttctactgttgtagat

SEQ ID NO: 162 - DNA, 44 bp, synthetic construct
  source 1..44: mol_type = other DNA; note = primer sequence for experimental example
cagggagggaggggcacagatgaatctacaacagtagaaattat

SEQ ID NO: 163 - DNA, 45 bp, synthetic construct; identical to SEQ ID NO: 147
  source 1..45: mol_type = other DNA; note = primer sequence for experimental example

SEQ ID NO: 164 - DNA, 44 bp, synthetic construct
  source 1..44: mol_type = other DNA; note = primer sequence for experimental example
tgacatgtggggattattggggcatctacgaatagtagaaatta

SEQ ID NO: 165 - DNA, 44 bp, synthetic construct
  source 1..44: mol_type = other DNA; note = primer sequence for experimental example
ttagaatgtccttgatgtgcttcatctacgaatagtagaaatta

SEQ ID NO: 166 - DNA, 45 bp, synthetic construct; identical to SEQ ID NO: 149
  source 1..45: mol_type = other DNA; note = primer sequence for experimental example

SEQ ID NO: 167 - DNA, 44 bp, synthetic construct
  source 1..44: mol_type = other DNA; note = primer sequence for experimental example
tgacatgtggggattattggggcatctacgaaagtagaaattaa

SEQ ID NO: 168 - DNA, 44 bp, synthetic construct
  source 1..44: mol_type = other DNA; note = primer sequence for experimental example
ttagaatgtccttgatgtgcttcatctacgaaagtagaaattaa

SEQ ID NO: 169 - DNA, 45 bp, synthetic construct; identical to SEQ ID NO: 151
  source 1..45: mol_type = other DNA; note = primer sequence for experimental example

SEQ ID NO: 170 - DNA, 44 bp, synthetic construct
  source 1..44: mol_type = other DNA; note = primer sequence for experimental example
tgacatgtggggattattggggcatctactgaaagtagaaattg

SEQ ID NO: 171 - DNA, 44 bp, synthetic construct
  source 1..44: mol_type = other DNA; note = primer sequence for experimental example
ttagaatgtccttgatgtgcttcatctactgaaagtagaaattg

SEQ ID NO: 172 - DNA, 45 bp, synthetic construct; identical to SEQ ID NO: 153
  source 1..45: mol_type = other DNA; note = primer sequence for experimental example

SEQ ID NO: 173 - DNA, 44 bp, synthetic construct
  source 1..44: mol_type = other DNA; note = primer sequence for experimental example
tgacatgtggggattattggggcatctacacaagtagaaattaa

SEQ ID NO: 174 - DNA, 44 bp, synthetic construct
  source 1..44: mol_type = other DNA; note = primer sequence for experimental example
ttagaatgtccttgatgtgcttcatctacacaagtagaaattaa

SEQ ID NO: 175 - DNA, 45 bp, synthetic construct
  source 1..45: mol_type = other DNA; note = primer sequence for experimental example
gaaattaatacgactcactataggaatttctactactatgtagat

SEQ ID NO: 176 - DNA, 44 bp, synthetic construct
  source 1..44: mol_type = other DNA; note = primer sequence for experimental example
tgacatgtggggattattggggcatctacatagtagtagaaatt

SEQ ID NO: 177 - DNA, 44 bp, synthetic construct
  source 1..44: mol_type = other DNA; note = primer sequence for experimental example
ttagaatgtccttgatgtgcttcatctacatagtagtagaaatt

SEQ ID NO: 178 - DNA, 45 bp, synthetic construct; identical to SEQ ID NO: 155
  source 1..45: mol_type = other DNA; note = primer sequence for experimental example

SEQ ID NO: 179 - DNA, 44 bp, synthetic construct
  source 1..44: mol_type = other DNA; note = primer sequence for experimental example
tgacatgtggggattattggggcatctacacttagtagaaatta

SEQ ID NO: 180 - DNA, 44 bp, synthetic construct
  source 1..44: mol_type = other DNA; note = primer sequence for experimental example
ttagaatgtccttgatgtgcttcatctacacttagtagaaatta

SEQ ID NO: 181 - DNA, 45 bp, synthetic construct; identical to SEQ ID NO: 157
  source 1..45: mol_type = other DNA; note = primer sequence for experimental example

SEQ ID NO: 182 - DNA, 44 bp, synthetic construct
  source 1..44: mol_type = other DNA; note = primer sequence for experimental example
tgacatgtggggattattggggcatctacaatagtagaaattta

SEQ ID NO: 183 - DNA, 44 bp, synthetic construct
  source 1..44: mol_type = other DNA; note = primer sequence for experimental example
ttagaatgtccttgatgtgcttcatctacaatagtagaaattta

SEQ ID NO: 184 - DNA, 45 bp, synthetic construct
  source 1..45: mol_type = other DNA; note = primer sequence for experimental example
gaaattaatacgactcactataggaaaatttctactcttgtagat

SEQ ID NO: 185 - DNA, 44 bp, synthetic construct
  source 1..44: mol_type = other DNA; note = primer sequence for experimental example
tgacatgtggggattattggggcatctacaagagtagaaatttt

SEQ ID NO: 186 - DNA, 44 bp, synthetic construct
  source 1..44: mol_type = other DNA; note = primer sequence for experimental example
ttagaatgtccttgatgtgcttcatctacaagagtagaaatttt

SEQ ID NO: 187 - DNA, 45 bp, synthetic construct
  source 1..45: mol_type = other DNA; note = primer sequence for experimental example
gaaattaatacgactcactataggtaaatttctactgttgtagat

SEQ ID NO: 188 - DNA, 44 bp, synthetic construct
  source 1..44: mol_type = other DNA; note = primer sequence for experimental example
tgacatgtggggattattggggcatctacaacagtagaaattta

SEQ ID NO: 189 - DNA, 44 bp, synthetic construct
  source 1..44: mol_type = other DNA; note = primer sequence for experimental example
ttagaatgtccttgatgtgcttcatctacaacagtagaaattta

SEQ ID NO: 190 - DNA, 45 bp, synthetic construct
  source 1..45: mol_type = other DNA; note = primer sequence for experimental example
gaaattaatacgactcactataggataatttctactattgtagat

SEQ ID NO: 191 - DNA, 44 bp, synthetic construct
  source 1..44: mol_type = other DNA; note = primer sequence for experimental example
tgacatgtggggattattggggcatctacaatagtagaaattat

SEQ ID NO: 192 - DNA, 44 bp, synthetic construct
  source 1..44: mol_type = other DNA; note = primer sequence for experimental example
ttagaatgtccttgatgtgcttcatctacaatagtagaaattat

SEQ ID NO: 193 - DNA, 79 bp, synthetic construct
  source 1..79: mol_type = other DNA; note = Wild type sequence of EMX1 for comparing, from examplary example 2
ctctgggggcctcctgagtttctcatctgtgcccctccctccctggcccaggtgaaggtgtggttccagaaccggagga

SEQ ID NO: 194 - DNA, 64 bp, synthetic construct
  source 1..64: mol_type = other DNA; note = Mutation sequence of EMX1 from examplary example 2, ID 218
ctctgggggcctcctgagtttctcatctgtgcccaggtgaaggtgtggttccagaaccggagga

SEQ ID NO: 195 - DNA, 75 bp, synthetic construct
  source 1..75: mol_type = other DNA; note = Mutation sequence of EMX1 from examplary example 2, ID 218
ctctgggggcctcctgagtttctcatctgtgcccctccctggcccaggtgaaggtgtggttccagaaccggagga

SEQ ID NO: 196 - DNA, 76 bp, synthetic construct
  source 1..76: mol_type = other DNA; note = Mutation sequence of EMX1 from examplary example 2, ID 218
ctctgggggcctcctgagtttctcatctgtgcccctcccctggcccaggtgaaggtgtggttccagaaccggagga

SEQ ID NO: 197 - DNA, 65 bp, synthetic construct
  source 1..65: mol_type = other DNA; note = Mutation sequence of EMX1 from examplary example 2, ID 218
ctctgggggcctcctgagtttctcatctgtgccccaggtgaaggtgtggttccagaaccggagga

SEQ ID NO: 198 - DNA, 63 bp, synthetic construct
  source 1..63: mol_type = other DNA; note = Mutation sequence of EMX1 from examplary example 2, ID 218
ctctgggggcctcctgagtttctcatctggcccaggtgaaggtgtggttccagaaccggagga

SEQ ID NO: 199 - DNA, 78 bp, synthetic construct
  source 1..78: mol_type = other DNA; note = Mutation sequence of EMX1 from examplary example 2, ID 218
ctctgggggcctcctgagtttctcatctgtgcccctccctcctggcccaggtgaaggtgtggttccagaaccggagga

SEQ ID NO: 200 - DNA, 64 bp, synthetic construct; identical to SEQ ID NO: 194
  source 1..64: mol_type = other DNA; note = Mutation sequence of EMX1 from examplary example 2, Lba

SEQ ID NO: 201 - DNA, 75 bp, synthetic construct; identical to SEQ ID NO: 195
  source 1..75: mol_type = other DNA; note = Mutation sequence of EMX1 from examplary example 2, Lba

SEQ ID NO: 202 - DNA, 65 bp, synthetic construct; identical to SEQ ID NO: 197
  source 1..65: mol_type = other DNA; note = Mutation sequence of EMX1 from examplary example 2, Lba

SEQ ID NO: 203 - DNA, 45 bp, synthetic construct
  source 1..45: mol_type = other DNA; note = Mutation sequence of EMX1 from examplary example 3, 218
ctctgggggcctcctgagtttctcatctgtgcccaggtgaaggtg

SEQ ID NO: 204 - DNA, 56 bp, synthetic construct
  source 1..56: mol_type = other DNA; note = Mutation sequence of EMX1 from examplary example 3, 218
ctctgggggcctcctgagtttctcatctgtgcccctccctggcccaggtgaaggtg

SEQ ID NO: 205 - DNA, 57 bp, synthetic construct
  source 1..57: mol_type = other DNA; note = Mutation sequence of EMX1 from examplary example 3, 218
ctctgggggcctcctgagtttctcatctgtgcccctcccctggcccaggtgaaggtg

SEQ ID NO: 206 - DNA, 46 bp, synthetic construct
  source 1..46: mol_type = other DNA; note = Mutation sequence of EMX1 from examplary example 3, 218
ctctgggggcctcctgagtttctcatctgtgccccaggtgaaggtg

SEQ ID NO: 207 - DNA, 55 bp, synthetic construct
  source 1..55: mol_type = other DNA; note = Mutation sequence of EMX1 from examplary example 3, 218
ctctgggggcctcctgagtttctcatctgtgcccctcctggcccaggtgaaggtg

SEQ ID NO: 208 - DNA, 59 bp, synthetic construct
  source 1..59: mol_type = other DNA; note = Mutation sequence of EMX1 from examplary example 3, 218
ctctgggggcctcctgagtttctcatctgtgcccctccctcctggcccaggtgaaggtg

SEQ ID NO: 209 - DNA, 45 bp, synthetic construct
  source 1..45: mol_type = other DNA; note = Mutation sequence of EMX1 from examplary example 3, 218
ctctgggggcctcctgagtttctcatctgggcccaggtgaaggtg

SEQ ID NO: 210 - DNA, 37 bp, synthetic construct
  source 1..37: mol_type = other DNA; note = Mutation sequence of EMX1 from examplary example 3, 218
ctctgggggcctcctgagtttctcatctgtgaaggtg

SEQ ID NO: 211 - DNA, 70 bp, synthetic construct
  source 1..70: mol_type = other DNA; note = Wild type sequence of MTAP for comparing, from examplary example 4
atcctcgcccaaatctcatttgtattttagccccaataatccccacatgtcatggaagggacctggtggc

SEQ ID NO: 212 - DNA, 50 bp, synthetic construct
  source 1..50: mol_type = other DNA; note = Mutation sequence of MTAP from examplary example 4, 218
atcctcgcccaaatctcatttgtattttagccccaataatccctggtggc

SEQ ID NO: 213 - DNA, 49 bp, synthetic construct
  source 1..49: mol_type = other DNA; note = Mutation sequence of MTAP from examplary example 4, 218
atcctcgcccaaatctcatttgtattttagccccaataatcctggtggc

SEQ ID NO: 214 - DNA, 51 bp, synthetic construct
  source 1..51: mol_type = other DNA; note = Mutation sequence of MTAP from examplary example 4, 218
atcctcgcccaaatctcatttgtattttagccccaataatcccctggtggc

SEQ ID NO: 215 - DNA, 67 bp, synthetic construct
  source 1..67: mol_type = other DNA; note = Mutation sequence of MTAP from examplary example 4, 218
atcctcgcccaaatctcatttgtattttagccccaataatcccctgtcatggaagggacctggtggc

SEQ ID NO: 216 - DNA, 58 bp, synthetic construct
  source 1..58: mol_type = other DNA; note = Mutation sequence of MTAP from examplary example 4, 218
atcctcgcccaaatctcatttgtattttagccccaataatccccagggacctggtggc

SEQ ID NO: 217 - DNA, 68 bp, synthetic construct
  source 1..68: mol_type = other DNA; note = Mutation sequence of MTAP from examplary example 4, 218
atcctcgcccaaatctcatttgtattttagccccaataatccccatgtcatggaagggacctggtggc

SEQ ID NO: 218 - DNA, 52 bp, synthetic construct
  source 1..52: mol_type = other DNA; note = Mutation sequence of MTAP from examplary example 4, 218
atcctcgcccaaatctcatttgtattttagccccaataagggacctggtggc

SEQ ID NO: 219 - DNA, 54 bp, synthetic construct
  source 1..54: mol_type = other DNA; note = Mutation sequence of MTAP from examplary example 4, 218
atcctcgcccaaatctcatttgtattttagccccaataatccccacctggtggc

SEQ ID NO: 220 - DNA, 60 bp, synthetic construct
  source 1..60: mol_type = other DNA; note = Mutation sequence of MTAP from examplary example 4, 218
atcctcgcccaaatctcatttgtattttagccccaataatccccacagggacctggtggc

SEQ ID NO: 221 - DNA, 69 bp, synthetic construct
  source 1..69: mol_type = other DNA; note = Mutation sequence of MTAP from examplary example 4, 218
atcctcgcccaaatctcatttgtattttagccccaataatccccactgtcatggaagggacctggtggc

SEQ ID NO: 222 - DNA, 49 bp, synthetic construct; identical to SEQ ID NO: 213
  source 1..49: mol_type = other DNA; note = Mutation sequence of MTAP from examplary example 4, 233

SEQ ID NO: 223 - DNA, 50 bp, synthetic construct; identical to SEQ ID NO: 212
  source 1..50: mol_type = other DNA; note = Mutation sequence of MTAP from examplary example 4, 233

SEQ ID NO: 224 - DNA, 65 bp, synthetic construct
  source 1..65: mol_type = other DNA; note = Mutation sequence of MTAP from examplary example 4, 233
atcctcgcccaaatctcatttgtattttagccccaataatccccacatggaagggacctggtggc

SEQ ID NO: 225 - DNA, 51 bp, synthetic construct; identical to SEQ ID NO: 214
  source 1..51: mol_type = other DNA; note = Mutation sequence of MTAP from examplary example 4, 233

SEQ ID NO: 226 - DNA, 47 bp, synthetic construct
  source 1..47: mol_type = other DNA; note = Mutation sequence of MTAP from examplary example 4, 233
atcctcgcccaaatctcatttgtattttagccccaatacctggtggc

SEQ ID NO: 227 - DNA, 65 bp, synthetic construct; identical to SEQ ID NO: 224
  source 1..65: mol_type = other DNA; note = Mutation sequence of MTAP from examplary example 4, 236

SEQ ID NO: 228 - DNA, 49 bp, synthetic construct; identical to SEQ ID NO: 213
  source 1..49: mol_type = other DNA; note = Mutation sequence of MTAP from examplary example 4, 236

SEQ ID NO: 229 - DNA, 68 bp, synthetic construct; identical to SEQ ID NO: 217
  source 1..68: mol_type = other DNA; note = Mutation sequence of MTAP from examplary example 4, 236

SEQ ID NO: 230 - DNA, 68 bp, synthetic construct
  source 1..68: mol_type = other DNA; note = Mutation sequence of MTAP from examplary example 4, 236
atcctcgcccaaatctcatttgtattttagccccaataatccccacgtcatggaagggacctggtggc

SEQ ID NO: 231 - DNA, 69 bp, synthetic construct; identical to SEQ ID NO: 221
  source 1..69: mol_type = other DNA; note = Mutation sequence of MTAP from examplary example 4, 236

SEQ ID NO: 232 - DNA, 70 bp, synthetic construct
  source 1..70: mol_type = other DNA; note = Wild type sequence of DYRK1A for comparing, from examplary example 4
aggtgacccatttccattcaagggttttagaagcacatcaaggacattctaaggatgattgacttacaca

SEQ ID NO: 233 - DNA, 59 bp, synthetic construct
  source 1..59: mol_type = other DNA; note = Mutation sequence of DYRK1A from examplary example 4, 218
aggtgacccatttccattcaagggttttagaagcacatcaaggatgattgacttacaca

SEQ ID NO: 234 - DNA, 65 bp, synthetic construct
  source 1..65: mol_type = other DNA; note = Mutation sequence of DYRK1A from examplary example 4, 218
aggtgacccatttccattcaagggttttagaagcacatcaagtctaaggatgattgacttacaca

SEQ ID NO: 235 - DNA, 66 bp, synthetic construct
  source 1..66: mol_type = other DNA; note = Mutation sequence of DYRK1A from examplary example 4, 218
aggtgacccatttccattcaagggttttagaagcacatcaaggactaaggatgattgacttacaca

SEQ ID NO: 236 - DNA, 64 bp, synthetic construct
  source 1..64: mol_type = other DNA; note = Mutation sequence of DYRK1A from examplary example 4, 218
aggtgacccatttccattcaagggttttagaagcacatcaagctaaggatgattgacttacaca

SEQ ID NO: 237 - DNA, 67 bp, synthetic construct
  source 1..67: mol_type = other DNA; note = Mutation sequence of DYRK1A from examplary example 4, 218
aggtgacccatttccattcaagggttttagaagcacatcaaggacataaggatgattgacttacaca

SEQ ID NO: 238 - DNA, 69 bp, synthetic construct
  source 1..69: mol_type = other DNA; note = Mutation sequence of DYRK1A from examplary example 4, 218
aggtgacccatttccattcaagggttttagaagcacatcaaggacatctaaggatgattgacttacaca

SEQ ID NO: 239 - DNA, 66 bp, synthetic construct
  source 1..66: mol_type = other DNA; note = Mutation sequence of DYRK1A from examplary example 4, 218
aggtgacccatttccattcaagggttttagaagcacatcaaggacaaaggatgattgacttacaca

SEQ ID NO: 240 - DNA, 66 bp, synthetic construct
  source 1..66: mol_type = other DNA; note = Mutation sequence of DYRK1A from examplary example 4, 218
aggtgacccatttccattcaagggttttagaagcacatcaagttctaaggatgattgacttacaca

SEQ ID NO: 241 - DNA, 59 bp, synthetic construct; identical to SEQ ID NO: 233
  source 1..59: mol_type = other DNA; note = Mutation sequence of DYRK1A from examplary example 4, 233

SEQ ID NO: 242 - DNA, 64 bp, synthetic construct; identical to SEQ ID NO: 236
  source 1..64: mol_type = other DNA; note = Mutation sequence of DYRK1A from examplary example 4, 233

SEQ ID NO: 243 - DNA, 66 bp, synthetic construct; identical to SEQ ID NO: 235
  source 1..66: mol_type = other DNA; note = Mutation sequence of DYRK1A from examplary example 4, 233

SEQ ID NO: 244 - DNA, 65 bp, synthetic construct; identical to SEQ ID NO: 234
  source 1..65: mol_type = other DNA; note = Mutation sequence of DYRK1A from examplary example 4, 233

SEQ ID NO: 245 - DNA, 66 bp, synthetic construct; identical to SEQ ID NO: 239
  source 1..66: mol_type = other DNA; note = Mutation sequence of DYRK1A from examplary example 4, 233

SEQ ID NO: 246 - DNA, 67 bp, synthetic construct; identical to SEQ ID NO: 237
  source 1..67: mol_type = other DNA; note = Mutation sequence of DYRK1A from examplary example 4, 233

SEQ ID NO: 247 - DNA, 50 bp, synthetic construct
  source 1..50: mol_type = other DNA; note = Mutation sequence of DYRK1A from examplary example 4, 233
aggtgacccatttccattcaagggttttagaaggatgattgacttacaca

SEQ ID NO: 248 - DNA, 59 bp, synthetic construct; identical to SEQ ID NO: 233
  source 1..59: mol_type = other DNA; note = Mutation sequence of DYRK1A from examplary example 4, 236

SEQ ID NO: 249 - DNA, 64 bp, synthetic construct; identical to SEQ ID NO: 236
  source 1..64: mol_type = other DNA; note = Mutation sequence of DYRK1A from examplary example 4, 236

SEQ ID NO: 250 - DNA, 66 bp, synthetic construct; identical to SEQ ID NO: 235
  source 1..66: mol_type = other DNA; note = Mutation sequence of DYRK1A from examplary example 4, 236

SEQ ID NO: 251 - DNA, 65 bp, synthetic construct; identical to SEQ ID NO: 234
  source 1..65: mol_type = other DNA; note = Mutation sequence of DYRK1A from examplary example 4, 236

SEQ ID NO: 252 - DNA, 66 bp, synthetic construct; identical to SEQ ID NO: 239
  source 1..66: mol_type = other DNA; note = Mutation sequence of DYRK1A from examplary example 4, 236

SEQ ID NO: 253 - protein, 1290 aa, synthetic construct
  source 1..1290: note = Full CRISPR enzyme sequence used in examplary example 4
MKIEEPKKKRKVKKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQKKRPAATKKAGQAKKKKPAAKRVKLDGGGGSGGGGSGGGGSPAAKRVKLDLEHHHHHH

SEQ ID NO: 254 - protein, 1391 aa, synthetic construct
  source 1..1391: note = Full CRISPR enzyme sequence used in examplary example 4
MKIEEPKKKRKVTFYNIILKKYKNIEGKMNQNTSISQFTGLYPVSKTLRFELKPMGKTLEKIKETGIIENDKRRHNDYFDAKKIIDTYHKYFIDAALSKFSRIDWNPLKEAIEGSLDKSDASKKKLEKIQTEFRKKIAKALTTHDHYKELTASTPKDLFLKVFPDHFGKQPAIDTFDGFSSYFTGFQENRQNIYSDEAISTAIPYRLVHDNFPKFLSNIEVYKTLKDNAPSVLSDAENELKDFLNGKSLANIFELNAYNDVLTQSGIDFFNQVIGGISGEGGEKKTRGINEFSNLYRQQHPEFAQKRLATKMIPLYKQILSDRETKSFILESYSTDSQVQESVKEFFESQILNCDIAGRKVNVLKELSSLIKRITEFDLGSIYVNQEELSNISLELFKSWNTINAVLFKDAENRIGSAEKAANKKKIDAWMKSNEFSIATLNLAIAESDSEEISRVKIESYWNDFEAKVQSILCGDNRRNLDEFLSATFNENNALREDSEIIGKLKAFLDALIEIMHSIKPLISDAENRDLSFYNELMPLYDQLSLVVPLYNKIRNYATQKLTESEKFKLNFDCPTLADGWDQNKEKDNKSILLRKDGLYYLGVMNTNDMPKIEEINALGNEDCYEKMIYKQFDCLKQIPKCTTQTKAAKAHFEAGKTEDFVIKDKSFNGPFVISEYIWKLNNYVWNGEKFVLKFGDADKRPKQFQMGYYKETNDLLGYKKALADWIDFCKKFVKTYISASGYNYDFLDSDKYNSLDEFFSYLKTICYKITFSKIPTSQIDEWVNEGKLFLFQIYNKDFAPGAKGSPNLHTLYWKSVFSPENLKDVVVKLNGEAELFYRPSSVKKPYSHKVGEKLVNRIGKDGLPLPESVFGELFRYFNGKLDGELSDEAKKYLDVAVVKDVKHEIVKDRRYTQDKFEFHVPLTLNFKADSKNEYMNERVRHFLKDNPDVNIIGIDRGERHLLYMTLINQKGEILKQKSFNIVESVNYQAKLIQREKERDAARRSWSSVGKIKDLKEGFLSQVIHEITTTMIENNAIVVLEDLNFGFKRGRFCVERQVYQKFEKMLIDKLNYLVFKNKPEGDVGGVLKGYQLAEKFDSFQKLGKQSGFLFYIPAAYTSKIDPTTGFANLFNMTELTSAEKKKEFLSHFEDITYDGKNDRFLFSFDYKNFKCFQTDYIKKWTVYSQGKRIVYDKESKSAKEISPVEIIKAALAKQNIALTDQLDVLSAINSAEASPKSASFFGDICYAFEKTLQMRNSIPNTDEDYLVSPVLNKKGEFYDSRSCGDTLPKNADANGAYHIALKGLYLIKNVFDAGGKDLKISHEDWFKFAQSRNSKRPAATKKAGQAKKKKPAAKRVKLDGGGGSGGGGSGGGGSPAAKRVKLDLEHHHHHH

SEQ ID NO: 255 - protein, 1408 aa, synthetic construct
  source 1..1408: note = Full CRISPR enzyme sequence used in examplary example 4
MKIEEPKKKRKVAFVICYNTYSRTYLEKEIISMKTIESFCGQKKGYSRSITLRNRLIPIGKTEENIRKLKLLDKDIDRSKAYVEVKALIDDFHRVFIEDVLSKTELPWEPLYDQFELYQNEKDKQKKNKIKKDLEELQKGLRKSIVKQFKADDRFDKLFKKELLTEFVPSVIKNDDSGTISDKQAALDIFKGFATYFTGFHQNRQNMYSEEAQSTAISNRIVNENFPKFYANIQTFKYLEENFPQIISDTEQSLSDFLGDKKLKDIFSIDGFNKVLSQSGIDFYNTVIGGISEEAGTQKVQGLNEKINLASQQLSSEEKHKLKKKMTVLYKQILSDRSTASFIPVGFEKSEEVYDSVKEFKELSLDKNIAAINTIFSRDDYDLTRIFVPAKEITEFSLKLFGHWSILQDGLFLLGKDNAKKDLSEKQITELIKEIAKKDYSLAELQNAYERWTKENDIPVEKTVKNYFRLAELRTDEKTKEKNFTDIQKELEKAFVQIDFDKKENLIKEKEAATPIKNFLDEVQNLFHYLKLVDYRGEEEKDSDFYAKYDQVLQALAEIIPLYNKVRNFVTKKPNEVKKVKLNFECSSFLSGWGTDYGTKEAHIFIDDGKYYLGIVNEKLSKEDIKFLEEKSPRMIKKVVYDFQKPDNKNTPRLFIRSKGTSYAPSVSQYDLPIESVIEIYDKGLFKTEYRKVNPSVYKESLVKMIDYFKLGFTRHESYKHYNFSWKDSKDYNDISEFYADVMNSCYQLKFEYINYDNLLSLVDAGKLFLFQIYNKDFSTGKDGANGSTGKKNLHTLYWENLFSEENLKDICLKLNGEAELFWRDVNPNIKNICHKKGSILVNRTTSDGKVIPEDIYQEIYKFKNPDKQEKDFKISDEAKALLENGKVVCKEAKFDITKDRHFTQQTYLFHCPITMSFKAPEITGRKFNEKVQSILKANPDVKIIGLDRGERHLIYLSLINQKGEIELQKTLNLVDQVRNDKTVSVNYQEKLVHKEGERDKARKNWQSISNIKELKEGYLSNVVHEIAKLMVENNAIVVMEDLNFGFKRGRFAVERQVYQKFENMLIEKLNYLVFKDKAVTEPGGVLNAYQLTDKSANVSDVGKQCGWIFYVPAAYTSKIDPKTGFANLFYTAGLTNVEKKKEFFDKFEAIRYDSKTDSFVLSFDYKDFSDNADYKKKWSLYSRGERLVFSKAEKNVISVNPTENLKALFDKQGINWKSEENFIDQINAVQAERENVSFFDGLYRSFTAILQMRNSVPNSSKMEDDYLISPVMAEDGTFYDSRKEAAKGKDEQGKWISKLPVDADANGAYHIALKGLYLLQNDFNVNDKGYIDNISNADWFEFAQEKNYAKKRPAATKKAGQAKKKKPAAKRVKLDGGGGSGGGGSGGGGSPAAKRVKLDLEHHHHHH

SEQ ID NO: 256 - protein, 1440 aa, synthetic construct
  source 1..1440: note = Full CRISPR enzyme sequence used in examplary example 4
MKIEEPKKKRKVQNIESFTNLYSLTKVVKFNLDPIPETKKWLKAFGKVFTHNHSENLEDNFFKLDLKIEDAKRIIQDVLNSVHEAIITQALESEEAKSINISSYYELSNQRNHDLSDLEQRLREQIKVCFIKQLKDFCSEVSPKKKNSDDENGLPDLLKMLGSSNMLDYLSKSASKYSSPIHDKKSIEDACKDLKGFWGVLDKYIKNRENYYVFDKEQDTAVATRIVSDLLPMFCKNCKQYLTNKALYDSIFTSLKSSGIIMQMINPKTNEMRSIESFDSSIYNISGFYKYISQSGIETYNAIISRNNILVNLYNQQQKKANRINGFVELHKQIGCRESESTERIVICKDWDSTESKELGISMESMIKRIICMVNETIVPTEPNILHPRVYNLISWLENAVDWDGIYVSDKALSIISSHYLEDWYSLQQLLLNKESVGTYNQRKESNEQFKLNSAVELMPVFRALDCKDFDSVFKKSIYNDYSDILNKNLPPSRNLINMLCHDVKQIINNELPIYTKCITQLLTTKISKVQREGFFKQEEIIDIIRDYLDCVLSIARFVYFFSVRQNKIKGAPYNMELLQIVSSLLSTELGNRRLWYDEVRNYLLKSPQDEVKENKLKLNFNSSSLLKGWSIGEERTKLSLLLKNEGCYYLCILKNDKTCKKLFDINTQNNIYIEHGNAYRMMLRNLSFKTLVGKGYVSMFGTKYSEEDDEVAIQHAKQLIRQNYLKNYPELSAVITKPHSTKSAFFADVDSILKDYAQCYYNPINWNLIEKFANEGNIYVFRIHAKDYKDTSKGKKDLQTIYWEDVLSGHSSHSLSAYGEVFRRDAISTDKTKPFIHKKGSVLINKRDIDGNSIPNSIYIIIYALLNKVPLPKTAEEKDIERAKSLIEKNIIISKESWMNIIKDARFYGEEKYFLHNPICFNYKSIGFKSEDFSKDIPYHVINPIIQELMHNSCAPTSFIGIDRGEKNLIYACKINSEGSILFCNHYNIVHGINYLEKLEERADSRLKAKRSWKQQESIKNLKEGYISHVVHQLLEDVISVGNENLTPSYIVLEKLSREMKRGRQKIEKQVYQKFELALASKLGFYVNKDIEEEKPGSIQMPLQFVPPVNTFDQIDKKDSFGIMLYTRANYTSVTDPLTGWRKTIYITNGNNEDILKELTEAFDDIFFDGVDYVFSYVEKNSGQRWNIYSGNHGKSLDRFEYNKDTKCYESYNIVEVLDVLFADFNKSSSLLEQMRRGVKLKKAEENRTAYDSLRKAINMIQQIRNTGNEIKDNNFLQSPIRKDGKHFDTRNANDFRDLNLHFIKDADANGAYNIARKGLIMDAHYKYWMEQGKPNVKKKNKKEKEVSALSLYVSDREWDMWLLNREMWEKHLSEFSLRKQDKRPAATKKAGQAKKKKPAAKRVKLDGGGGSGGGGSGGGGSPAAKRVKLDLEHHHHHH

SEQ ID NO: 257 - protein, 1368 aa, synthetic construct
  source 1..1368: note = Full CRISPR enzyme sequence used in examplary example 4
MKIEEPKKKRKVSRDFSQFTNQYKLSQTLQFSLIPQGRTREFMDSFLIHDQSRSDAYPEVKKILDNAHKALIERTLGSISQRIHSLSDKEKKKFASILNGDDINWLQLDKAVENEAENGKDATDAVRLEYCRLLAMLFSYDDAFESLVKNATPDEYFKNELKKMALSDDDTARFLEKFKGFACYFKGFQENRRNIYSQEAQSTAAPHRAIYDNFPKMKRCIKIFNHIKDAYPEILTTATTELAELLNGQSIDSVFSIQEYSNYLSQKGIDKFNSILGGYTTKDGQKVKGINECINLYRQQHEDARKDRSFSTMPLLFKQILSDRSSLSFIPQMFQNDQELFNTLKEFIQQLDHVTTADGIDRPLIPSFRDCLDNISIDDGILVSASSLTEISMRASGSWNAIPDAMENWAAVNISTKALREKWLKKNSYSLTELDALHLEKETDEKRISCDILDYWKGKKALDCYDRYDALLKDITSLMSAPVNDIPLAERKDDVALIKDYLDSLMDILHLGKPLYCQDILDRNEQFYSLFDGLYEQLQIVIPLYNRIRNYLTQKADTTKKVKLMFDSPTLADGWDQNKEQTNNAIILLKDGKYYLGIMNPAKKPVMSKMVVQKAESEVEVYQKMVYKYLPGPNKMLPKVFFSTKGQKDFNPPKSLLEKYEAKMHIKSSPDFSLAFLHELIDYFKASIAKHPDWSKFGFKFSPTASYQGIDEFYREISNQGYRLSFEIIPKEKIDDLVNNEQLLLFQIYNKDFAPGATGTPDKFTIYWKMLFSPENLSNVILKLNGEAELFWRKQVIAKPFSHKLNEKMVNRTYVDGTDSTGRPIVKHMPEKVHAVIFDYVNGKNDDIHKKEYQDALALYDSGKVIVKDVTHPIIKDRRYTQESFSFHVPITINFTAPDKPSKFNDSIIAFLKNNPDVKIIGIDRGERNLIYFSLIDQQGNILKQGSLNTVNPRTEARIEQVNYHELLNQKEKERDEARKSWSSIGKIKDLKAGYLSQVIHQIAPLMVENNAIVILEDLNFGFKRGRFAIEKQVYQNFEKALITKLNYLVFKEIQDYNTAGSVLIGYQLTDKFVSFEKMGKQTGALFYVPAAYTSKIDPTTGFANLFNTRKCTNVESRKVFLMTFDAIKYNAVRKSFAFSFDYAKFQTSAKSFQNKWTVYTSQKRLVFNPLTRQDTAVNPTQMIVDALNKRGVSLSDEFDLKAYLQNTDAIKANAEFFKTIFIALEYTLQMRNSNSSSGEDYIESPVLNKSGEFFRSSEDNPALPIDADANGAYHIAMKGLFLLLNVFNQGKKDLKIEHQAWFEFMQKRNMKRPAATKKAGQAKKKKPAAKRVKLDGGGGSGGGGSGGGGSPAAKRVKLDLEHHHHHH

SEQ ID NO: 258 - protein, 1300 aa, synthetic construct
  source 1..1300: note = Full CRISPR enzyme sequence used in examplary example 4
MKIEEPKKKRKVAYSDFIGMYPLQKTLRFELKPQGRTMETLKEKDYLKNDLYRASQYAGMKKLIDDYHRSFIDESLMTSEIDWQPLADALDARKNNDEKSKKQLEILQDQYRRQVSKLFTGKEVFKSLFSKEVLDLVREKCVKERDDSKIQICDAFNRYSVYFTGFHENRRNFYVSDKQSTSISKRLVDDNFPKFLENIRIYEKIEVECPEIIEKVKNELKLTDLKKYFSVSEFNRFVRQCGIDDYNWILGGHVGEEAHVKGFNGFLNEAYQQGLLKTRVLMRPLYKMILSISESKSVLPYQFKDEKEMMDAVVSFIQKANEGIIAQAGDLLKSVESTDLSRVFIPDKQLTELSQILFGDWGALGTMMREYKAYDLGDPTMSKTMKKVDNWMKSSEFSLEDVKKAVLHADQEFTLENYEVRVDQVCSKLIETNAVSELKSVKIDGTDGETRNRSAECLRCILEGYQDLLHCMNMFKVSDELDRDEAFYSEFDLILGELQEIIPLFNKVRNYCTKKAYNETKVKLKLSTPTLADGWDLDKEKDNKAVILRSGGQYYLGVNCDKGFLDFSKYETDDEECFEKLNYKLLPGPNKMLPKVFFSKSWKSEHTIPERIQDGYDRGIFKKGPEFDLMFCHELIDFYKESISIHPVWSKFDFRFAETSDYQDISQFYADVQRQGFKMSFSKVSKSEVYKLVDEGKLMLFKIHSKDFSENSRGMPNMHTLYWRALFSEENLANIRIKLNGEAELFFREKSIDTPVVHKSGSVLVNKTFNGKHVPDKIYYELFRYRNGIITALSPEAKEYEGAIVSSVANYDIVKDRRYTVDKLFFHVPLTFNFGVPDRSKDINRMVLEHAIKSKDTKIIGIDRGERNLIYISMIDKEGKILLQKSLNVIDGVDYHGNLDVREHENKEARRNWTKVAGIKDLKEGYLSAAVSQIARMVVENDAIVVMENLNYGFKHGRFKIEKQVYQKFESMLIDKLNYLVFKDKEDFTSDGRAMRGYQLTKSIDSVKEIGTQTGVIFYVNAAYTSKIDPTTGFINQIQFSTKSTLSAKRDLLCNIDSIRYDPSEECYIFRIDYTKVGDRTKEFKNVWDIYTRGDRIVYSTKERCDKHLHPTEIMTKALESAGIGKEGELKEKICSADSKVIEAVVYALDLTLRLRNEDRETDYILSPVRNADGKFFCTLDGDLSLPLDSDANGAYNIALKGLLMLKMTEESNGPDADKYDISLVTNSDWLTFCQTGHRTWKSKRPAATKKAGQAKKKKPAAKRVKLDGGGGSGGGGSGGGGSPAAKRVKLDLEHHHHHH

SEQ ID NO: 259 - protein, 1300 aa, synthetic construct
  source 1..1300: note = Full CRISPR enzyme sequence used in examplary example 4
MKIEEPKKKRKVTNFYSSFTNLRSVSKTLRFSLSPVGKTRENIRSANIIPQDEKRMQDYLVLKNVFDAYHKAYIDQHLCRIQGDWSELANALDERQSAPDADALNKMQETYRKEISNLLSGKGTGKNQYLFPADGDGGSLLEMLDPADMIKQTVKEKKDAEKGNRPPQYLIPEFGQNIKEVEQFNRFAGYFEDYKKTRENIYASEGSTSVAHRIVHENFPKFMSNVHTFRRLPESLRQKFEHDLSELLDGASLETMFDVDYFNVVLAQAGIERYNTLLGGKKEESGVKLQGLNELCNLEFQQGNLPKKVVFVPLYKQILSDREKASFLPEKFKSVEEMLESIADYRQSLSNLMASQHCLLDENMSTGYCDAGHVYIRKSQLASLSMLCFNEWSTFRTAVEAKDVYTLSELLQAAGKDILEDVRRELNFSLDSIDEAGEALLPVLEKQTLTEDDYAIIKEYLDAVIAAQHILKVFEAEDDAEKDPVFYGVADILNDALRQGVRLYDRVRNYATGKPYAEDKFRLTFFAPTLLNGWDKNKERDNCCMLFRKDGLYYLGILNAKNKPLLDEEAEIEGASYEKMVYKYLPEPNKMLPKVFNKSQKWIAAHGLDDDIRDGYEKGLHKVGQSFDKAFMHKLIDYYKACISEYEDWATFRFRFSATDTYQTMADFYNEIAAQSYKVFFTKVSEQQINAAVEAGQLYLFQIYNKDFSPYSHGAPNLHTLYWRELFSAENLQNPLVKLNGEAEIFYRHPSIANPFRHQKGDVLINKTDAAGRTVPEDRYRAAATDAANGMPVQALRASYPELIFKVADREIVKDKRYTKESFAFHVPLTLNNDNNAGFKLNMAVCEKLQSDGEYYIMGIDRGERHLLYISVIDRNGRIVEQRSLNVVGKNRVDYQAKLDALERQRQEARKNWRAITAIKELKEGYLSMAIHEITELMLQYPCILIMEDLNFGFKRGRFRVEKQVYQKFEKMLIEKLGFWMQKDKAADAAGGIRHAYQLTESFQSFQKLGKQSGVIFYVPANYTSKIDPKTGFVNLFGSRQLTYTSVSAAKAFFGTMREIGYDAQRDCYRFAFRYSDYDLYQQDHRDSWTVFTAGKGRIAHTVKNGRHTFEEIDVTERMKELFQRFGLDPYAADLRGAIDAADKKDFFSELLWLFKVTVQLRYEDSEQDFILSPIEKDGRFFDSRQAKKDEPQDGDANGAYHIALQGLRLARERIKDGRVQKDPKGKQTLNWLRFAQRKDYLEKRPAATKKAGQAKKKKPAAKRVKLDGGGGSGGGGSGGGGSPAAKRVKLDLEHHHHHH

SEQ ID NO: 260 - protein, 1288 aa, synthetic construct
  source 1..1288: note = Full CRISPR enzyme sequence used in examplary example 4
MKIEEPKKKRKVKKIDHFTGCYSVSKTLRFKLIPVGKTQENIVAKRLLEEDENRAEDYKAAKKIIDRYHKSFIDDVLKSVKLSHLNEYASLFLKGNRSSDENETLEKLEETLRNEISKAFTENEGFALLFKKELIEDELPERLEGDELRIINSFKGFTTAFSGFHENRRNMYAADKKSTAIAFRCVNENLQRFLQNIACFEKIKCILDADMCKTIESDILSGDYSIRDFFVVDFYNLVLTQQGIDLYNALIGGVVKEDGRKIQGLNECINLHNQQNKDRLPHLKPLYKQVLSDRTGESFYGEGLTSDEELYDAMRNTVGFESMIYKNYRKAEELFASLDLGAFDGIYVKNGPAVTNISNDAYGHWGNFTEKWNEQYDLANEKSNAKDLEKFEEKRRKDFKKIESFSLGEIIKLMEDNEKLPTAVVKGIKDRIAAVEMAYANAKTVLTEGFKTEKQLKKDEVTVGRIKALLDSLKELESFLKQFKGTGKESGRNELFYGDYTLYCDMLSEVDELYNSVRNYVTQKPYSTDKFKLYFQNPQFLNGWDRNKETDYQSVILRNNDQYYLGIMDSKSRDIFRRVPESDEKGYEKMVYKQIPNASKYLSSKQINPQNPPEEIVRILEKKKTDNRSLTADEIKTFISYIQDDFLKNYSFLRDEKGKEYFDFKFKKPEAYRRLNDFFDDVDKQGYSVKFSDVPKTYVDSLVEKGELYLFRIYNKDFSEASHGTPNLHTMYFRSLFDGANQGVIRLAGEAEMFMRRASIKKDELIVHPANTAINNKNPLNPRKTRTLPYDVIKDRRFSEDQYELHMAISINKIPENGIKINHDVRKFLREDENPYVIGIDRGERNLLYVVVIDGKGKIVEQHSLNEIINEIHDVKIVTDYHFLLDQKEKDRLAARQDWTTIENIKELKEGYISQVVHKICELVEKYDAMIALEDLNSGFKNSRVKVEKQVYQKFEKMLIDKLNYLVNKKTPIEEKGSVIHGYQLTNKFESFKTMGTQNGIMFYIPAWLTSKIDPTTGFSDLLKPKYTNMESARSFVGTFDFIRYNGDEGYYEFGIDYSKFGCDADHRKKWTLCSQGSRIRTFRNPLKNNEWDNEEVNLTEMFDTLFEKYKVNGKSGDLRDKICGVTEADFYKEFYGLLRLMLQMRNSITGRTDVDYLISPVKNRHNDFFDTRNADKTQPMDADANGAYNIARKVLWAIEVFKKTEEDKLDKTKIAISNKEWLKFAQTMVDKRPAATKKAGQAKKKKPAAKRVKLDGGGGSGGGGSGGGGSPAAKRVKLDLEHHHHHH

SEQ ID NO: 261 - protein, 1319 aa, synthetic construct
  source 1..1319: note = Full CRISPR enzyme sequence used in examplary example 4
MKIEEPKKKRKVKDLKQFIGIYPVSKTLRFELKPIGKTLDWIRERNVVEKDERKSQDYPVVKQHIDEYHKVCISESLKNVCFDWKPLKEAIEKHHKLKSDESKKQLETEQTNMRKQIVLAIKRFKHYPELTAATPKDLIQNVLPEITNDSSLNSFNRFASYFKGFQENRKNIYSLDAISTGVPFRLVHDNFPKFLSNIDIYENIKSTAPETLELAAKELQPFLEGVMLDDIFSLDFYNSLLTQDGIDFFNRVIGGVSEEGKQKFRGINEFTNLYRQQHPELSANKKAMTMIPLFKQILSERETLSYIPKQIENENELTETIKQFYSHITNFERNGKTINILNELVRLLGQVTSYNQDRIFVSSKYITDISQKLYGRWNAINDYLYERAVEMFGDVSVAKNKKLVDNYIDKDVYSASELGFDENNNLSTFYSELPQIVGSVNGNWIQFCEWCKIDKKQKFLNNAEGTEIIKKLLDAIMEVMHKCNVLVVSEEYDLDKDFYNNFFPLFTELENIVFVYNRVRNFLTKKPSDVKKFKLNFGVPTLCDGWDLNKEKDNKAILLFKDNRSYLGIMNPEDYPKIKESNDSGDGYFSKMVYKLLPGPNKMLPKVFFSKKGRETFSPSDRIIDIYESGAFKKGSSNFSIVCLHELIDFYKDAINRHEDWSKYNFKFSPTDSYKDIGAFYNEISKQAYKIRFTNISEQEINQLVDKGQLYLFQLYNKDYASGAHGRKNLHTLYWENLFKEENLNNLVLKLNGQAELFYRPQSINEPVAHKVGSIMLNRRDKSGMPIPETVYRSLYLYFNGKKKEEELDSVEKSYIDNVIVKKVTHEIVKDKRFTKPEFFFHVPITFNSNADGNEFINENVIDYITDNPDVNIIGIDRGERHLIYLTLINQQGEILKQKTFNTVGTYNYHAKLEQREQERQEARKSWQSIGKIKDLKEGFLSAVIHEIALMMAEYNAIVVLEDLNFGFKRGRFKVERQVYQKFEKMLIDKLNYLAFKDRKSDETGGILRGYQFAQKFVSFQRLGKQSGFLFYVPAAYTSKIDPVTGFVNHFNFNDITNAEKRKQFFCKMERIEMKNDNIEFEFDYRKFKTYQTDFQNVWTASTFGKRIVMKTDESGRKQMADYYPTNEILAAFKKYGYELTNGTDIKSLLQDIEADQKSASLFSNLFYAFQKTLQMRNSNHATEEDYILSPIAKHGYHFCSTEEANKGKDEFGNWLSKLPVDADANGAYHIALKGLYLLKNPQTKKIEHEKWFKFMVEKPYKKRPAATKKAGQAKKKKPAAKRVKLDGGGGSGGGGSGGGGSPAAKRVKLDLEHHHHHH

SEQ ID NO: 262 - protein, 1332 aa, synthetic construct
  source 1..1332: note = Full CRISPR enzyme sequence used in examplary example 4
MKIEEPKKKRKVKEFTNLYSLSKTLRFELQPIGKTLENINANGFLTNDAHRAESYKKVKKLIDEYHKDFIDKVLGDFKLNAEHLQLYYDLYSQTTKDKQFKDVQDKLRKAIAGALKADDRYKTIDKKELIRQDMKKFLNNDTDKALLDEFYEFTTYFTGFHENRKNMYSDEAKSTAIAYRLIHDNLPKFIDNMAVFKKIANTSVADNFSAIYKAFEECLNVNSIDEMFSLDYYNDILTQTQIEVYNSIIGGRTLEDGTKIQGINEYVNLYNQQPANKKERLPKLKPLFKQILSDRVQLSWLQEEFNTGADVINAVKEYCVSYFDNVEESIKVLLTSISDYDLSKIYITNDLAITDVSQRIFGEWSIIPNAIDFRIRKDNPQRASEKEEKYSERISKIKKQSKSFSLAYINECLSELKDTDIADYYTTLGAINTESKQEPSIPTSIQVHYNALKPILDADYPREKNLSQDKLTVMQLKDLLDDFKALQRFIKPLLGNGDEAEKDEKFYGELMQLWEMIDSITPLYNKVRNFCTRKPFSTEKIKVNFENAQLLDGWDENKENTNASIILRKDGMYYLGIMKKEHRNILAQTMPSNGDCYDKVVYKFFKDITTMVPKCTTQIKSVKEHFANSNEDYTLFDKDKFLTPVVITKEIFDLNNVLYDGVKKFQIGYLNNTGDNQGYNHAVEIWKSFCMQFLKAYKSTSIYNFSSIEQNIDKYNDLNSFYGAVNLLLYSLTYRKVSVDYIHQLVDDDKMYLFKIYNKDFSPFSKGTPNMHTLYWKMLFDECNLSDVVYKLNGQAEVFFRKKSINCQHPTHPANKPIDNKNANNPKKQSTFEYDLIKDKRYTVDKFMFHVPITLNFKGIGNGDINMQVREYIKASDNLHFIGIDRGERHLLYICVINSKGKIVEQYSLNEIINNYKGTEYSTDYHTLLSEREKKRKEERSSWQTIEGIKELKAGYLSQVIHKISQLMIKYNAIVLLEDLNPGFKQGRQKVESSVYQQFEKALIEKLNYLVDKNKDVNEIGGLLHAYQLTNDPKLPNKNSKQSGFLFYVPAWNTSKIDPVTGFVNLLDTRYENVAKAQTFFNKFDSIRYNKENDCFEFCFDYTNFTAKAEETRTKWTLCTYGTRIETFRNAEKNSNWDSKEIDLTTEWKSLFAKHNISLNANLKEAILLQANKNFYADILHLMKLTLQMRNSVTGTDIDYMVSPVANESGEFFDSRNAKEGLPVNADANGAYNIARKGLWLAQQIKDANDISDVKLAITNKEWLQFAQTKPYLKDKRPAATKKAGQAKKKKPAAKRVKLDGGGGSGGGGSGGGGSPAAKRVKLDLEHHHHHH

SEQ ID NO: 263 - protein, 1353 aa, synthetic construct
  source 1..1353: note = Full CRISPR enzyme sequence used in examplary example 4
MKIEEPKKKRKVKDLKQFSNLYKVSKTLRFELQPIGKTKENIEKTGILARDEQMAKDYMLVKGFIDEYHKQFIKDRLWNFKIPLKSEGHLDSLEEYQMFYELSKRDDAQEAAFTEVKDNLRSIIAKRLTKSSSAFDRIFKKELIREDLINFLENEEDKEIVRQFADFTTYFTGFHENRRNMYDAEEKSTAIAYRLIHQNLPKFMDNLKAFAKIAETPVAEHFADIYEGWKERLNVGSIDEIFQLDYFSETLTQSHIEVYNYIIGKKILEDGTEIKGINEYVNLYNQQQKDKSKRLPFLVPLYKQILSDREKLSWMAEEFDSDDKMLSAINESYSHLHDLLMGEENESLRSLLMHIKDFNLEQINITNDISLTDISQHLFNRYDVFTNGIKDELRIIIPRKKKETDEVFEDRISKIFKNRKSFSIAFLDKLPQPVMEDGKPRSIEEYFLSLGAVNTGTTQKENLFARIENAYEDARTVLQMKDTNDTLSQNKPAVAKIKTLLDAIKDLQHFIKPLLGSGEENEKDELFYGVFQIIWDELDTITPLYNKVRNWLTRKPYSTEKIKLNFDNAQLLDGWDENKETANASIILCKNGQFYLGILNKDYRKLLGMPMPSEGECYDKVVYKFFKDITTMVPKCTTQKKDVVAHFAQSTENYILFDKKTFNAPVTITKEIYDLNNVLYNGIKKFQIEYLRSTGDKSGYEHAVFTWKSFCMQFLKAYKSTSIYNLALVEQNIDSYSDLSSFYNAVNLLLYNLSYRKVSVDYIHTLVEEGKLYLFRIWNKDFSEYSKGTPNLHTLYWKMLFDERNLCDVIYKLNGQAEVFYRKASIKPENRIIHPAHHPITNKNELNDKRTSTFEYDIIKDRRYTVDKFQFHVPITINFKAIGQSNINSIVQDTIRGGGFTHIIGIDRGERHLLYLSMIDLKGNIVKQMTLNDIINEYNGHKYKTNYHDLLTKREGDRTDARRSWDTIENIKELKEGYLSQVIHIISKMMVEYNAIVVLEDLNTGFMRGRQKIERQVYEKFEKMLIEKLNCYIDKHADPLEDGGVLHPLQLTCDAQKWKRSHQCGCLFYIPAWNTSKIDPVTGFVNLFDTRYDTREKARLFFSKFQRISYNSEKDWFEFTFDYNNFTSKAEGTRTEWTLCTYGERIHTFRNPKKNHQWDDEQIVLTENFKQLFDKYGIDIHGNLKEAISTQSDSQFFRDLLGLMKLLLQMRNSITNSEVDYLLSPVADETGHFFDSREEIESLPSNADANGAYNIARKGLWVIKKIQETAENEKFSLAISNKEWLQFAQTKPYLNEKRPAATKKAGQAKKKKPAAKRVKLDGGGGSGGGGSGGGGSPAAKRVKLDLEHHHHHH

SEQ ID NO: 264 - protein, 1221 aa, synthetic construct
  source 1..1221: note = E155R variant of NSG-01
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWRNRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQK

SEQ ID NO: 265 - protein, 1221 aa, synthetic construct
  source 1..1221: note = G531R variant of NSG-01
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMRGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQK

SEQ ID NO: 266 - protein, 1221 aa, synthetic construct
  source 1..1221: note = E155R G531R variant of NSG-01
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWRNRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMRGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQK

SEQ ID NO: 267 - protein, 1221 aa, synthetic construct
  source 1..1221: note = E155R K537R variant of NSG-01
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWRNRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNREADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQK

SEQ ID NO: 268 - protein, 1221 aa, synthetic construct
  source 1..1221: note = S1137A variant of NSG-01
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNAITGRTDIDYLISPVKNDTGNFYDSRNFEALENAVLPKDADANGAYNIARKVLWAIEQFRESDESKLEKVSIAISNKKWLEYAQK

SEQ ID NO: 269 - protein, 1221 aa, synthetic construct
  source 1..1221: note = E791L variant of NSG-01
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYLIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQK

SEQ ID NO: 270 - protein, 1221 aa, synthetic construct
  source 1..1221: note = E155R E791L variant of NSG-01
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWRNRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYLIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQK

SEQ ID NO: 271 - protein, 1221 aa, synthetic construct
  source 1..1221: note = E155R E526R E791L variant of NSG-01
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWRNRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQRPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYLIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQK

SEQ ID NO: 272 - protein, 1221 aa, synthetic construct
  source 1..1221: note = N526R variant of NSG-01
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQRPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQK

SEQ ID NO: 273 - protein, 1221 aa, synthetic construct
  source 1..1221: note = N526R E791L variant of NSG-01
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQRPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYLIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQK

SEQ ID NO: 274 - protein, 1221 aa, synthetic construct
  source 1..1221: note = E155R N526R variant of NSG-01
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWRNRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQRPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQK

SEQ ID NO: 275 - protein, 1339 aa, synthetic construct
  source 1..1339: note = Q190R S579R variant of NSG-02
AFVICYNTYSRTYLEKEIISMKTIESFCGQKKGYSRSITLRNRLIPIGKTEENIRKLKLLDKDIDRSKAYVEVKALIDDFHRVFIEDVLSKTELPWEPLYDQFELYQNEKDKQKKNKIKKDLEELQKGLRKSIVKQFKADDRFDKLFKKELLTEFVPSVIKNDDSGTISDKQAALDIFKGFATYFTGFHRNRQNMYSEEAQSTAISNRIVNENFPKFYANIQTFKYLEENFPQIISDTEQSLSDFLGDKKLKDIFSIDGFNKVLSQSGIDFYNTVIGGISEEAGTQKVQGLNEKINLASQQLSSEEKHKLKKKMTVLYKQILSDRSTASFIPVGFEKSEEVYDSVKEFKELSLDKNIAAINTIFSRDDYDLTRIFVPAKEITEFSLKLFGHWSILQDGLFLLGKDNAKKDLSEKQITELIKEIAKKDYSLAELQNAYERWTKENDIPVEKTVKNYFRLAELRTDEKTKEKNFTDIQKELEKAFVQIDFDKKENLIKEKEAATPIKNFLDEVQNLFHYLKLVDYRGEEEKDSDFYAKYDQVLQALAEIIPLYNKVRNFVTKKPNEVKKVKLNFECSSFLRGWGTDYGTKEAHIFIDDGKYYLGIVNEKLSKEDIKFLEEKSPRMIKKVVYDFQKPDNKNTPRLFIRSKGTSYAPSVSQYDLPIESVIEIYDKGLFKTEYRKVNPSVYKESLVKMIDYFKLGFTRHESYKHYNFSWKDSKDYNDISEFYADVMNSCYQLKFEYINYDNLLSLVDAGKLFLFQIYNKDFSTGKDGANGSTGKKNLHTLYWENLFSEENLKDICLKLNGEAELFWRDVNPNIKNICHKKGSILVNRTTSDGKVIPEDIYQEIYKFKNPDKQEKDFKISDEAKALLENGKVVCKEAKFDITKDRHFTQQTYLFHCPITMSFKAPEITGRKFNEKVQSILKANPDVKIIGLDRGERHLIYLSLINQKGEIELQKTLNLVDQVRNDKTVSVNYQEKLVHKEGERDKARKNWQSISNIKELKEGYLSNVVHEIAKLMVENNAIVVMEDLNFGFKRGRFAVERQVYQKFENMLIEKLNYLVFKDKAVTEPGGVLNAYQLTDKSANVSDVGKQCGWIFYVPAAYTSKIDPKTGFANLFYTAGLTNVEKKKEFFDKFEAIRYDSKTDSFVLSFDYKDFSDNADYKKKWSLYSRGERLVFSKAEKNVISVNPTENLKALFDKQGINWKSEENFIDQINAVQAERENVSFFDGLYRSFTAILQMRNSVPNSSKMEDDYLISPVMAEDGTFYDSRKEAAKGKDEQGKWISKLPVDADANGAYHIALKGLYLLQNDFNVNDKGYIDNISNADWFEFAQEKNYAK

SEQ ID NO: 276 - protein, 1339 aa, synthetic construct
  source 1..1339: note = Q190R variant of NSG-02
AFVICYNTYSRTYLEKEIISMKTIESFCGQKKGYSRSITLRNRLIPIGKTEENIRKLKLLDKDIDRSKAYVEVKALIDDFHRVFIEDVLSKTELPWEPLYDQFELYQNEKDKQKKNKIKKDLEELQKGLRKSIVKQFKADDRFDKLFKKELLTEFVPSVIKNDDSGTISDKQAALDIFKGFATYFTGFHRNRQNMYSEEAQSTAISNRIVNENFPKFYANIQTFKYLEENFPQIISDTEQSLSDFLGDKKLKDIFSIDGFNKVLSQSGIDFYNTVIGGISEEAGTQKVQGLNEKINLASQQLSSEEKHKLKKKMTVLYKQILSDRSTASFIPVGFEKSEEVYDSVKEFKELSLDKNIAAINTIFSRDDYDLTRIFVPAKEITEFSLKLFGHWSILQDGLFLLGKDNAKKDLSEKQITELIKEIAKKDYSLAELQNAYERWTKENDIPVEKTVKNYFRLAELRTDEKTKEKNFTDIQKELEKAFVQIDFDKKENLIKEKEAATPIKNFLDEVQNLFHYLKLVDYRGEEEKDSDFYAKYDQVLQALAEIIPLYNKVRNFVTKKPNEVKKVKLNFECSSFLSGWGTDYGTKEAHIFIDDGKYYLGIVNEKLSKEDIKFLEEKSPRMIKKVVYDFQKPDNKNTPRLFIRSKGTSYAPSVSQYDLPIESVIEIYDKGLFKTEYRKVNPSVYKESLVKMIDYFKLGFTRHESYKHYNFSWKDSKDYNDISEFYADVMNSCYQLKFEYINYDNLLSLVDAGKLFLFQIYNKDFSTGKDGANGSTGKKNLHTLYWENLFSEENLKDICLKLNGEAELFWRDVNPNIKNICHKKGSILVNRTTSDGKVIPEDIYQEIYKFKNPDKQEKDFKISDEAKALLENGKVVCKEAKFDITKDRHFTQQTYLFHCPITMSFKAPEITGRKFNEKVQSILKANPDVKIIGLDRGERHLIYLSLINQKGEIELQKTLNLVDQVRNDKTVSVNYQEKLVHKEGERDKARKNWQSISNIKELKEGYLSNVVHEIAKLMVENNAIVVMEDLNFGFKRGRFAVERQVYQKFENMLIEKLNYLVFKDKAVTEPGGVLNAYQLTDKSANVSDVGKQCGWIFYVPAAYTSKIDPKTGFANLFYTAGLTNVEKKKEFFDKFEAIRYDSKTDSFVLSFDYKDFSDNADYKKKWSLYSRGERLVFSKAEKNVISVNPTENLKALFDKQGINWKSEENFIDQINAVQAERENVSFFDGLYRSFTAILQMRNSVPNSSKMEDDYLISPVMAEDGTFYDSRKEAAKGKDEQGKWISKLPVDADANGAYHIALKGLYLLQNDFNVNDKGYIDNISNADWFEFAQEKNYAK

SEQ ID NO: 277 - protein, 1339 aa, synthetic construct
  source 1..1339: note = S579R variant of NSG-02
AFVICYNTYSRTYLEKEIISMKTIESFCGQKKGYSRSITLRNRLIPIGKTEENIRKLKLLDKDIDRSKAYVEVKALIDDFHRVFIEDVLSKTELPWEPLYDQFELYQNEKDKQKKNKIKKDLEELQKGLRKSIVKQFKADDRFDKLFKKELLTEFVPSVIKNDDSGTISDKQAALDIFKGFATYFTGFHQNRQNMYSEEAQSTAISNRIVNENFPKFYANIQTFKYLEENFPQIISDTEQSLSDFLGDKKLKDIFSIDGFNKVLSQSGIDFYNTVIGGISEEAGTQKVQGLNEKINLASQQLSSEEKHKLKKKMTVLYKQILSDRSTASFIPVGFEKSEEVYDSVKEFKELSLDKNIAAINTIFSRDDYDLTRIFVPAKEITEFSLKLFGHWSILQDGLFLLGKDNAKKDLSEKQITELIKEIAKKDYSLAELQNAYERWTKENDIPVEKTVKNYFRLAELRTDEKTKEKNFTDIQKELEKAFVQIDFDKKENLIKEKEAATPIKNFLDEVQNLFHYLKLVDYRGEEEKDSDFYAKYDQVLQALAEIIPLYNKVRNFVTKKPNEVKKVKLNFECSSFLRGWGTDYGTKEAHIFIDDGKYYLGIVNEKLSKEDIKFLEEKSPRMIKKVVYDFQKPDNKNTPRLFIRSKGTSYAPSVSQYDLPIESVIEIYDKGLFKTEYRKVNPSVYKESLVKMIDYFKLGFTRHESYKHYNFSWKDSKDYNDISEFYADVMNSCYQLKFEYINYDNLLSLVDAGKLFLFQIYNKDFSTGKDGANGSTGKKNLHTLYWENLFSEENLKDICLKLNGEAELFWRDVNPNIKNICHKKGSILVNRTTSDGKVIPEDIYQEIYKFKNPDKQEKDFKISDEAKALLENGKVVCKEAKFDITKDRHFTQQTYLFHCPITMSFKAPEITGRKFNEKVQSILKANPDVKIIGLDRGERHLIYLSLINQKGEIELQKTLNLVDQVRNDKTVSVNYQEKLVHKEGERDKARKNWQSISNIKELKEGYLSNVVHEIAKLMVENNAIVVMEDLNFGFKRGRFAVERQVYQKFENMLIEKLNYLVFKDKAVTEPGGVLNAYQLTDKSANVSDVGKQCGWIFYVPAAYTSKIDPKTGFANLFYTAGLTNVEKKKEFFDKFEAIRYDSKTDSFVLSFDYKDFSDNADYKKKWSLYSRGERLVFSKAEKNVISVNPTENLKALFDKQGINWKSEENFIDQINAVQAERENVSFFDGLYRSFTAILQMRNSVPNSSKMEDDYLISPVMAEDGTFYDSRKEAAKGKDEQGKWISKLPVDADANGAYHIALKGLYLLQNDFNVNDKGYIDNISNADWFEFAQEKNYAK

SEQ ID NO: 278 - protein, 1339 aa, synthetic construct
  source 1..1339: note = Q190R Y585R variant of NSG-02
AFVICYNTYSRTYLEKEIISMKTIESFCGQKKGYSRSITLRNRLIPIGKTEENIRKLKLLDKDIDRSKAYVEVKALIDDFHRVFIEDVLSKTELPWEPLYDQFELYQNEKDKQKKNKIKKDLEELQKGLRKSIVKQFKADDRFDKLFKKELLTEFVPSVIKNDDSGTISDKQAALDIFKGFATYFTGFHRNRQNMYSEEAQSTAISNRIVNENFPKFYANIQTFKYLEENFPQIISDTEQSLSDFLGDKKLKDIFSIDGFNKVLSQSGIDFYNTVIGGISEEAGTQKVQGLNEKINLASQQLSSEEKHKLKKKMTVLYKQILSDRSTASFIPVGFEKSEEVYDSVKEFKELSLDKNIAAINTIFSRDDYDLTRIFVPAKEITEFSLKLFGHWSILQDGLFLLGKDNAKKDLSEKQITELIKEIAKKDYSLAELQNAYERWTKENDIPVEKTVKNYFRLAELRTDEKTKEKNFTDIQKELEKAFVQIDFDKKENLIKEKEAATPIKNFLDEVQNLFHYLKLVDYRGEEEKDSDFYAKYDQVLQALAEIIPLYNKVRNFVTKKPNEVKKVKLNFECSSFLSGWGTDRGTKEAHIFIDDGKYYLGIVNEKLSKEDIKFLEEKSPRMIKKVVYDFQKPDNKNTPRLFIRSKGTSYAPSVSQYDLPIESVIEIYDKGLFKTEYRKVNPSVYKESLVKMIDYFKLGFTRHESYKHYNFSWKDSKDYNDISEFYADVMNSCYQLKFEYINYDNLLSLVDAGKLFLFQIYNKDFSTGKDGANGSTGKKNLHTLYWENLFSEENLKDICLKLNGEAELFWRDVNPNIKNICHKKGSILVNRTTSDGKVIPEDIYQEIYKFKNPDKQEKDFKISDEAKALLENGKVVCKEAKFDITKDRHFTQQTYLFHCPITMSFKAPEITGRKFNEKVQSILKANPDVKIIGLDRGERHLIYLSLINQKGEIELQKTLNLVDQVRNDKTVSVNYQEKLVHKEGERDKARKNWQSISNIKELKEGYLSNVVHEIAKLMVENNAIVVMEDLNFGFKRGRFAVERQVYQKFENMLIEKLNYLVFKDKAVTEPGGVLNAYQLTDKSANVSDVGKQCGWIFYVPAAYTSKIDPKTGFANLFYTAGLTNVEKKKEFFDKFEAIRYDSKTDSFVLSFDYKDFSDNADYKKKWSLYSRGERLVFSKAEKNVISVNPTENLKALFDKQGINWKSEENFIDQINAVQAERENVSFFDGLYRSFTAILQMRNSVPNSSKMEDDYLISPVMAEDGTFYDSRKEAAKGKDEQGKWISKLPVDADANGAYHIALKGLYLLQNDFNVNDKGYIDNISNADWFEFAQEKNYAK

SEQ ID NO: 279 - protein, 1339 aa, synthetic construct
  source 1..1339: note = Q190R S579R Y585R variant of NSG-02
AFVICYNTYSRTYLEKEIISMKTIESFCGQKKGYSRSITLRNRLIPIGKTEENIRKLKLLDKDIDRSKAYVEVKALIDDFHRVFIEDVLSKTELPWEPLYDQFELYQNEKDKQKKNKIKKDLEELQKGLRKSIVKQFKADDRFDKLFKKELLTEFVPSVIKNDDSGTISDKQAALDIFKGFATYFTGFHRNRQNMYSEEAQSTAISNRIVNENFPKFYANIQTFKYLEENFPQIISDTEQSLSDFLGDKKLKDIFSIDGFNKVLSQSGIDFYNTVIGGISEEAGTQKVQGLNEKINLASQQLSSEEKHKLKKKMTVLYKQILSDRSTASFIPVGFEKSEEVYDSVKEFKELSLDKNIAAINTIFSRDDYDLTRIFVPAKEITEFSLKLFGHWSILQDGLFLLGKDNAKKDLSEKQITELIKEIAKKDYSLAELQNAYERWTKENDIPVEKTVKNYFRLAELRTDEKTKEKNFTDIQKELEKAFVQIDFDKKENLIKEKEAATPIKNFLDEVQNLFHYLKLVDYRGEEEKDSDFYAKYDQVLQALAEIIPLYNKVRNFVTKKPNEVKKVKLNFECSSFLRGWGTDRGTKEAHIFIDDGKYYLGIVNEKLSKEDIKFLEEKSPRMIKKVVYDFQKPDNKNTPRLFIRSKGTSYAPSVSQYDLPIESVIEIYDKGLFKTEYRKVNPSVYKESLVKMIDYFKLGFTRHESYKHYNFSWKDSKDYNDISEFYADVMNSCYQLKFEYINYDNLLSLVDAGKLFLFQIYNKDFSTGKDGANGSTGKKNLHTLYWENLFSEENLKDICLKLNGEAELFWRDVNPNIKNICHKKGSILVNRTTSDGKVIPEDIYQEIYKFKNPDKQEKDFKISDEAKALLENGKVVCKEAKFDITKDRHFTQQTYLFHCPITMSFKAPEITGRKFNEKVQSILKANPDVKIIGLDRGERHLIYLSLINQKGEIELQKTLNLVDQVRNDKTVSVNYQEKLVHKEGERDKARKNWQSISNIKELKEGYLSNVVHEIAKLMVENNAIVVMEDLNFGFKRGRFAVERQVYQKFENMLIEKLNYLVFKDKAVTEPGGVLNAYQLTDKSANVSDVGKQCGWIFYVPAAYTSKIDPKTGFANLFYTAGLTNVEKKKEFFDKFEAIRYDSKTDSFVLSFDYKDFSDNADYKKKWSLYSRGERLVFSKAEKNVISVNPTENLKALFDKQGINWKSEENFIDQINAVQAERENVSFFDGLYRSFTAILQMRNSVPNSSKMEDDYLISPVMAEDGTFYDSRKEAAKGKDEQGKWISKLPVDADANGAYHIALKGLYLLQNDFNVNDKGYIDNISNADWFEFAQEKNYAK

SEQ ID NO: 280 - protein, 1235 aa, synthetic construct
  source 1..1235: note = NSG-01 Cas12a protein with NLS
PKKKRKVKKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQKPKKKRKV

SEQ ID NO: 281 - protein, 1270 aa, synthetic construct; identical to SEQ ID NO: 123
  source 1..1270: note = NSG-01 Cas12a protein with NLS

SEQ ID NO: 282 - protein, 1235 aa, synthetic construct
  source 1..1235: note = NSG-01 Cas12a variant protein with NLS
PKKKRKVKKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWRNRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQKPKKKRKV

SEQ ID NO: 283 - protein, 1235 aa, synthetic construct
  source 1..1235: note = NSG-01 Cas12a variant protein with NLS
PKKKRKVKKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMRGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQKPKKKRKV

SEQ ID NO: 284 - protein, 1235 aa, synthetic construct
  source 1..1235: note = NSG-01 Cas12a variant protein with NLS
PKKKRKVKKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWRNRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMRGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQKPKKKRKV

SEQ ID NO: 285 - protein, 1235 aa, synthetic construct
  source 1..1235: note = NSG-01 Cas12a variant protein with NLS
PKKKRKVKKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWRNRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNREADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQKPKKKRKV

SEQ ID NO: 286 - protein, 1270 aa, synthetic construct
  source 1..1270: note = NSG-01 Cas12a variant protein with NLS
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNAITGRTDIDYLISPVKNDTGNFYDSRNFEALENAVLPKDADANGAYNIARKVLWAIEQFRESDESKLEKVSIAISNKKWLEYAQKKRPAATKKAGQAKKKKPAAKRVKLDGGGGSGGGGSGGGGSPAAKRVKLD

SEQ ID NO: 287 - protein, 1270 aa, synthetic construct
  source 1..1270: note = NSG-01 Cas12a variant protein with NLS
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYLIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQKKRPAATKKAGQAKKKKPAAKRVKLDGGGGSGGGGSGGGGSPAAKRVKLD

SEQ ID NO: 288 - protein, 1270 aa, synthetic construct
  source 1..1270: note = NSG-01 Cas12a variant protein with NLS
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWRNRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYLIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQKKRPAATKKAGQAKKKKPAAKRVKLDGGGGSGGGGSGGGGSPAAKRVKLD

SEQ ID NO: 289 - protein, 1270 aa, synthetic construct
  source 1..1270: note = NSG-01 Cas12a variant protein with NLS
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWRNRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQRPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYLIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQKKRPAATKKAGQAKKKKPAAKRVKLDGGGGSGGGGSGGGGSPAAKRVKLD

SEQ ID NO: 290 - protein, 1270 aa, synthetic construct
  source 1..1270: note = NSG-01 Cas12a variant protein with NLS
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQRPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQKKRPAATKKAGQAKKKKPAAKRVKLDGGGGSGGGGSGGGGSPAAKRVKLD

SEQ ID NO: 291 - protein, 1270 aa, synthetic construct
  source 1..1270: note = NSG-01 Cas12a variant protein with NLS
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQRPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYLIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQKKRPAATKKAGQAKKKKPAAKRVKLDGGGGSGGGGSGGGGSPAAKRVKLD

SEQ ID NO: 292 - protein, 1270 aa, synthetic construct
  source 1..1270: note = NSG-01 Cas12a variant protein with NLS
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWRNRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQRPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQKKRPAATKKAGQAKKKKPAAKRVKLDGGGGSGGGGSGGGGSPAAKRVKLD

SEQ ID NO: 293 - protein, 1388 aa, synthetic construct
  source 1..1388: note = NSG-02 Cas12a protein with NLS
AFVICYNTYSRTYLEKEIISMKTIESFCGQKKGYSRSITLRNRLIPIGKTEENIRKLKLLDKDIDRSKAYVEVKALIDDFHRVFIEDVLSKTELPWEPLYDQFELYQNEKDKQKKNKIKKDLEELQKGLRKSIVKQFKADDRFDKLFKKELLTEFVPSVIKNDDSGTISDKQAALDIFKGFATYFTGFHQNRQNMYSEEAQSTAISNRIVNENFPKFYANIQTFKYLEENFPQIISDTEQSLSDFLGDKKLKDIFSIDGFNKVLSQSGIDFYNTVIGGISEEAGTQKVQGLNEKINLASQQLSSEEKHKLKKKMTVLYKQILSDRSTASFIPVGFEKSEEVYDSVKEFKELSLDKNIAAINTIFSRDDYDLTRIFVPAKEITEFSLKLFGHWSILQDGLFLLGKDNAKKDLSEKQITELIKEIAKKDYSLAELQNAYERWTKENDIPVEKTVKNYFRLAELRTDEKTKEKNFTDIQKELEKAFVQIDFDKKENLIKEKEAATPIKNFLDEVQNLFHYLKLVDYRGEEEKDSDFYAKYDQVLQALAEIIPLYNKVRNFVTKKPNEVKKVKLNFECSSFLSGWGTDYGTKEAHIFIDDGKYYLGIVNEKLSKEDIKFLEEKSPRMIKKVVYDFQKPDNKNTPRLFIRSKGTSYAPSVSQYDLPIESVIEIYDKGLFKTEYRKVNPSVYKESLVKMIDYFKLGFTRHESYKHYNFSWKDSKDYNDISEFYADVMNSCYQLKFEYINYDNLLSLVDAGKLFLFQIYNKDFSTGKDGANGSTGKKNLHTLYWENLFSEENLKDICLKLNGEAELFWRDVNPNIKNICHKKGSILVNRTTSDGKVIPEDIYQEIYKFKNPDKQEKDFKISDEAKALLENGKVVCKEAKFDITKDRHFTQQTYLFHCPITMSFKAPEITGRKFNEKVQSILKANPDVKIIGLDRGERHLIYLSLINQKGEIELQKTLNLVDQVRNDKTVSVNYQEKLVHKEGERDKARKNWQSISNIKELKEGYLSNVVHEIAKLMVENNAIVVMEDLNFGFKRGRFAVERQVYQKFENMLIEKLNYLVFKDKAVTEPGGVLNAYQLTDKSANVSDVGKQCGWIFYVPAAYTSKIDPKTGFANLFYTAGLTNVEKKKEFFDKFEAIRYDSKTDSFVLSFDYKDFSDNADYKKKWSLYSRGERLVFSKAEKNVISVNPTENLKALFDKQGINWKSEENFIDQINAVQAERENVSFFDGLYRSFTAILQMRNSVPNSSKMEDDYLISPVMAEDGTFYDSRKEAAKGKDEQGKWISKLPVDADANGAYHIALKGLYLLQNDFNVNDKGYIDNISNADWFEFAQEKNYAKKRPAATKKAGQAKKKKPAAKRVKLDGGGGSGGGGSGGGGSPAAKRVKLD

SEQ ID NO: 294 - protein, 1388 aa, synthetic construct
  source 1..1388: note = NSG-02 Cas12a variant protein with NLS
AFVICYNTYSRTYLEKEIISMKTIESFCGQKKGYSRSITLRNRLIPIGKTEENIRKLKLLDKDIDRSKAYVEVKALIDDFHRVFIEDVLSKTELPWEPLYDQFELYQNEKDKQKKNKIKKDLEELQKGLRKSIVKQFKADDRFDKLFKKELLTEFVPSVIKNDDSGTISDKQAALDIFKGFATYFTGFHRNRQNMYSEEAQSTAISNRIVNENFPKFYANIQTFKYLEENFPQIISDTEQSLSDFLGDKKLKDIFSIDGFNKVLSQSGIDFYNTVIGGISEEAGTQKVQGLNEKINLASQQLSSEEKHKLKKKMTVLYKQILSDRSTASFIPVGFEKSEEVYDSVKEFKELSLDKNIAAINTIFSRDDYDLTRIFVPAKEITEFSLKLFGHWSILQDGLFLLGKDNAKKDLSEKQITELIKEIAKKDYSLAELQNAYERWTKENDIPVEKTVKNYFRLAELRTDEKTKEKNFTDIQKELEKAFVQIDFDKKENLIKEKEAATPIKNFLDEVQNLFHYLKLVDYRGEEEKDSDFYAKYDQVLQALAEIIPLYNKVRNFVTKKPNEVKKVKLNFECSSFLRGWGTDYGTKEAHIFIDDGKYYLGIVNEKLSKEDIKFLEEKSPRMIKKVVYDFQKPDNKNTPRLFIRSKGTSYAPSVSQYDLPIESVIEIYDKGLFKTEYRKVNPSVYKESLVKMIDYFKLGFTRHESYKHYNFSWKDSKDYNDISEFYADVMNSCYQLKFEYINYDNLLSLVDAGKLFLFQIYNKDFSTGKDGANGSTGKKNLHTLYWENLFSEENLKDICLKLNGEAELFWRDVNPNIKNICHKKGSILVNRTTSDGKVIPEDIYQEIYKFKNPDKQEKDFKISDEAKALLENGKVVCKEAKFDITKDRHFTQQTYLFHCPITMSFKAPEITGRKFNEKVQSILKANPDVKIIGLDRGERHLIYLSLINQKGEIELQKTLNLVDQVRNDKTVSVNYQEKLVHKEGERDKARKNWQSISNIKELKEGYLSNVVHEIAKLMVENNAIVVMEDLNFGFKRGRFAVERQVYQKFENMLIEKLNYLVFKDKAVTEPGGVLNAYQLTDKSANVSDVGKQCGWIFYVPAAYTSKIDPKTGFANLFYTAGLTNVEKKKEFFDKFEAIRYDSKTDSFVLSFDYKDFSDNADYKKKWSLYSRGERLVFSKAEKNVISVNPTENLKALFDKQGINWKSEENFIDQINAVQAERENVSFFDGLYRSFTAILQMRNSVPNSSKMEDDYLISPVMAEDGTFYDSRKEAAKGKDEQGKWISKLPVDADANGAYHIALKGLYLLQNDFNVNDKGYIDNISNADWFEFAQEKNYAKKRPAATKKAGQAKKKKPAAKRVKLDGGGGSGGGGSGGGGSPAAKRVKLD

SEQ ID NO: 295 - protein, 1388 aa, synthetic construct
  source 1..1388: note = NSG-02 Cas12a variant protein with NLS
AFVICYNTYSRTYLEKEIISMKTIESFCGQKKGYSRSITLRNRLIPIGKTEENIRKLKLLDKDIDRSKAYVEVKALIDDFHRVFIEDVLSKTELPWEPLYDQFELYQNEKDKQKKNKIKKDLEELQKGLRKSIVKQFKADDRFDKLFKKELLTEFVPSVIKNDDSGTISDKQAALDIFKGFATYFTGFHRNRQNMYSEEAQSTAISNRIVNENFPKFYANIQTFKYLEENFPQIISDTEQSLSDFLGDKKLKDIFSIDGFNKVLSQSGIDFYNTVIGGISEEAGTQKVQGLNEKINLASQQLSSEEKHKLKKKMTVLYKQILSDRSTASFIPVGFEKSEEVYDSVKEFKELSLDKNIAAINTIFSRDDYDLTRIFVPAKEITEFSLKLFGHWSILQDGLFLLGKDNAKKDLSEKQITELIKEIAKKDYSLAELQNAYERWTKENDIPVEKTVKNYFRLAELRTDEKTKEKNFTDIQKELEKAFVQIDFDKKENLIKEKEAATPIKNFLDEVQNLFHYLKLVDYRGEEEKDSDFYAKYDQVLQALAEIIPLYNKVRNFVTKKPNEVKKVKLNFECSSFLSGWGTDYGTKEAHIFIDDGKYYLGIVNEKLSKEDIKFLEEKSPRMIKKVVYDFQKPDNKNTPRLFIRSKGTSYAPSVSQYDLPIESVIEIYDKGLFKTEYRKVNPSVYKESLVKMIDYFKLGFTRHESYKHYNFSWKDSKDYNDISEFYADVMNSCYQLKFEYINYDNLLSLVDAGKLFLFQIYNKDFSTGKDGANGSTGKKNLHTLYWENLFSEENLKDICLKLNGEAELFWRDVNPNIKNICHKKGSILVNRTTSDGKVIPEDIYQEIYKFKNPDKQEKDFKISDEAKALLENGKVVCKEAKFDITKDRHFTQQTYLFHCPITMSFKAPEITGRKFNEKVQSILKANPDVKIIGLDRGERHLIYLSLINQKGEIELQKTLNLVDQVRNDKTVSVNYQEKLVHKEGERDKARKNWQSISNIKELKEGYLSNVVHEIAKLMVENNAIVVMEDLNFGFKRGRFAVERQVYQKFENMLIEKLNYLVFKDKAVTEPGGVLNAYQLTDKSANVSDVGKQCGWIFYVPAAYTSKIDPKTGFANLFYTAGLTNVEKKKEFFDKFEAIRYDSKTDSFVLSFDYKDFSDNADYKKKWSLYSRGERLVFSKAEKNVISVNPTENLKALFDKQGINWKSEENFIDQINAVQAERENVSFFDGLYRSFTAILQMRNSVPNSSKMEDDYLISPVMAEDGTFYDSRKEAAKGKDEQGKWISKLPVDADANGAYHIALKGLYLLQNDFNVNDKGYIDNISNADWFEFAQEKNYAKKRPAATKKAGQAKKKKPAAKRVKLDGGGGSGGGGSGGGGSPAAKRVKLD

SEQ ID NO: 296 - protein, 1388 aa, synthetic construct
  source 1..1388: note = NSG-02 Cas12a variant protein with NLS
AFVICYNTYSRTYLEKEIISMKTIESFCGQKKGYSRSITLRNRLIPIGKTEENIRKLKLLDKDIDRSKAYVEVKALIDDFHRVFIEDVLSKTELPWEPLYDQFELYQNEKDKQKKNKIKKDLEELQKGLRKSIVKQFKADDRFDKLFKKELLTEFVPSVIKNDDSGTISDKQAALDIFKGFATYFTGFHQNRQNMYSEEAQSTAISNRIVNENFPKFYANIQTFKYLEENFPQIISDTEQSLSDFLGDKKLKDIFSIDGFNKVLSQSGIDFYNTVIGGISEEAGTQKVQGLNEKINLASQQLSSEEKHKLKKKMTVLYKQILSDRSTASFIPVGFEKSEEVYDSVKEFKELSLDKNIAAINTIFSRDDYDLTRIFVPAKEITEFSLKLFGHWSILQDGLFLLGKDNAKKDLSEKQITELIKEIAKKDYSLAELQNAYERWTKENDIPVEKTVKNYFRLAELRTDEKTKEKNFTDIQKELEKAFVQIDFDKKENLIKEKEAATPIKNFLDEVQNLFHYLKLVDYRGEEEKDSDFYAKYDQVLQALAEIIPLYNKVRNFVTKKPNEVKKVKLNFECSSFLRGWGTDYGTKEAHIFIDDGKYYLGIVNEKLSKEDIKFLEEKSPRMIKKVVYDFQKPDNKNTPRLFIRSKGTSYAPSVSQYDLPIESVIEIYDKGLFKTEYRKVNPSVYKESLVKMIDYFKLGFTRHESYKHYNFSWKDSKDYNDISEFYADVMNSCYQLKFEYINYDNLLSLVDAGKLFLFQIYNKDFSTGKDGANGSTGKKNLHTLYWENLFSEENLKDICLKLNGEAELFWRDVNPNIKNICHKKGSILVNRTTSDGKVIPEDIYQEIYKFKNPDKQEKDFKISDEAKALLENGKVVCKEAKFDITKDRHFTQQTYLFHCPITMSFKAPEITGRKFNEKVQSILKANPDVKIIGLDRGERHLIYLSLINQKGEIELQKTLNLVDQVRNDKTVSVNYQEKLVHKEGERDKARKNWQSISNIKELKEGYLSNVVHEIAKLMVENNAIVVMEDLNFGFKRGRFAVERQVYQKFENMLIEKLNYLVFKDKAVTEPGGVLNAYQLTDKSANVSDVGKQCGWIFYVPAAYTSKIDPKTGFANLFYTAGLTNVEKKKEFFDKFEAIRYDSKTDSFVLSFDYKDFSDNADYKKKWSLYSRGERLVFSKAEKNVISVNPTENLKALFDKQGINWKSEENFIDQINAVQAERENVSFFDGLYRSFTAILQMRNSVPNSSKMEDDYLISPVMAEDGTFYDSRKEAAKGKDEQGKWISKLPVDADANGAYHIALKGLYLLQNDFNVNDKGYIDNISNADWFEFAQEKNYAKKRPAATKKAGQAKKKKPAAKRVKLDGGGGSGGGGSGGGGSPAAKRVKLD

SEQ ID NO: 297 - protein, 1388 aa, synthetic construct
  source 1..1388: note = NSG-02 Cas12a variant protein with NLS
AFVICYNTYSRTYLEKEIISMKTIESFCGQKKGYSRSITLRNRLIPIGKTEENIRKLKLLDKDIDRSKAYVEVKALIDDFHRVFIEDVLSKTELPWEPLYDQFELYQNEKDKQKKNKIKKDLEELQKGLRKSIVKQFKADDRFDKLFKKELLTEFVPSVIKNDDSGTISDKQAALDIFKGFATYFTGFHRNRQNMYSEEAQSTAISNRIVNENFPKFYANIQTFKYLEENFPQIISDTEQSLSDFLGDKKLKDIFSIDGFNKVLSQSGIDFYNTVIGGISEEAGTQKVQGLNEKINLASQQLSSEEKHKLKKKMTVLYKQILSDRSTASFIPVGFEKSEEVYDSVKEFKELSLDKNIAAINTIFSRDDYDLTRIFVPAKEITEFSLKLFGHWSILQDGLFLLGKDNAKKDLSEKQITELIKEIAKKDYSLAELQNAYERWTKENDIPVEKTVKNYFRLAELRTDEKTKEKNFTDIQKELEKAFVQIDFDKKENLIKEKEAATPIKNFLDEVQNLFHYLKLVDYRGEEEKDSDFYAKYDQVLQALAEIIPLYNKVRNFVTKKPNEVKKVKLNFECSSFLSGWGTDRGTKEAHIFIDDGKYYLGIVNEKLSKEDIKFLEEKSPRMIKKVVYDFQKPDNKNTPRLFIRSKGTSYAPSVSQYDLPIESVIEIYDKGLFKTEYRKVNPSVYKESLVKMIDYFKLGFTRHESYKHYNFSWKDSKDYNDISEFYADVMNSCYQLKFEYINYDNLLSLVDAGKLFLFQIYNKDFSTGKDGANGSTGKKNLHTLYWENLFSEENLKDICLKLNGEAELFWRDVNPNIKNICHKKGSILVNRTTSDGKVIPEDIYQEIYKFKNPDKQEKDFKISDEAKALLENGKVVCKEAKFDITKDRHFTQQTYLFHCPITMSFKAPEITGRKFNEKVQSILKANPDVKIIGLDRGERHLIYLSLINQKGEIELQKTLNLVDQVRNDKTVSVNYQEKLVHKEGERDKARKNWQSISNIKELKEGYLSNVVHEIAKLMVENNAIVVMEDLNFGFKRGRFAVERQVYQKFENMLIEKLNYLVFKDKAVTEPGGVLNAYQLTDKSANVSDVGKQCGWIFYVPAAYTSKIDPKTGFANLFYTAGLTNVEKKKEFFDKFEAIRYDSKTDSFVLSFDYKDFSDNADYKKKWSLYSRGERLVFSKAEKNVISVNPTENLKALFDKQGINWKSEENFIDQINAVQAERENVSFFDGLYRSFTAILQMRNSVPNSSKMEDDYLISPVMAEDGTFYDSRKEAAKGKDEQGKWISKLPVDADANGAYHIALKGLYLLQNDFNVNDKGYIDNISNADWFEFAQEKNYAKKRPAATKKAGQAKKKKPAAKRVKLDGGGGSGGGGSGGGGSPAAKRVKLD

SEQ ID NO: 298 - protein, 1388 aa, synthetic construct
  source 1..1388: note = NSG-02 Cas12a variant protein with NLS
AFVICYNTYSRTYLEKEIISMKTIESFCGQKKGYSRSITLRNRLIPIGKTEENIRKLKLLDKDIDRSKAYVEVKALIDDFHRVFIEDVLSKTELPWEPLYDQFELYQNEKDKQKKNKIKKDLEELQKGLRKSIVKQFKADDRFDKLFKKELLTEFVPSVIKNDDSGTISDKQAALDIFKGFATYFTGFHRNRQNMYSEEAQSTAISNRIVNENFPKFYANIQTFKYLEENFPQIISDTEQSLSDFLGDKKLKDIFSIDGFNKVLSQSGIDFYNTVIGGISEEAGTQKVQGLNEKINLASQQLSSEEKHKLKKKMTVLYKQILSDRSTASFIPVGFEKSEEVYDSVKEFKELSLDKNIAAINTIFSRDDYDLTRIFVPAKEITEFSLKLFGHWSILQDGLFLLGKDNAKKDLSEKQITELIKEIAKKDYSLAELQNAYERWTKENDIPVEKTVKNYFRLAELRTDEKTKEKNFTDIQKELEKAFVQIDFDKKENLIKEKEAATPIKNFLDEVQNLFHYLKLVDYRGEEEKDSDFYAKYDQVLQALAEIIPLYNKVRNFVTKKPNEVKKVKLNFECSSFLRGWGTDRGTKEAHIFIDDGKYYLGIVNEKLSKEDIKFLEEKSPRMIKKVVYDFQKPDNKNTPRLFIRSKGTSYAPSVSQYDLPIESVIEIYDKGLFKTEYRKVNPSVYKESLVKMIDYFKLGFTRHESYKHYNFSWKDSKDYNDISEFYADVMNSCYQLKFEYINYDNLLSLVDAGKLFLFQIYNKDFSTGKDGANGSTGKKNLHTLYWENLFSEENLKDICLKLNGEAELFWRDVNPNIKNICHKKGSILVNRTTSDGKVIPEDIYQEIYKFKNPDKQEKDFKISDEAKALLENGKVVCKEAKFDITKDRHFTQQTYLFHCPITMSFKAPEITGRKFNEKVQSILKANPDVKIIGLDRGERHLIYLSLINQKGEIELQKTLNLVDQVRNDKTVSVNYQEKLVHKEGERDKARKNWQSISNIKELKEGYLSNVVHEIAKLMVENNAIVVMEDLNFGFKRGRFAVERQVYQKFENMLIEKLNYLVFKDKAVTEPGGVLNAYQLTDKSANVSDVGKQCGWIFYVPAAYTSKIDPKTGFANLFYTAGLTNVEKKKEFFDKFEAIRYDSKTDSFVLSFDYKDFSDNADYKKKWSLYSRGERLVFSKAEKNVISVNPTENLKALFDKQGINWKSEENFIDQINAVQAERENVSFFDGLYRSFTAILQMRNSVPNSSKMEDDYLISPVMAEDGTFYDSRKEAAKGKDEQGKWISKLPVDADANGAYHIALKGLYLLQNDFNVNDKGYIDNISNADWFEFAQEKNYAKKRPAATKKAGQAKKKKPAAKRVKLDGGGGSGGGGSGGGGSPAAKRVKLD

SEQ ID NO: 299 - protein, 1221 aa, synthetic construct
  source 1..1221: note = Variant of NSG-01
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNREADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQK

SEQ ID NO: 300 - protein, 1221 aa, synthetic construct
  source 1..1221: note = Variant of NSG-01
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMRGWDRNREADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQK

SEQ ID NO: 301 - protein, 1221 aa, synthetic construct
  source 1..1221: note = Variant of NSG-01
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWRNRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMRGWDRNREADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQK

SEQ ID NO: 302 - protein, 1221 aa, synthetic construct
  source 1..1221: note = Variant of NSG-01
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWENRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSASYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQK

SEQ ID NO: 303 - protein, 1221 aa, synthetic construct
  source 1..1221: note = Variant of NSG-01
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWRNRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSASYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMGGWDRNKEADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNSITGRTDIDYLISPVKNDTGNFYDSRNFEALENAVLPKDADANGAYNIARKVLWAIEQFRESDESKLEKVSIAISNKKWLEYAQK

SEQ ID NO: 304 - protein, 1221 aa, synthetic construct
  source 1..1221: note = Variant of NSG-01
KKIDNFTGCYSLSRTLRFKLIPQGKTQEHIELKRLLDEDEKRAEDYKKAKKIIDKYHITFIDRILHNIKLTELQSYIELDEVSNKDEKQKKELLKIEGLLRKEISNSFKKDDEYKKIFGKEIIETILPEYLDDQEEIEIIKSFKGFSTAFVGYWRNRKNLYTDEEIGSSIAYRAINDNLPKFMRNASIFKSVEKIFSEEELREIKKEVLNDEYDVQDFFRKDFYDFLLPQDGIDIYNAILGGLVKEDGTKIRGLNEYINLYNQKNKEKNAKLKPLFKQVLTESESKSFYIDEFAKDEEVIEAFQNTFAEEGEVCLAIDQIQKLFNQYEIYDSNGIFIKNGLAVTALANDIYGSWSYIKEKWNTLYDAENTGRTSKESESYITKRENIYKKIESFSLNEIETITEGEEKLLSEISNIISEKIQLYFEKYNSAKYLLNSDYKLDKKLQKNNKVVEVIKELMDSVKDIERYLKPFMGTGKEEGRDEYFYSEFENTLEVLSGIDRLYNKIRNYVTKEPFSKKKFKLYFQNPQFMRGWDRNREADYRVALLRKKQKYYLAIMDKSDSKCLQRVEQGNGDLQKMNYKLISGASKSLPHVFFSKKYLSNNNVEDEILRIYKSKTFIKGESFDIDDCHKLINYYKESIKSHPWGSAFDFSFSDTNEYADIHTFYQEVDDMGYKIVFDNVSEEAVNKLVDEGKLYLFQIYNKDFSEKSHGTENLHTMYFKSLFDENNRGNIRLCGGAEMFLRKASLKKQELVVHPANVPMKNKNKNNPKKTTTLPYDVYKDKRYSEDQYEIHIPIIINKATTNFYKLNLEIRKLLREDTNPYVIGIDRGERNLLYMVVIDGKGRIVEQYSINEIVNECKGITIKTDYHSLLDEKEKERMAARKSWKTVENIKELKEGYISQVVHKICELVRKYDAVIALEDLNSGFKNSRVKVEKQVYQKFEKMLIDKLNYMVDKKIPWYENGGVLKGYQLTNKFESFKKMGTQNGMMFYIPAWLTSKIDPSTGFVNLLRTRYKSVQDSKNFINKISSIKYDKDEEMFCFAIDYSNFEHTNADYKKKWILYSNGERIKHFRNPEKNSEFDYKIINLTTEFKNLFEEYNIAYELGEDIKEQINVINEKKFFEKFMSTVTLMLQMRNAITGRTDIDYLISPVKNDTGNFYDSRNFEALENAVLPKDADANGAYNIARKVLWAIEQFRESDESKLEKVSIAISNKKWLEYAQK

SEQ ID NO: 305 - protein, 1339 aa, synthetic construct
  source 1..1339: note = Variant of NSG-02
AFVICYNTYSRTYLEKEIISMKTIESFCGQKKGYSRSITLRNRLIPIGKTEENIRKLKLLDKDIDRSKAYVEVKALIDDFHRVFIEDVLSKTELPWEPLYDQFELYQNEKDKQKKNKIKKDLEELQKGLRKSIVKQFKADDRFDKLFKKELLTEFVPSVIKNDDSGTISDKQAALDIFKGFATYFTGFHQNRQNMYSEEAQSTAISNRIVNENFPKFYANIQTFKYLEENFPQIISDTEQSLSDFLGDKKLKDIFSIDGFNKVLSQSGIDFYNTVIGGISEEAGTQKVQGLNEKINLASQQLSSEEKHKLKKKMTVLYKQILSDRSTASFIPVGFEKSEEVYDSVKEFKELSLDKNIAAINTIFSRDDYDLTRIFVPAKEITEFSLKLFGHWSILQDGLFLLGKDNAKKDLSEKQITELIKEIAKKDYSLAELQNAYERWTKENDIPVEKTVKNYFRLAELRTDEKTKEKNFTDIQKELEKAFVQIDFDKKENLIKEKEAATPIKNFLDEVQNLFHYLKLVDYRGEEEKDSDFYAKYDQVLQALAEIIPLYNKVRNFVTKKPNEVKKVKLNFECSSFLSGWGTDRGTKEAHIFIDDGKYYLGIVNEKLSKEDIKFLEEKSPRMIKKVVYDFQKPDNKNTPRLFIRSKGTSYAPSVSQYDLPIESVIEIYDKGLFKTEYRKVNPSVYKESLVKMIDYFKLGFTRHESYKHYNFSWKDSKDYNDISEFYADVMNSCYQLKFEYINYDNLLSLVDAGKLFLFQIYNKDFSTGKDGANGSTGKKNLHTLYWENLFSEENLKDICLKLNGEAELFWRDVNPNIKNICHKKGSILVNRTTSDGKVIPEDIYQEIYKFKNPDKQEKDFKISDEAKALLENGKVVCKEAKFDITKDRHFTQQTYLFHCPITMSFKAPEITGRKFNEKVQSILKANPDVKIIGLDRGERHLIYLSLINQKGEIELQKTLNLVDQVRNDKTVSVNYQEKLVHKEGERDKARKNWQSISNIKELKEGYLSNVVHEIAKLMVENNAIVVMEDLNFGFKRGRFAVERQVYQKFENMLIEKLNYLVFKDKAVTEPGGVLNAYQLTDKSANVSDVGKQCGWIFYVPAAYTSKIDPKTGFANLFYTAGLTNVEKKKEFFDKFEAIRYDSKTDSFVLSFDYKDFSDNADYKKKWSLYSRGERLVFSKAEKNVISVNPTENLKALFDKQGINWKSEENFIDQINAVQAERENVSFFDGLYRSFTAILQMRNSVPNSSKMEDDYLISPVMAEDGTFYDSRKEAAKGKDEQGKWISKLPVDADANGAYHIALKGLYLLQNDFNVNDKGYIDNISNADWFEFAQEKNYAK

SEQ ID NO: 306 - protein, 1339 aa, synthetic construct
  source 1..1339: note = Variant of NSG-02
AFVICYNTYSRTYLEKEIISMKTIESFCGQKKGYSRSITLRNRLIPIGKTEENIRKLKLLDKDIDRSKAYVEVKALIDDFHRVFIEDVLSKTELPWEPLYDQFELYQNEKDKQKKNKIKKDLEELQKGLRKSIVKQFKADDRFDKLFKKELLTEFVPSVIKNDDSGTISDKQAALDIFKGFATYFTGFHQNRQNMYSEEAQSTAISNRIVNENFPKFYANIQTFKYLEENFPQIISDTEQSLSDFLGDKKLKDIFSIDGFNKVLSQSGIDFYNTVIGGISEEAGTQKVQGLNEKINLASQQLSSEEKHKLKKKMTVLYKQILSDRSTASFIPVGFEKSEEVYDSVKEFKELSLDKNIAAINTIFSRDDYDLTRIFVPAKEITEFSLKLFGHWSILQDGLFLLGKDNAKKDLSEKQITELIKEIAKKDYSLAELQNAYERWTKENDIPVEKTVKNYFRLAELRTDEKTKEKNFTDIQKELEKAFVQIDFDKKENLIKEKEAATPIKNFLDEVQNLFHYLKLVDYRGEEEKDSDFYAKYDQVLQALAEIIPLYNKVRNFVTKKPNEVKKVKLNFECSSFLRGWGTDRGTKEAHIFIDDGKYYLGIVNEKLSKEDIKFLEEKSPRMIKKVVYDFQKPDNKNTPRLFIRSKGTSYAPSVSQYDLPIESVIEIYDKGLFKTEYRKVNPSVYKESLVKMIDYFKLGFTRHESYKHYNFSWKDSKDYNDISEFYADVMNSCYQLKFEYINYDNLLSLVDAGKLFLFQIYNKDFSTGKDGANGSTGKKNLHTLYWENLFSEENLKDICLKLNGEAELFWRDVNPNIKNICHKKGSILVNRTTSDGKVIPEDIYQEIYKFKNPDKQEKDFKISDEAKALLENGKVVCKEAKFDITKDRHFTQQTYLFHCPITMSFKAPEITGRKFNEKVQSILKANPDVKIIGLDRGERHLIYLSLINQKGEIELQKTLNLVDQVRNDKTVSVNYQEKLVHKEGERDKARKNWQSISNIKELKEGYLSNVVHEIAKLMVENNAIVVMEDLNFGFKRGRFAVERQVYQKFENMLIEKLNYLVFKDKAVTEPGGVLNAYQLTDKSANVSDVGKQCGWIFYVPAAYTSKIDPKTGFANLFYTAGLTNVEKKKEFFDKFEAIRYDSKTDSFVLSFDYKDFSDNADYKKKWSLYSRGERLVFSKAEKNVISVNPTENLKALFDKQGINWKSEENFIDQINAVQAERENVSFFDGLYRSFTAILQMRNSVPNSSKMEDDYLISPVMAEDGTFYDSRKEAAKGKDEQGKWISKLPVDADANGAYHIALKGLYLLQNDFNVNDKGYIDNISNADWFEFAQEKNYAK

SEQ ID NO: 307 - DNA, 20 bp, synthetic construct
  source 1..20: mol_type = other DNA; note = Primer sequence for experimental example, FANCF_T7_FWD
accgttacaaagctagtccc

SEQ ID NO: 308 - DNA, 20 bp, synthetic construct
  source 1..20: mol_type = other DNA; note = Primer sequence for experimental example, FANCF_T7_REV
ccaaagttaatgcaccgagg

SEQ ID NO: 309 - DNA, 20 bp, synthetic construct
  source 1..20: mol_type = other DNA; note = Primer sequence for experimental example, PD1_T7_FWD
acgtcgtaaagccaaggtta

SEQ ID NO: 310 - DNA, 19 bp, synthetic construct
  source 1..19: mol_type = other DNA; note = Primer sequence for experimental example, PD1_T7_REV
ctcccttcaacctgacctg

SEQ ID NO: 311 - DNA, 20 bp, synthetic construct
  source 1..20: mol_type = other DNA; note = Primer sequence for experimental example, VEGFA_T7_FWD
aagcgactcaacctggtaaa

SEQ ID NO: 312 - DNA, 20 bp, synthetic construct
  source 1..20: mol_type = other DNA; note = Primer sequence for experimental example, VEGFA_T7_REV
agtcaagccatgcctttttc

SEQ ID NO: 313 - DNA, 20 bp, synthetic construct
  source 1..20: mol_type = other DNA; note = Primer sequence for experimental example, Site4_T7_FWD
ccaaacaaggaagccaaagt

SEQ ID NO: 314 - DNA, 20 bp, synthetic construct
  source 1..20: mol_type = other DNA; note = Primer sequence for experimental example, Site4_T7_REV
aaagagcaaacctgaactgg

SEQ ID NO: 315 - DNA, 20 bp, synthetic construct
  source 1..20: mol_type = other DNA; note = Primer sequence for experimental example, CFTR_T7_FWD
gcttggacttcatgtgtgtc

SEQ ID NO: 316 - DNA, 20 bp, synthetic construct
  source 1..20: mol_type = other DNA; note = Primer sequence for experimental example, CFTR_T7_REV
taactggaaacttcagcggt

SEQ ID NO: 317 - DNA, 20 bp, synthetic construct
  source 1..20: mol_type = other DNA; note = Primer sequence for experimental example, Site6_T7_FWD
cctgtatgtagcgcaagaga

SEQ ID NO: 318 - DNA, 20 bp, synthetic construct
  source 1..20: mol_type = other DNA; note = Primer sequence for experimental example, Site6_T7_REV
cccgagccctgtctataaat

SEQ ID NO: 319 - DNA, 20 bp, synthetic construct
  source 1..20: mol_type = other DNA; note = Primer sequence for experimental example, IFTAP_T7_FWD
aacccacccacgattgtaaa

SEQ ID NO: 320 - DNA, 20 bp, synthetic construct
  source 1..20: mol_type = other DNA; note = Primer sequence for experimental example, IFTAP_T7_REV
gctgacagtattgtgcagtg

SEQ ID NO: 321 - DNA, 20 bp, synthetic construct
  source 1..20: mol_type = other DNA; note = Primer sequence for experimental example, Site6-T_T7_FWD
gtccataggcgagaagaaca

SEQ ID NO: 322 - DNA, 20 bp, synthetic construct
  source 1..20: mol_type = other DNA; note = Primer sequence for experimental example, Site6-T_T7_REV
gagttgagaggatgggatgg

SEQ ID NO: 323 - DNA, 20 bp, synthetic construct
  source 1..20: mol_type = other DNA; note = Primer sequence for experimental example, VEGF-T_T7_FWD
ccagagtcccccagaaatac

SEQ ID NO: 324 - DNA, 20 bp, synthetic construct
  source 1..20: mol_type = other DNA; note = Primer sequence for experimental example, VEGF-T_T7_REV
gagcccgatacacacactta

SEQ ID NO: 325 - DNA, 20 bp, synthetic construct
  source 1..20: mol_type = other DNA; note = Primer sequence for experimental example, DNMT1_T7E1_FWD
gaacgttcccttagcactct

SEQ ID NO: 326 - DNA, 20 bp, synthetic construct
  source 1..20: mol_type = other DNA; note = Primer sequence for experimental example, DNMT1_T7E1_REV
gcaaatcacgaatacccacc

SEQ ID NO: 327 - DNA, 20 bp, synthetic construct
  source 1..20: mol_type = other DNA; note = Primer sequence for experimental example, DYRK1A_T7E1_FWD
agtacaccttctttcgcagt

SEQ ID NO: 328 - DNA, 20 bp, synthetic construct
  source 1..20: mol_type = other DNA; note = Primer sequence for experimental example, DYRK1A_T7E1_REV
gagacgttatgcattcacca

SEQ ID NO: 329 - DNA, 20 bp, synthetic construct
  source 1..20: mol_type = other DNA; note = Primer sequence for experimental example, EMX1_T7E1_FWD
tgggtcataggctctctcat

SEQ ID NO: 330 - DNA, 20 bp, synthetic construct
  source 1..20: mol_type = other DNA; note = Primer sequence for experimental example, EMX1_T7E1_REV
gagattggagacacggagag

SEQ ID NO: 331 - DNA, 20 bp, synthetic construct
  source 1..20: mol_type = other DNA; note = Primer sequence for experimental example, DNMT3B_T7E1_FWD
aagtttcttcagcggtctct

SEQ ID NO: 332 - DNA, 20 bp, synthetic construct
  source 1..20: mol_type = other DNA; note = Primer sequence for experimental example, DNMT3B_T7E1_REV
ttcgctgactctcttttgga

SEQ ID NO: 333 - DNA, 20 bp, synthetic construct
  source 1..20: mol_type = other DNA; note = Primer sequence for experimental example, MTAP_T7E1_FWD
gaggtttggaggggttaagg

SEQ ID NO: 334 - DNA, 20 bp, synthetic construct
  source 1..20: mol_type = other DNA; note = Primer sequence for experimental example, MTAP_T7E1_REV
taaagacacccccaagactg

SEQ ID NO: 335 - DNA, 20 bp, synthetic construct
  source 1..20: mol_type = other DNA; note = Primer sequence for experimental example, RUNX1_T7E1_FWD
ggcctcataaacaaccacag

SEQ ID NO: 336 - DNA, 20 bp, synthetic construct
  source 1..20: mol_type = other DNA; note = Primer sequence for experimental example, RUNX1_T7E1_REV
aagaccagcatgtactcacc

SEQ ID NO: 337 - DNA, 60 bp, synthetic construct
  source 1..60: mol_type = other DNA; note = Primer sequence for experimental example, FANCF_Adapt_FWD
acactctttccctacacgacgctcttccgatctcacttttataacgaagaactctttgtg

SEQ ID NO: 338 - DNA, 57 bp, synthetic construct
  source 1..57: mol_type = other DNA; note = Primer sequence for experimental example, FANCF_Adapt_REV
gtgactggagttcagacgtgtgctcttccgatctcatcattagaagcttggatgctc

SEQ ID NO: 339 - DNA, 53 bp, synthetic construct
  source 1..53: mol_type = other DNA; note = Primer sequence for experimental example, PD1_Adap_FWD
acactctttccctacacgacgctcttccgatctagagaggaacccaggagttc

SEQ ID NO: 340 - DNA, 54 bp, synthetic construct
  source 1..54: mol_type = other DNA; note = Primer sequence for experimental example, PD1_Adap_REV
gtgactggagttcagacgtgtgctcttccgatctgttcttaggtaggtggggtc

SEQ ID NO: 341 - DNA, 51 bp, synthetic construct
  source 1..51: mol_type = other DNA; note = Primer sequence for experimental example, VEGFA_Adap_FWD
acactctttccctacacgacgctcttccgatctgccaggcattgaagaagg

SEQ ID NO: 342 - DNA, 54 bp, synthetic construct
  source 1..54: mol_type = other DNA; note = Primer sequence for experimental example, VEGFA_Adap_REV
gtgactggagttcagacgtgtgctcttccgatctgaaccatgccatgtccctag

SEQ ID NO: 343 - DNA, 60 bp, synthetic construct
  source 1..60: mol_type = other DNA; note = Primer sequence for experimental example, Site4_Adap_FWD
acactctttccctacacgacgctcttccgatctgtggagtttgtattttgcatactcaag

SEQ ID NO: 344 - DNA, 61 bp, synthetic construct
  source 1..61: mol_type = other DNA; note = Primer sequence for experimental example, Site4_Adap_REV
gtgactggagttcagacgtgtgctcttccgatctcaagagttctcagccctatattgatag

SEQ ID NO: 345 - DNA, 52 bp, synthetic construct
  source 1..52: mol_type = other DNA; note = Primer sequence for experimental example, CFTR_Adap_FWD
acactctttccctacacgacgctcttccgatctcagccctttcccaggtaag

SEQ ID NO: 346 - DNA, 53 bp, synthetic construct
  source 1..53: mol_type = other DNA; note = Primer sequence for experimental example, CFTR_Adap_REV
gtgactggagttcagacgtgtgctcttccgatctctctcctctccccatgatg

SEQ ID NO: 347 - DNA, 51 bp, synthetic construct
  source 1..51: mol_type = other DNA; note = Primer sequence for experimental example, Site6_Adap_FWD
acactctttccctacacgacgctcttccgatctctgaggggctctgcattg

SEQ ID NO: 348 - DNA, 54 bp, synthetic construct
  source 1..54: mol_type = other DNA; note = Primer sequence for experimental example, Site6_Adap_REV
gtgactggagttcagacgtgtgctcttccgatctagggaggaaaccctgacttc

SEQ ID NO: 349 - DNA, 51 bp, synthetic construct
  source 1..51: mol_type = other DNA; note = Primer sequence for experimental example, IFTAP_Adap_FWD
acactctttccctacacgacgctcttccgatctggcaggcagactcgatac

SEQ ID NO: 350 - DNA, 58 bp, synthetic construct
  source 1..58: mol_type = other DNA; note = Primer sequence for experimental example, IFTAP_Adap_REV
gtgactggagttcagacgtgtgctcttccgatctctctaacctatttgccaaggtagg

SEQ ID NO: 351 - DNA, 51 bp, synthetic construct
  source 1..51: mol_type = other DNA; note = Primer sequence for experimental example, Site6-T_Adap_FWD
acactctttccctacacgacgctcttccgatcttacccaggcatggtggtg

SEQ ID NO: 352 - DNA, 55 bp, synthetic construct
  source 1..55: mol_type = other DNA; note = Primer sequence for experimental example, Site6-T_Adap_REV
gtgactggagttcagacgtgtgctcttccgatctgttgagaggatgggatggaac

SEQ ID NO: 353 - DNA, 51 bp, synthetic construct
  source 1..51: mol_type = other DNA; note = Primer sequence for experimental example, VEGF-T_Adap_FWD
acactctttccctacacgacgctcttccgatctcttggctagggggcaatg

SEQ ID NO: 354 - DNA, 57 bp, synthetic construct
  source 1..57: mol_type = other DNA; note = Primer sequence for experimental example, VEGF-T_Adap_REV
gtgactggagttcagacgtgtgctcttccgatctcttgggcacaatgcttatatgtg

SEQ ID NO: 355 - DNA, 53 bp, synthetic construct
  source 1..53: mol_type = other DNA; note = Primer sequence for experimental example, DNMT1_ADAPT_FWD
acactctttccctacacgacgctcttccgatctccacttattgggtcagctgt

SEQ ID NO: 356 - DNA, 54 bp, synthetic construct
  source 1..54: mol_type = other DNA; note = Primer sequence for experimental example, DNMT1_ADAPT_REV
gtgactggagttcagacgtgtgctcttccgatctatcagtgcatgttggggatt

SEQ ID NO: 357 - DNA, 55 bp, synthetic construct
  source 1..55: mol_type = other DNA; note = Primer sequence for experimental example, DYRK1A_ADAPT_FWD
acactctttccctacacgacgctcttccgatcttcttaaaaccttgtcacacaca

SEQ ID NO: 358 - DNA, 54 bp, synthetic construct
  source 1..54: mol_type = other DNA; note = Primer sequence for experimental example, DYRK1A_ADAPT_FWD
gtgactggagttcagacgtgtgctcttccgatctgctcccctaagcaaatactc

SEQ ID NO: 359 - DNA, 53 bp, synthetic construct
  source 1..53: mol_type = other DNA; note = Primer sequence for experimental example, EMX1_ADAPT_FWD
acactctttccctacacgacgctcttccgatctgtagcctcagtcttcccatc

SEQ ID NO: 360 - DNA, 54 bp, synthetic construct
  source 1..54: mol_type = other DNA; note = Primer sequence for experimental example, EMX1_ADAPT_REV
gtgactggagttcagacgtgtgctcttccgatctttcttcttctgctcggactc

SEQ ID NO: 361 - DNA, 53 bp, synthetic construct
  source 1..53: mol_type = other DNA; note = Primer sequence for experimental example, DNMT3B_Adapt_FWD
acactctttccctacacgacgctcttccgatctggtaacagagcgacaacagt

SEQ ID NO: 362 - DNA, 54 bp, synthetic construct
  source 1..54: mol_type = other DNA; note = Primer sequence for experimental example, DNMT3B_Adapt_REV
gtgactggagttcagacgtgtgctcttccgatcttgtttggtggtctcttcaca

SEQ ID NO: 363 - DNA, 53 bp, synthetic construct
  source 1..53: mol_type = other DNA; note = Primer sequence for experimental example, MTAP_ADAPT_FWD
acactctttccctacacgacgctcttccgatctccatctgtacccccaaaaac

SEQ ID NO: 364 - DNA, 52 bp, synthetic construct
  source 1..52: mol_type = other DNA; note = Primer sequence for experimental example, MTAP_ADAPT_REV
gtgactggagttcagacgtgtgctcttccgatctgcatgtgcaggggaactc

SEQ ID NO: 365 - DNA, 51 bp, synthetic construct
  source 1..51: mol_type = other DNA; note = Primer sequence for experimental example, RUNX1_ADAPT_FWD
acactctttccctacacgacgctcttccgatctagcatcaccaacccacag

SEQ ID NO: 366 - DNA, 54 bp, synthetic construct
  source 1..54: mol_type = other DNA; note = Primer sequence for experimental example, RUNX1_ADAPT_REV
gtgactggagttcagacgtgtgctcttccgatctcgggggactcaatgatttct

SEQ ID NO: 367 - DNA, 57 bp, synthetic construct
  source 1..57: mol_type = other DNA; note = Primer sequence for experimental example, D501
aatgatacggcgaccaccgagatctacactatagcctacactctttccctacacgac

SEQ ID NO: 368 - DNA, 57 bp, synthetic construct
  source 1..57: mol_type = other DNA; note = Primer sequence for experimental example, D502
aatgatacggcgaccaccgagatctacacatagaggcacactctttccctacacgac

SEQ ID NO: 369 - DNA, 57 bp, synthetic construct
  source 1..57: mol_type = other DNA; note = Primer sequence for experimental example, D503
aatgatacggcgaccaccgagatctacaccctatcctacactctttccctacacgac

SEQ ID NO: 370 - DNA, 57 bp, synthetic construct
  source 1..57: mol_type = other DNA; note = Primer sequence for experimental example, D504
aatgatacggcgaccaccgagatctacacggctctgaacactctttccctacacgac

SEQ ID NO: 371 - DNA, 57 bp, synthetic construct
  source 1..57: mol_type = other DNA; note = Primer sequence for experimental example, D505
aatgatacggcgaccaccgagatctacacaggcgaagacactctttccctacacgac

SEQ ID NO: 372 - DNA, 57 bp, synthetic construct
  source 1..57: mol_type = other DNA; note = Primer sequence for experimental example, D506
aatgatacggcgaccaccgagatctacactaatcttaacactctttccctacacgac

SEQ ID NO: 373 - DNA, 57 bp, synthetic construct
  source 1..57: mol_type = other DNA; note = Primer sequence for experimental example, D507
aatgatacggcgaccaccgagatctacaccaggacgtacactctttccctacacgac

SEQ ID NO: 374 - DNA, 57 bp, synthetic construct
  source 1..57: mol_type = other DNA; note = Primer sequence for experimental example, D508
aatgatacggcgaccaccgagatctacacgtactgacacactctttccctacacgac

SEQ ID NO: 375 - DNA, 53 bp, synthetic construct
  source 1..53: mol_type = other DNA; note = Primer sequence for experimental example, D701
caagcagaagacggcatacgagatcgagtaatgtgactggagttcagacgtgt

SEQ ID NO: 376 - DNA, 53 bp, synthetic construct
  source 1..53: mol_type = other DNA; note = Primer sequence for experimental example, D702
caagcagaagacggcatacgagattctccggagtgactggagttcagacgtgt

SEQ ID NO: 377 - DNA, 53 bp, synthetic construct
  source 1..53: mol_type = other DNA; note = Primer sequence for experimental example, D703
caagcagaagacggcatacgagataatgagcggtgactggagttcagacgtgt

SEQ ID NO: 378 - DNA, 53 bp, synthetic construct
  source 1..53: mol_type = other DNA; note = Primer sequence for experimental example, D704
caagcagaagacggcatacgagatggaatctcgtgactggagttcagacgtgt

SEQ ID NO: 379 - DNA, 53 bp, synthetic construct
  source 1..53: mol_type = other DNA; note = Primer sequence for experimental example, D705
caagcagaagacggcatacgagatttctgaatgtgactggagttcagacgtgt

SEQ ID NO: 380 - DNA, 53 bp, synthetic construct
  source 1..53: mol_type = other DNA; note = Primer sequence for experimental example, D706
caagcagaagacggcatacgagatacgaattcgtgactggagttcagacgtgt

SEQ ID NO: 381 - DNA, 53 bp, synthetic construct
  source 1..53: mol_type = other DNA; note = Primer sequence for experimental example, D707
caagcagaagacggcatacgagatagcttcaggtgactggagttcagacgtgt

SEQ ID NO: 382 - DNA, 53 bp, synthetic construct
  source 1..53: mol_type = other DNA; note = Primer sequence for experimental example, D708
caagcagaagacggcatacgagatgcgcattagtgactggagttcagacgtgt

SEQ ID NO: 383 - DNA, 53 bp, synthetic construct
  source 1..53: mol_type = other DNA; note = Primer sequence for experimental example, D709
caagcagaagacggcatacgagatcatagccggtgactggagttcagacgtgt

SEQ ID NO: 384 - DNA, 53 bp, synthetic construct
  source 1..53: mol_type = other DNA; note = Primer sequence for experimental example, D710
caagcagaagacggcatacgagatttcgcggagtgactggagttcagacgtgt

SEQ ID NO: 385 - DNA, 53 bp, synthetic construct
  source 1..53: mol_type = other DNA; note = Primer sequence for experimental example, D711
caagcagaagacggcatacgagatgcgcgagagtgactggagttcagacgtgt

SEQ ID NO: 386 - DNA, 53 bp, synthetic construct
  source 1..53: mol_type = other DNA; note = Primer sequence for experimental example, D712
caagcagaagacggcatacgagatctatcgctgtgactggagttcagacgtgt

SEQ ID NO: 387 - DNA, 44 bp, synthetic construct
  source 1..44: mol_type = other DNA; note = Primer sequence for experimental example, ID_218_DNMT1_REV
cctttattttagctgaagggaaaatctacacttagtagaaatta

SEQ ID NO: 388 - DNA, 44 bp, synthetic construct
  source 1..44: mol_type = other DNA; note = Primer sequence for experimental example, ID_218_DNMT3B_REV
gaggtatccagcagaggggagaaatctacacttagtagaaatta

SEQ ID NO: 389 - DNA, 44 bp, synthetic construct; identical to SEQ ID NO: 180
  source 1..44: mol_type = other DNA; note = Primer sequence for experimental example, ID_218_DYRK1A_REV

SEQ ID NO: 390 - DNA, 44 bp, synthetic construct; identical to SEQ ID NO: 156
  source 1..44: mol_type = other DNA; note = Primer sequence for experimental example, ID_218_EMX1_REV

SEQ ID NO: 391 - DNA, 45 bp, synthetic construct; identical to SEQ ID NO: 157
  source 1..45: mol_type = other DNA; note = Primer sequence for experimental example, ID_230_FWD

SEQ ID NO: 392 - DNA, 44 bp, synthetic construct; identical to SEQ ID NO: 182
  source 1..44: mol_type = other DNA; note = Primer sequence for experimental example, ID_230_MTAP_REV

SEQ ID NO: 393 - DNA, 44 bp, synthetic construct
  source 1..44: mol_type = other DNA; note = Primer sequence for experimental example, ID_230_DNMT1_REV
cctttattttagctgaagggaaaatctacaatagtagaaattta

SEQ ID NO: 394 - DNA, 44 bp, synthetic construct
  source 1..44: mol_type = other DNA; note = Primer sequence for experimental example, ID_230_DNMT3B_REV
gaggtatccagcagaggggagaaatctacaatagtagaaattta

SEQ ID NO: 395 - DNA, 44 bp, synthetic construct; identical to SEQ ID NO: 183
  source 1..44: mol_type = other DNA; note = Primer sequence for experimental example, ID_230_DYRK1A_REV

SEQ ID NO: 396 - DNA, 44 bp, synthetic construct; identical to SEQ ID NO: 158
  source 1..44: mol_type = other DNA; note = Primer sequence for experimental example, ID_230_EMX1_REV

SEQ ID NO: 397 - DNA, 45 bp, synthetic construct; identical to SEQ ID NO: 184
  source 1..45: mol_type = other DNA; note = Primer sequence for experimental example, ID_233_FWD

SEQ ID NO: 398 - DNA, 44 bp, synthetic construct; identical to SEQ ID NO: 185
  source 1..44: mol_type = other DNA; note = Primer sequence for experimental example, ID_233_MTAP_REV

SEQ ID NO: 399 - DNA, 44 bp, synthetic construct
  source 1..44: mol_type = other DNA; note = Primer sequence for experimental example, ID_233_DNMT1_REV
cctttattttagctgaagggaaaatctacaagagtagaaatttt

SEQ ID NO: 400 - DNA, 44 bp, synthetic construct
  source 1..44: mol_type = other DNA; note = Primer sequence for experimental example, ID_233_DNMT3B_REV
gaggtatccagcagaggggagaaatctacaagagtagaaatttt

SEQ ID NO: 401 - DNA, 44 bp, synthetic construct; identical to SEQ ID NO: 186
  source 1..44: mol_type = other DNA; note = Primer sequence for experimental example, ID_233_DYRK1A_REV

SEQ ID NO: 402 - DNA, 44 bp, synthetic construct
  source 1..44: mol_type = other DNA; note = Primer sequence for experimental example, ID_233_EMX1_REV
cagggagggaggggcacagatgaatctacaagagtagaaatttt

SEQ ID NO: 403 - DNA, 45 bp, synthetic construct; identical to SEQ ID NO: 187
  source 1..45: mol_type = other DNA; note = Primer sequence for experimental example, ID_236_FWD

SEQ ID NO: 404 - DNA, 44 bp, synthetic construct; identical to SEQ ID NO: 188
  source 1..44: mol_type = other DNA; note = Primer sequence for experimental example, ID_236_MTAP_REV

SEQ ID NO: 405 - DNA, 44 bp, synthetic construct
  source 1..44: mol_type = other DNA; note = Primer sequence for experimental example, ID_236_DNMT1_REV
cctttattttagctgaagggaaaatctacaacagtagaaattta

SEQ ID NO: 406 - DNA, 44 bp, synthetic construct
  source 1..44: mol_type = other DNA; note = Primer sequence for experimental example, ID_236_DNMT3B_REV
gaggtatccagcagaggggagaaatctacaacagtagaaattta

SEQ ID NO: 407 - DNA, 44 bp, synthetic construct; identical to SEQ ID NO: 189
  source 1..44: mol_type = other DNA; note = Primer sequence for experimental example, ID_236_DYRK1A_REV

SEQ ID NO: 408 - DNA, 44 bp, synthetic construct
  source 1..44: mol_type = other DNA; note = Primer sequence for experimental example, ID_236_EMX1_REV
cagggagggaggggcacagatgaatctacaacagtagaaattta

SEQ ID NO: 409 - DNA, 45 bp, synthetic construct; identical to SEQ ID NO: 190
  source 1..45: mol_type = other DNA; note = Primer sequence for experimental example, ID_239_FWD

SEQ ID NO: 410 - DNA, 44 bp, synthetic construct; identical to SEQ ID NO: 191
  source 1..44: mol_type = other DNA; note = Primer sequence for experimental example, ID_239_MTAP_REV

SEQ ID NO: 411 - DNA, 44 bp, synthetic construct
  source 1..44: mol_type = other DNA; note = Primer sequence for experimental example, ID_239_DNMT1_REV
cctttattttagctgaagggaaaatctacaatagtagaaattat

SEQ ID NO: 412 - DNA, 44 bp, synthetic construct
  source 1..44: mol_type = other DNA; note = Primer sequence for experimental example, ID_239_DNMT3B_REV
gaggtatccagcagaggggagaaatctacaatagtagaaattat

SEQ ID NO: 413 - DNA, 44 bp, synthetic construct; identical to SEQ ID NO: 192
  source 1..44: mol_type = other DNA; note = Primer sequence for experimental example, ID_239_DYRK1A_REV

SEQ ID NO: 414 - DNA, 44 bp, synthetic construct
  source 1..44: mol_type = other DNA; note = Primer sequence for experimental example, ID_239_EMX1_REV
cagggagggaggggcacagatgaatctacaatagtagaaattat

SEQ ID NO: 415 - DNA, 23 bp, synthetic construct
  source 1..23: mol_type = other DNA; note = Protospacer sequence of VEGFA
ctctcaagacccacaatccaggc

SEQ ID NO: 416 - DNA, 22 bp, synthetic construct
  source 1..22: mol_type = other DNA; note = Protospacer sequence of FANCF
gataagcacagttttaaatagt

SEQ ID NO: 417 - DNA, 23 bp, synthetic construct
  source 1..23: mol_type = other DNA; note = Protospacer sequence of CTFR
ctgactgctcactagatgttgaa

SEQ ID NO: 418 - DNA, 23 bp, synthetic construct
  source 1..23: mol_type = other DNA; note = Protospacer sequence of SITE3
gtttaaacacaccgggttaataa

SEQ ID NO: 419 - RNA, 23 nt, synthetic construct
  source 1..23: mol_type = other RNA; note = Guide domain sequence targeting VEGFA
ctctcaagacccacaatccaggc

SEQ ID NO: 420 - RNA, 22 nt, synthetic construct
  source 1..22: mol_type = other RNA; note = Guide domain sequence targeting FANCF
gataagcacagttttaaatagt

SEQ ID NO: 421 - RNA, 23 nt, synthetic construct
  source 1..23: mol_type = other RNA; note = Guide domain sequence targeting CTFR
ctgactgctcactagatgttgaa

SEQ ID NO: 422 - RNA, 23 nt, synthetic construct
  source 1..23: mol_type = other RNA; note = Guide domain sequence targeting SITE3
gtttaaacacaccgggttaataa

SEQ ID NO: 423 - DNA, 241 bp, synthetic construct
  source 1..241: mol_type = other DNA; note = Sequence for U6 promoter
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac